(19) United States
(12) Patent Application Publication
(10) Pub. No.: US 2023/0130802 A1
(43) Pub. Date: Apr. 27, 2023

(54) ORGANIC ELECTROLUMINESCENT COMPOUND, A PLURALITY OF HOST MATERIALS, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Ji-Song JUN, Gyeonggi-do (KR); Su-Hyun Lee, Gyeonggi-do (KR); Tae-Jun Han, Gyeonggi-do (KR); Dong-Hyung Lee, Gyeonggi-do (KR); Sang-Hee Cho, Gyeonggi-do (KR); Hong-Se Oh, Gyeonggi-do (KR)

(21) Appl. No.: 17/877,404

(22) Filed: Jul. 29, 2022

(30) Foreign Application Priority Data
Jul. 30, 2021 (KR) 10-2021-0100462
Jun. 29, 2022 (KR) 10-2022-0079955

(51) Int. Cl. C07D 421/04; C07D 421/14; C07D 421/10; C07D 345/00; C07D 421/12; H01L 51/50; H01L 51/00

(52) U.S. Cl. CPC C07D 421/04; C07D 421/14; C07D 421/10; C07D 345/00; C07D 421/12; H01L 51/5036; H01L 51/0059; H01L 51/006; H01L 51/0067; H01L 51/0069; H01L 51/0071; H01L 51/0073; H01L 51/0074; H01L 51/0054; C07B 2200/05

(57) ABSTRACT

The present disclosure relates to an organic electroluminescent compound, an organic electroluminescent material and an organic electroluminescent device comprising the same, a plurality of host materials comprising at least one first host material and at least one second host material, and an organic electroluminescent device comprising the same. By comprising the compound according to the present disclosure as a single host material or as a plurality of host materials, it is possible to produce an organic electroluminescent device having improved driving voltage, luminous efficiency, and/or lifetime properties compared to the conventional organic electroluminescent devices.

### TECHNICAL FIELD

The present disclosure relates to an organic electroluminescent compound, a plurality of host materials, and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. An OLED having low driving voltage, high luminous efficiency and/or long lifetime characteristics is required for long time use and high resolution of a display.

Korean Patert Application Laying-Open No. 2016-0078526 discloses organic selenium compounds comprising dibenzoselenophene, benzo[b]selenophene or benzo[c]selenophene, and the use thereof in organic light-emitting devices. However, the aforementioned reference does not specifically disclose an organic selenium compound and a plurality of materials comprising the same claimed in the present disclosure. In addition, there has been a need to develop a light-emitting material having more improved performances, for example, improved driving voltage, luminous efficiency, and/or lifetime properties.

### DISCLOSURE OF INVENTION

### Technical Problem

The objective of the present disclosure is to provide an organic electroluminescent compound having a new structure suitable for applying it to an organic electroluminescent device. Another objective of the present disclosure is to provide an organic electroluminescent material capable of providing an organic electroluminescent device having improved driving voltage, luminous efficiency and/or lifetime properties. Still another objective of the present disclosure is to provide an organic electroluminescent device having improved driving voltage, luminous efficiency and/or lifetime properties by comprising the compound according to the present disclosure as a single host material or the plurality of compounds according to the present disclosure as host materials.

### Solution to Problem

As a result of intensive studies to solve the technical problems, the present inventors found that the above objective can be achieved by an organic electroluminescent compound represented by the following formula 1,

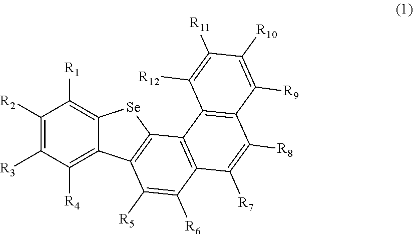

In formula 1,

R₁ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *—L₁—Ar₁;

with the proviso that at least one of R₁ to R₁₂ represents *—L₁—Ar₁;

L₁, each independently, represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁, each independently, represents a halogen, a cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, or a substituted or unsubstituted (C1-C30)alkoxy, or is represented by the following formula a:

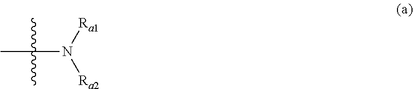

in formula a,

Rₐ₁ and Rₐ₂, each independently, represent a substituted or unsubstituted (C6-C30)aryl, a substituted or unsubstituted (3- to 30-membered)heteroaryl, or a substituted or unsubstituted (C3-C30)cycloalkyl; and [symbol] represents a bonding position with L₁.

In addition, the present inventors found that the above objective can be achieved by a plurality of host materials comprising at least one first host compound represented by the following formula 1′ and at least one second host compound represented by the following formula 1′,

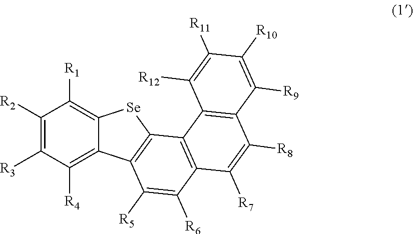

In formula 1′,

R₁ to R₁₂, L₁, Rₐ₁, and Rₐ₂ are as defined in formula 1;

Ar₁ in the first host compound is represented by

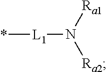

and

Ar₁ in the second host compound represents a substituted or unsubstituted (3- to 30-membered)heteroaryl.

Further, the present inventors found that the above objective can be achieved by a plurality of host materials comprising at least one first host material comprising the compound represented by formula 1 and at least one second host material different from the first host material.

The second host material may be represented by the following formula 2 or 3.

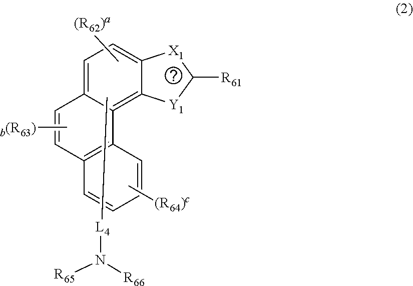

In formula 2,

X₁ and Y₁, each independently, represent —N═, —NR₆₇—, —O— or —S—, with the proviso that any one of X₁, and Y₁ represents —N═, and the other one represents —NR₅₇—, —O— or —S—;

R₆₁ represents a substituted or unsubstituted (C6-C30)aryl, or a substituted or unsubstituted (3- to 30-membered)heteroaryl;

R₆₂ to R₆₄ and R₆₇, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₃″—N(Ar₃″)(Ar₄″); or may be linked to an adjacent substituent(s) to form a ring(s);

L₃″, each independently, represents a single bond, a substituted or unsubstituted (C6-C30)arylene, or a substituted or unsubstituted (3- to 30-membered)heteroarylene;

Ar₃″ and Ar₄″, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl;

R₆₅ and R₆₆, each independently, represent a substituted or unsubstituted (C6-C30)aryl, or a substituted or unsubstituted (3- to 30-membered)heteroaryl;

L₄ represents a single bond, a substituted or unsubstituted (C6-C30)arylene, or a substituted or unsubstituted (3- to 30-membered)heteroarylene; and

a and b, each independently, represent an integer of 1 or 2, and c represents an integer of 1 to 4, where if a to c are each an integer of 2 or more, each of R₆₂, to each of R₆₄ may be the same or different from each other.

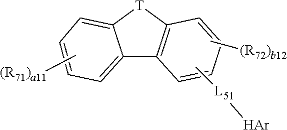

In formula 3,

T represents —O— or —S—;

L₅₁ represents a single bond, a substituted or unsubstituted (C6-C30)arylene, or a substituted or unsubstituted (3- to 30-membered)heteroarylene;

HAr represents a substituted or unsubstituted (3- to 30-membered)heteroaryl containing a nitrogen(s);

R₇₁ and R₇₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino, or may be linked to an adjacent substituent(s) to form a ring(s); and

a₁₁ represents an integer of 1 to 4, and b₁₂ represents an integer of 1 to 3, where if a₁₁ and b₁₂ are each an integer of 2 or more, each of R₇₁ and each of R₇₂ may be the same or different from each other.

### Advantageous Effects of Invention

The organic electroluminescent compound according to the present disclosure exhibits performances suitable for using it in an organic electroluminescent device. In addition, an organic electroluminescent device having improved driving voltage, luminous efficiency and/or lifetime properties compared to conventional organic electroluminescent devices is provided by comprising the compound according to the present disclosure as a single host material, or a plurality of compounds as host materials, and it is possible to produce a display system or a lighting system using the same.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any organic layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials of the present disclosure may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The at least two compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device, and at least two compounds comprised in the plurality of host materials may be comprised together in one light-emitting layer, or each may be comprised in different light-emitting layers. When the at least two host materials are comprised in one layer, for example, they may be mixture-evaporated to form a layer, or separately co-evaporated at the same time to form a layer.

Herein, the term “(C1-C30)alkyl(ene)” is meant to be a linear or branched alkylene) having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butyryl, 2-butynyl, 3-butynyl, 1-methylpent-2-ynyl, etc. The term “(C3-C30)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, preferably the group consisting of 0, 5, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, and may be partially saturated. The number of ring backbone carbon atoms is preferably 6 to 28, and more preferably 6 to 25. The above aryl may comprise a spirt structure. The above aryl may include phenyl, biphenyl, terphenyl, naphthyl, binaphthyl, phenylnaphthyl, naphthylphenyl, phenylterphenyl, fluorenyl, phenylfluorenyl, diphenylfluorenyl, dimethylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-y, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11, 11-dimethyl-3-benzo[a]fluorenyl, 11, 11-dimethyl-4-benzo[a]fluorenyl, 11,11-dimethyl-5-benzo[a]fluorenyl, 11,11-dimethyl-6-benzo[a]fluorenyl, 11, 11-dimethyl-7-benzo[a]fluorenyl, 11, 11-dimethyl- 8-benzo[a]fluorenyl, 11, 11-dimethyl-9-benzo[a]fluorenyl, 11,11-dimethyl-10-benzo[a]fluorenyl, 11,11-dimethyl-1-benzo[b]fluorenyl, 11, 11-dimethyl-2-benzo[b]fluorenyl 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11 ,11-dimethyl-8-benzo[b]fluorenyl, 11,11-dimethyl-9-benzo[b]fluorenyl, 11, 11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11, 11-dimethyl-4-benzo[c]fluorenyl, 11,11-dimethyl-5-benzo[c]fluorenyl, 11, 11-dimethyl-6-benzo[c]fluorenyl, 11,11-dimethyl-7-benzo[c]fluorenyl, 11, 11-dimethyl-8-benzo[c]fluorenyl, 11,11-dimethyl-9-benzo[c]fluorenyl, 11, 11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl benzo[c]fluorenyl, 11,11-diphenyl-6-benzo[c]fluorenyl, 11, 11-diphenyl-7-benzo[c]fluorenyl, 11,11-diphenyl-8-benzo[c]fluorenyl, 11,11-diphenyl-9-benzo[c]fluorenyl, 11,11-diphenyl benzo[c]fluorenyl, 9,9,10, 10-tetramethyl-9,10-dihydro-1-phenanthrenyl, 9,9, 10, 10-tetramethyl-9, 10-dihydro-2-phenanthrenyl, 9,9,10, 10-tetramethyl-9,10-dihydro phenanthrenyl, 9,9,10,10-tetramethyl-9,10-dihydro-4-phenanthrenyl, etc.

The term “(3- to 30-membered)heteroaryl(ene)” is meant to be an aryl(ene) having 3 to 30 ring backbone atoms, and including at least one heteroatoms selected from the group consisting of B, N, O, S, Si, and P, in which the number of ring backbone atoms is preferably 3 to 25, and more preferably 5 to 20. The number of heteroatoms is preferably 1 to 4. The above heteroaryl(ene) may be a monocyclic ring, or a fused ring condensed with at least one benzene ring; may be partially saturated. In addition, The above heteroaryl(ene) may be one formed by linking at least one heteroaryl or aryl group to a heteroaryl(ene) group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl; benzothiophenyl, isobenzofuranyl, dibenzofuranyl, benzonaphthofuranyl, benzophenanthrofuranyl, dibenzothiophenyl, benzonaphthothiophenyl, benzothiozolyl, benzoisothiozolyl, benzophenanthrothiophenyl, benzoisoxazolyl, benzoxazolyl, phenanthroxazolyl, phenanthrothiazolyl, isoindolyl, indolyl, benzoindolyl, indazolyl, benzimidazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, etc. More specifically; the above heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-Indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9 acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-y, 2-methylpyrrol-5-yl, 3-methylpyrrol-yl, 3-methylpyrrol-2-yl, 3-methylpyrrol-4-yl, 3-methylpyrrol-5-yl, 2-tert-butylpyrrol-4-yl, 34,2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtha-[1,2-b]-benzofuranyl, 5-naphtho-[1,2—N-benzofuranyl, 6-naphtho-[1,2—N-benzofuranyl, 7-naphtho-[1,2—N-benzofuranyl, 8-naphtha-[1,2-b]-benzofuranyl, 9-naphtho-[1,2-b]-benzofuranyl, 10-naphtho-[1,2—N-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtha-[2,3-b]-benzofuranyl, 6-naphtho-[2,3—N-benzofuranyl, naphtho-[2,3-b ]-benzofuranyl, 8-naphtha-[2,3—N-benzofuranyl, 9-naphtho-[2,3—N-benzofuranyl, 10-naphtho-[2,3-b]-benzofuranyl, 1-naphtha-[2.1—N-benzofuranyl, 2-naphtho-[2,1-b]-benzofuranyl, 3-naphtho-[2,1-b]-benzofuranyl, 4-naphtho-[2,1—N-benzofuranyl 5-naphtho-[2,1-b]-benzofuranyl, 6-naphtha-[2,1-b]-benzofuranyl, 7-naphtho-[2,1—N-benzofuranyl, 8-naphtha-[2,1-b]-benzofuranyl, 9-naphtha-[2,1-b]-benzofuranyl, 10-naphtho-[2,1-b]-benzofuranyl, 1-naphtho-[1,2-b]-benzothiophenyl, 2-naphtha-[1,2-b]-benzothiophenyl, 3-naphtho-[1,2—N-benzothiophenyl, 4-naphtho-[1,2—N-benzothiophenyl, 5-naphtho-[1,2—N-benzothiophenyl, 6-naphtha-[1,2—N-benzothiophenyl, 7-naphtho-[1,2-b]-benzothiophenyl, 8-naphtho-[1,2-b]-benzothiophenyl, 9-naphtha-[1,2—N-benzothiophenyl, 10-naphtho-[1,2-13]-benzothiophenyl, 1-naphtha-[2,3-b]-benzothiophenyl, 2-naphtha-[2,3—N-benzothiophenyl, 3-naphtho-[2,3—N-benzothiophenyl, 4-naphtha-[2,3—N-benzothiophenyl, 5-naphtho-[2,3—N-benzothiophenyl, 1-naphtho-[2,1—N-benzothiophenyl, 2-naphtho-[2,1-b]-benzothiophenyl, 3-naphtho-[2,1-b]-benzothiophenyl, 4-naphtho-[2,1—N-benzothiophenyl, 5-naphtho-[2,1—N-benzothiophenyl, 6-naphtho-[2,1-b]-benzothiophenyl, 7-naphtho-[2,1—N-benzothiophenyl, 8-naphtho-[2,1-b]-benzothiophenyl, 9-naphtha-[2,1—N-benzothiophenyl, 10-naphtho-[2,1-b]-benzothiophenyl, 2-benzofuro[3,2-d]pyrimidinyl, 6-benzofuro[3,2-d]pyrimidinyl, benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-dipyrazinyl, 6-benzothio[3,2-d]pyrazinyl, 7-benzothio[3,2-d]pyrazinyl, 8-benzothio[3,2-d]pyrazinyl, 9-benzothio[3,2-dipyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, C1, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

In the present disclosure, a ring formed by a linkage of adjacent substituents means that at least two adjacent substituents are linked or fused to each other to form a substituted or unsubstituted, mono- or polycyclic, (3- to 30-membered) alicyclic or aromatic ring, or the combination thereof, preferably a substituted or unsubstituted, mono- or polycyclic, (3- to 26-membered) alicyclic or aromatic ring, or the combination thereof, and more preferably an unsubstituted, mono- or polycyclic, (5- to 20-membered) aromatic ring. In addition, the formed ring may contain at least one heteroatom selected from B, N, O, 5, Si, and P, preferably at least one heteroatom selected from N, O, and S. For example, the ring may be a substituted or unsubstituted, benzene ring, naphthalene ring, phenanthrene ring, fluorene ring, indene ring, indole ring, benzoindole ring, benzofuran ring, benzothiophene ring, dibenzothiophene ring, dibenzofuran ring, carbazole ring, etc.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine, That is, pyridine-triazine may be interpreted as a heteroaryl substituent, or as substituents in which two heteroaryl substituents are linked. Herein, the substituent(s) of the substituted alkyl, the substituted alkylene, the substituted alkenyl, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl, the substituted aryl, the substituted arylene, the substituted heteroaryl, the substituted heteroarylene, the substituted fused ring group of an aliphatic ring(s) and an aromatic ring(s), the substituted dialkylamino, the substituted diarylamino, and the substituted alkylarylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C6-C30)aryl(s) and a (3- to 30-membered)heteroaryl(s); a (C6-C30)aryl unsubstituted or substituted with at least one of deuterium, a (C1-C30)alkyl(s), a (C6-C30)aryl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di- (C2-C30)alkenylamino; a mono- or di- (C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino unsubstituted or substituted with a (C6-C30)aryl(s); a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a (C1-C10)alkyl; a (C3-C18)cycloalkyl; a (C6-C30)aryl unsubstituted or substituted with at least one of deuterium, a (C1-C10)alkyl(s), and a (C6-C20)aryl(s); a (3- to 30-membered)heteroaryl unsubstituted or substituted with a (C6-C18)aryl(s); a (C6-C30)aryl(C1-C30)alkyl; a di(C6-C18)arylamino unsubstituted or substituted with a (C1-C10)alkyl(s); and a (C6-C18)aryl(6- to 20-membered)heteroarylamino unsubstituted or substituted with a (C6-C20)aryl(s). According to another embodiment of the present disclosure, the substituent(s), each independently, are at least one selected from the group consisting of deuterium; a (C1-C10)alkyl; a (C3-C12)cycloalkyl; a (C6-C28)aryl unsubstituted or substituted with at least one of deuterium, a (C1-C10)alkyl(s), and a (C6-C15)aryl(s); a (3- to 18-membered)heteroaryl unsubstituted or substituted a (C6-C18)aryl(s); a (C6-C18)aryl(C1-C12)alkyl; a di(C6-C18)arylamino unsubstituted or substituted with a (C1-C10)alkyl(s); and a (C6-C12)aryl(6- to 15-membered)heteroarylamino unsubstituted or substituted with a (C6-C12)aryl(s). For example, the substituent(s), each independently, may be at least one selected from the group consisting of deuterium; a methyl; a butyl; a cyclohexyl; a phenyl unsubstituted or substituted with deuterium; a biphenyl; a terphenyl; a naphthyl; a naphthylphenyl; a dimethylphenyl; a butylphenyl; an anthracenyl; a dimethylfluorenyl; a diphenylfluorenyl, a fluoranthenyl; a chrysenyl; a chrysenylnaphthyl; a benzophenanthrenyl; a benzoimidazolyl substituted with a phenyl(s); a dimethylbenzofluorenyl; a dibenzofuranyl; a dibenzothiophenyl; a phenylpyridinyl; phenoxazinyl; a phenylpropyl; a diphenylamino; a (phenylcarbazolyl)phenylamino; (dimethylfluorenyl)phenylamino; and a (biphenyl)phenylamino.

Hereinafter, the compound represented by formula 1 will be described in more detail.

In formula 1, R₁ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *—L₁—Ar₁, with the proviso that at least one of R₁ to R₁₂ represents *—L₁—Ar₁. According to one embodiment of the present disclosure, R₁ to R₁₂, each independently, represent hydrogen, deuterium, a substituted or unsubstituted (C6-C18)aryl, or *—L₁—Ar₁ with the proviso that at least one of R₁ to R₁₂ represents *—L₁—Ar₁. According to another embodiment of the present disclosure, any one of R₁ to r₁₂ represents *—L₁—Ar₁, and the others, each independently, represent hydrogen, deuterium, or an unsubstituted (C6-C18)aryl. For example, any one of R₁ to R₁₂ may be *—L₁—Ar₁, and the others, each independently, may be hydrogen, deuterium, a phenyl, etc.

In formula 1, L₁, each independently, represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene. According to one embodiment of the present disclosure, L₁, each independently, represents a single bond, or a substituted or unsubstituted (C6-C18)arylene. According to another embodiment of the present disclosure, L₁, each independently, represents a single bond, or an unsubstituted (C6-C12)arylene. For example, L₁, each independently, may be a single bond, a phenylene, a naphthylene, etc.

In formula 1, Ar₁, each independently, represents a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, or a substituted or unsubstituted (C1-C30)alkoxy, or is represented by the following formula a.

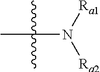

According to one embodiment of the present disclosure, Ar₁, each independently, represents a substituted or unsubstituted (3- to 30-membered)heteroaryl, or is represented by formula a. According to another embodiment of the present disclosure, Ar₁ each independently, represents a (5- to 15-membered)heteroaryl substituted with at least one of a (C6-C18)aryl(s) unsubstituted or substituted with deuterium, a (C1-C₆)alkyl(s), or a (C6-C18)aryl(C1-C12)alkyl(s), and a (5- to 15-membered)heteroaryl(s); or is represented by formula a. For example, Ar₁, each independently, may be a triazinyl substituted with at least one of a phenyl(s) unsubstituted or substituted with deuterium, a naphthyl(s), a biphenyl(s), a dimethylfluorenyl(s), a phenyl(s) substituted with a phenylpropyl(s), and a dibenzofuranyl(s), etc.; or may be represented by formula a.

In formula a, Rₐ₁ and Rₐ₂, each independently, represent a substituted or unsubstituted (C6-C30)aryl, a substituted or unsubstituted (3- to 30-membered)heteroaryl, or a substituted or unsubstituted (C3-C30)cycloalkyl; and [symbol] represents a bonding position with L₁. According to one embodiment of the present disclosure, Rₐ₁ and Rₐ₂, each independently, represent a substituted or unsubstituted (C6-C28)aryl, or a substituted or unsubstituted (5- to 20-membered)heteroaryl. According to another embodiment of the present disclosure, Rₐ₁ and Rₐ₂, each independently, represent a (C6-C25)aryl unsubstituted or substituted with at least one of a (C1-C10)alkyl(s), a (C6-C18)aryl(s), a (C6-C18)aryl(C1-C12)alkyl(s), a di(C6-C20)arylamino(s) unsubstituted or substituted with a (C1-C10)alkyl(s), and a (C6-C18)aryl(6-to 20-membered)heteroarylamino(s) unsubstituted or substituted with a (C6-C20)aryl(s); or an unsubstituted (5- to 13-membered)heteroaryl. For example, R₃₁ and Rₐz, each independently, may be a phenyl unsubstituted or substituted with at least one of a naphthyl(s), a phenylnaphthyl(s), a (phenylcarbazolyl)phenylamino(s), a (dimethylfluorenyl)phenylamino(s), and a (biphenyl)phenylamino(s); a biphenyl; a terphenyl; a naphthyl; a dimethylfluorenyl; a diphenylfluorenyl; a dibenzofuranyl; a dibenzothiophenyl, etc.

The compound represented by formula 1 may be at least one selected from the group consisting of the following compounds, but is not limited thereto.

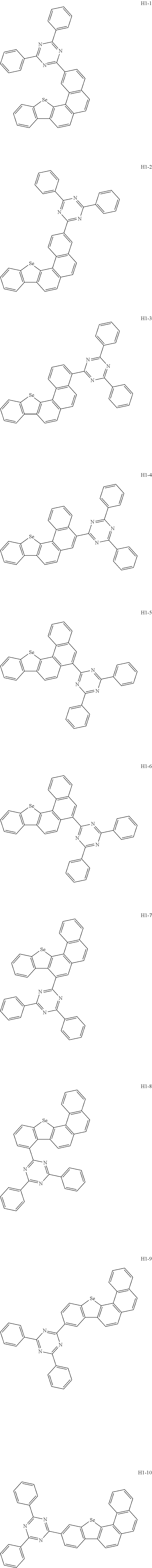

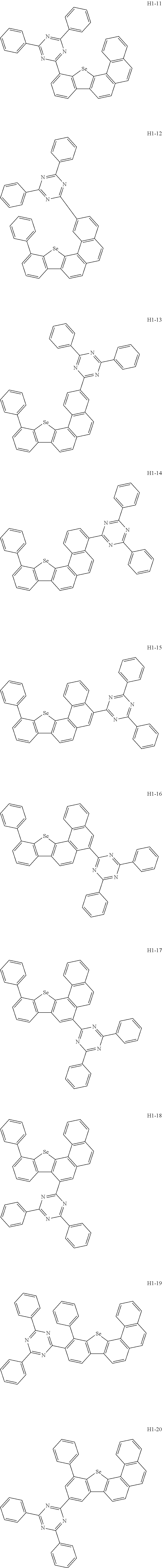

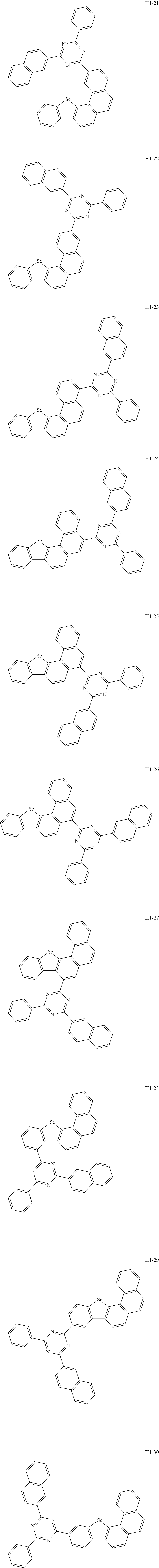

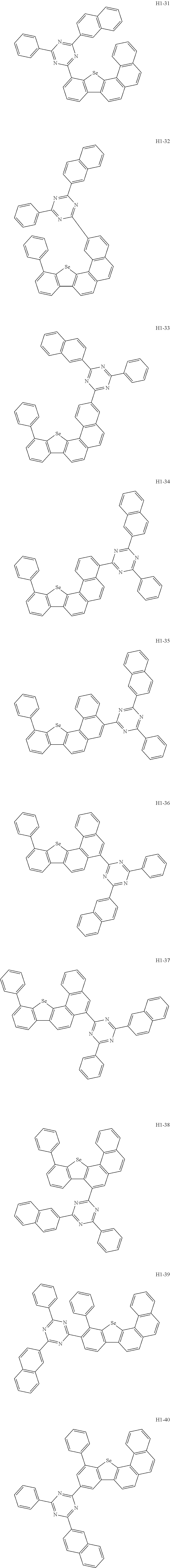

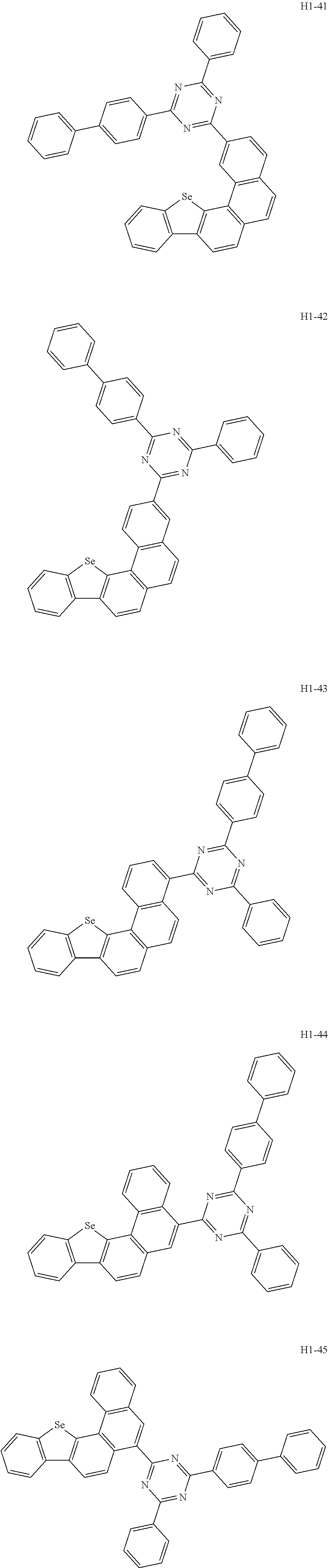 [SMILES: c1ccc(-c2ccc(-c3nc(-c4ccccc4)nc(-c4ccc5ccc6ccc7c8ccccc8[se]c7c6c5c4)n3)cc2)cc1 ; c1ccc(-c2ccc(-c3nc(-c4ccccc4)nc(-c4ccc5c(ccc6ccc7c8ccccc8[se]c7c65)c4)n3)cc2)cc1 ; c1ccc(-c2ccc(-c3nc(-c4ccccc4)nc(-c4cccc5c4ccc4ccc6c7ccccc7[se]c6c45)n3)cc2)cc1 ; c1ccc(-c2ccc(-c3nc(-c4ccccc4)nc(-c4cc5ccc6c7ccccc7[se]c6c5c5ccccc45)n3)cc2)cc1 ; c1ccc(-c2ccc(-c3nc(-c4ccccc4)nc(-c4cc5ccccc5c5c4ccc4c6ccccc6[se]c45)n3)cc2)cc1]

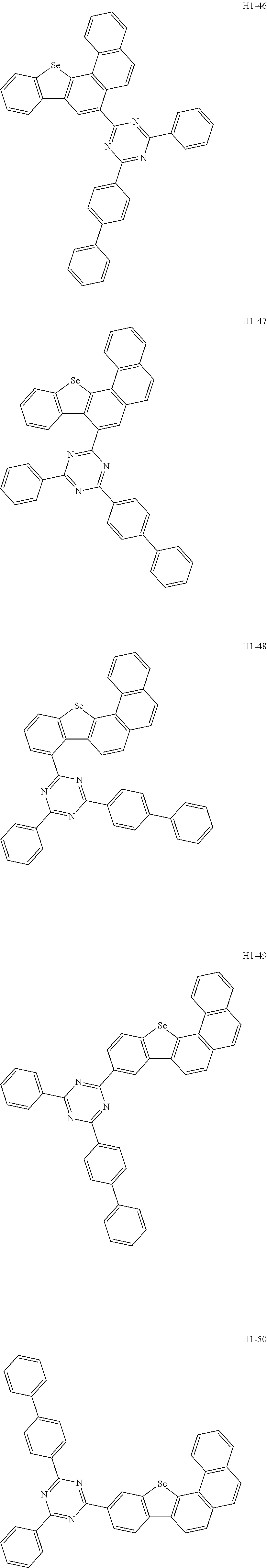 [SMILES: c1ccc(-c2ccc(-c3nc(-c4ccccc4)nc(-c4cc5c6ccccc6[se]c5c5c4ccc4ccccc45)n3)cc2)cc1 ; c1ccc(-c2ccc(-c3nc(-c4ccccc4)nc(-c4cc5ccc6ccccc6c5c5[se]c6ccccc6c45)n3)cc2)cc1 ; c1ccc(-c2ccc(-c3nc(-c4ccccc4)nc(-c4cccc5[se]c6c(ccc7ccc8ccccc8c76)c45)n3)cc2)cc1 ; c1ccc(-c2ccc(-c3nc(-c4ccccc4)nc(-c4ccc5[se]c6c(ccc7ccc8ccccc8c76)c5c4)n3)cc2)cc1 ; c1ccc(-c2ccc(-c3nc(-c4ccccc4)nc(-c4ccc5c(c4)[se]c4c5ccc5ccc6ccccc6c54)n3)cc2)cc1]

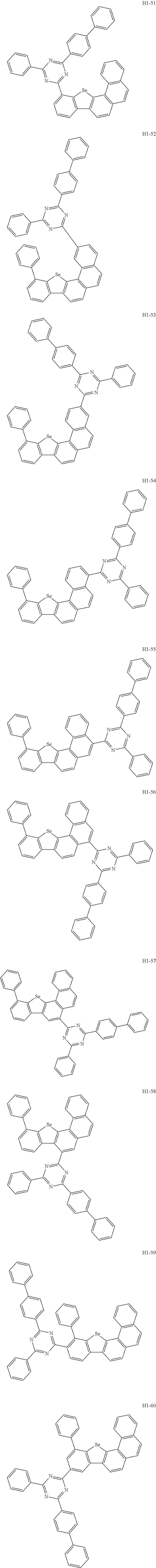

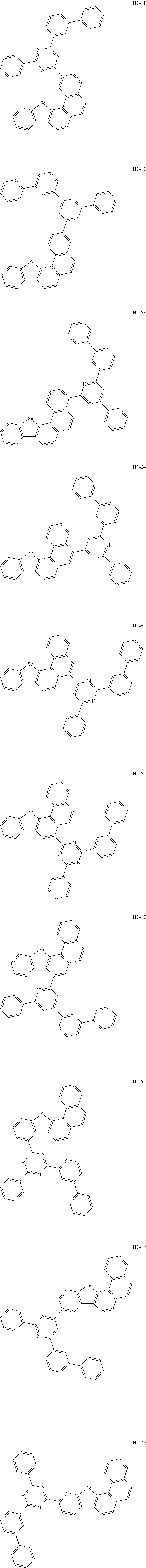

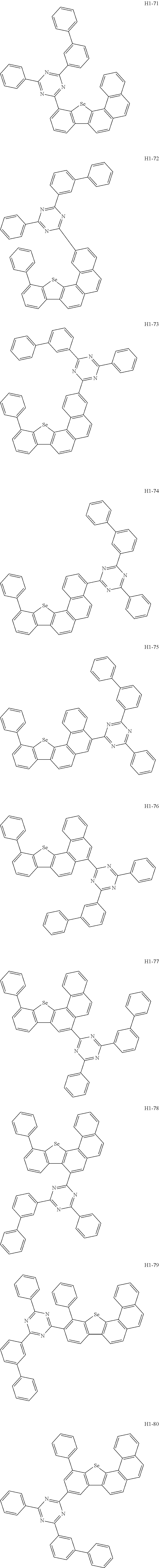

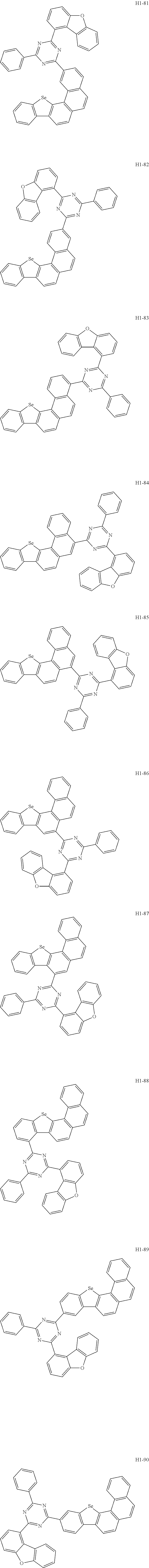

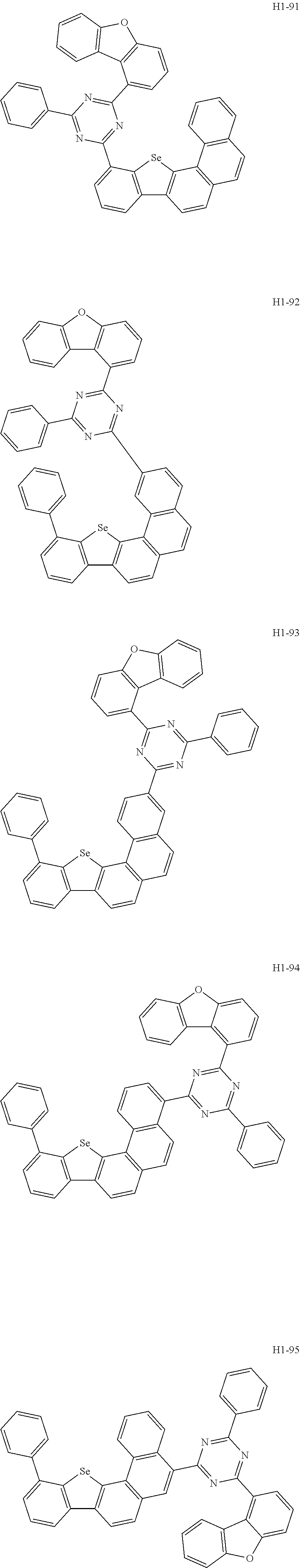 [SMILES: c1ccc(-c2nc(-c3cccc4c3[se]c3c4ccc4ccc5ccccc5c43)nc(-c3cccc4oc5ccccc5c34)n2)cc1 ; c1ccc(-c2nc(-c3ccc4ccc5ccc6c7cccc(-c8ccccc8)c7[se]c6c5c4c3)nc(-c3cccc4oc5ccccc5c34)n2)cc1 ; c1ccc(-c2nc(-c3ccc4c(ccc5ccc6c7cccc(-c8ccccc8)c7[se]c6c54)c3)nc(-c3cccc4oc5ccccc5c34)n2)cc1 ; c1ccc(-c2nc(-c3cccc4c3ccc3ccc5c6cccc(-c7ccccc7)c6[se]c5c34)nc(-c3cccc4oc5ccccc5c34)n2)cc1 ; c1ccc(-c2nc(-c3cc4ccc5c6cccc(-c7ccccc7)c6[se]c5c4c4ccccc34)nc(-c3cccc4oc5ccccc5c34)n2)cc1]

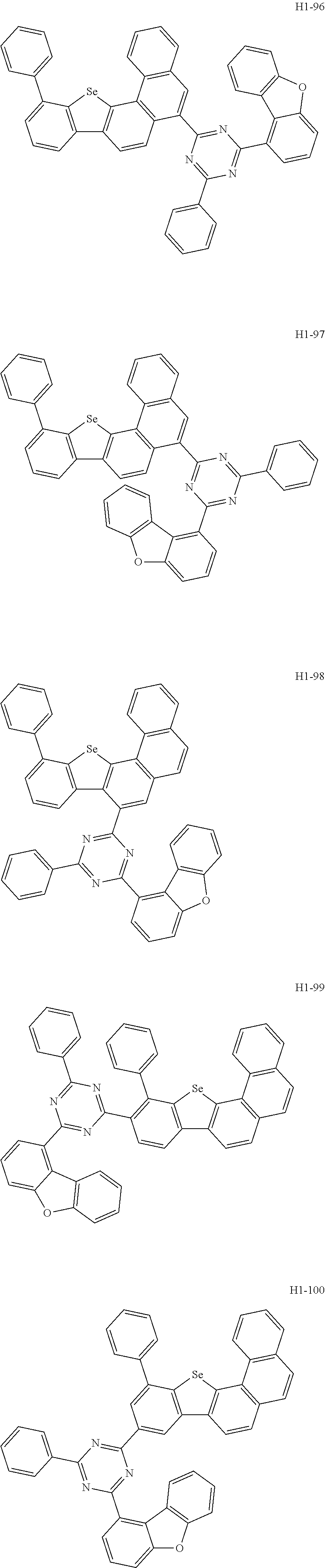 [SMILES: c1ccc(-c2nc(-c3cc4ccccc4c4c3ccc3c5cccc(-c6ccccc6)c5[se]c34)nc(-c3cccc4oc5ccccc5c34)n2)cc1 ; c1ccc(-c2nc(-c3cc4ccccc4c4c3ccc3c5cccc(-c6ccccc6)c5[se]c34)nc(-c3cccc4oc5ccccc5c34)n2)cc1 ; c1ccc(-c2nc(-c3cccc4oc5ccccc5c34)nc(-c3cc4ccc5ccccc5c4c4[se]c5c(-c6ccccc6)cccc5c34)n2)cc1 ; c1ccc(-c2nc(-c3ccc4c([se]c5c4ccc4ccc6ccccc6c45)c3-c3ccccc3)nc(-c3cccc4oc5ccccc5c34)n2)cc1 ; c1ccc(-c2nc(-c3cc(-c4ccccc4)c4[se]c5c(ccc6ccc7ccccc7c65)c4c3)nc(-c3cccc4oc5ccccc5c34)n2)cc1]

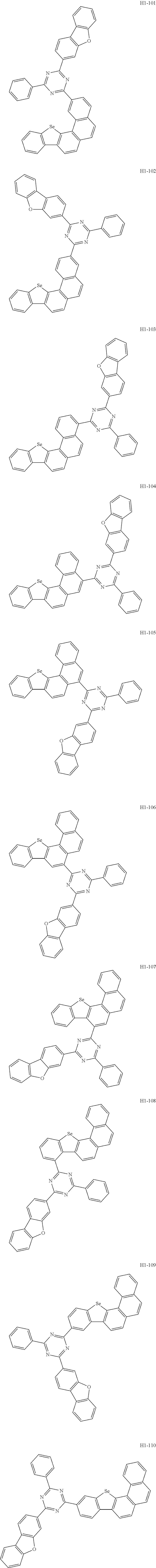

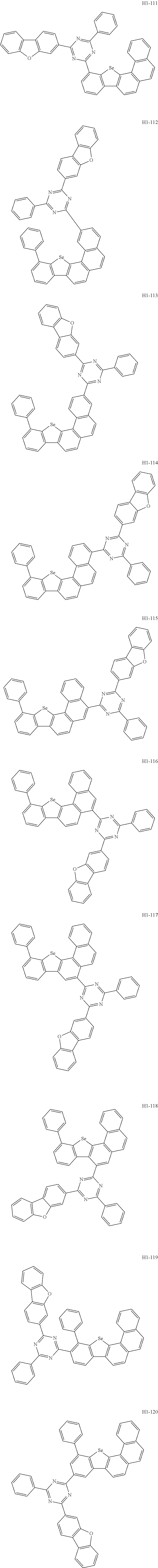

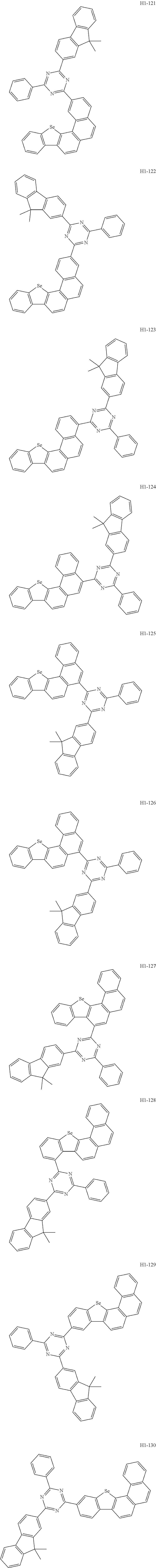

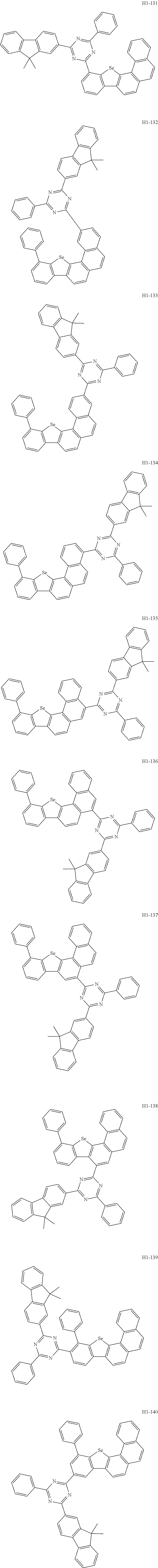

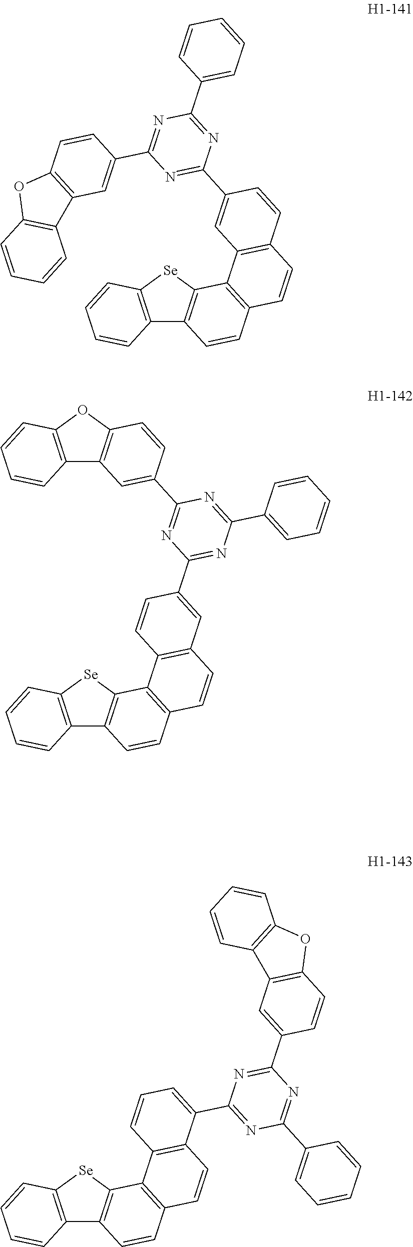 [SMILES: c1ccc(-c2nc(-c3ccc4oc5ccccc5c4c3)nc(-c3ccc4ccc5ccc6c7ccccc7[se]c6c5c4c3)n2)cc1 ; c1ccc(-c2nc(-c3ccc4c(ccc5ccc6c7ccccc7[se]c6c54)c3)nc(-c3ccc4oc5ccccc5c4c3)n2)cc1 ; c1ccc(-c2nc(-c3ccc4oc5ccccc5c4c3)nc(-c3cccc4c3ccc3ccc5c6ccccc6[se]c5c34)n2)cc1]

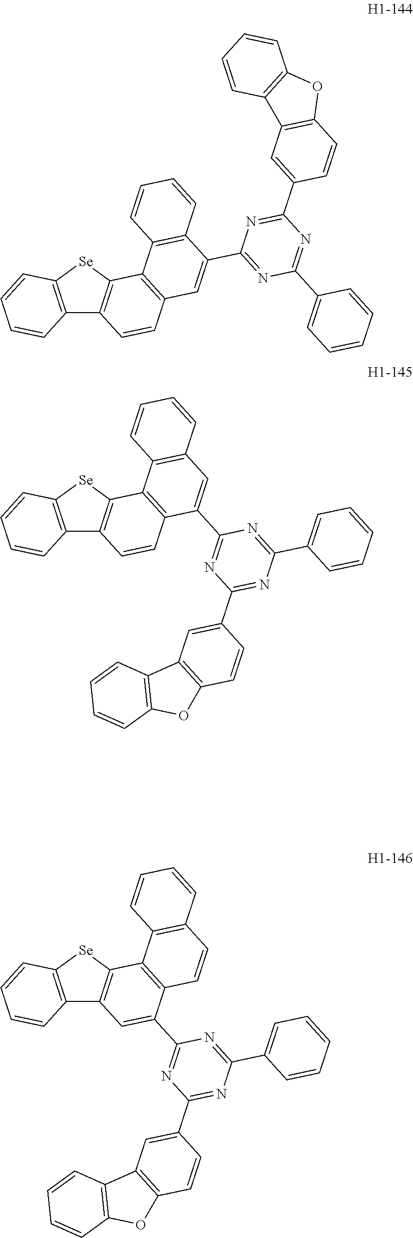 [SMILES: c1ccc(-c2nc(-c3ccc4oc5ccccc5c4c3)nc(-c3cc4ccc5c6ccccc6[se]c5c4c4ccccc34)n2)cc1 ; c1ccc(-c2nc(-c3ccc4oc5ccccc5c4c3)nc(-c3cc4ccccc4c4c3ccc3c5ccccc5[se]c34)n2)cc1 ; c1ccc(-c2nc(-c3ccc4oc5ccccc5c4c3)nc(-c3cc4c5ccccc5[se]c4c4c3ccc3ccccc34)n2)cc1]

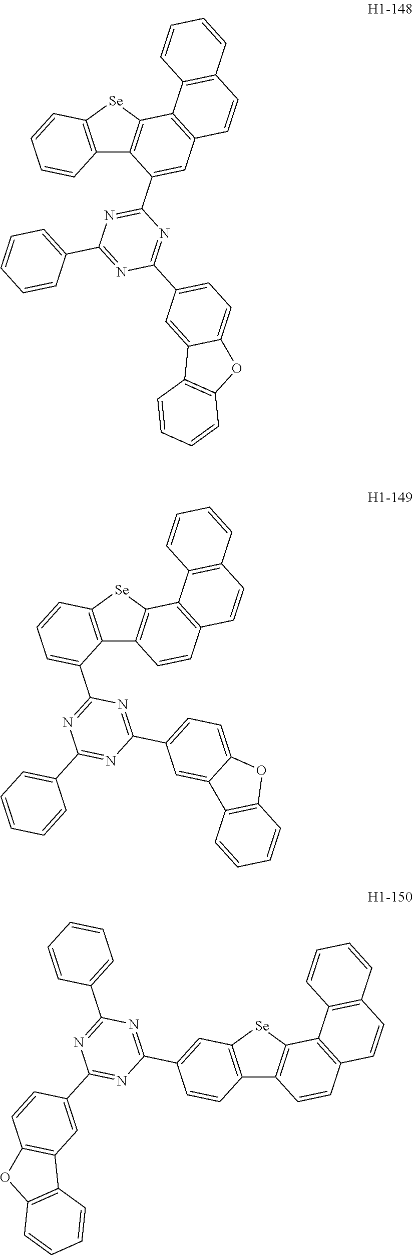 [SMILES: c1ccc(-c2nc(-c3ccc4oc5ccccc5c4c3)nc(-c3cc4ccc5ccccc5c4c4[se]c5ccccc5c34)n2)cc1 ; c1ccc(-c2nc(-c3ccc4oc5ccccc5c4c3)nc(-c3cccc4[se]c5c(ccc6ccc7ccccc7c65)c34)n2)cc1 ; c1ccc(-c2nc(-c3ccc4c(c3)[se]c3c4ccc4ccc5ccccc5c43)nc(-c3ccc4oc5ccccc5c4c3)n2)cc1]

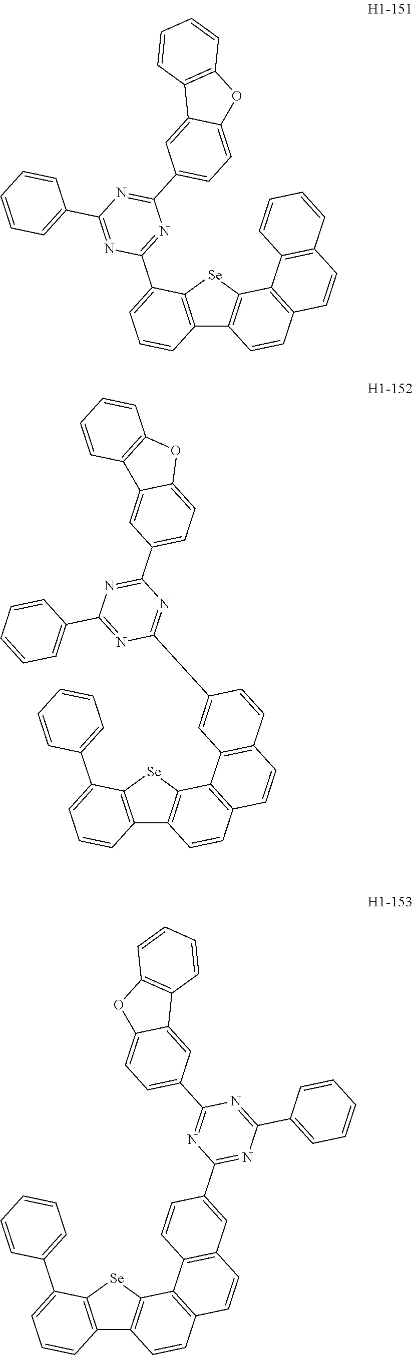 [SMILES: c1ccc(-c2nc(-c3ccc4oc5ccccc5c4c3)nc(-c3cccc4c3[se]c3c4ccc4ccc5ccccc5c43)n2)cc1 ; c1ccc(-c2nc(-c3ccc4oc5ccccc5c4c3)nc(-c3ccc4ccc5ccc6c7cccc(-c8ccccc8)c7[se]c6c5c4c3)n2)cc1 ; c1ccc(-c2nc(-c3ccc4c(ccc5ccc6c7cccc(-c8ccccc8)c7[se]c6c54)c3)nc(-c3ccc4oc5ccccc5c4c3)n2)cc1]

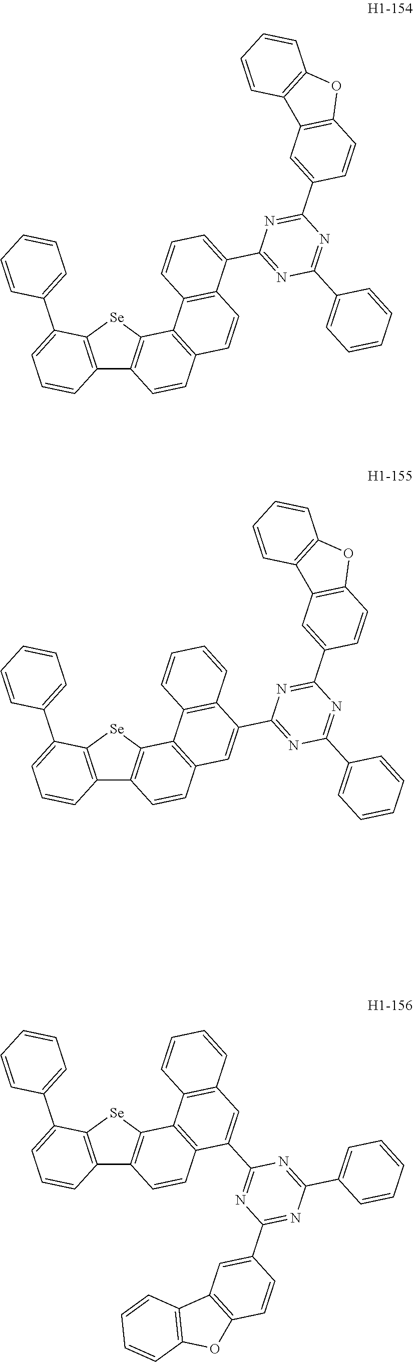 [SMILES: c1ccc(-c2nc(-c3ccc4oc5ccccc5c4c3)nc(-c3cccc4c3ccc3ccc5c6cccc(-c7ccccc7)c6[se]c5c34)n2)cc1 ; c1ccc(-c2nc(-c3ccc4oc5ccccc5c4c3)nc(-c3cc4ccc5c6cccc(-c7ccccc7)c6[se]c5c4c4ccccc34)n2)cc1 ; c1ccc(-c2nc(-c3ccc4oc5ccccc5c4c3)nc(-c3cc4ccccc4c4c3ccc3c5cccc(-c6ccccc6)c5[se]c34)n2)cc1]

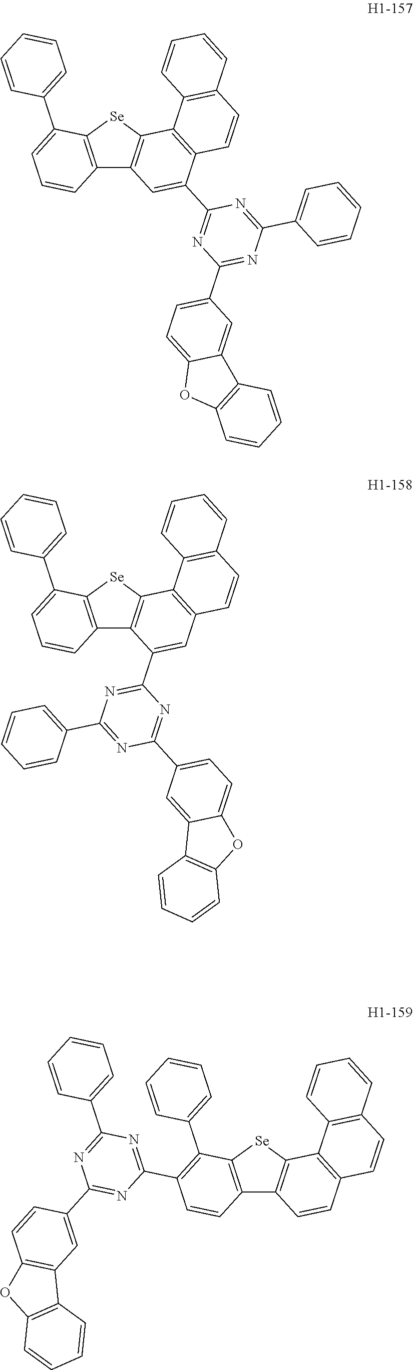 [SMILES: c1ccc(-c2nc(-c3ccc4oc5ccccc5c4c3)nc(-c3cc4c5cccc(-c6ccccc6)c5[se]c4c4c3ccc3ccccc34)n2)cc1 ; c1ccc(-c2nc(-c3ccc4oc5ccccc5c4c3)nc(-c3cc4ccc5ccccc5c4c4[se]c5c(-c6ccccc6)cccc5c34)n2)cc1 ; c1ccc(-c2nc(-c3ccc4oc5ccccc5c4c3)nc(-c3ccc4c([se]c5c4ccc4ccc6ccccc6c45)c3-c3ccccc3)n2)cc1]

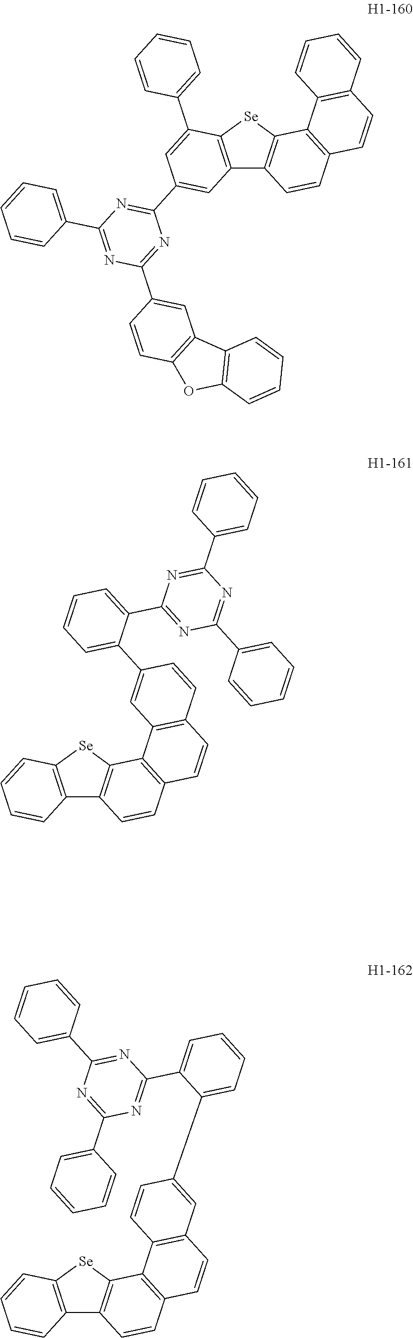 [SMILES: c1ccc(-c2nc(-c3ccc4oc5ccccc5c4c3)nc(-c3cc(-c4ccccc4)c4[se]c5c(ccc6ccc7ccccc7c65)c4c3)n2)cc1 ; c1ccc(-c2nc(-c3ccccc3)nc(-c3ccccc3-c3ccc4ccc5ccc6c7ccccc7[se]c6c5c4c3)n2)cc1 ; c1ccc(-c2nc(-c3ccccc3)nc(-c3ccccc3-c3ccc4c(ccc5ccc6c7ccccc7[se]c6c54)c3)n2)cc1]

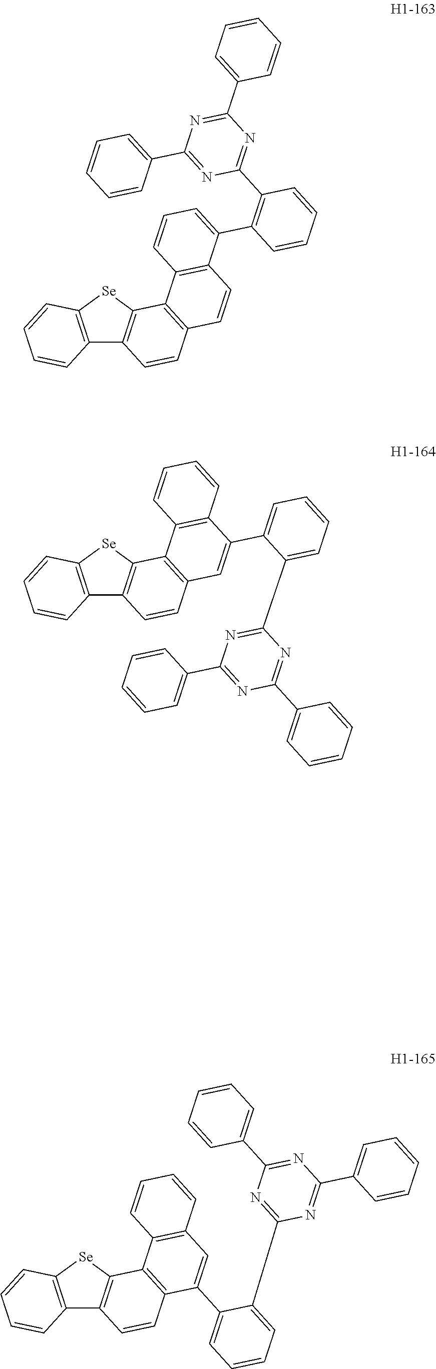 [SMILES: c1ccc(-c2nc(-c3ccccc3)nc(-c3ccccc3-c3cccc4c3ccc3ccc5c6ccccc6[se]c5c34)n2)cc1 ; c1ccc(-c2nc(-c3ccccc3)nc(-c3ccccc3-c3cc4ccc5c6ccccc6[se]c5c4c4ccccc34)n2)cc1 ; c1ccc(-c2nc(-c3ccccc3)nc(-c3ccccc3-c3cc4ccccc4c4c3ccc3c5ccccc5[se]c34)n2)cc1]

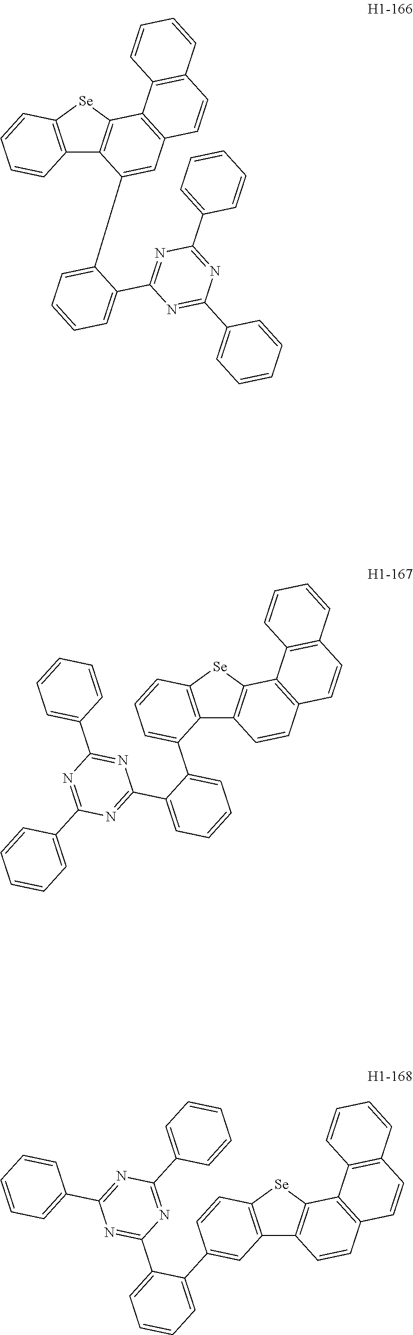 [SMILES: c1ccc(-c2nc(-c3ccccc3)nc(-c3ccccc3-c3cc4ccc5ccccc5c4c4[se]c5ccccc5c34)n2)cc1 ; c1ccc(-c2nc(-c3ccccc3)nc(-c3ccccc3-c3cccc4[se]c5c(ccc6ccc7ccccc7c65)c34)n2)cc1 ; c1ccc(-c2nc(-c3ccccc3)nc(-c3ccccc3-c3ccc4[se]c5c(ccc6ccc7ccccc7c65)c4c3)n2)cc1]

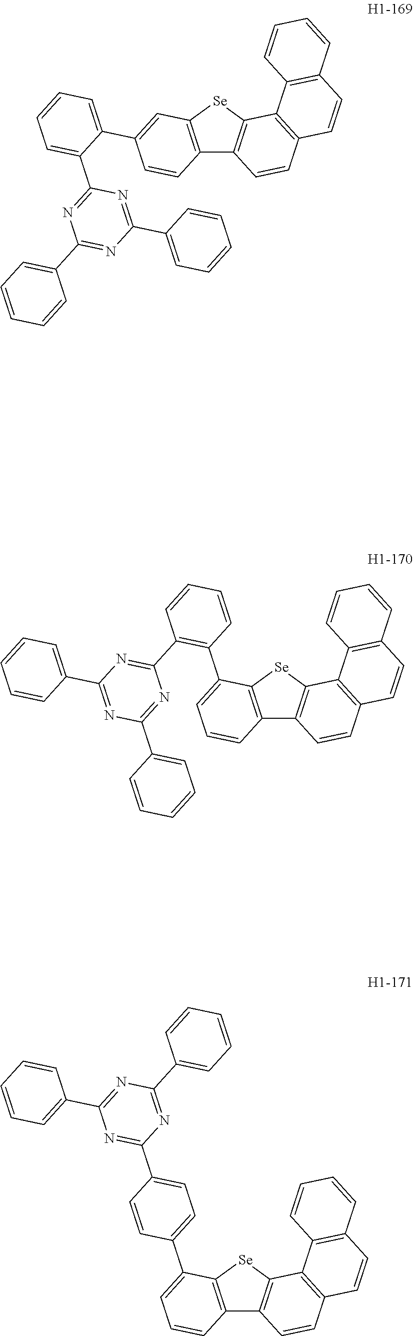 [SMILES: c1ccc(-c2nc(-c3ccccc3)nc(-c3ccccc3-c3ccc4c(c3)[se]c3c4ccc4ccc5ccccc5c43)n2)cc1 ; c1ccc(-c2nc(-c3ccccc3)nc(-c3ccccc3-c3cccc4c3[se]c3c4ccc4ccc5ccccc5c43)n2)cc1 ; c1ccc(-c2nc(-c3ccccc3)nc(-c3ccc(-c4cccc5c4[se]c4c5ccc5ccc6ccccc6c54)cc3)n2)cc1]

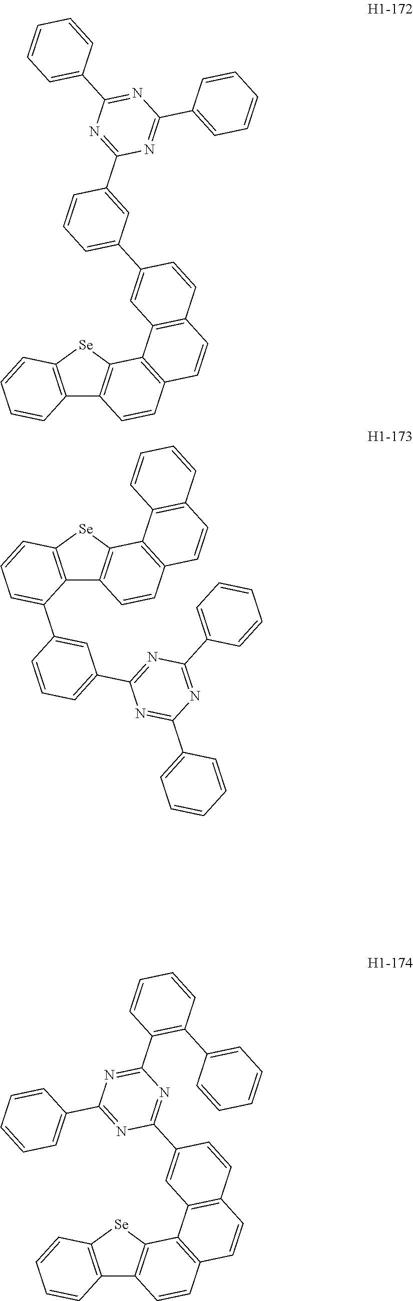

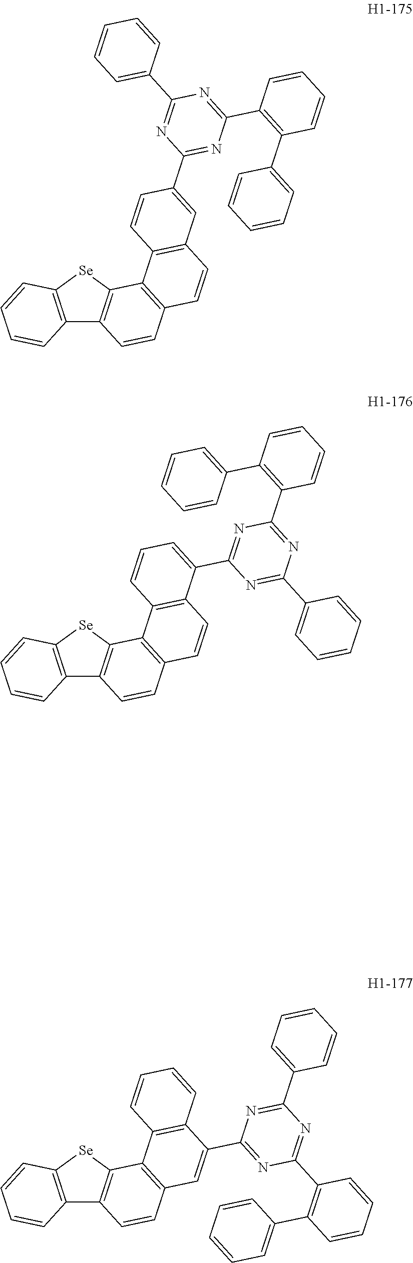 [SMILES: c1ccc(-c2nc(-c3ccc4c(ccc5ccc6c7ccccc7[se]c6c54)c3)nc(-c3ccccc3-c3ccccc3)n2)cc1 ; c1ccc(-c2nc(-c3ccccc3-c3ccccc3)nc(-c3cccc4c3ccc3ccc5c6ccccc6[se]c5c34)n2)cc1 ; c1ccc(-c2nc(-c3ccccc3-c3ccccc3)nc(-c3cc4ccc5c6ccccc6[se]c5c4c4ccccc34)n2)cc1]

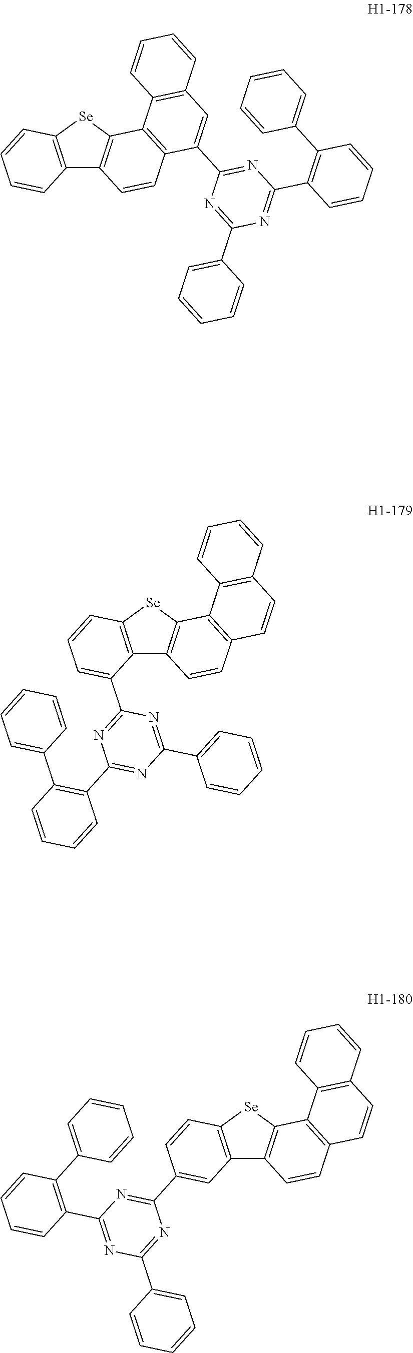 [SMILES: c1ccc(-c2nc(-c3ccccc3-c3ccccc3)nc(-c3cc4ccccc4c4c3ccc3c5ccccc5[se]c34)n2)cc1 ; c1ccc(-c2nc(-c3ccccc3-c3ccccc3)nc(-c3cccc4[se]c5c(ccc6ccc7ccccc7c65)c34)n2)cc1 ; c1ccc(-c2nc(-c3ccc4[se]c5c(ccc6ccc7ccccc7c65)c4c3)nc(-c3ccccc3-c3ccccc3)n2)cc1]

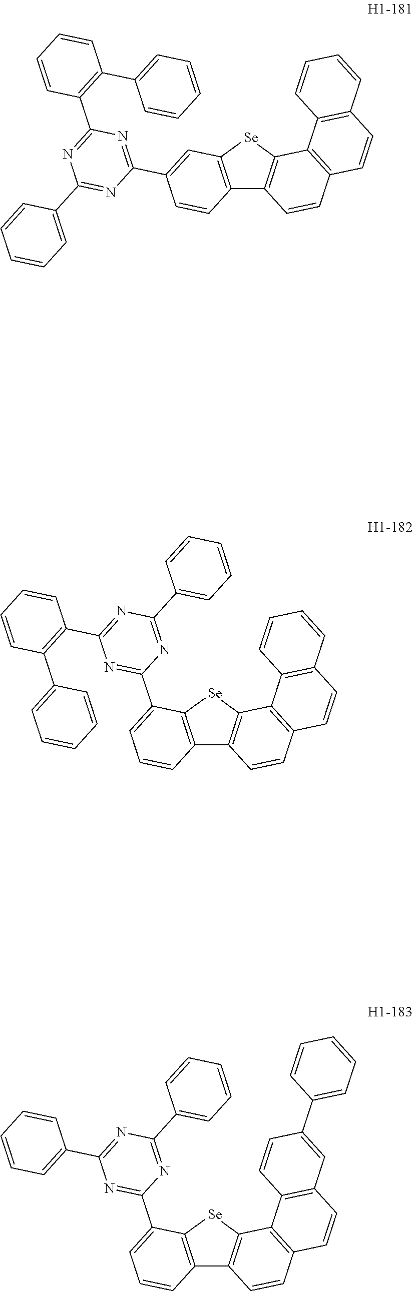 [SMILES: c1ccc(-c2nc(-c3ccc4c(c3)[se]c3c4ccc4ccc5ccccc5c43)nc(-c3ccccc3-c3ccccc3)n2)cc1 ; c1ccc(-c2nc(-c3ccccc3-c3ccccc3)nc(-c3cccc4c3[se]c3c4ccc4ccc5ccccc5c43)n2)cc1 ; c1ccc(-c2ccc3c(ccc4ccc5c6cccc(-c7nc(-c8ccccc8)nc(-c8ccccc8)n7)c6[se]c5c43)c2)cc1]

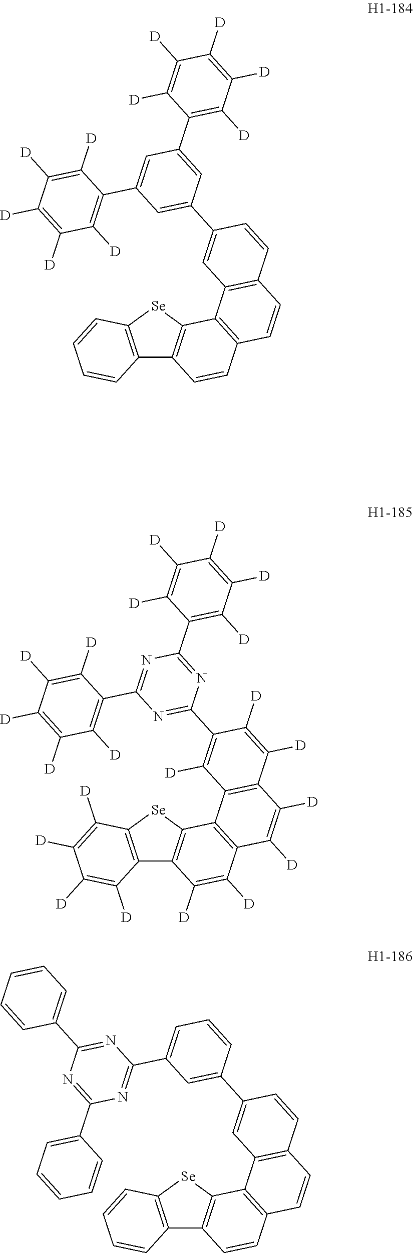 [SMILES: [2H]c1c([2H])c([2H])c(-c2cc(-c3ccc4ccc5ccc6c7ccccc7[se]c6c5c4c3)cc(-c3c([2H])c([2H])c([2H])c([2H])c3[2H])c2)c([2H])c1[2H] ; [2H]c1c([2H])c([2H])c(-c2nc(-c3c([2H])c([2H])c([2H])c([2H])c3[2H])nc(-c3c([2H])c([2H])c4c([2H])c([2H])c5c([2H])c([2H])c6c([se]c7c([2H])c([2H])c([2H])c([2H])c76)c5c4c3[2H])n2)c([2H])c1[2H] ; c1ccc(-c2nc(-c3ccccc3)nc(-c3cccc(-c4ccc5ccc6ccc7c8ccccc8[se]c7c6c5c4)c3)n2)cc1]

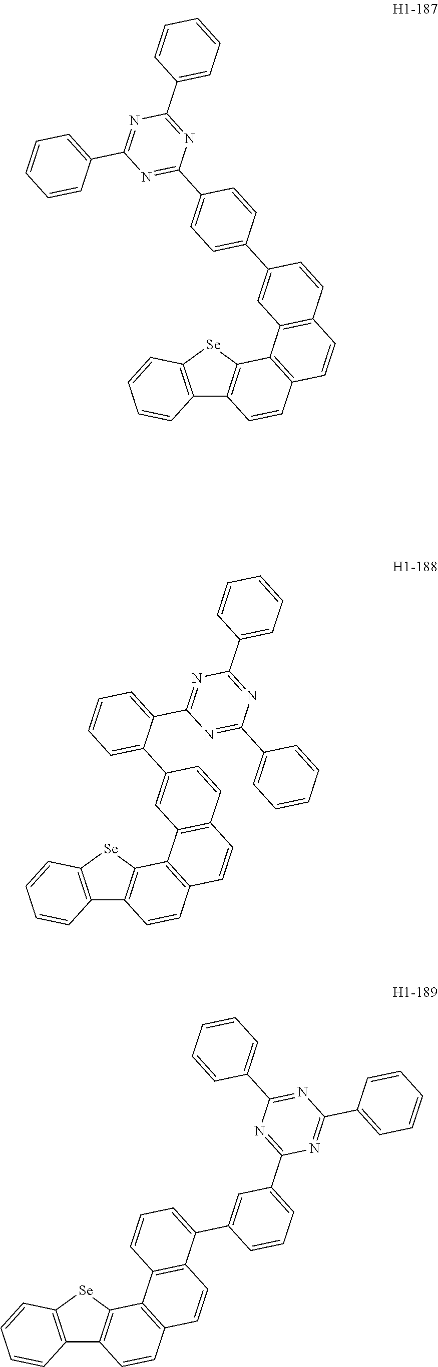 [SMILES: c1ccc(-c2nc(-c3ccccc3)nc(-c3ccc(-c4ccc5ccc6ccc7c8ccccc8[se]c7c6c5c4)cc3)n2)cc1 ; c1ccc(-c2nc(-c3ccccc3)nc(-c3ccccc3-c3ccc4ccc5ccc6c7ccccc7[se]c6c5c4c3)n2)cc1 ; c1ccc(-c2nc(-c3ccccc3)nc(-c3cccc(-c4cccc5c4ccc4ccc6c7ccccc7[se]c6c45)c3)n2)cc1]

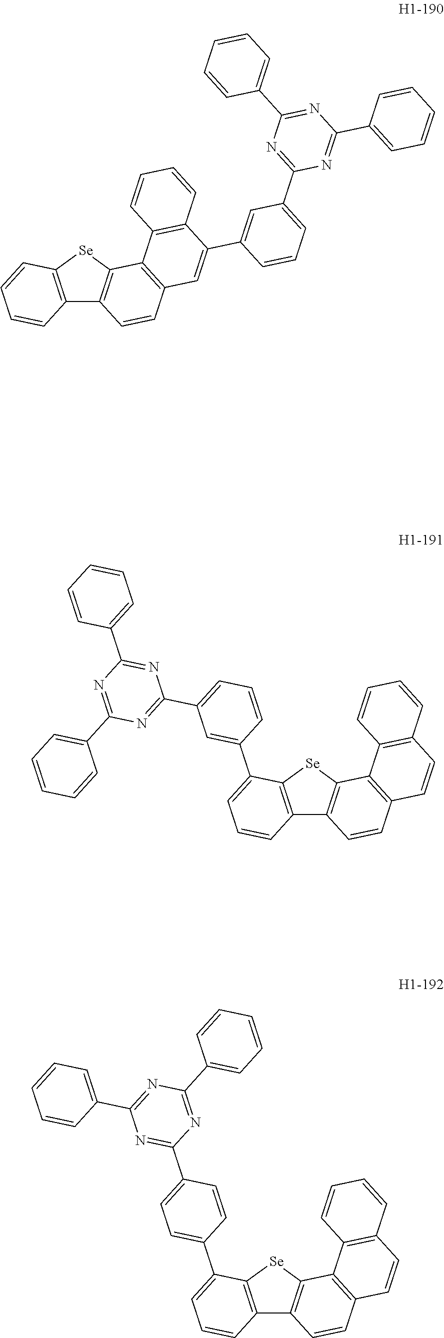 [SMILES: c1ccc(-c2nc(-c3ccccc3)nc(-c3cccc(-c4cc5ccc6c7ccccc7[se]c6c5c5ccccc45)c3)n2)cc1 ; c1ccc(-c2nc(-c3ccccc3)nc(-c3cccc(-c4cccc5c4[se]c4c5ccc5ccc6ccccc6c54)c3)n2)cc1 ; c1ccc(-c2nc(-c3ccccc3)nc(-c3ccc(-c4cccc5c4[se]c4c5ccc5ccc6ccccc6c54)cc3)n2)cc1]

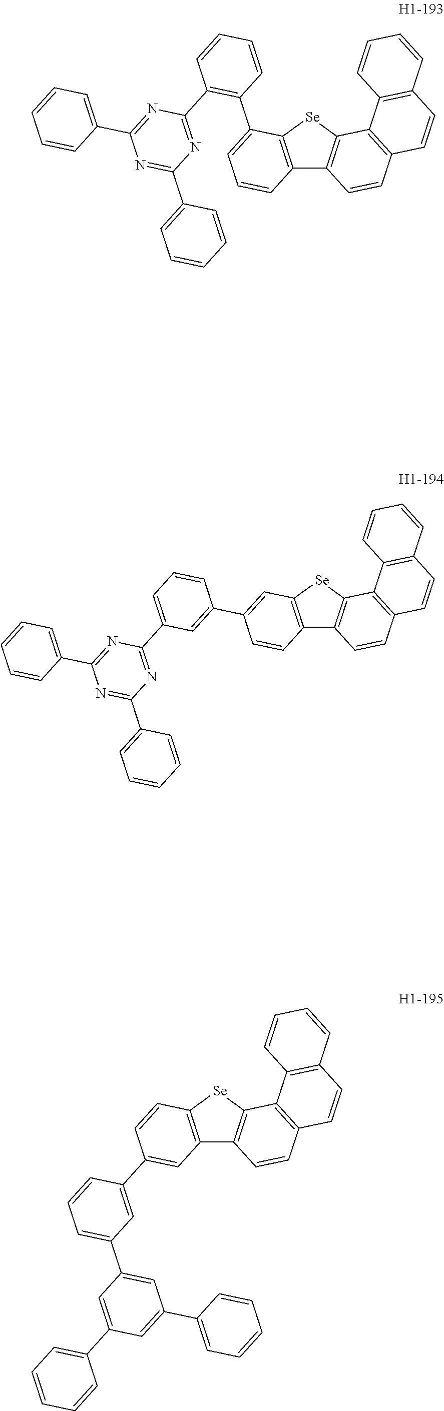 [SMILES: c1ccc(-c2nc(-c3ccccc3)nc(-c3ccccc3-c3cccc4c3[se]c3c4ccc4ccc5ccccc5c43)n2)cc1 ; c1ccc(-c2nc(-c3ccccc3)nc(-c3cccc(-c4ccc5c(c4)[se]c4c5ccc5ccc6ccccc6c54)c3)n2)cc1 ; c1ccc(-c2cc(-c3ccccc3)cc(-c3cccc(-c4ccc5[se]c6c(ccc7ccc8ccccc8c76)c5c4)c3)c2)cc1]

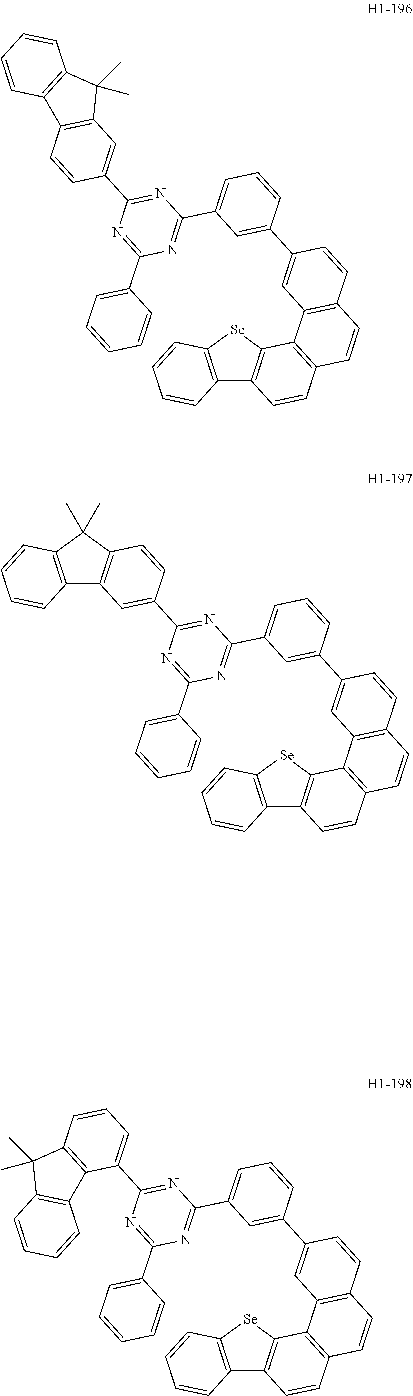 [SMILES: CC1(C)c2ccccc2-c2ccc(-c3nc(-c4ccccc4)nc(-c4cccc(-c5ccc6ccc7ccc8c9ccccc9[se]c8c7c6c5)c4)n3)cc21 ; CC1(C)c2ccccc2-c2cc(-c3nc(-c4ccccc4)nc(-c4cccc(-c5ccc6ccc7ccc8c9ccccc9[se]c8c7c6c5)c4)n3)ccc21 ; CC1(C)c2ccccc2-c2c(-c3nc(-c4ccccc4)nc(-c4cccc(-c5ccc6ccc7ccc8c9ccccc9[se]c8c7c6c5)c4)n3)cccc21]

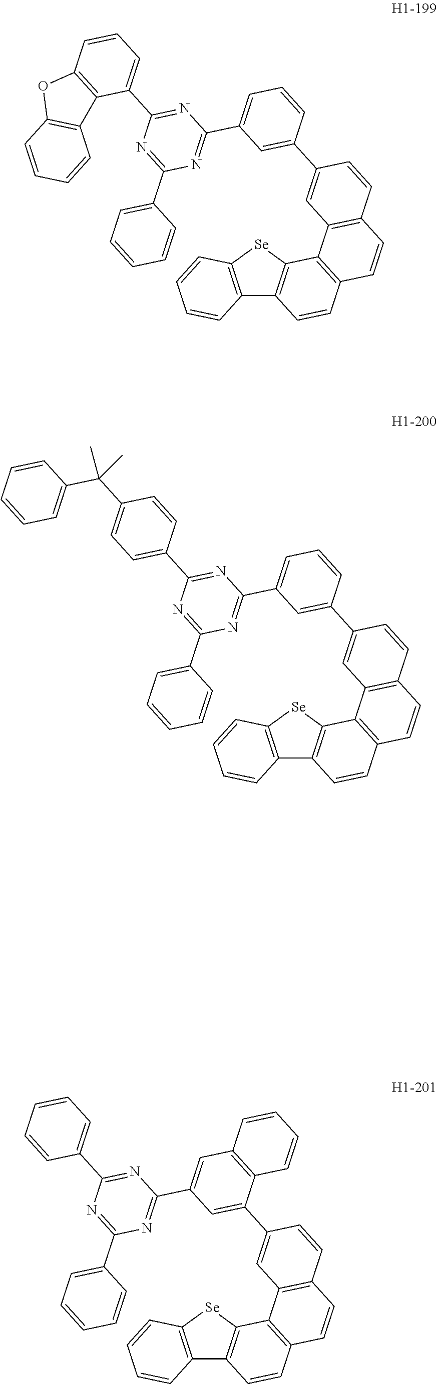 [SMILES: c1ccc(-c2nc(-c3cccc(-c4ccc5ccc6ccc7c8ccccc8[se]c7c6c5c4)c3)nc(-c3cccc4oc5ccccc5c34)n2)cc1 ; CC(C)(c1ccccc1)c1ccc(-c2nc(-c3ccccc3)nc(-c3cccc(-c4ccc5ccc6ccc7c8ccccc8[se]c7c6c5c4)c3)n2)cc1 ; c1ccc(-c2nc(-c3ccccc3)nc(-c3cc(-c4ccc5ccc6ccc7c8ccccc8[se]c7c6c5c4)c4ccccc4c3)n2)cc1]

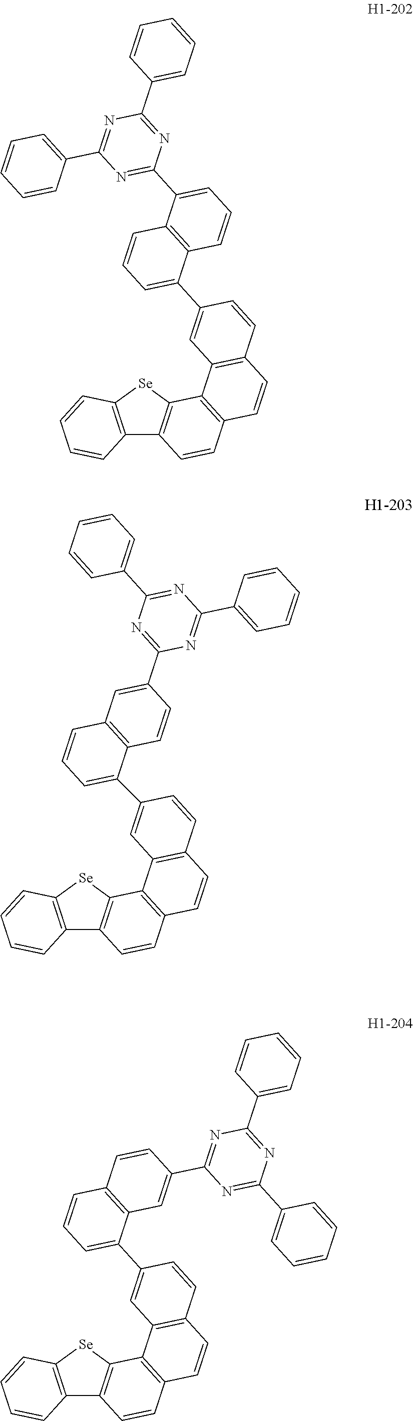 [SMILES: c1ccc(-c2nc(-c3ccccc3)nc(-c3cccc4c(-c5ccc6ccc7ccc8c9ccccc9[se]c8c7c6c5)cccc34)n2)cc1 ; c1ccc(-c2nc(-c3ccccc3)nc(-c3ccc4c(-c5ccc6ccc7ccc8c9ccccc9[se]c8c7c6c5)cccc4c3)n2)cc1 ; c1ccc(-c2nc(-c3ccccc3)nc(-c3ccc4cccc(-c5ccc6ccc7ccc8c9ccccc9[se]c8c7c6c5)c4c3)n2)cc1]

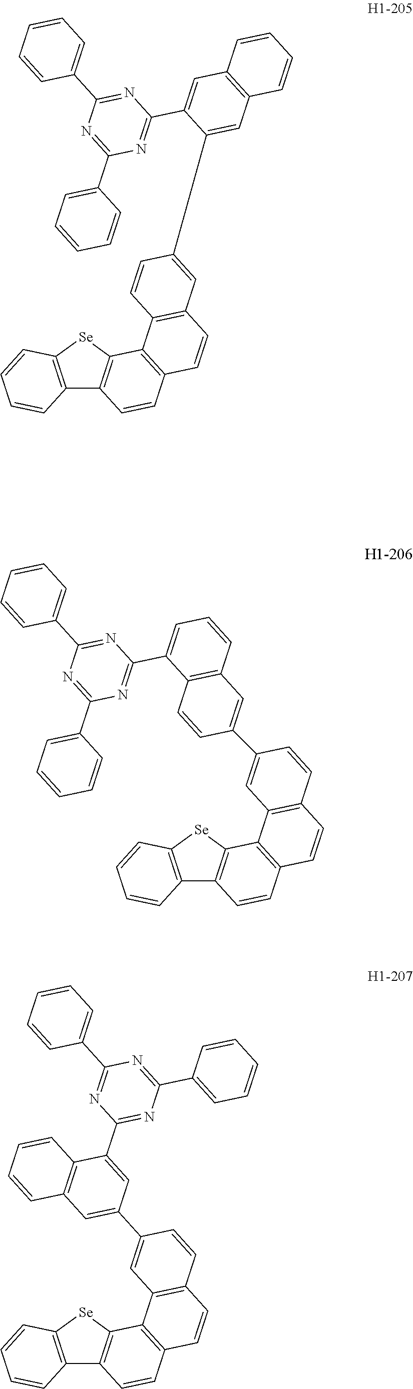 [SMILES: c1ccc(-c2nc(-c3ccccc3)nc(-c3cc4ccccc4cc3-c3ccc4c(ccc5ccc6c7ccccc7[se]c6c54)c3)n2)cc1 ; c1ccc(-c2nc(-c3ccccc3)nc(-c3cccc4cc(-c5ccc6ccc7ccc8c9ccccc9[se]c8c7c6c5)ccc34)n2)cc1 ; c1ccc(-c2nc(-c3ccccc3)nc(-c3cc(-c4ccc5ccc6ccc7c8ccccc8[se]c7c6c5c4)cc4ccccc34)n2)cc1]

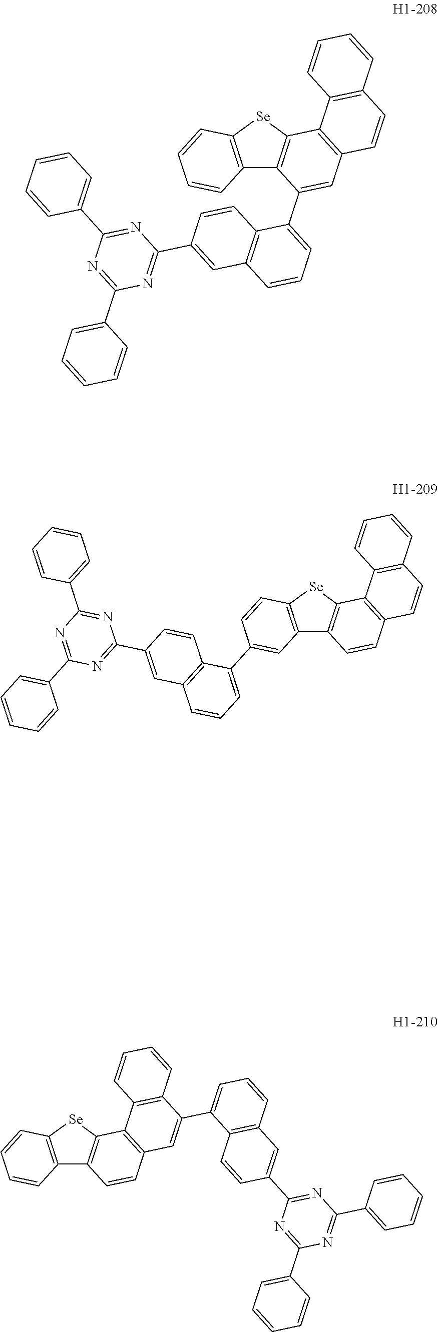 [SMILES: c1ccc(-c2nc(-c3ccccc3)nc(-c3ccc4c(-c5cc6ccc7ccccc7c6c6[se]c7ccccc7c56)cccc4c3)n2)cc1 ; c1ccc(-c2nc(-c3ccccc3)nc(-c3ccc4c(-c5ccc6[se]c7c(ccc8ccc9ccccc9c87)c6c5)cccc4c3)n2)cc1 ; c1ccc(-c2nc(-c3ccccc3)nc(-c3ccc4c(-c5cc6ccc7c8ccccc8[se]c7c6c6ccccc56)cccc4c3)n2)cc1]

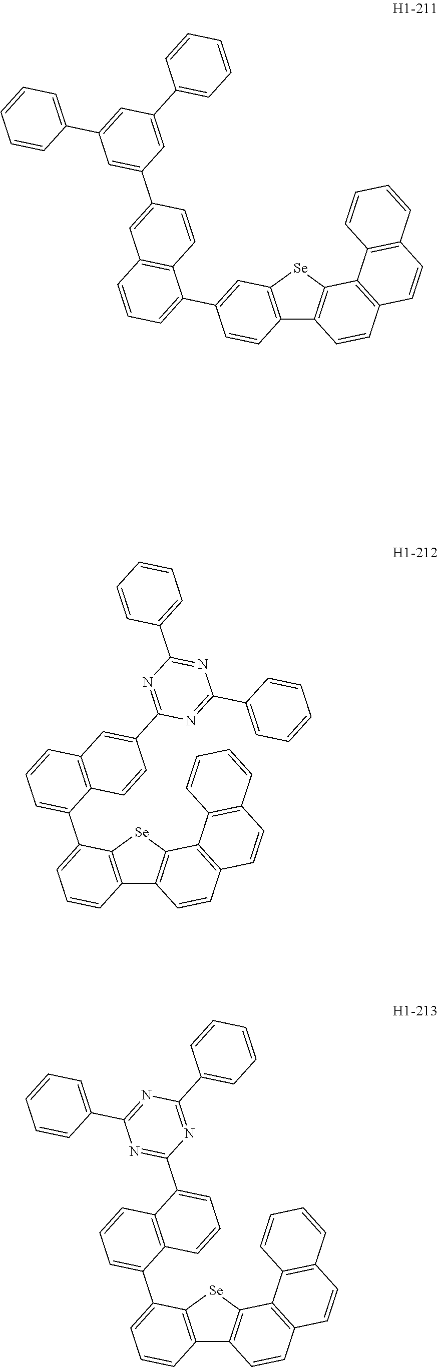 [SMILES: c1ccc(-c2cc(-c3ccccc3)cc(-c3ccc4c(-c5ccc6c(c5)[se]c5c6ccc6ccc7ccccc7c65)cccc4c3)c2)cc1 ; c1ccc(-c2nc(-c3ccccc3)nc(-c3ccc4c(-c5cccc6c5[se]c5c6ccc6ccc7ccccc7c65)cccc4c3)n2)cc1 ; c1ccc(-c2nc(-c3ccccc3)nc(-c3cccc4c(-c5cccc6c5[se]c5c6ccc6ccc7ccccc7c65)cccc34)n2)cc1]

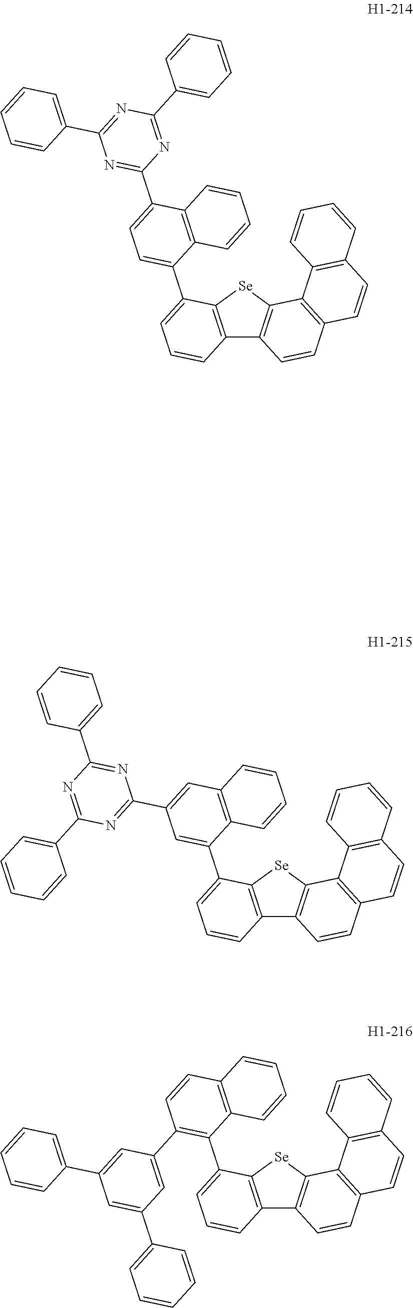 [SMILES: c1ccc(-c2nc(-c3ccccc3)nc(-c3ccc(-c4cccc5c4[se]c4c5ccc5ccc6ccccc6c54)c4ccccc34)n2)cc1 ; c1ccc(-c2nc(-c3ccccc3)nc(-c3cc(-c4cccc5c4[se]c4c5ccc5ccc6ccccc6c54)c4ccccc4c3)n2)cc1 ; c1ccc(-c2cc(-c3ccccc3)cc(-c3ccc4ccccc4c3-c3cccc4c3[se]c3c4ccc4ccc5ccccc5c43)c2)cc1]

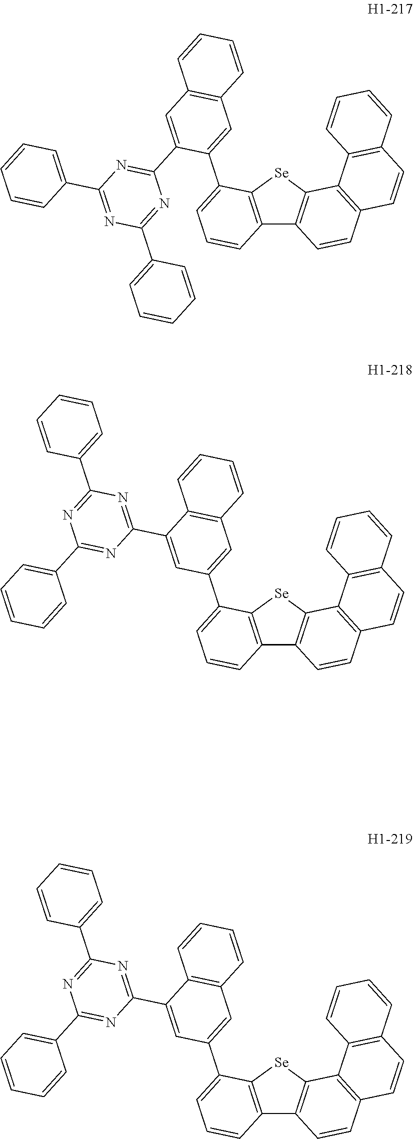 [SMILES: c1ccc(-c2nc(-c3ccccc3)nc(-c3cc4ccccc4cc3-c3cccc4c3[se]c3c4ccc4ccc5ccccc5c43)n2)cc1 ; c1ccc(-c2nc(-c3ccccc3)nc(-c3cc(-c4cccc5c4[se]c4c5ccc5ccc6ccccc6c54)cc4ccccc34)n2)cc1 ; c1ccc(-c2nc(-c3ccccc3)nc(-c3cc(-c4cccc5c4[se]c4c5ccc5ccc6ccccc6c54)cc4ccccc34)n2)cc1]

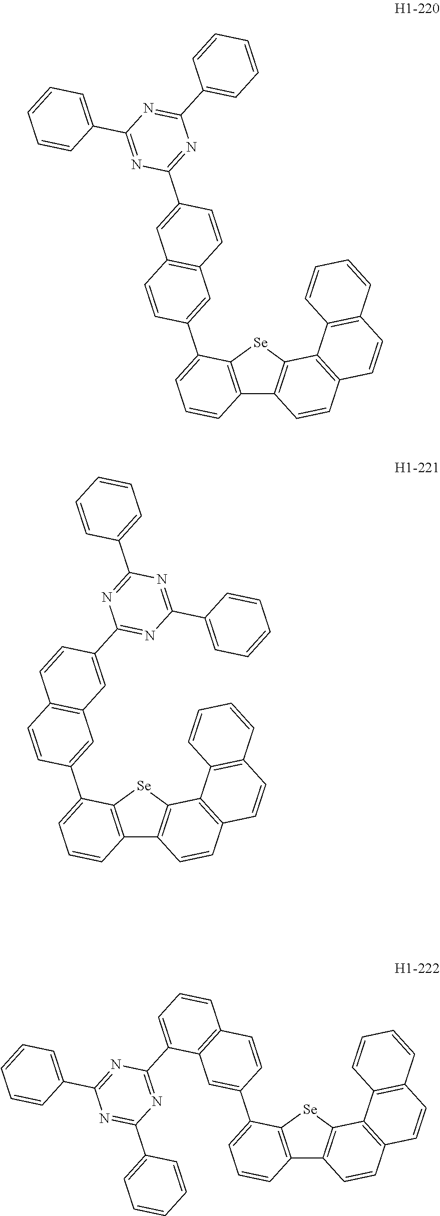 [SMILES: c1ccc(-c2nc(-c3ccccc3)nc(-c3ccc4cc(-c5cccc6c5[se]c5c6ccc6ccc7ccccc7c65)ccc4c3)n2)cc1 ; c1ccc(-c2nc(-c3ccccc3)nc(-c3ccc4ccc(-c5cccc6c5[se]c5c6ccc6ccc7ccccc7c65)cc4c3)n2)cc1 ; c1ccc(-c2nc(-c3ccccc3)nc(-c3cccc4ccc(-c5cccc6c5[se]c5c6ccc6ccc7ccccc7c65)cc34)n2)cc1]

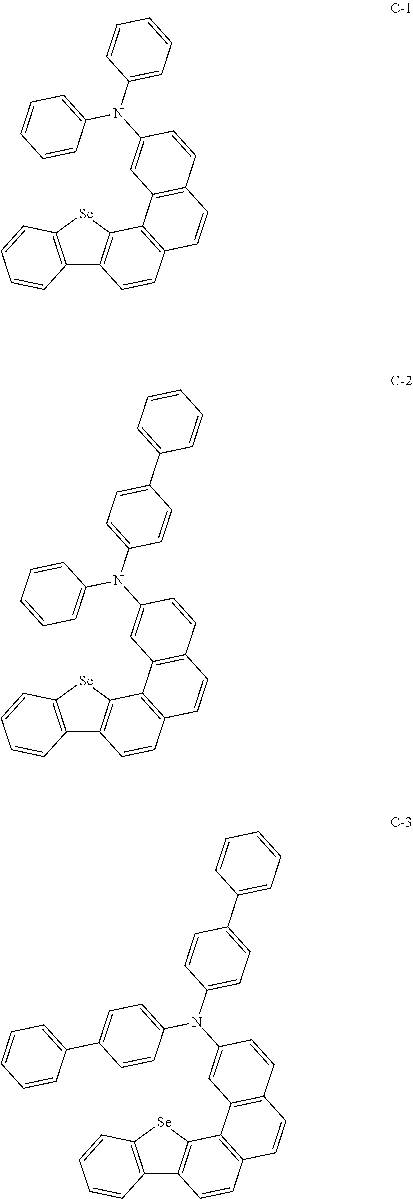 [SMILES: c1ccc(N(c2ccccc2)c2ccc3ccc4ccc5c6ccccc6[se]c5c4c3c2)cc1 ; c1ccc(-c2ccc(N(c3ccccc3)c3ccc4ccc5ccc6c7ccccc7[se]c6c5c4c3)cc2)cc1 ; c1ccc(-c2ccc(N(c3ccc(-c4ccccc4)cc3)c3ccc4ccc5ccc6c7ccccc7[se]c6c5c4c3)cc2)cc1]

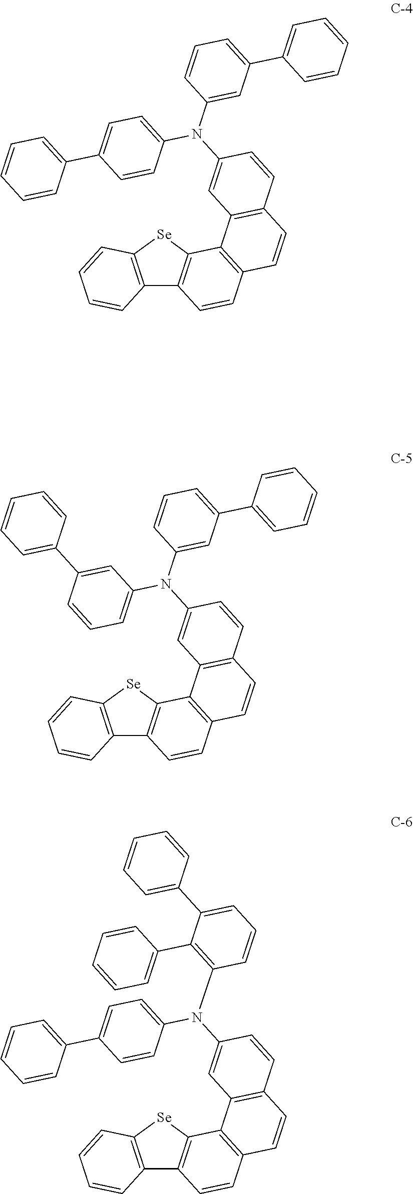 [SMILES: c1ccc(-c2ccc(N(c3cccc(-c4ccccc4)c3)c3ccc4ccc5ccc6c7ccccc7[se]c6c5c4c3)cc2)cc1 ; c1ccc(-c2cccc(N(c3cccc(-c4ccccc4)c3)c3ccc4ccc5ccc6c7ccccc7[se]c6c5c4c3)c2)cc1 ; c1ccc(-c2ccc(N(c3ccc4ccc5ccc6c7ccccc7[se]c6c5c4c3)c3cccc(-c4ccccc4)c3-c3ccccc3)cc2)cc1]

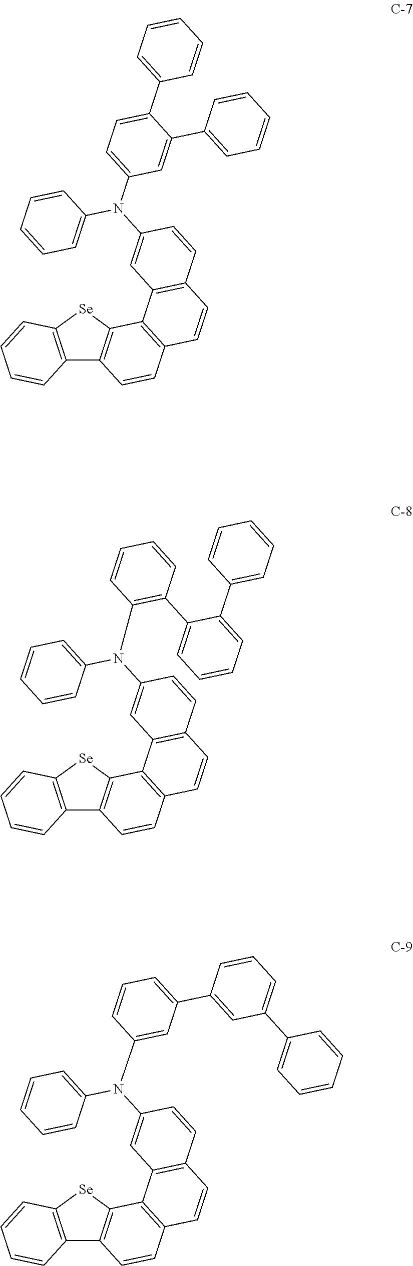 [SMILES: c1ccc(-c2ccc(N(c3ccccc3)c3ccc4ccc5ccc6c7ccccc7[se]c6c5c4c3)cc2-c2ccccc2)cc1 ; c1ccc(-c2ccccc2-c2ccccc2N(c2ccccc2)c2ccc3ccc4ccc5c6ccccc6[se]c5c4c3c2)cc1 ; c1ccc(-c2cccc(-c3cccc(N(c4ccccc4)c4ccc5ccc6ccc7c8ccccc8[se]c7c6c5c4)c3)c2)cc1]

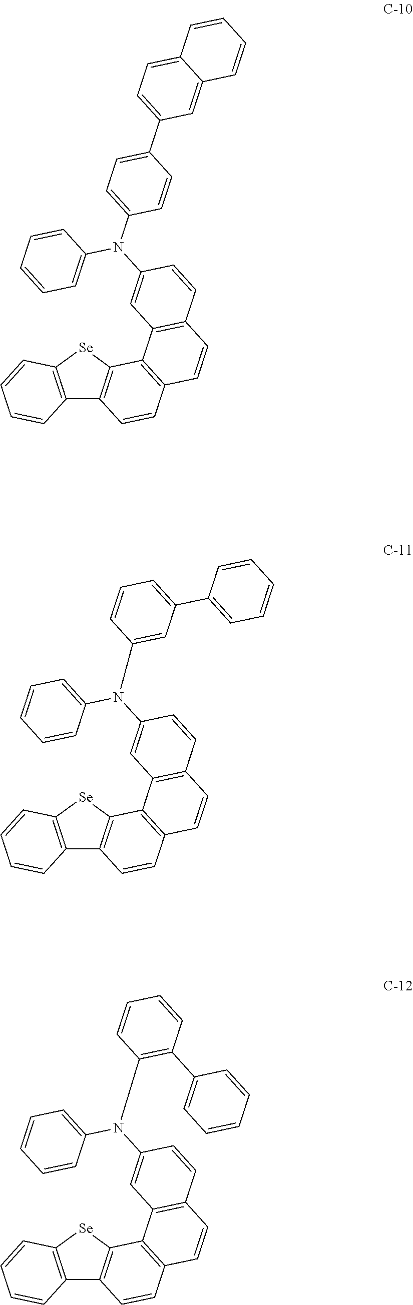 [SMILES: c1ccc(N(c2ccc(-c3ccc4ccccc4c3)cc2)c2ccc3ccc4ccc5c6ccccc6[se]c5c4c3c2)cc1 ; c1ccc(-c2cccc(N(c3ccccc3)c3ccc4ccc5ccc6c7ccccc7[se]c6c5c4c3)c2)cc1 ; c1ccc(-c2ccccc2N(c2ccccc2)c2ccc3ccc4ccc5c6ccccc6[se]c5c4c3c2)cc1]

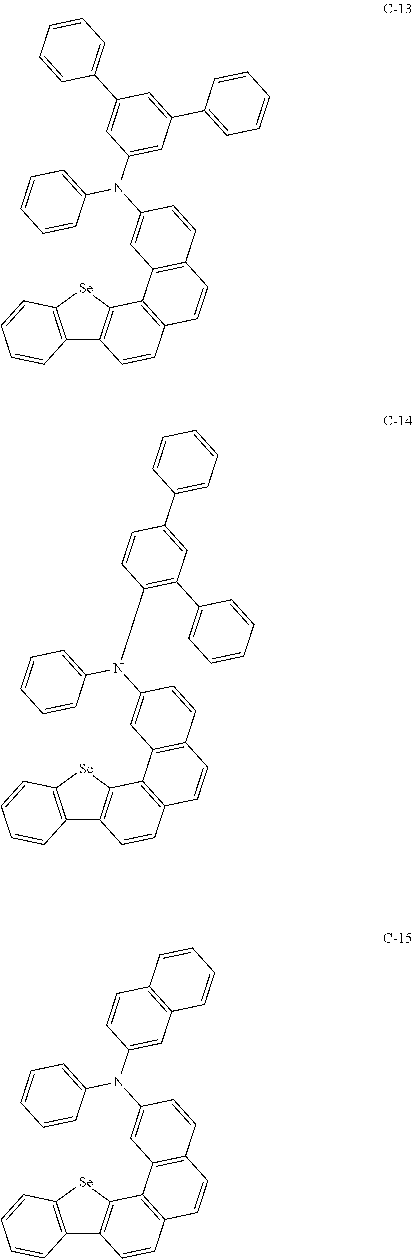 [SMILES: c1ccc(-c2cc(-c3ccccc3)cc(N(c3ccccc3)c3ccc4ccc5ccc6c7ccccc7[se]c6c5c4c3)c2)cc1 ; c1ccc(-c2ccc(N(c3ccccc3)c3ccc4ccc5ccc6c7ccccc7[se]c6c5c4c3)c(-c3ccccc3)c2)cc1 ; c1ccc(N(c2ccc3ccccc3c2)c2ccc3ccc4ccc5c6ccccc6[se]c5c4c3c2)cc1]

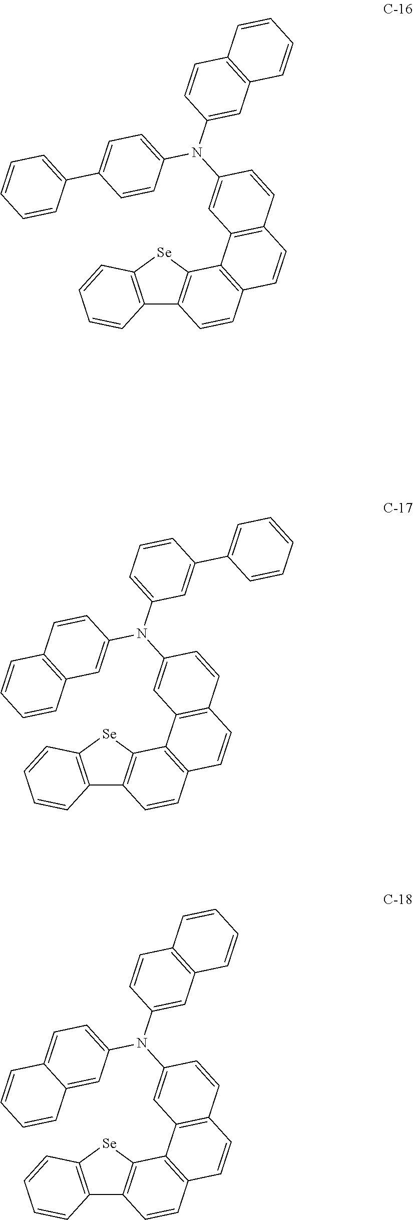 [SMILES: c1ccc(-c2ccc(N(c3ccc4ccccc4c3)c3ccc4ccc5ccc6c7ccccc7[se]c6c5c4c3)cc2)cc1 ; c1ccc(-c2cccc(N(c3ccc4ccccc4c3)c3ccc4ccc5ccc6c7ccccc7[se]c6c5c4c3)c2)cc1 ; c1ccc2cc(N(c3ccc4ccccc4c3)c3ccc4ccc5ccc6c7ccccc7[se]c6c5c4c3)ccc2c1]

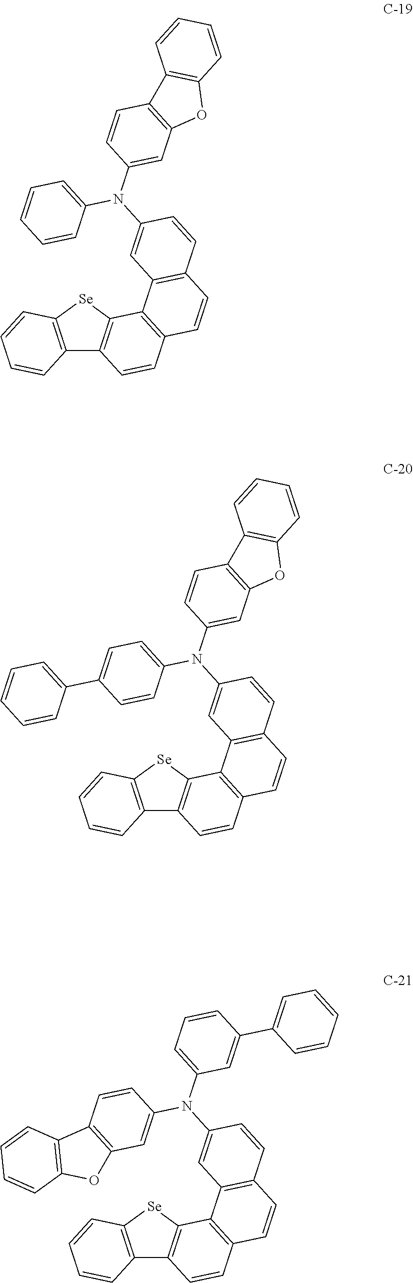 [SMILES: c1ccc(N(c2ccc3c(c2)oc2ccccc23)c2ccc3ccc4ccc5c6ccccc6[se]c5c4c3c2)cc1 ; c1ccc(-c2ccc(N(c3ccc4c(c3)oc3ccccc34)c3ccc4ccc5ccc6c7ccccc7[se]c6c5c4c3)cc2)cc1 ; c1ccc(-c2cccc(N(c3ccc4c(c3)oc3ccccc34)c3ccc4ccc5ccc6c7ccccc7[se]c6c5c4c3)c2)cc1]

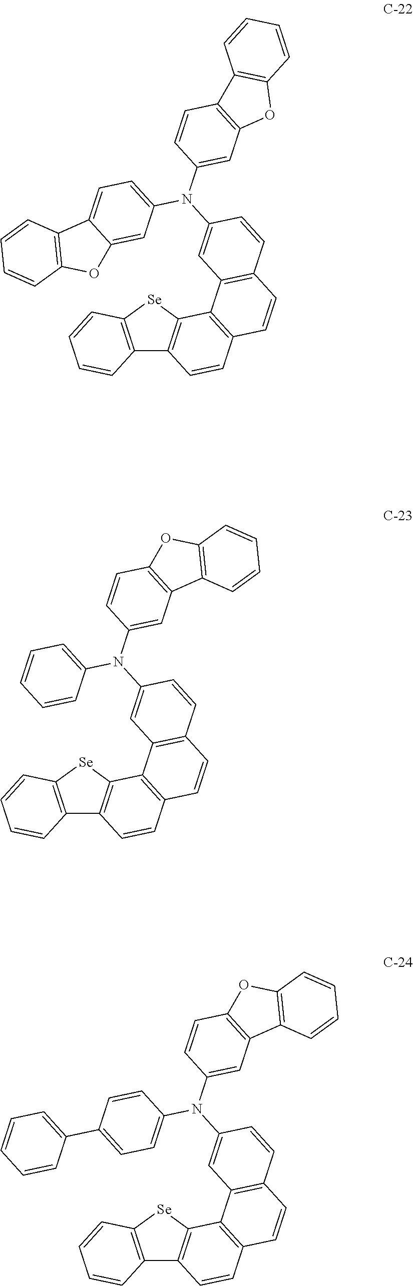 [SMILES: c1ccc2c(c1)oc1cc(N(c3ccc4c(c3)oc3ccccc34)c3ccc4ccc5ccc6c7ccccc7[se]c6c5c4c3)ccc12 ; c1ccc(N(c2ccc3oc4ccccc4c3c2)c2ccc3ccc4ccc5c6ccccc6[se]c5c4c3c2)cc1 ; c1ccc(-c2ccc(N(c3ccc4oc5ccccc5c4c3)c3ccc4ccc5ccc6c7ccccc7[se]c6c5c4c3)cc2)cc1]

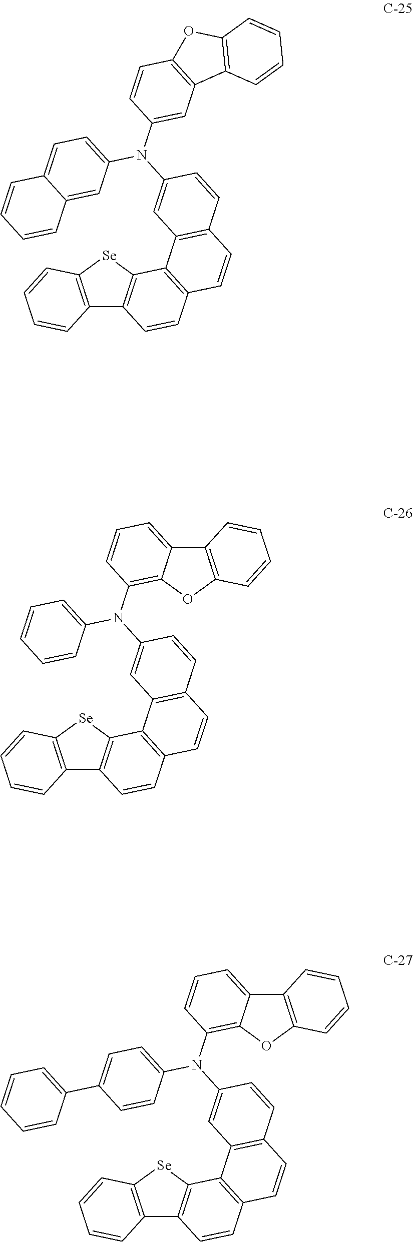 [SMILES: c1ccc2cc(N(c3ccc4oc5ccccc5c4c3)c3ccc4ccc5ccc6c7ccccc7[se]c6c5c4c3)ccc2c1 ; c1ccc(N(c2ccc3ccc4ccc5c6ccccc6[se]c5c4c3c2)c2cccc3c2oc2ccccc23)cc1 ; c1ccc(-c2ccc(N(c3ccc4ccc5ccc6c7ccccc7[se]c6c5c4c3)c3cccc4c3oc3ccccc34)cc2)cc1]

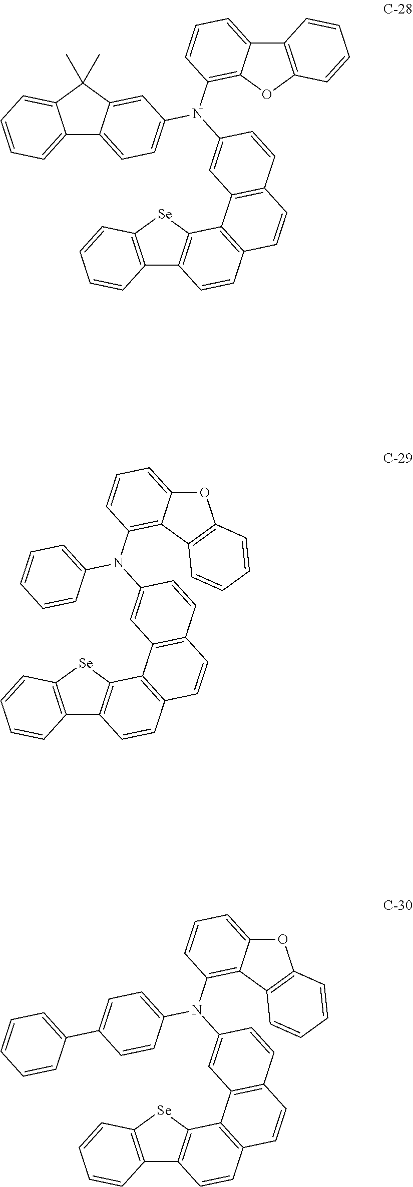 [SMILES: CC1(C)c2ccccc2-c2ccc(N(c3ccc4ccc5ccc6c7ccccc7[se]c6c5c4c3)c3cccc4c3oc3ccccc34)cc21 ; c1ccc(N(c2ccc3ccc4ccc5c6ccccc6[se]c5c4c3c2)c2cccc3oc4ccccc4c23)cc1 ; c1ccc(-c2ccc(N(c3ccc4ccc5ccc6c7ccccc7[se]c6c5c4c3)c3cccc4oc5ccccc5c34)cc2)cc1]

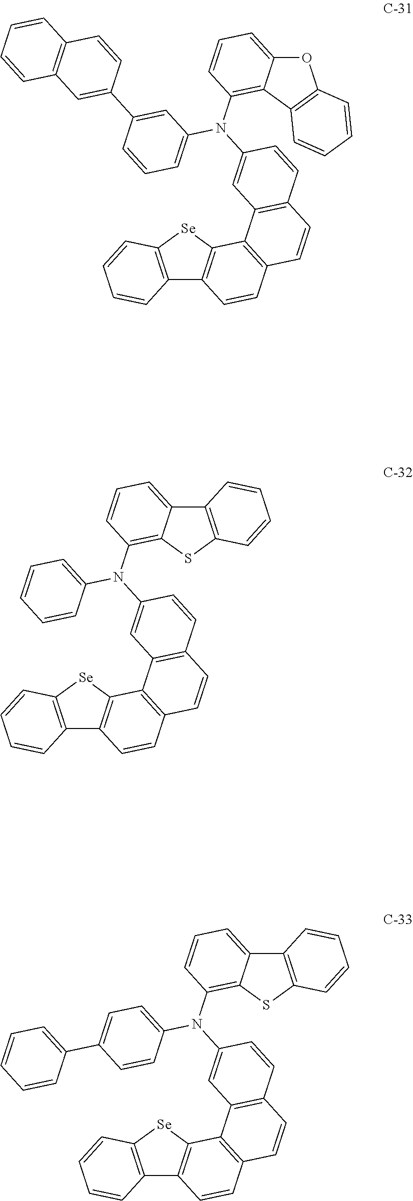 [SMILES: c1cc(-c2ccc3ccccc3c2)cc(N(c2ccc3ccc4ccc5c6ccccc6[se]c5c4c3c2)c2cccc3oc4ccccc4c23)c1 ; c1ccc(N(c2ccc3ccc4ccc5c6ccccc6[se]c5c4c3c2)c2cccc3c2sc2ccccc23)cc1 ; c1ccc(-c2ccc(N(c3ccc4ccc5ccc6c7ccccc7[se]c6c5c4c3)c3cccc4c3sc3ccccc34)cc2)cc1]

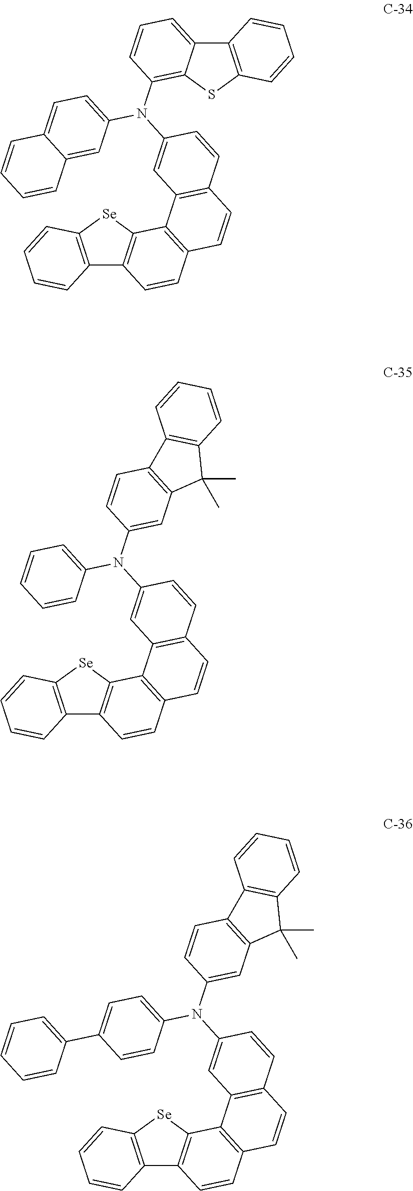 [SMILES: c1ccc2cc(N(c3ccc4ccc5ccc6c7ccccc7[se]c6c5c4c3)c3cccc4c3sc3ccccc34)ccc2c1 ; CC1(C)c2ccccc2-c2ccc(N(c3ccccc3)c3ccc4ccc5ccc6c7ccccc7[se]c6c5c4c3)cc21 ; CC1(C)c2ccccc2-c2ccc(N(c3ccc(-c4ccccc4)cc3)c3ccc4ccc5ccc6c7ccccc7[se]c6c5c4c3)cc21]

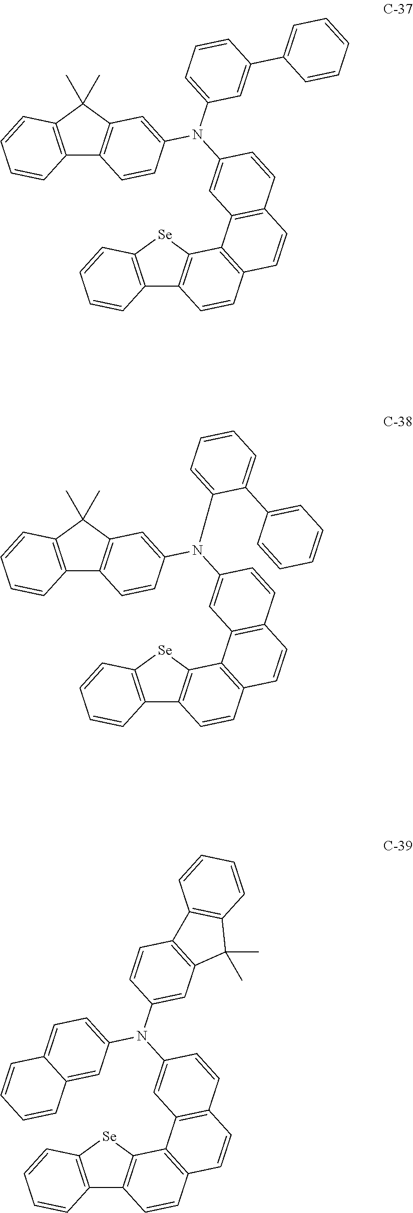 [SMILES: CC1(C)c2ccccc2-c2ccc(N(c3cccc(-c4ccccc4)c3)c3ccc4ccc5ccc6c7ccccc7[se]c6c5c4c3)cc21 ; CC1(C)c2ccccc2-c2ccc(N(c3ccc4ccc5ccc6c7ccccc7[se]c6c5c4c3)c3ccccc3-c3ccccc3)cc21 ; CC1(C)c2ccccc2-c2ccc(N(c3ccc4ccccc4c3)c3ccc4ccc5ccc6c7ccccc7[se]c6c5c4c3)cc21]

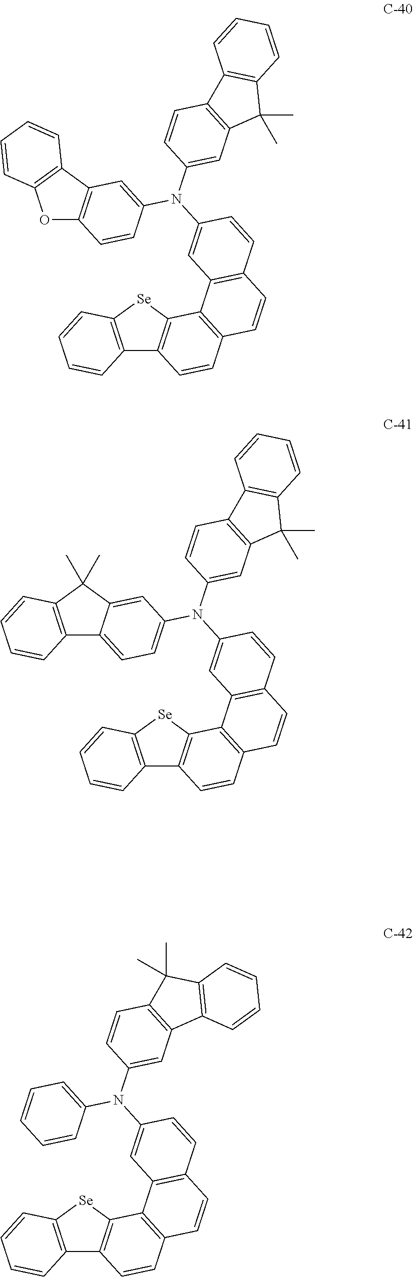 [SMILES: CC1(C)c2ccccc2-c2ccc(N(c3ccc4oc5ccccc5c4c3)c3ccc4ccc5ccc6c7ccccc7[se]c6c5c4c3)cc21 ; CC1(C)c2ccccc2-c2ccc(N(c3ccc4c(c3)C(C)(C)c3ccccc3-4)c3ccc4ccc5ccc6c7ccccc7[se]c6c5c4c3)cc21 ; CC1(C)c2ccccc2-c2cc(N(c3ccccc3)c3ccc4ccc5ccc6c7ccccc7[se]c6c5c4c3)ccc21]

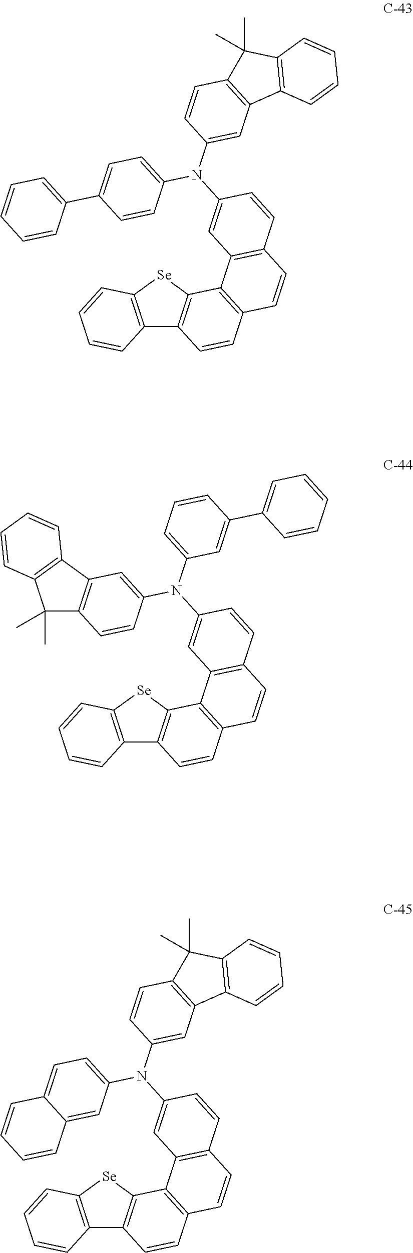 [SMILES: CC1(C)c2ccccc2-c2cc(N(c3ccc(-c4ccccc4)cc3)c3ccc4ccc5ccc6c7ccccc7[se]c6c5c4c3)ccc21 ; CC1(C)c2ccccc2-c2cc(N(c3cccc(-c4ccccc4)c3)c3ccc4ccc5ccc6c7ccccc7[se]c6c5c4c3)ccc21 ; CC1(C)c2ccccc2-c2cc(N(c3ccc4ccccc4c3)c3ccc4ccc5ccc6c7ccccc7[se]c6c5c4c3)ccc21]

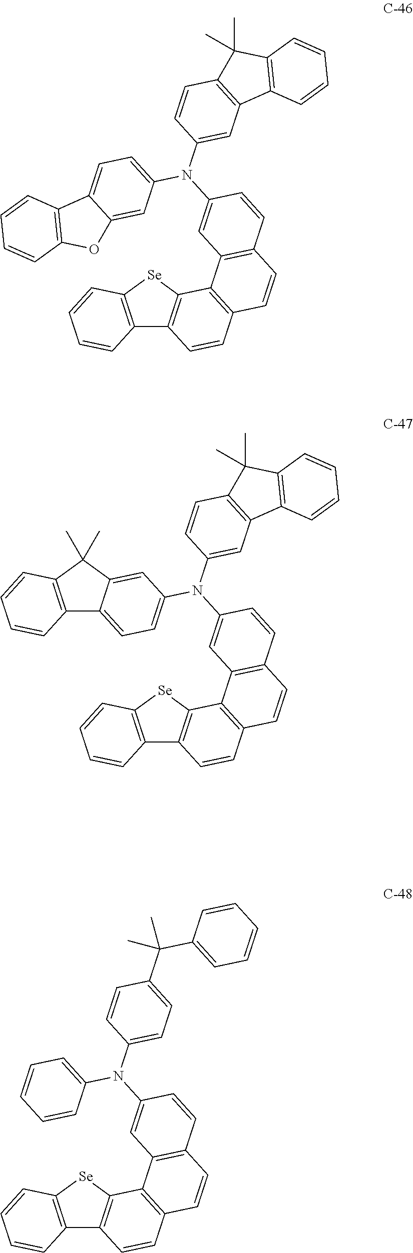 [SMILES: CC1(C)c2ccccc2-c2cc(N(c3ccc4c(c3)oc3ccccc34)c3ccc4ccc5ccc6c7ccccc7[se]c6c5c4c3)ccc21 ; CC1(C)c2ccccc2-c2cc(N(c3ccc4c(c3)C(C)(C)c3ccccc3-4)c3ccc4ccc5ccc6c7ccccc7[se]c6c5c4c3)ccc21 ; CC(C)(c1ccccc1)c1ccc(N(c2ccccc2)c2ccc3ccc4ccc5c6ccccc6[se]c5c4c3c2)cc1]

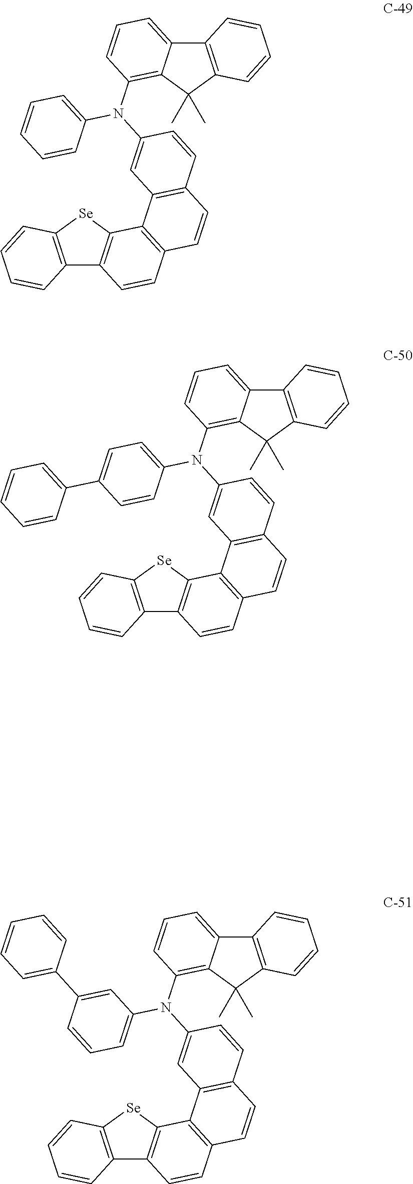 [SMILES: CC1(C)c2ccccc2-c2cccc(N(c3ccccc3)c3ccc4ccc5ccc6c7ccccc7[se]c6c5c4c3)c21 ; CC1(C)c2ccccc2-c2cccc(N(c3ccc(-c4ccccc4)cc3)c3ccc4ccc5ccc6c7ccccc7[se]c6c5c4c3)c21 ; CC1(C)c2ccccc2-c2cccc(N(c3cccc(-c4ccccc4)c3)c3ccc4ccc5ccc6c7ccccc7[se]c6c5c4c3)c21]

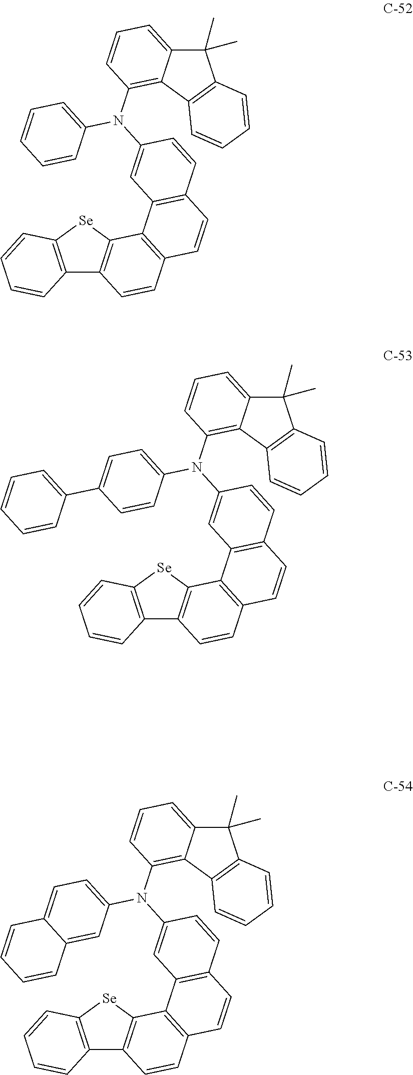 [SMILES: CC1(C)c2ccccc2-c2c(N(c3ccccc3)c3ccc4ccc5ccc6c7ccccc7[se]c6c5c4c3)cccc21 ; CC1(C)c2ccccc2-c2c(N(c3ccc(-c4ccccc4)cc3)c3ccc4ccc5ccc6c7ccccc7[se]c6c5c4c3)cccc21 ; CC1(C)c2ccccc2-c2c(N(c3ccc4ccccc4c3)c3ccc4ccc5ccc6c7ccccc7[se]c6c5c4c3)cccc21]

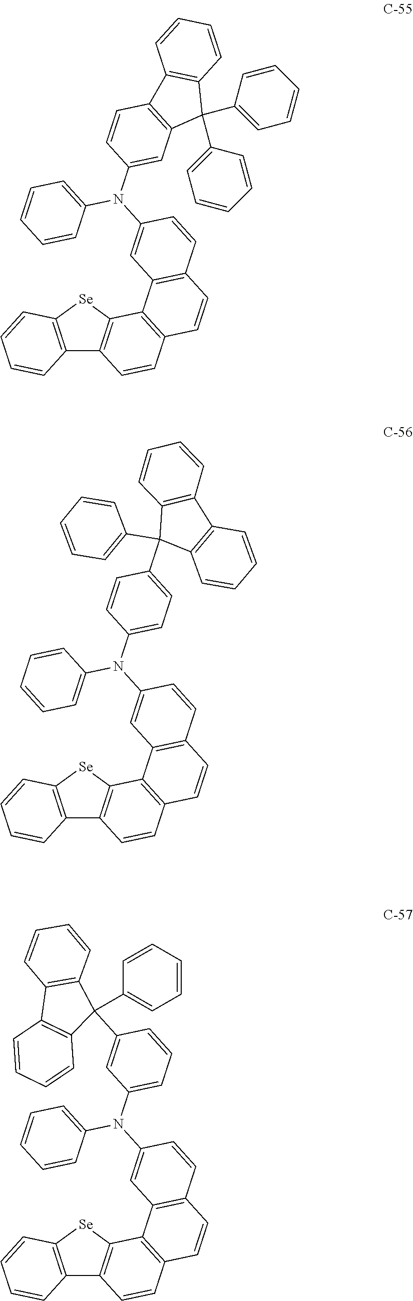 [SMILES: c1ccc(N(c2ccc3c(c2)C(c2ccccc2)(c2ccccc2)c2ccccc2-3)c2ccc3ccc4ccc5c6ccccc6[se]c5c4c3c2)cc1 ; c1ccc(N(c2ccc(C3(c4ccccc4)c4ccccc4-c4ccccc43)cc2)c2ccc3ccc4ccc5c6ccccc6[se]c5c4c3c2)cc1 ; c1ccc(N(c2cccc(C3(c4ccccc4)c4ccccc4-c4ccccc43)c2)c2ccc3ccc4ccc5c6ccccc6[se]c5c4c3c2)cc1]

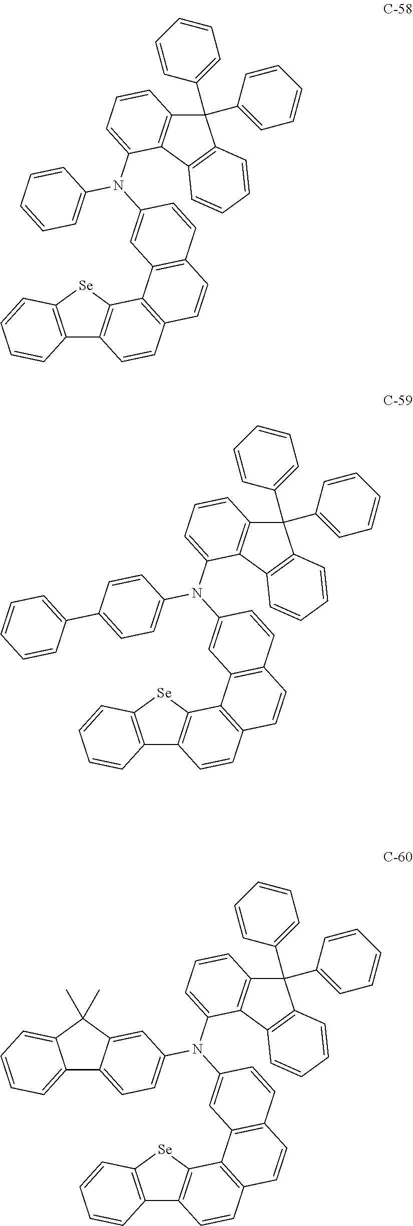 [SMILES: c1ccc(N(c2ccc3ccc4ccc5c6ccccc6[se]c5c4c3c2)c2cccc3c2-c2ccccc2C3(c2ccccc2)c2ccccc2)cc1 ; c1ccc(-c2ccc(N(c3ccc4ccc5ccc6c7ccccc7[se]c6c5c4c3)c3cccc4c3-c3ccccc3C4(c3ccccc3)c3ccccc3)cc2)cc1 ; CC1(C)c2ccccc2-c2ccc(N(c3ccc4ccc5ccc6c7ccccc7[se]c6c5c4c3)c3cccc4c3-c3ccccc3C4(c3ccccc3)c3ccccc3)cc21]

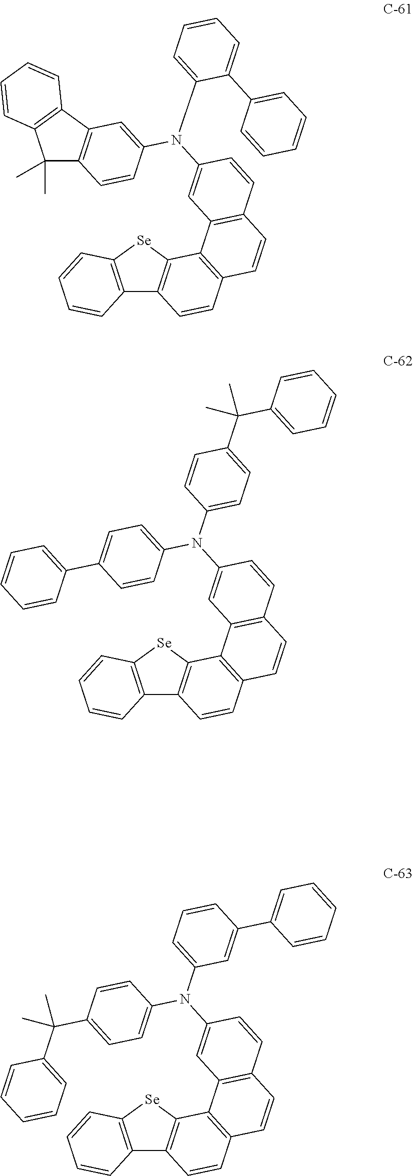 [SMILES: CC1(C)c2ccccc2-c2cc(N(c3ccc4ccc5ccc6c7ccccc7[se]c6c5c4c3)c3ccccc3-c3ccccc3)ccc21 ; CC(C)(c1ccccc1)c1ccc(N(c2ccc(-c3ccccc3)cc2)c2ccc3ccc4ccc5c6ccccc6[se]c5c4c3c2)cc1 ; CC(C)(c1ccccc1)c1ccc(N(c2cccc(-c3ccccc3)c2)c2ccc3ccc4ccc5c6ccccc6[se]c5c4c3c2)cc1]

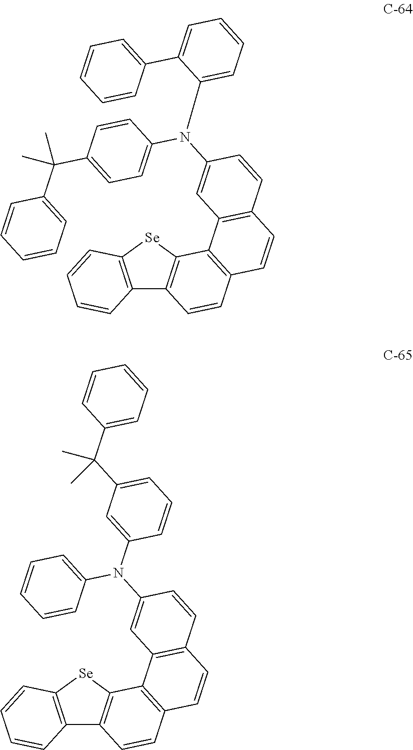 [SMILES: CC(C)(c1ccccc1)c1ccc(N(c2ccc3ccc4ccc5c6ccccc6[se]c5c4c3c2)c2ccccc2-c2ccccc2)cc1 ; CC(C)(c1ccccc1)c1cccc(N(c2ccccc2)c2ccc3ccc4ccc5c6ccccc6[se]c5c4c3c2)c1]

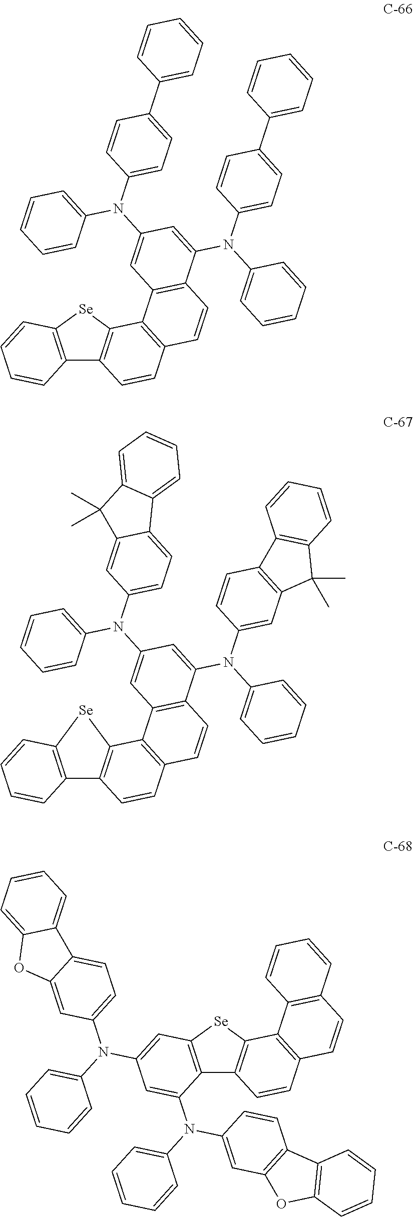 [SMILES: c1ccc(-c2ccc(N(c3ccccc3)c3cc(N(c4ccccc4)c4ccc(-c5ccccc5)cc4)c4ccc5ccc6c7ccccc7[se]c6c5c4c3)cc2)cc1 ; CC1(C)c2ccccc2-c2ccc(N(c3ccccc3)c3cc(N(c4ccccc4)c4ccc5c(c4)C(C)(C)c4ccccc4-5)c4ccc5ccc6c7ccccc7[se]c6c5c4c3)cc21 ; c1ccc(N(c2ccc3c(c2)oc2ccccc23)c2cc(N(c3ccccc3)c3ccc4c(c3)oc3ccccc34)c3c(c2)[se]c2c3ccc3ccc4ccccc4c32)cc1]

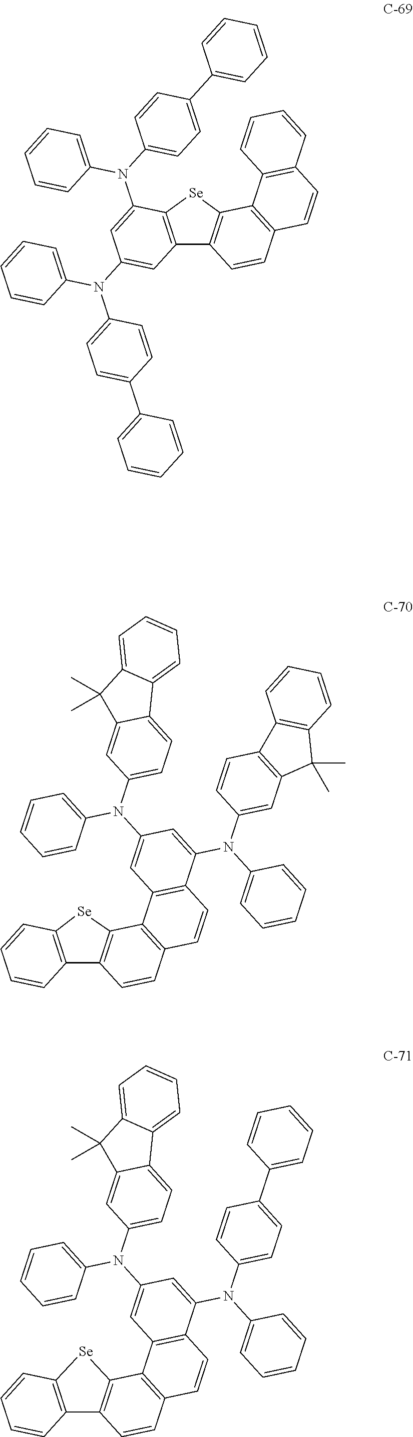 [SMILES: c1ccc(-c2ccc(N(c3ccccc3)c3cc(N(c4ccccc4)c4ccc(-c5ccccc5)cc4)c4[se]c5c(ccc6ccc7ccccc7c65)c4c3)cc2)cc1 ; CC1(C)c2ccccc2-c2ccc(N(c3ccccc3)c3cc(N(c4ccccc4)c4ccc5c(c4)C(C)(C)c4ccccc4-5)c4ccc5ccc6c7ccccc7[se]c6c5c4c3)cc21 ; CC1(C)c2ccccc2-c2ccc(N(c3ccccc3)c3cc(N(c4ccccc4)c4ccc(-c5ccccc5)cc4)c4ccc5ccc6c7ccccc7[se]c6c5c4c3)cc21]

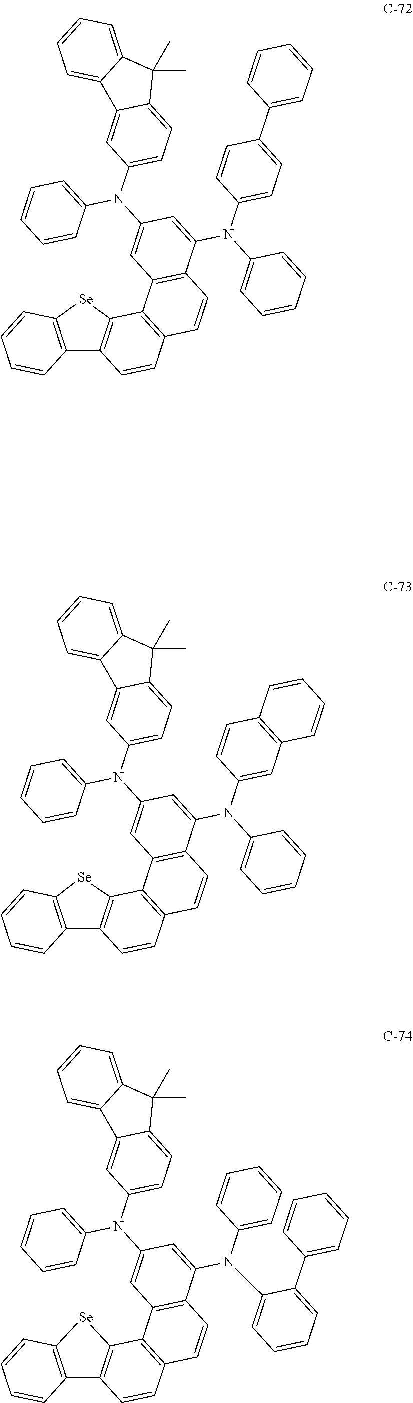 [SMILES: CC1(C)c2ccccc2-c2cc(N(c3ccccc3)c3cc(N(c4ccccc4)c4ccc(-c5ccccc5)cc4)c4ccc5ccc6c7ccccc7[se]c6c5c4c3)ccc21 ; CC1(C)c2ccccc2-c2cc(N(c3ccccc3)c3cc(N(c4ccccc4)c4ccc5ccccc5c4)c4ccc5ccc6c7ccccc7[se]c6c5c4c3)ccc21 ; CC1(C)c2ccccc2-c2cc(N(c3ccccc3)c3cc(N(c4ccccc4)c4ccccc4-c4ccccc4)c4ccc5ccc6c7ccccc7[se]c6c5c4c3)ccc21]

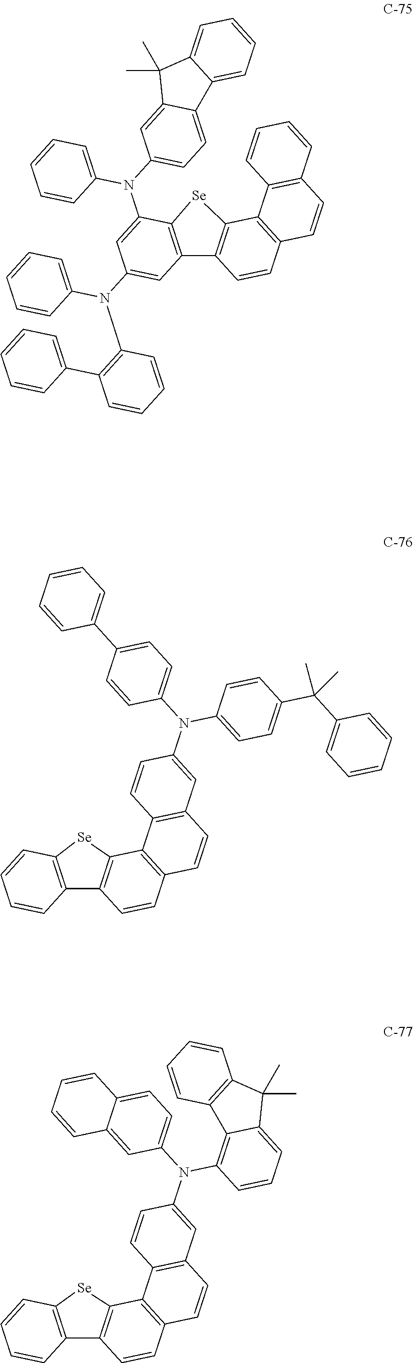 [SMILES: CC1(C)c2ccccc2-c2ccc(N(c3ccccc3)c3cc(N(c4ccccc4)c4ccccc4-c4ccccc4)cc4c3[se]c3c4ccc4ccc5ccccc5c43)cc21 ; CC(C)(c1ccccc1)c1ccc(N(c2ccc(-c3ccccc3)cc2)c2ccc3c(ccc4ccc5c6ccccc6[se]c5c43)c2)cc1 ; CC1(C)c2ccccc2-c2c(N(c3ccc4ccccc4c3)c3ccc4c(ccc5ccc6c7ccccc7[se]c6c54)c3)cccc21]

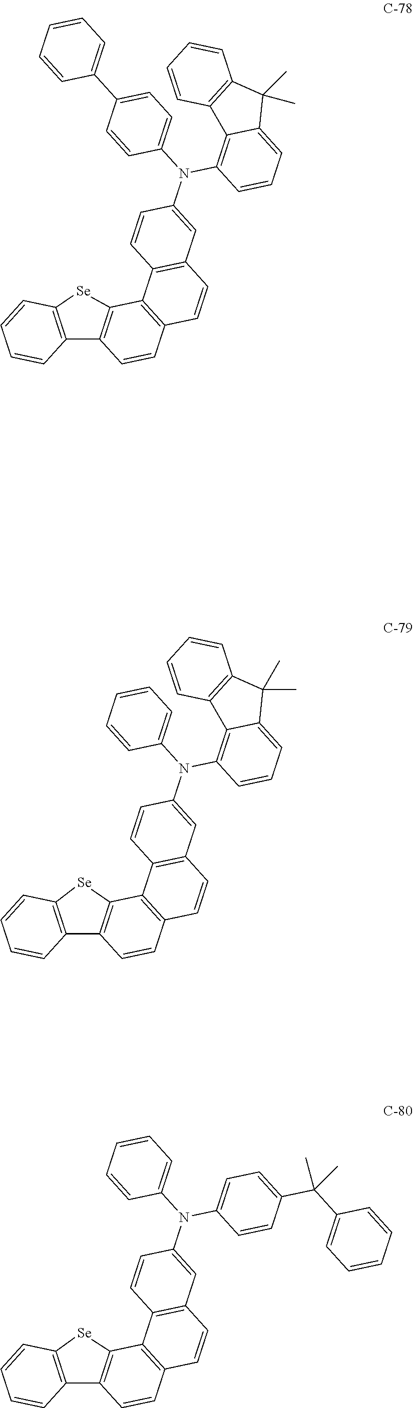 [SMILES: CC1(C)c2ccccc2-c2c(N(c3ccc(-c4ccccc4)cc3)c3ccc4c(ccc5ccc6c7ccccc7[se]c6c54)c3)cccc21 ; CC1(C)c2ccccc2-c2c(N(c3ccccc3)c3ccc4c(ccc5ccc6c7ccccc7[se]c6c54)c3)cccc21 ; CC(C)(c1ccccc1)c1ccc(N(c2ccccc2)c2ccc3c(ccc4ccc5c6ccccc6[se]c5c43)c2)cc1]

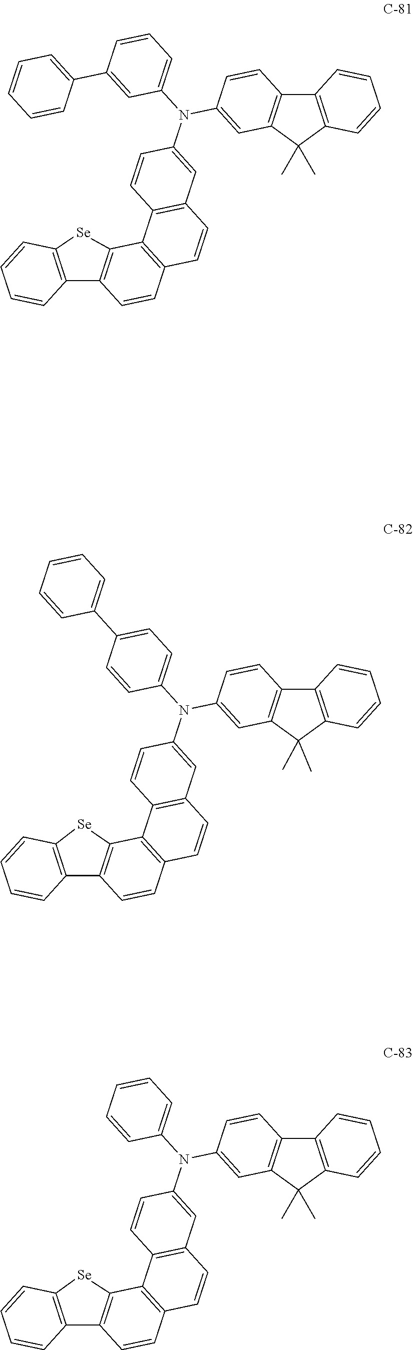 [SMILES: CC1(C)c2ccccc2-c2ccc(N(c3cccc(-c4ccccc4)c3)c3ccc4c(ccc5ccc6c7ccccc7[se]c6c54)c3)cc21 ; CC1(C)c2ccccc2-c2ccc(N(c3ccc(-c4ccccc4)cc3)c3ccc4c(ccc5ccc6c7ccccc7[se]c6c54)c3)cc21 ; CC1(C)c2ccccc2-c2ccc(N(c3ccccc3)c3ccc4c(ccc5ccc6c7ccccc7[se]c6c54)c3)cc21]

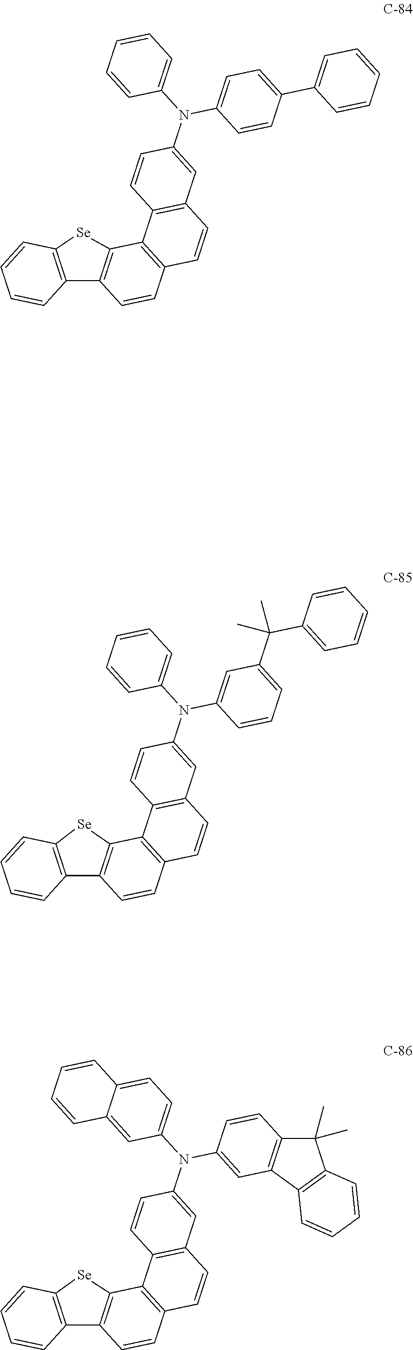 [SMILES: c1ccc(-c2ccc(N(c3ccccc3)c3ccc4c(ccc5ccc6c7ccccc7[se]c6c54)c3)cc2)cc1 ; CC(C)(c1ccccc1)c1cccc(N(c2ccccc2)c2ccc3c(ccc4ccc5c6ccccc6[se]c5c43)c2)c1 ; CC1(C)c2ccccc2-c2cc(N(c3ccc4ccccc4c3)c3ccc4c(ccc5ccc6c7ccccc7[se]c6c54)c3)ccc21]

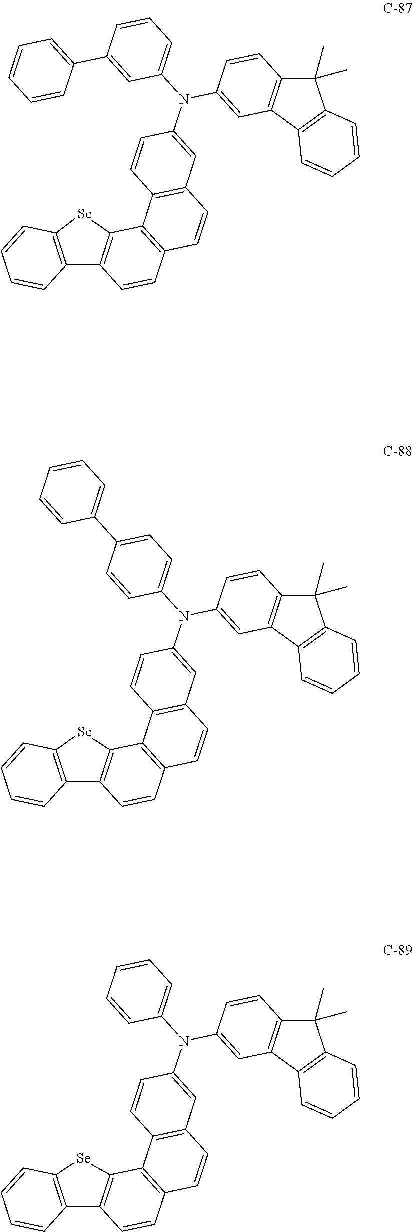 [SMILES: CC1(C)c2ccccc2-c2cc(N(c3cccc(-c4ccccc4)c3)c3ccc4c(ccc5ccc6c7ccccc7[se]c6c54)c3)ccc21 ; CC1(C)c2ccccc2-c2cc(N(c3ccc(-c4ccccc4)cc3)c3ccc4c(ccc5ccc6c7ccccc7[se]c6c54)c3)ccc21 ; CC1(C)c2ccccc2-c2cc(N(c3ccccc3)c3ccc4c(ccc5ccc6c7ccccc7[se]c6c54)c3)ccc21]

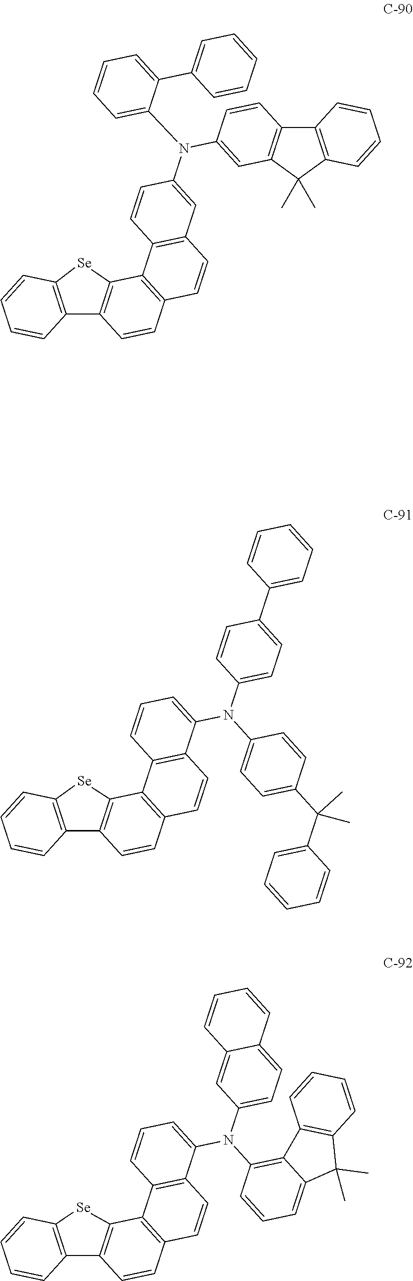 [SMILES: CC1(C)c2ccccc2-c2ccc(N(c3ccc4c(ccc5ccc6c7ccccc7[se]c6c54)c3)c3ccccc3-c3ccccc3)cc21 ; CC(C)(c1ccccc1)c1ccc(N(c2ccc(-c3ccccc3)cc2)c2cccc3c2ccc2ccc4c5ccccc5[se]c4c23)cc1 ; CC1(C)c2ccccc2-c2c(N(c3ccc4ccccc4c3)c3cccc4c3ccc3ccc5c6ccccc6[se]c5c34)cccc21]

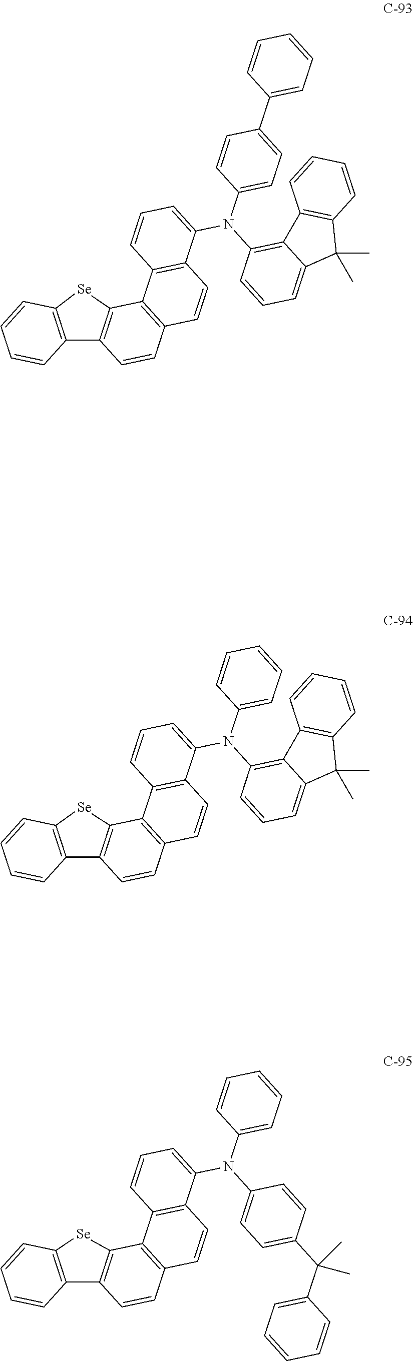 [SMILES: CC1(C)c2ccccc2-c2c(N(c3ccc(-c4ccccc4)cc3)c3cccc4c3ccc3ccc5c6ccccc6[se]c5c34)cccc21 ; CC1(C)c2ccccc2-c2c(N(c3ccccc3)c3cccc4c3ccc3ccc5c6ccccc6[se]c5c34)cccc21 ; CC(C)(c1ccccc1)c1ccc(N(c2ccccc2)c2cccc3c2ccc2ccc4c5ccccc5[se]c4c23)cc1]

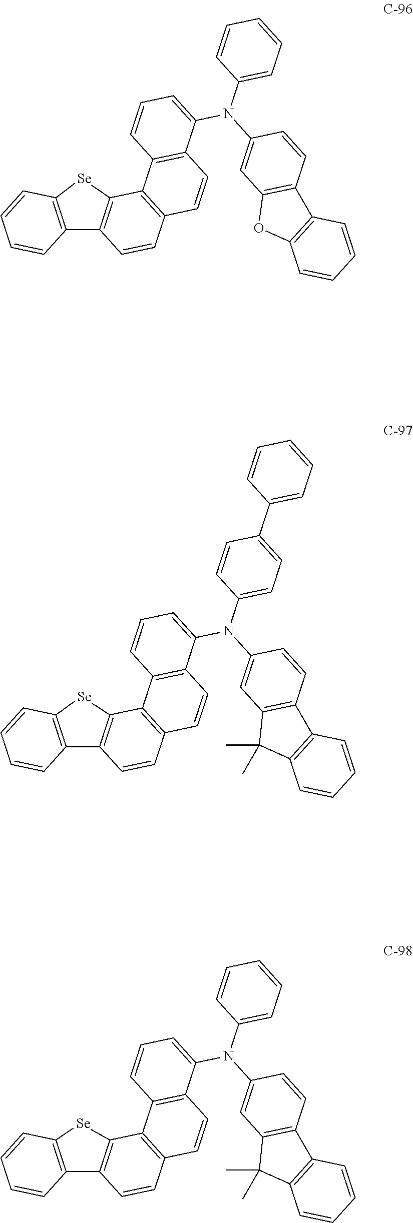 [SMILES: c1ccc(N(c2ccc3c(c2)oc2ccccc23)c2cccc3c2ccc2ccc4c5ccccc5[se]c4c23)cc1 ; CC1(C)c2ccccc2-c2ccc(N(c3ccc(-c4ccccc4)cc3)c3cccc4c3ccc3ccc5c6ccccc6[se]c5c34)cc21 ; CC1(C)c2ccccc2-c2ccc(N(c3ccccc3)c3cccc4c3ccc3ccc5c6ccccc6[se]c5c34)cc21]

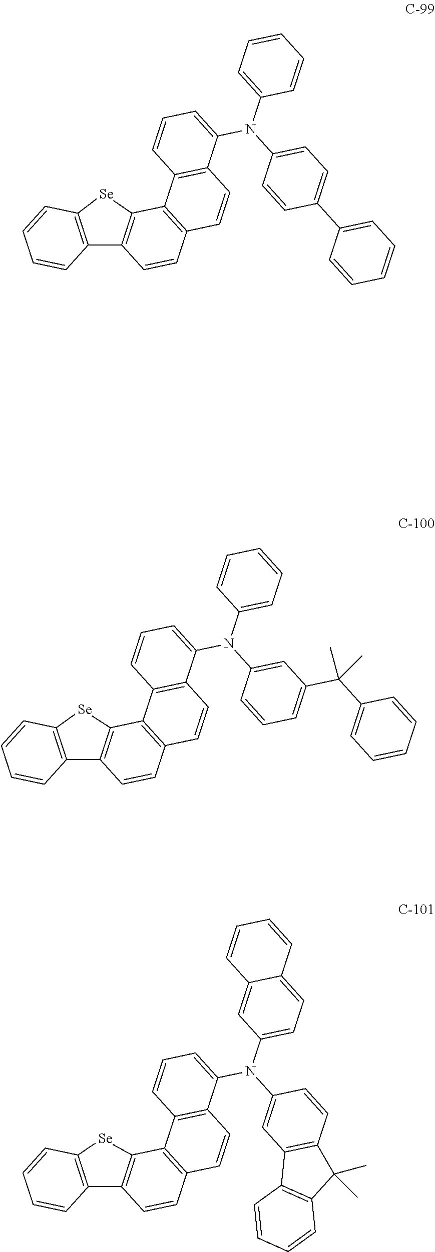 [SMILES: c1ccc(-c2ccc(N(c3ccccc3)c3cccc4c3ccc3ccc5c6ccccc6[se]c5c34)cc2)cc1 ; CC(C)(c1ccccc1)c1cccc(N(c2ccccc2)c2cccc3c2ccc2ccc4c5ccccc5[se]c4c23)c1 ; CC1(C)c2ccccc2-c2cc(N(c3ccc4ccccc4c3)c3cccc4c3ccc3ccc5c6ccccc6[se]c5c34)ccc21]

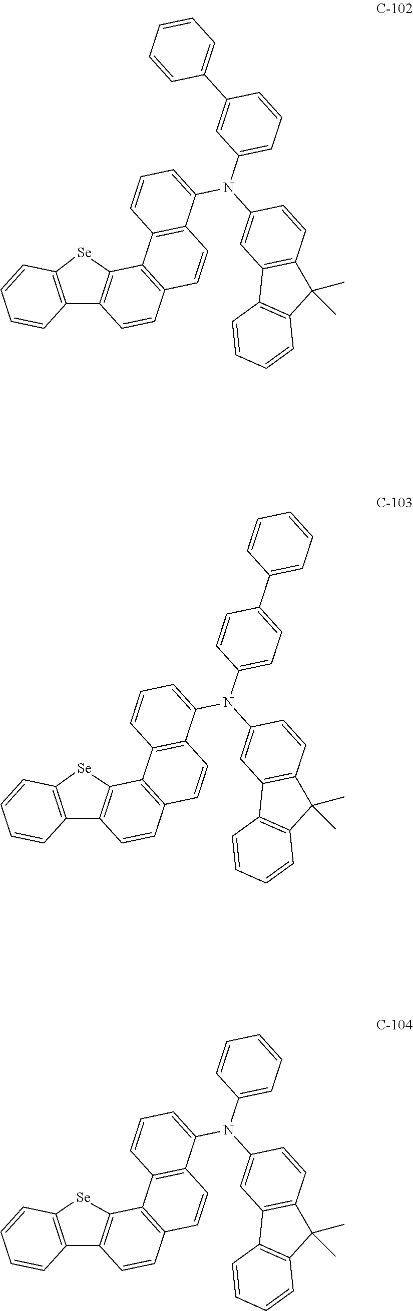 [SMILES: CC1(C)c2ccccc2-c2cc(N(c3cccc(-c4ccccc4)c3)c3cccc4c3ccc3ccc5c6ccccc6[se]c5c34)ccc21 ; CC1(C)c2ccccc2-c2cc(N(c3ccc(-c4ccccc4)cc3)c3cccc4c3ccc3ccc5c6ccccc6[se]c5c34)ccc21 ; CC1(C)c2ccccc2-c2cc(N(c3ccccc3)c3cccc4c3ccc3ccc5c6ccccc6[se]c5c34)ccc21]

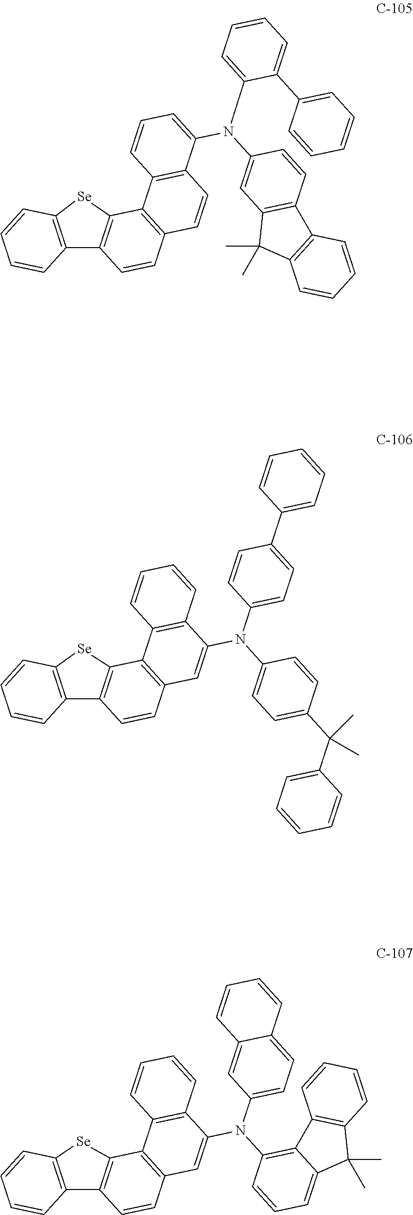 [SMILES: CC1(C)c2ccccc2-c2ccc(N(c3ccccc3-c3ccccc3)c3cccc4c3ccc3ccc5c6ccccc6[se]c5c34)cc21 ; CC(C)(c1ccccc1)c1ccc(N(c2ccc(-c3ccccc3)cc2)c2cc3ccc4c5ccccc5[se]c4c3c3ccccc23)cc1 ; CC1(C)c2ccccc2-c2c(N(c3ccc4ccccc4c3)c3cc4ccc5c6ccccc6[se]c5c4c4ccccc34)cccc21]

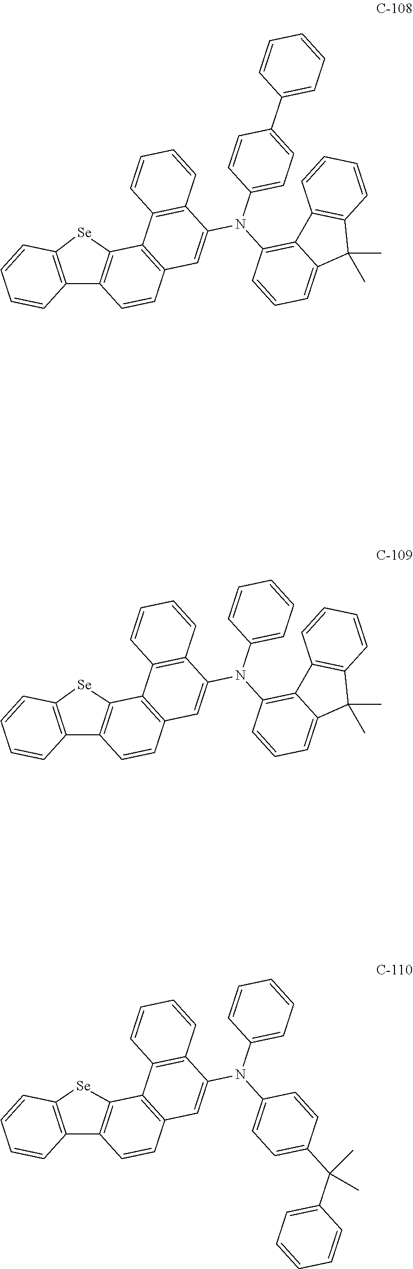 [SMILES: CC1(C)c2ccccc2-c2c(N(c3ccc(-c4ccccc4)cc3)c3cc4ccc5c6ccccc6[se]c5c4c4ccccc34)cccc21 ; CC1(C)c2ccccc2-c2c(N(c3ccccc3)c3cc4ccc5c6ccccc6[se]c5c4c4ccccc34)cccc21 ; CC(C)(c1ccccc1)c1ccc(N(c2ccccc2)c2cc3ccc4c5ccccc5[se]c4c3c3ccccc23)cc1]

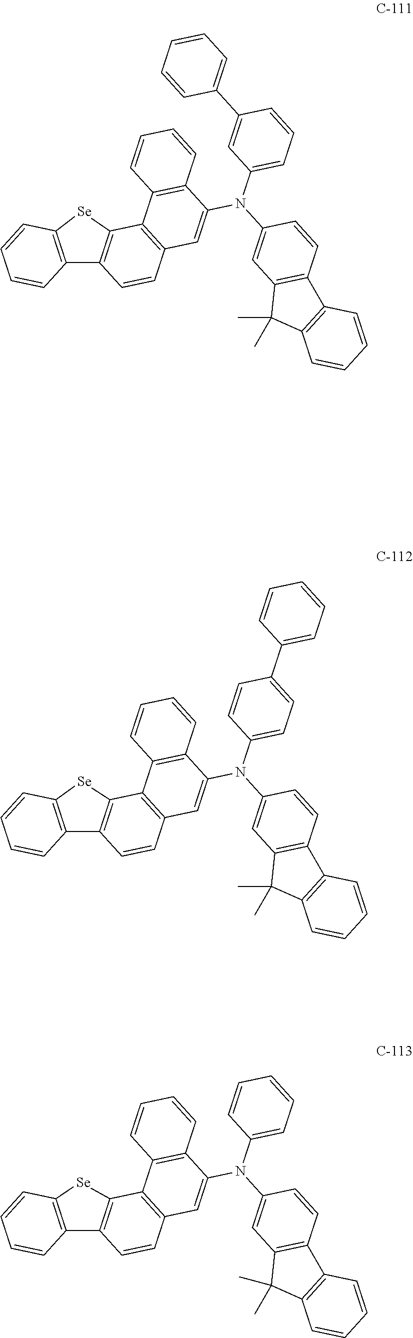 [SMILES: CC1(C)c2ccccc2-c2ccc(N(c3cccc(-c4ccccc4)c3)c3cc4ccc5c6ccccc6[se]c5c4c4ccccc34)cc21 ; CC1(C)c2ccccc2-c2ccc(N(c3ccc(-c4ccccc4)cc3)c3cc4ccc5c6ccccc6[se]c5c4c4ccccc34)cc21 ; CC1(C)c2ccccc2-c2ccc(N(c3ccccc3)c3cc4ccc5c6ccccc6[se]c5c4c4ccccc34)cc21]

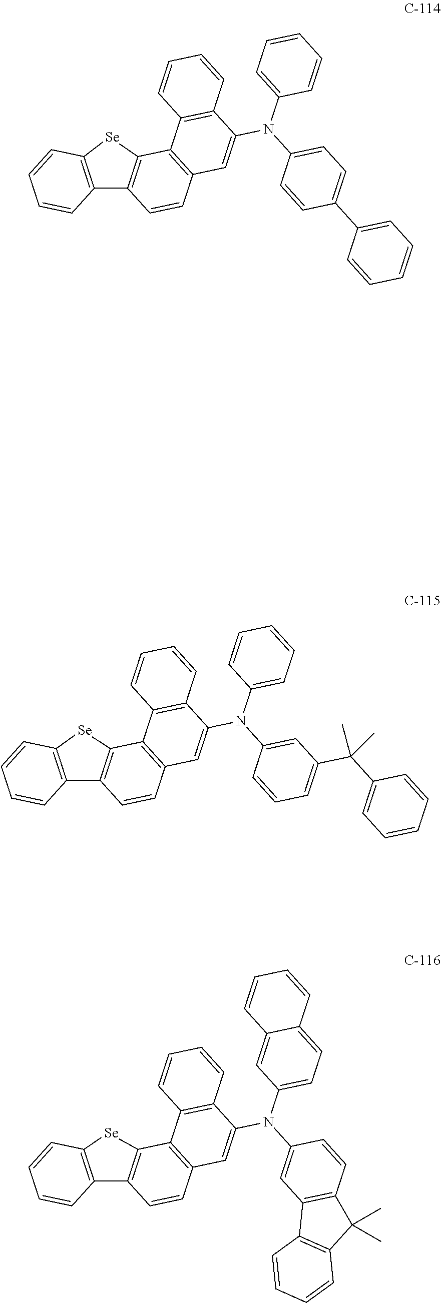 [SMILES: c1ccc(-c2ccc(N(c3ccccc3)c3cc4ccc5c6ccccc6[se]c5c4c4ccccc34)cc2)cc1 ; CC(C)(c1ccccc1)c1cccc(N(c2ccccc2)c2cc3ccc4c5ccccc5[se]c4c3c3ccccc23)c1 ; CC1(C)c2ccccc2-c2cc(N(c3ccc4ccccc4c3)c3cc4ccc5c6ccccc6[se]c5c4c4ccccc34)ccc21]

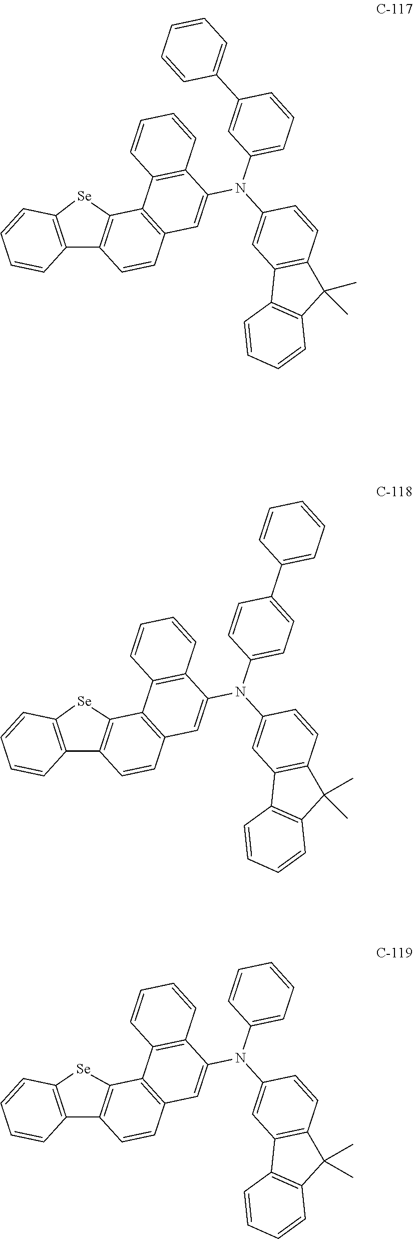 [SMILES: CC1(C)c2ccccc2-c2cc(N(c3cccc(-c4ccccc4)c3)c3cc4ccc5c6ccccc6[se]c5c4c4ccccc34)ccc21 ; CC1(C)c2ccccc2-c2cc(N(c3ccc(-c4ccccc4)cc3)c3cc4ccc5c6ccccc6[se]c5c4c4ccccc34)ccc21 ; CC1(C)c2ccccc2-c2cc(N(c3ccccc3)c3cc4ccc5c6ccccc6[se]c5c4c4ccccc34)ccc21]

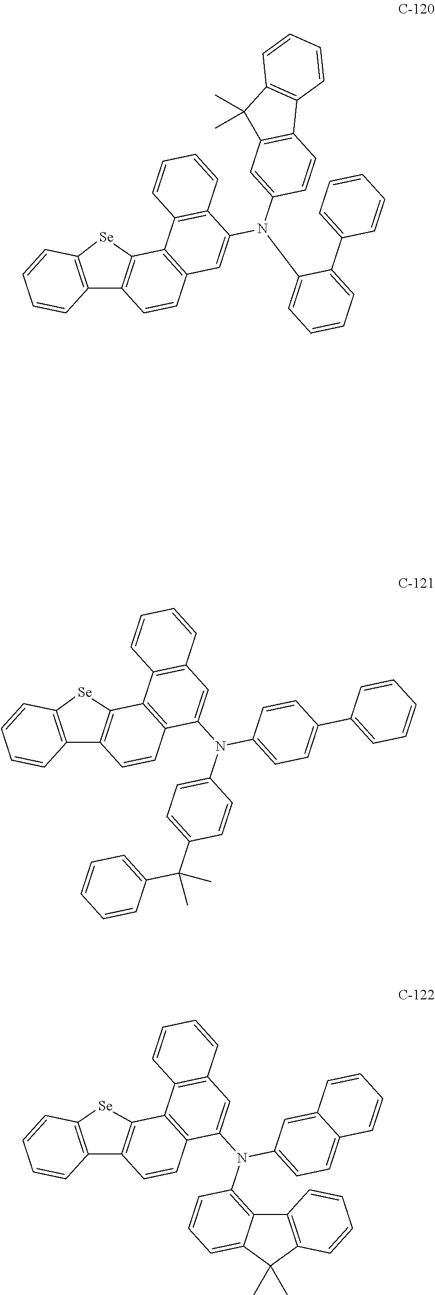 [SMILES: CC1(C)c2ccccc2-c2ccc(N(c3ccccc3-c3ccccc3)c3cc4ccc5c6ccccc6[se]c5c4c4ccccc34)cc21 ; CC(C)(c1ccccc1)c1ccc(N(c2ccc(-c3ccccc3)cc2)c2cc3ccccc3c3c2ccc2c4ccccc4[se]c23)cc1 ; CC1(C)c2ccccc2-c2c(N(c3ccc4ccccc4c3)c3cc4ccccc4c4c3ccc3c5ccccc5[se]c34)cccc21]

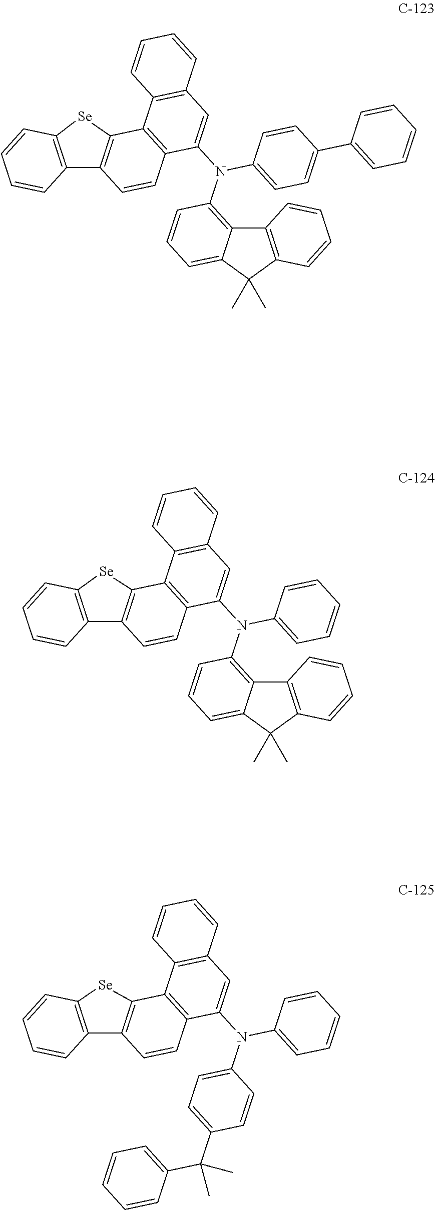 [SMILES: CC1(C)c2ccccc2-c2c(N(c3ccc(-c4ccccc4)cc3)c3cc4ccccc4c4c3ccc3c5ccccc5[se]c34)cccc21 ; CC1(C)c2ccccc2-c2c(N(c3ccccc3)c3cc4ccccc4c4c3ccc3c5ccccc5[se]c34)cccc21 ; CC(C)(c1ccccc1)c1ccc(N(c2ccccc2)c2cc3ccccc3c3c2ccc2c4ccccc4[se]c23)cc1]

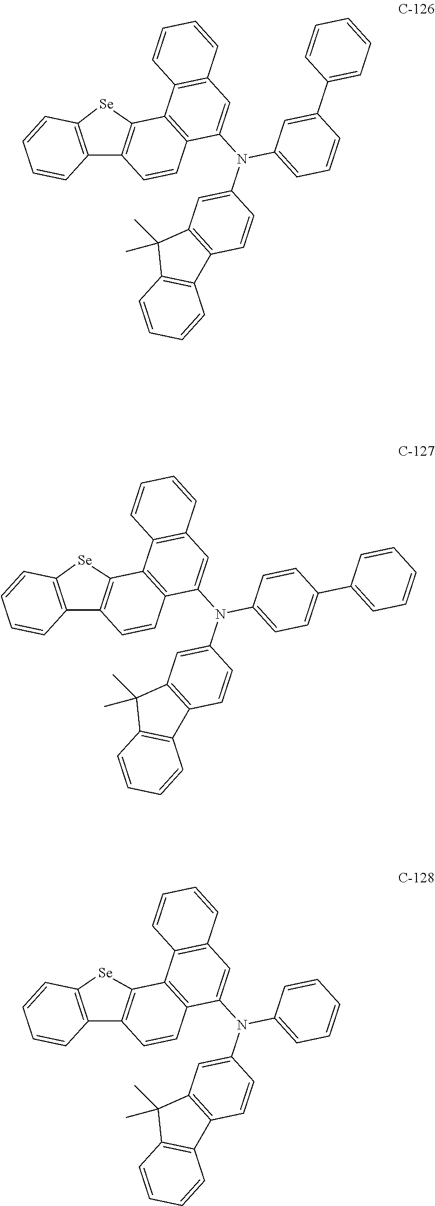 [SMILES: CC1(C)c2ccccc2-c2ccc(N(c3cccc(-c4ccccc4)c3)c3cc4ccccc4c4c3ccc3c5ccccc5[se]c34)cc21 ; CC1(C)c2ccccc2-c2ccc(N(c3ccc(-c4ccccc4)cc3)c3cc4ccccc4c4c3ccc3c5ccccc5[se]c34)cc21 ; CC1(C)c2ccccc2-c2ccc(N(c3ccccc3)c3cc4ccccc4c4c3ccc3c5ccccc5[se]c34)cc21]

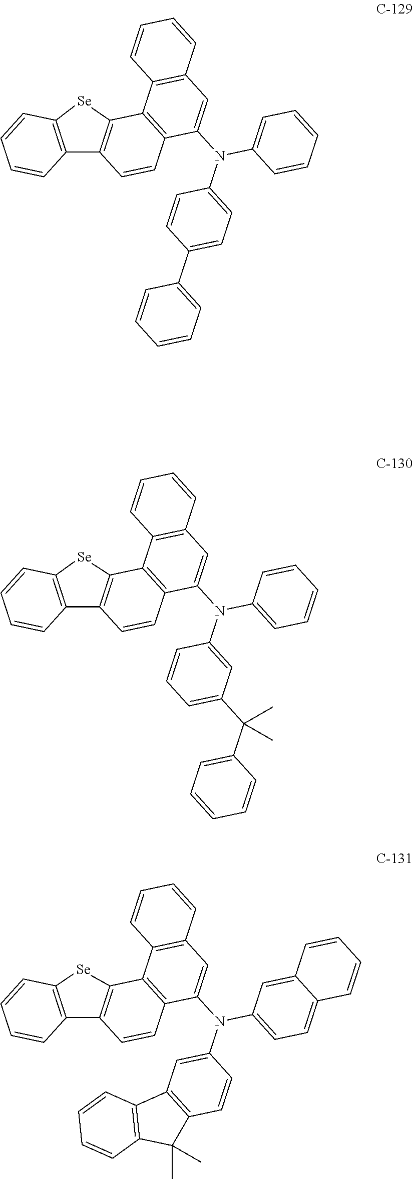 [SMILES: c1ccc(-c2ccc(N(c3ccccc3)c3cc4ccccc4c4c3ccc3c5ccccc5[se]c34)cc2)cc1 ; CC(C)(c1ccccc1)c1cccc(N(c2ccccc2)c2cc3ccccc3c3c2ccc2c4ccccc4[se]c23)c1 ; CC1(C)c2ccccc2-c2cc(N(c3ccc4ccccc4c3)c3cc4ccccc4c4c3ccc3c5ccccc5[se]c34)ccc21]

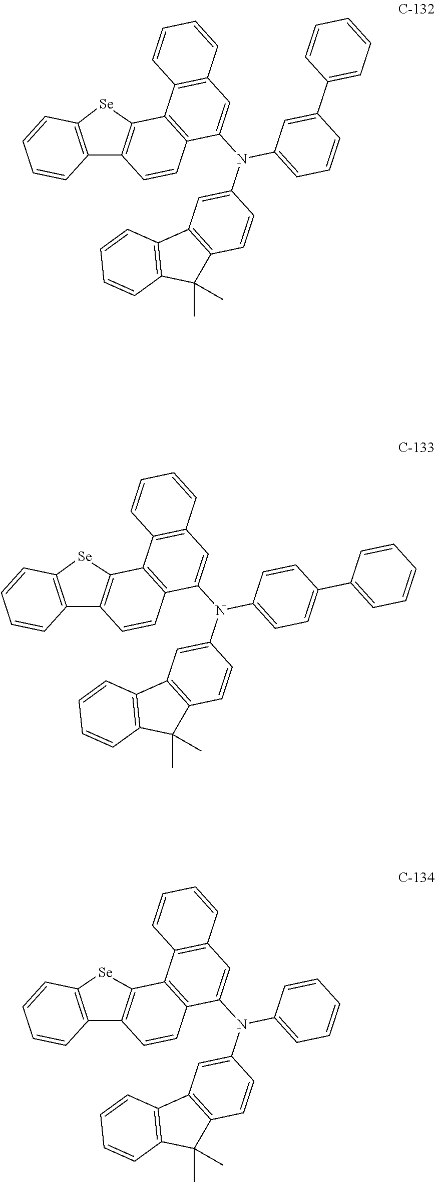 [SMILES: CC1(C)c2ccccc2-c2cc(N(c3cccc(-c4ccccc4)c3)c3cc4ccccc4c4c3ccc3c5ccccc5[se]c34)ccc21 ; CC1(C)c2ccccc2-c2cc(N(c3ccc(-c4ccccc4)cc3)c3cc4ccccc4c4c3ccc3c5ccccc5[se]c34)ccc21 ; CC1(C)c2ccccc2-c2cc(N(c3ccccc3)c3cc4ccccc4c4c3ccc3c5ccccc5[se]c34)ccc21]

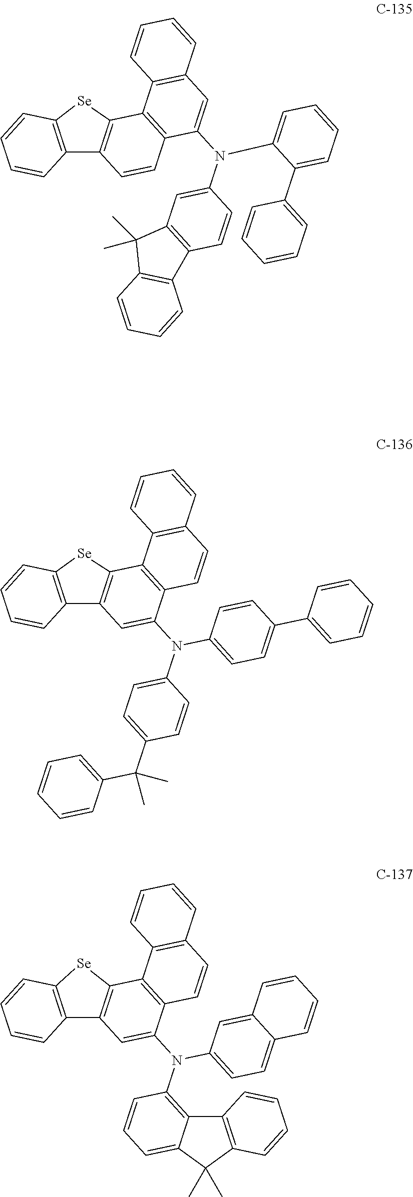 [SMILES: CC1(C)c2ccccc2-c2ccc(N(c3ccccc3-c3ccccc3)c3cc4ccccc4c4c3ccc3c5ccccc5[se]c34)cc21 ; CC(C)(c1ccccc1)c1ccc(N(c2ccc(-c3ccccc3)cc2)c2cc3c4ccccc4[se]c3c3c2ccc2ccccc23)cc1 ; CC1(C)c2ccccc2-c2c(N(c3ccc4ccccc4c3)c3cc4c5ccccc5[se]c4c4c3ccc3ccccc34)cccc21]

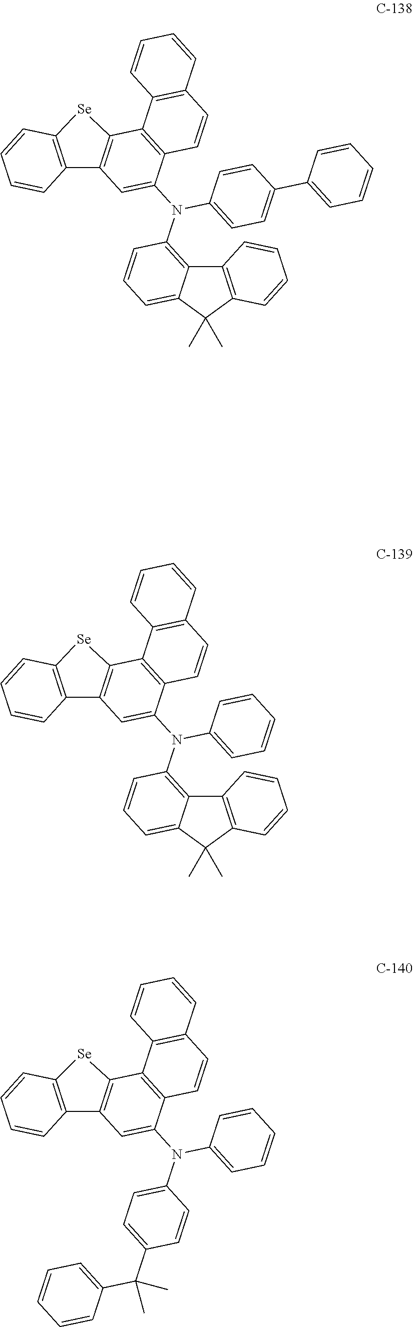 [SMILES: CC1(C)c2ccccc2-c2c(N(c3ccc(-c4ccccc4)cc3)c3cc4c5ccccc5[se]c4c4c3ccc3ccccc34)cccc21 ; CC1(C)c2ccccc2-c2c(N(c3ccccc3)c3cc4c5ccccc5[se]c4c4c3ccc3ccccc34)cccc21 ; CC(C)(c1ccccc1)c1ccc(N(c2ccccc2)c2cc3c4ccccc4[se]c3c3c2ccc2ccccc23)cc1]

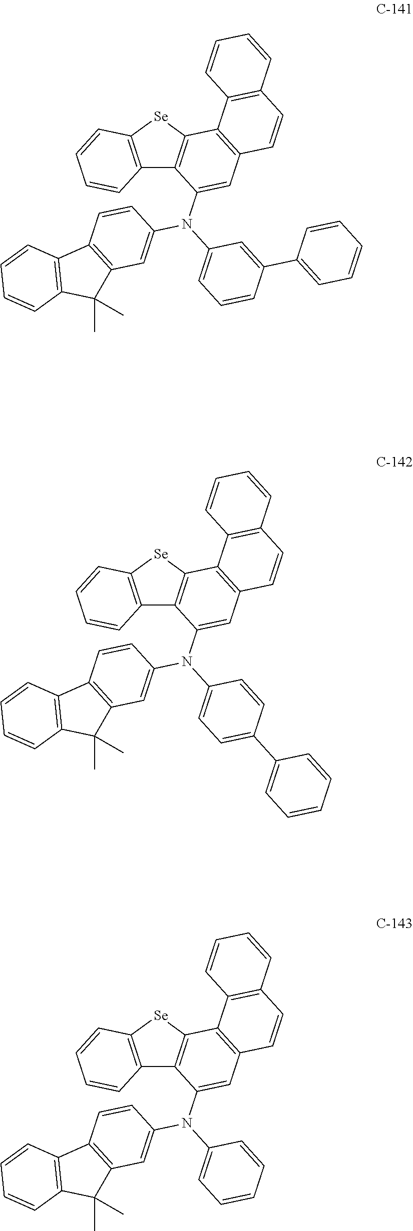 [SMILES: CC1(C)c2ccccc2-c2ccc(N(c3cccc(-c4ccccc4)c3)c3cc4ccc5ccccc5c4c4[se]c5ccccc5c34)cc21 ; CC1(C)c2ccccc2-c2ccc(N(c3ccc(-c4ccccc4)cc3)c3cc4ccc5ccccc5c4c4[se]c5ccccc5c34)cc21 ; CC1(C)c2ccccc2-c2ccc(N(c3ccccc3)c3cc4ccc5ccccc5c4c4[se]c5ccccc5c34)cc21]

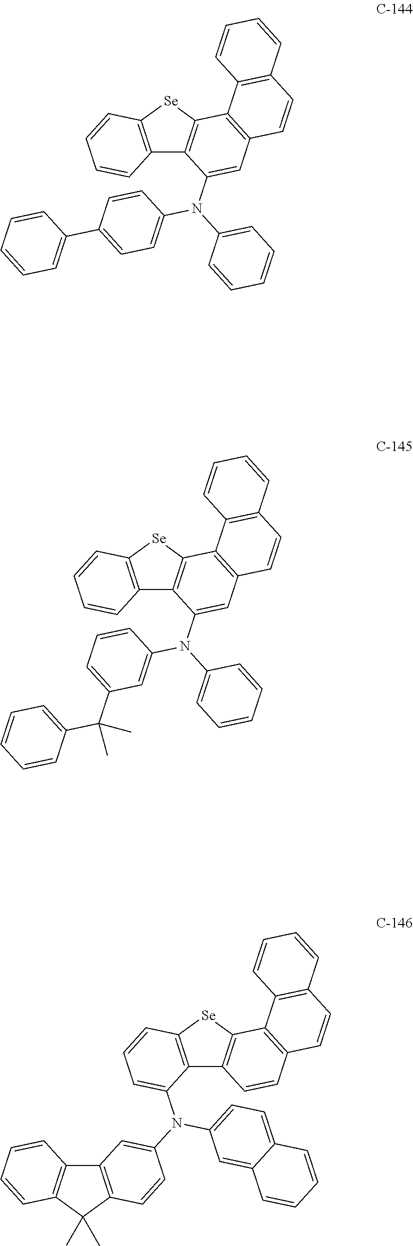 [SMILES: c1ccc(-c2ccc(N(c3ccccc3)c3cc4ccc5ccccc5c4c4[se]c5ccccc5c34)cc2)cc1 ; CC(C)(c1ccccc1)c1cccc(N(c2ccccc2)c2cc3ccc4ccccc4c3c3[se]c4ccccc4c23)c1 ; CC1(C)c2ccccc2-c2cc(N(c3ccc4ccccc4c3)c3cccc4[se]c5c(ccc6ccc7ccccc7c65)c34)ccc21]

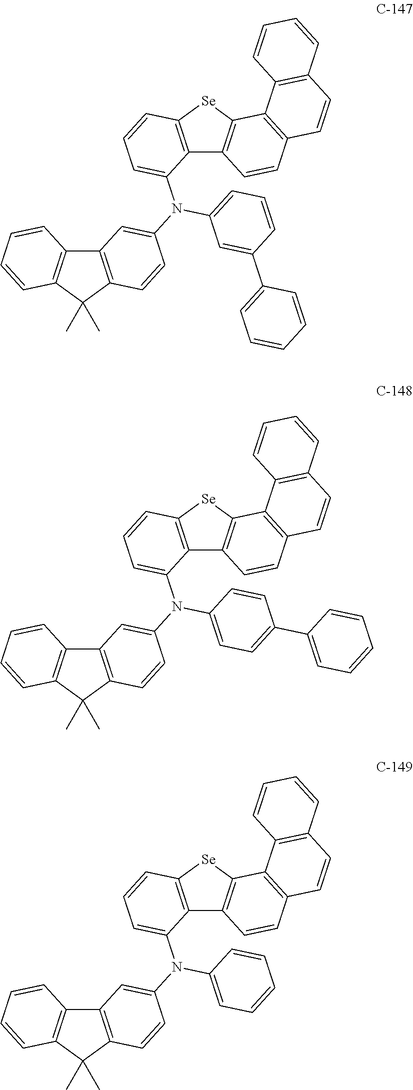 [SMILES: CC1(C)c2ccccc2-c2cc(N(c3cccc(-c4ccccc4)c3)c3cccc4[se]c5c(ccc6ccc7ccccc7c65)c34)ccc21 ; CC1(C)c2ccccc2-c2cc(N(c3ccc(-c4ccccc4)cc3)c3cccc4[se]c5c(ccc6ccc7ccccc7c65)c34)ccc21 ; CC1(C)c2ccccc2-c2cc(N(c3ccccc3)c3cccc4[se]c5c(ccc6ccc7ccccc7c65)c34)ccc21]

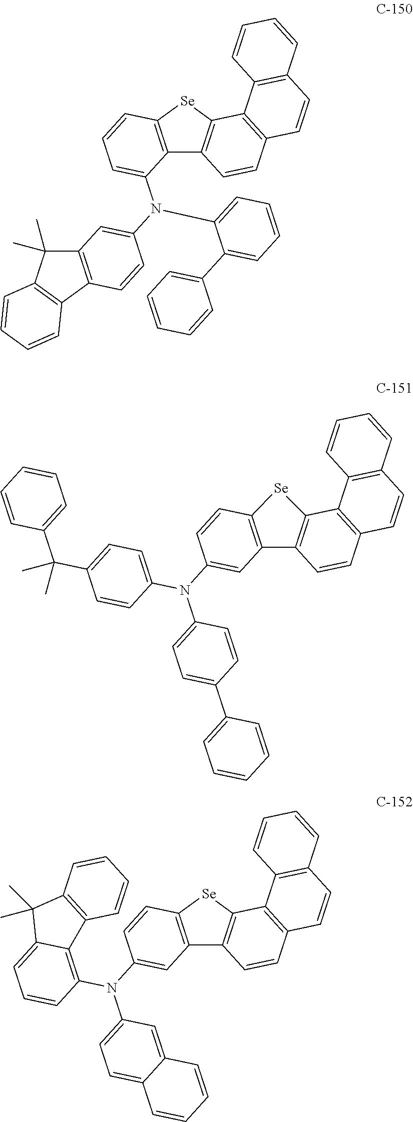

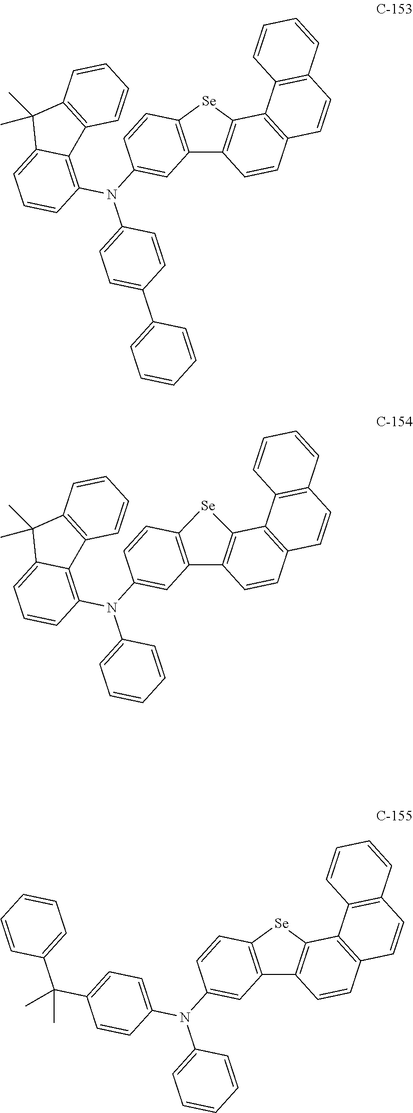 [SMILES: CC1(C)c2ccccc2-c2c(N(c3ccc(-c4ccccc4)cc3)c3ccc4[se]c5c(ccc6ccc7ccccc7c65)c4c3)cccc21 ; CC1(C)c2ccccc2-c2c(N(c3ccccc3)c3ccc4[se]c5c(ccc6ccc7ccccc7c65)c4c3)cccc21 ; CC(C)(c1ccccc1)c1ccc(N(c2ccccc2)c2ccc3[se]c4c(ccc5ccc6ccccc6c54)c3c2)cc1]

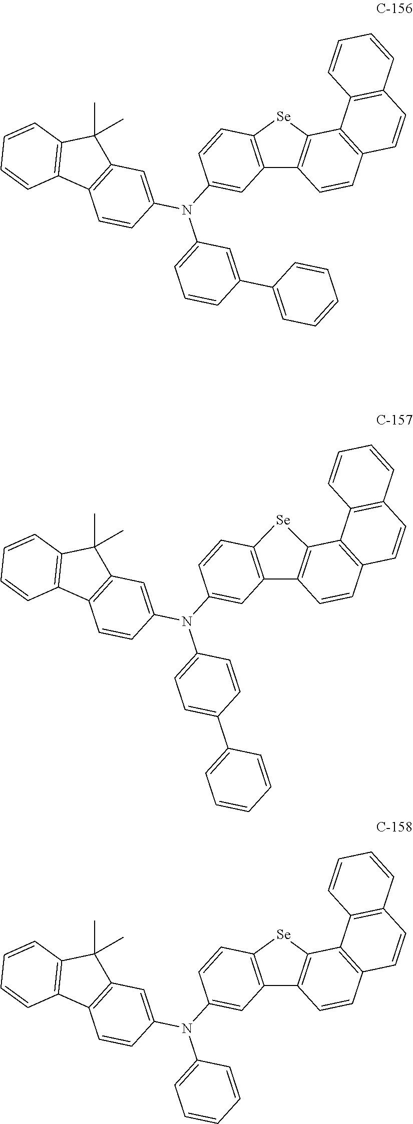 [SMILES: CC1(C)c2ccccc2-c2ccc(N(c3cccc(-c4ccccc4)c3)c3ccc4[se]c5c(ccc6ccc7ccccc7c65)c4c3)cc21 ; CC1(C)c2ccccc2-c2ccc(N(c3ccc(-c4ccccc4)cc3)c3ccc4[se]c5c(ccc6ccc7ccccc7c65)c4c3)cc21 ; CC1(C)c2ccccc2-c2ccc(N(c3ccccc3)c3ccc4[se]c5c(ccc6ccc7ccccc7c65)c4c3)cc21]

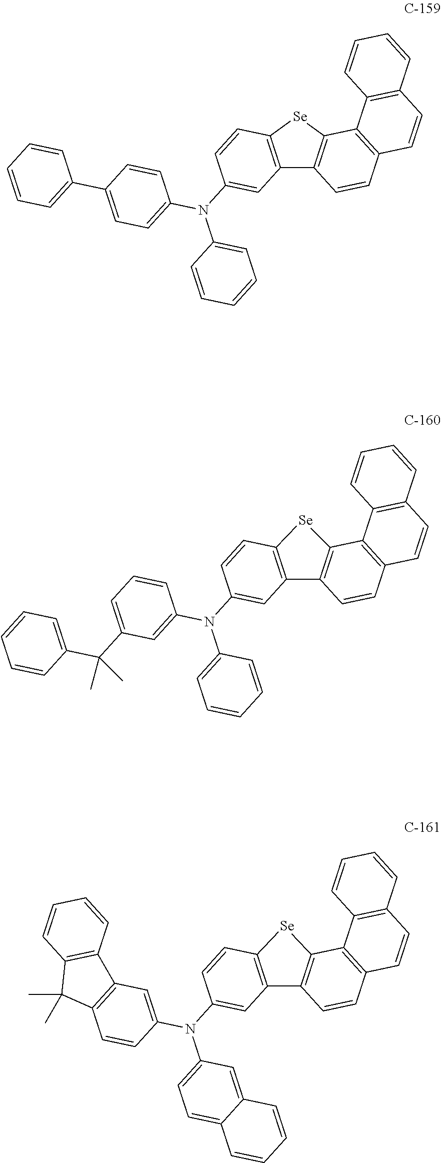 [SMILES: c1ccc(-c2ccc(N(c3ccccc3)c3ccc4[se]c5c(ccc6ccc7ccccc7c65)c4c3)cc2)cc1 ; CC(C)(c1ccccc1)c1cccc(N(c2ccccc2)c2ccc3[se]c4c(ccc5ccc6ccccc6c54)c3c2)c1 ; CC1(C)c2ccccc2-c2cc(N(c3ccc4ccccc4c3)c3ccc4[se]c5c(ccc6ccc7ccccc7c65)c4c3)ccc21]

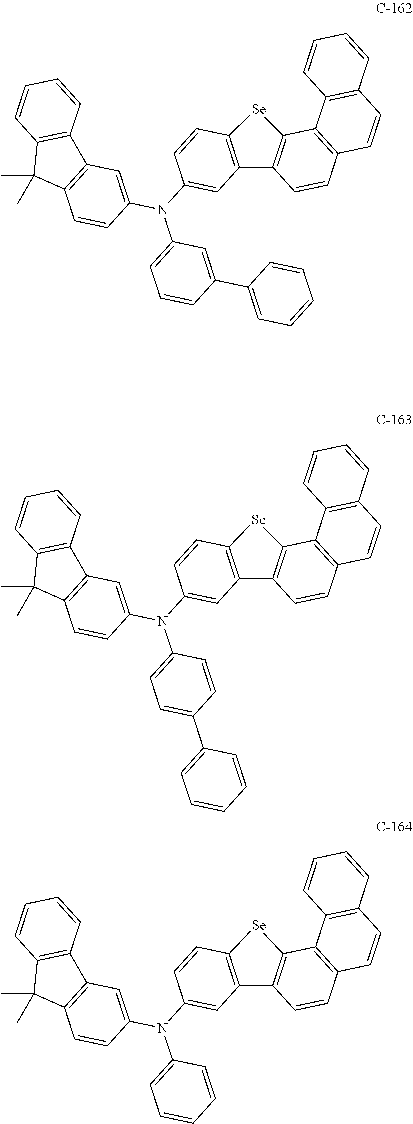 [SMILES: CC1(C)c2ccccc2-c2cc(N(c3cccc(-c4ccccc4)c3)c3ccc4[se]c5c(ccc6ccc7ccccc7c65)c4c3)ccc21 ; CC1(C)c2ccccc2-c2cc(N(c3ccc(-c4ccccc4)cc3)c3ccc4[se]c5c(ccc6ccc7ccccc7c65)c4c3)ccc21 ; CC1(C)c2ccccc2-c2cc(N(c3ccccc3)c3ccc4[se]c5c(ccc6ccc7ccccc7c65)c4c3)ccc21]

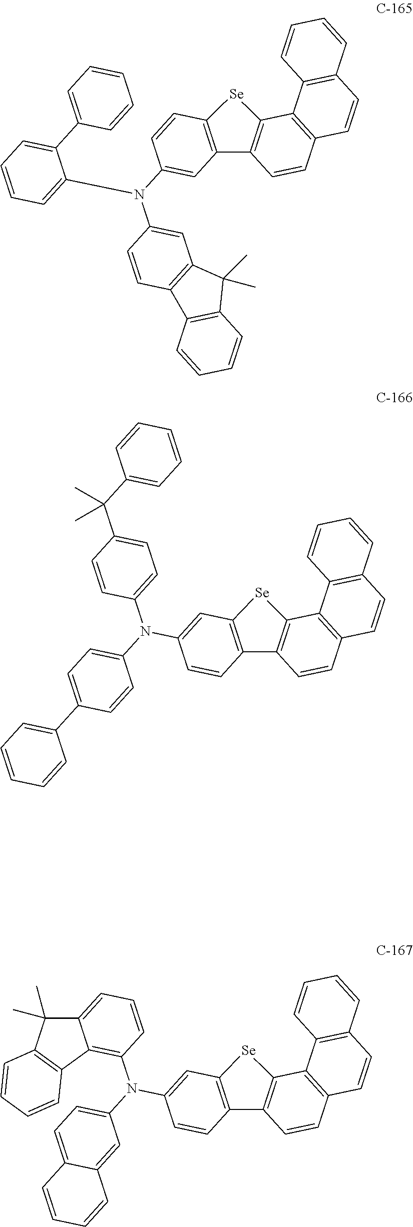 [SMILES: CC1(C)c2ccccc2-c2ccc(N(c3ccc4[se]c5c(ccc6ccc7ccccc7c65)c4c3)c3ccccc3-c3ccccc3)cc21 ; CC(C)(c1ccccc1)c1ccc(N(c2ccc(-c3ccccc3)cc2)c2ccc3c(c2)[se]c2c3ccc3ccc4ccccc4c32)cc1 ; CC1(C)c2ccccc2-c2c(N(c3ccc4ccccc4c3)c3ccc4c(c3)[se]c3c4ccc4ccc5ccccc5c43)cccc21]

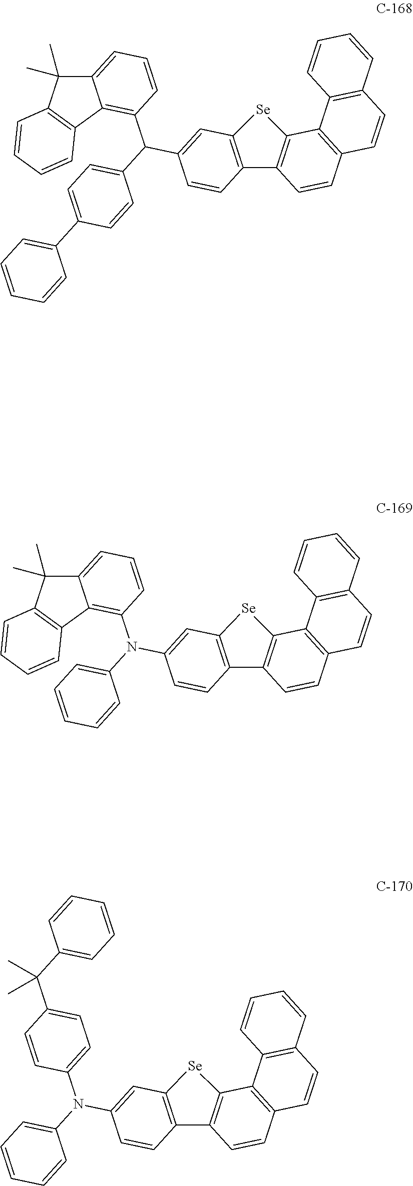 [SMILES: CC1(C)c2ccccc2-c2c(C(c3ccc(-c4ccccc4)cc3)c3ccc4c(c3)[se]c3c4ccc4ccc5ccccc5c43)cccc21 ; CC1(C)c2ccccc2-c2c(N(c3ccccc3)c3ccc4c(c3)[se]c3c4ccc4ccc5ccccc5c43)cccc21 ; CC(C)(c1ccccc1)c1ccc(N(c2ccccc2)c2ccc3c(c2)[se]c2c3ccc3ccc4ccccc4c32)cc1]

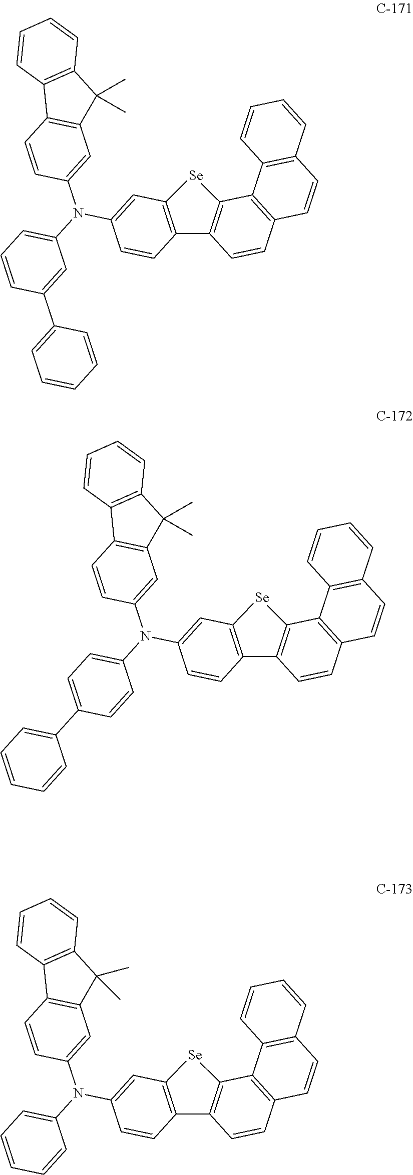 [SMILES: CC1(C)c2ccccc2-c2ccc(N(c3cccc(-c4ccccc4)c3)c3ccc4c(c3)[se]c3c4ccc4ccc5ccccc5c43)cc21 ; CC1(C)c2ccccc2-c2ccc(N(c3ccc(-c4ccccc4)cc3)c3ccc4c(c3)[se]c3c4ccc4ccc5ccccc5c43)cc21 ; CC1(C)c2ccccc2-c2ccc(N(c3ccccc3)c3ccc4c(c3)[se]c3c4ccc4ccc5ccccc5c43)cc21]

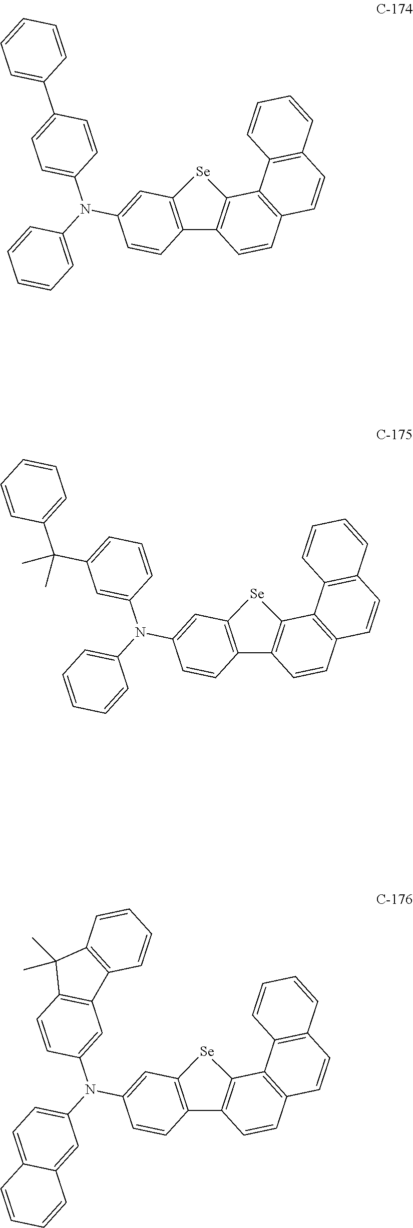 [SMILES: c1ccc(-c2ccc(N(c3ccccc3)c3ccc4c(c3)[se]c3c4ccc4ccc5ccccc5c43)cc2)cc1 ; CC(C)(c1ccccc1)c1cccc(N(c2ccccc2)c2ccc3c(c2)[se]c2c3ccc3ccc4ccccc4c32)c1 ; CC1(C)c2ccccc2-c2cc(N(c3ccc4ccccc4c3)c3ccc4c(c3)[se]c3c4ccc4ccc5ccccc5c43)ccc21]

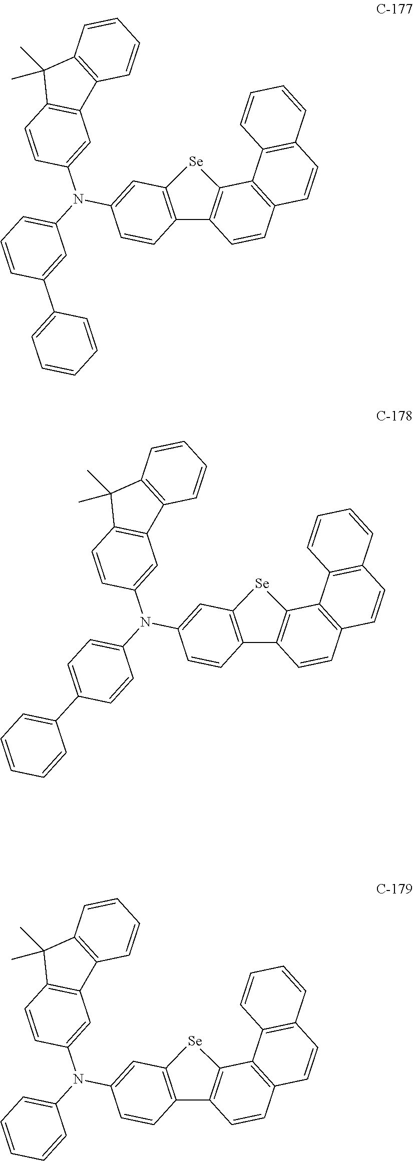 [SMILES: CC1(C)c2ccccc2-c2cc(N(c3cccc(-c4ccccc4)c3)c3ccc4c(c3)[se]c3c4ccc4ccc5ccccc5c43)ccc21 ; CC1(C)c2ccccc2-c2cc(N(c3ccc(-c4ccccc4)cc3)c3ccc4c(c3)[se]c3c4ccc4ccc5ccccc5c43)ccc21 ; CC1(C)c2ccccc2-c2cc(N(c3ccccc3)c3ccc4c(c3)[se]c3c4ccc4ccc5ccccc5c43)ccc21]

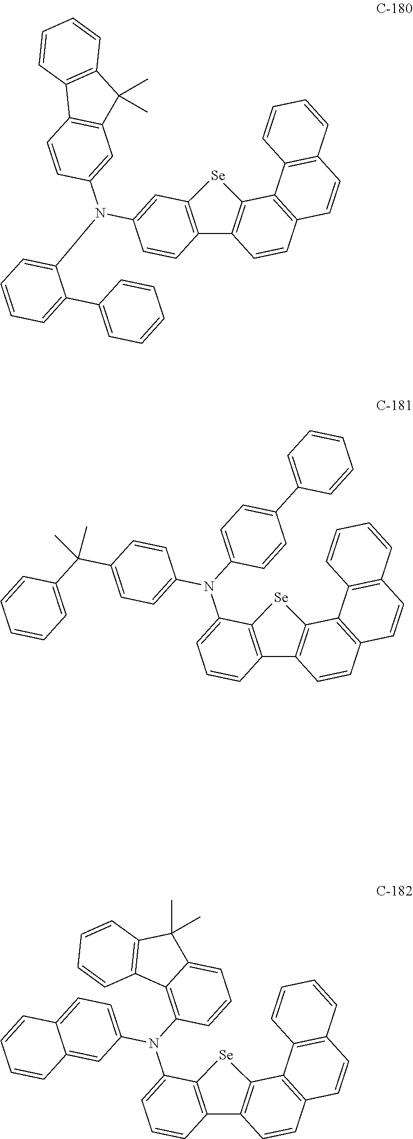 [SMILES: CC1(C)c2ccccc2-c2ccc(N(c3ccc4c(c3)[se]c3c4ccc4ccc5ccccc5c43)c3ccccc3-c3ccccc3)cc21 ; CC(C)(c1ccccc1)c1ccc(N(c2ccc(-c3ccccc3)cc2)c2cccc3c2[se]c2c3ccc3ccc4ccccc4c32)cc1 ; CC1(C)c2ccccc2-c2c(N(c3ccc4ccccc4c3)c3cccc4c3[se]c3c4ccc4ccc5ccccc5c43)cccc21]

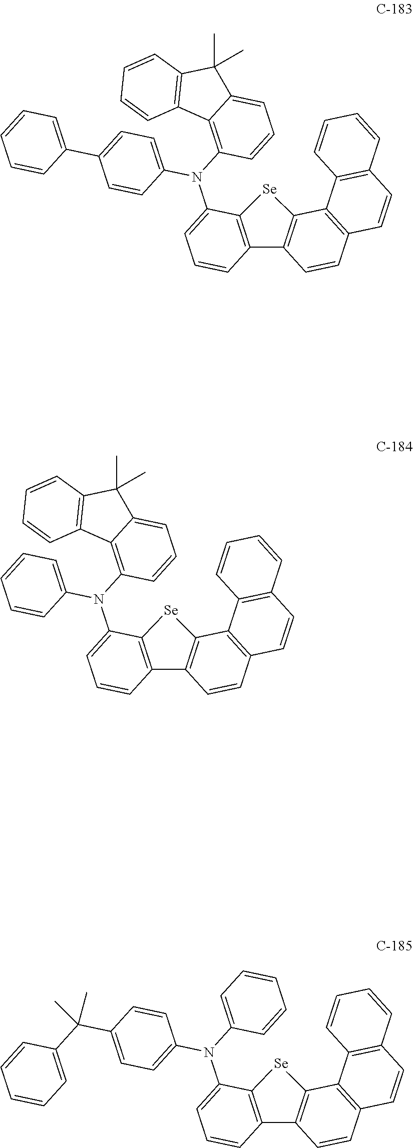 [SMILES: CC1(C)c2ccccc2-c2c(N(c3ccc(-c4ccccc4)cc3)c3cccc4c3[se]c3c4ccc4ccc5ccccc5c43)cccc21 ; CC1(C)c2ccccc2-c2c(N(c3ccccc3)c3cccc4c3[se]c3c4ccc4ccc5ccccc5c43)cccc21 ; CC(C)(c1ccccc1)c1ccc(N(c2ccccc2)c2cccc3c2[se]c2c3ccc3ccc4ccccc4c32)cc1]

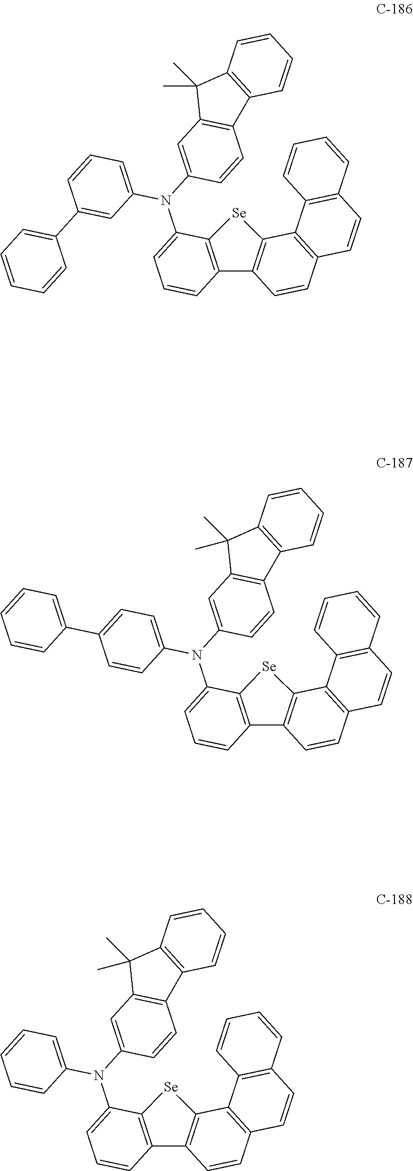 [SMILES: CC1(C)c2ccccc2-c2ccc(N(c3cccc(-c4ccccc4)c3)c3cccc4c3[se]c3c4ccc4ccc5ccccc5c43)cc21 ; CC1(C)c2ccccc2-c2ccc(N(c3ccc(-c4ccccc4)cc3)c3cccc4c3[se]c3c4ccc4ccc5ccccc5c43)cc21 ; CC1(C)c2ccccc2-c2ccc(N(c3ccccc3)c3cccc4c3[se]c3c4ccc4ccc5ccccc5c43)cc21]

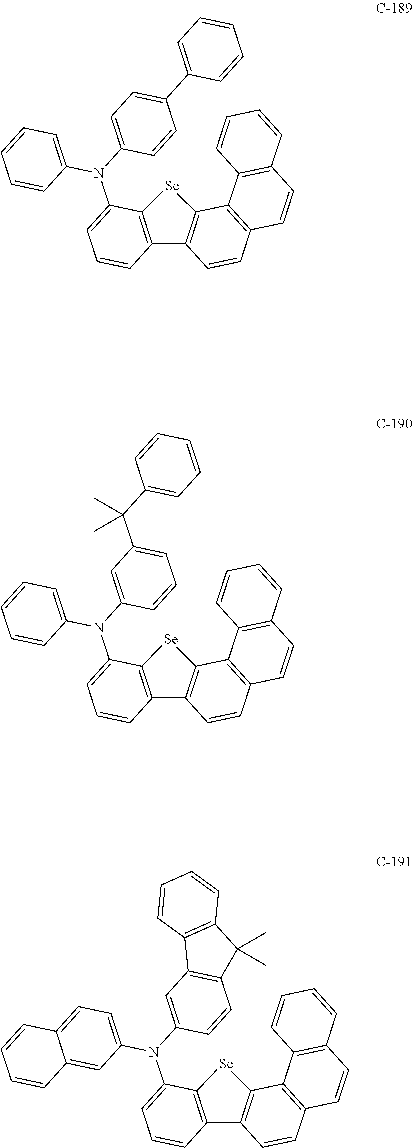 [SMILES: c1ccc(-c2ccc(N(c3ccccc3)c3cccc4c3[se]c3c4ccc4ccc5ccccc5c43)cc2)cc1 ; CC(C)(c1ccccc1)c1cccc(N(c2ccccc2)c2cccc3c2[se]c2c3ccc3ccc4ccccc4c32)c1 ; CC1(C)c2ccccc2-c2cc(N(c3ccc4ccccc4c3)c3cccc4c3[se]c3c4ccc4ccc5ccccc5c43)ccc21]

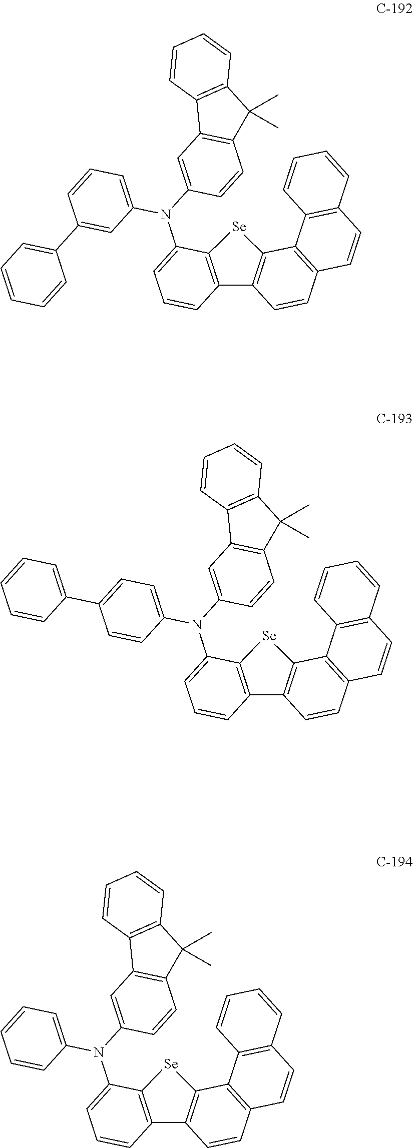 [SMILES: CC1(C)c2ccccc2-c2cc(N(c3cccc(-c4ccccc4)c3)c3cccc4c3[se]c3c4ccc4ccc5ccccc5c43)ccc21 ; CC1(C)c2ccccc2-c2cc(N(c3ccc(-c4ccccc4)cc3)c3cccc4c3[se]c3c4ccc4ccc5ccccc5c43)ccc21 ; CC1(C)c2ccccc2-c2cc(N(c3ccccc3)c3cccc4c3[se]c3c4ccc4ccc5ccccc5c43)ccc21]

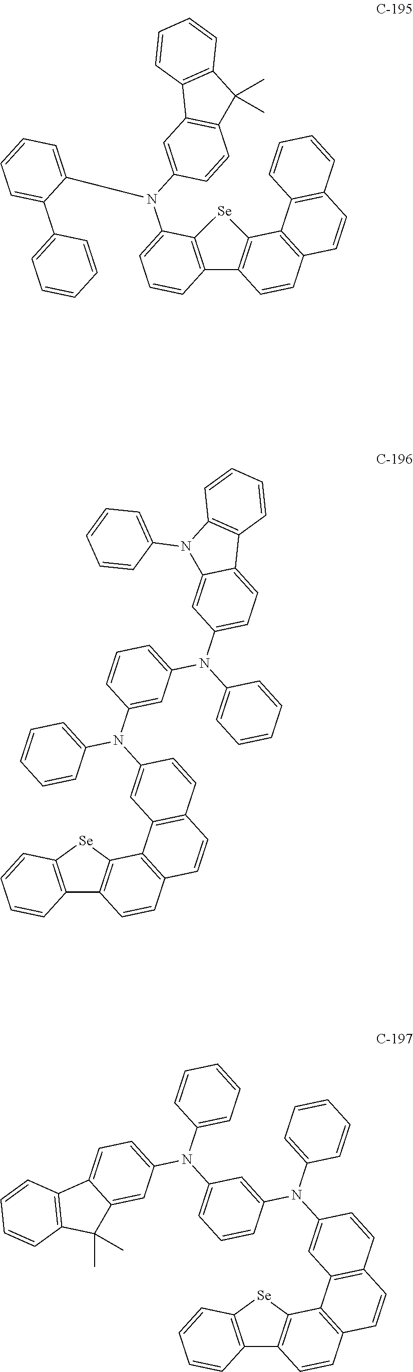 [SMILES: CC1(C)c2ccccc2-c2cc(N(c3ccccc3-c3ccccc3)c3cccc4c3[se]c3c4ccc4ccc5ccccc5c43)ccc21 ; c1ccc(N(c2cccc(N(c3ccccc3)c3ccc4c5ccccc5n(-c5ccccc5)c4c3)c2)c2ccc3ccc4ccc5c6ccccc6[se]c5c4c3c2)cc1 ; CC1(C)c2ccccc2-c2ccc(N(c3ccccc3)c3cccc(N(c4ccccc4)c4ccc5ccc6ccc7c8ccccc8[se]c7c6c5c4)c3)cc21]

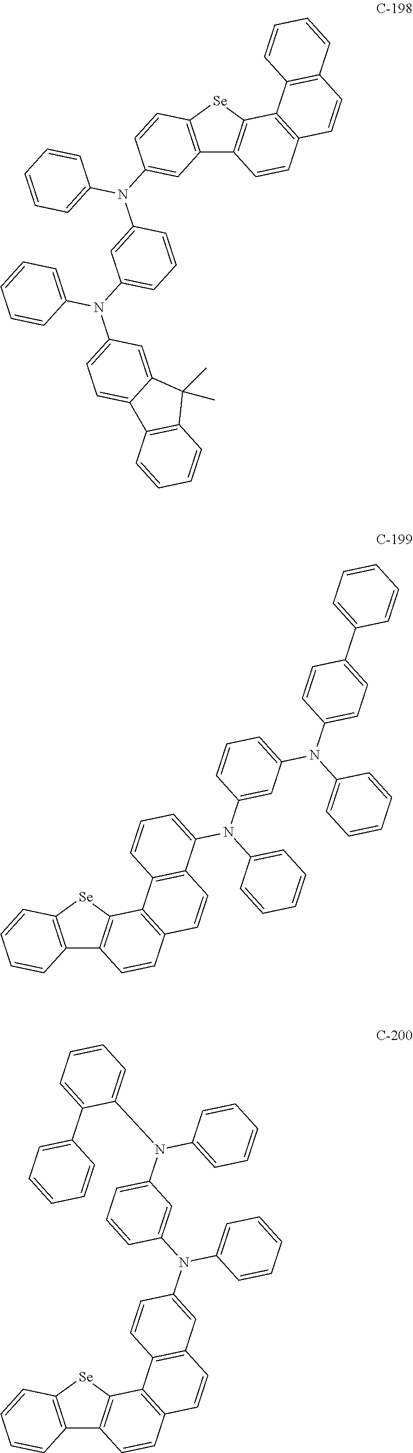 [SMILES: CC1(C)c2ccccc2-c2ccc(N(c3ccccc3)c3cccc(N(c4ccccc4)c4ccc5[se]c6c(ccc7ccc8ccccc8c76)c5c4)c3)cc21 ; c1ccc(-c2ccc(N(c3ccccc3)c3cccc(N(c4ccccc4)c4cccc5c4ccc4ccc6c7ccccc7[se]c6c45)c3)cc2)cc1 ; c1ccc(-c2ccccc2N(c2ccccc2)c2cccc(N(c3ccccc3)c3ccc4c(ccc5ccc6c7ccccc7[se]c6c54)c3)c2)cc1]

The present disclosure provides an organic electroluminescent material comprising the organic electroluminescent compound represented by formula 1, and an organic electroluminescent device comprising the same.

The organic electroluminescent material may solely consist of the organic electroluminescent compound of the present disclosure, or may further comprise conventional materials included in the organic electroluminescent material.

The present disclosure provides a plurality of host materials comprising the at least one first host compound represented by formula 1′ and the at least one second host compound represented by formula 1′, wherein Ar₁ in the first host compound is represented by formula a, and Ar₁ in the second host compound represents a substituted or unsubstituted (3- to 30-membered)heteroaryl.

According to one embodiment of the present disclosure, Ar₁ in the second host compound represented by formula 1′ represents a (5- to 15-membered)heteroaryl substituted with at least one of a (C6-C18)aryl(s) unsubstituted or substituted with deuterium, a (C1-C₆)alkyl(s) or a (C6-C18)aryl(C1-C12)alkyl(s), and a (5- to 15-membered)heteroaryl(s). For example, An may be a triazinyl substituted with at least one of a phenyl(s) unsubstituted or substituted with deuterium, a naphthyl(s), a biphenyl(s), a dimethylfluorenyl(s), a phenyl(s) substituted with a phenylpropyl(s), and a dibenzofuranyl(s), etc.

In formula 1′, the definitions and specific embodiments for R₁ to R₁₂, L₁, Rₐ₁, and Rₐ₂ are the same as in formula 1.

In addition, the present disclosure provides a plurality of host materials comprising at least one first host material comprising the compound represented by formula 1 and at least one second host material different from the first host material. According to one embodiment of the present disclosure, the second host material may comprise the organic electroluminescent compound represented by formula 2 or the organic electroluminescent compound represented by formula 3.

Hereinafter, the compound represented by formula 2 will be described in more detail.

In formula 2, X₁ and Y₁, each independently, represent —N═, —O— or -5-, with the proviso that any one of X; and Y₁ represents —N═, and the other one represents —NR₆₇-, or —S—. According to one embodiment of the present disclosure, any one of X₁ and Y₁ represents —N═, and the other one represents —O— or —S—.

In formula 2, R61 represents a substituted or unsubstituted (C6-C30)aryl, or a substituted or unsubstituted (3- to 30-membered)heteroaryl. According to one embodiment of the present disclosure, R₆₁ represents an unsubstituted (C6-018)aryl or an unsubstituted (3- to 7-membered)heteroaryl, Specifically, R₆₁ may be a phenyl, a biphenyl, a naphthyl, a pyridyl, etc.

In formula 2, R₆₂ to R₆₄ and R₆₇, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted unsubstituted fused ring group of a (C3-C30) aliphatic ring(s) and a (C6-C30) aromatic ring(s), or -L₃″—N(Ar₃″)(Ar₄″); or may be linked to an adjacent substituent(s) to form a ring(s). According to one embodiment of the present disclosure, Rₐ₂ to R₆4 and R₆₇, each independently, represent hydrogen or an unsubstituted (C6-C12)aryl. For example, R₅₂ to R₆₄ and R₅₇, each independently, may be hydrogen, a phenyl, etc.

L₃″, each independently, represents a single bond, a substituted or unsubstituted (C6-C30)arylene, or a substituted or unsubstituted (3- to 30-membered)heteroarylene.

Ar₃″ and Ar₄″, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl.

In formula 2, R₅₅ and R₃₆, each independently, represent a substituted or unsubstituted (C6-C30)aryl, or a substituted or unsubstituted (3- to 30-membered)heteroaryl. According to one embodiment of the present disclosure, R₆₅ and R₆₆, each independently, represent a (C6-C30)aryl unsubstituted or substituted with at least one of deuterium, a (C1-C10)alkyl(s), a (C3-C10)cycloalkyl(s), a (C6-C25)aryl(s), a (3- to 18-membered)heteroaryl(s), and a di(C6-018)arylamino(s); or a (3- to 18-membered)heteroaryl unsubstituted or substituted with a (C6-C20)aryl(s). According to another embodiment of the present disclosure, R₆₅ and R₆₆, each independently, represent a (C6-C28)aryl unsubstituted or substituted with at least one of deuterium, a (C1-C₆)alkyl(s), a (C3-C8)cycloalkyl(s), a (C6-C22)aryl(s), a (3- to 14-membered)heteroaryl(s), and a di(C6-C12)arylamino(s); or a (3- to 17-membered)heteroaryl unsubstituted or substituted with a (C6-C12)aryl(s). For example, R65 and R₆₆, each independently, may be a phenyl unsubstituted or substituted with a pyridyl(s) substituted with a phenyl(s), a phenoxazinyl(s), a diphenylamino(s), an anthracenyl(s), a fluoranthenyl(s), a phenylfluorenyl(s), a phenylbenzimidazolyl(s), a cyclohexyl(s), or a diphenylamino; a biphenyl unsubstituted or substituted with deuterium, a methyl(s), or a tart-butyl(s); a terphenyl; a naphthyl; a naphthylphenyl; a phenylnaphthyl; a dimethylbenzofluorenyl; a dimethylfluorenyl; a phenanthrenyl; an anthracenyl; a diphenylfluorenyl; a phenylanthracenyl; a spirobifluorenyl; a dimethylphenyl; a butylphenyl; a pyridyl; a dibenzofuranyl unsubstituted or substituted with a phenyl(s); a dibenzothiophenyl; a phenylcarbazolyl; a dibenzonaphthocycloheptene; a benzothiophenyl; a benzonaphthofuranyl; a benzoimidazolyl, etc.

In formula 2, L₄ represents a single bond, a substituted or unsubstituted (C6-C30)arylene, or a substituted or unsubstituted (3- to 30-membered)heteroarylene. According to one embodiment of the present disclosure, L₄ represents a single bond or an unsubstituted (C6-C12)arylene. Specifically, L₄ may be a single bond or a phenylene.

In formula 2, a and b, each independently, represent an integer of 1 or 2, and c represents an integer of 1 to 4, where if a to c are each an integer of 2 or more, each of Rₐ₂ to each of R₆₄ may be the same or different each other.

The compound represented by formula 2 may be at least one selected from the group consisting of the following compounds, but is not limited thereto.

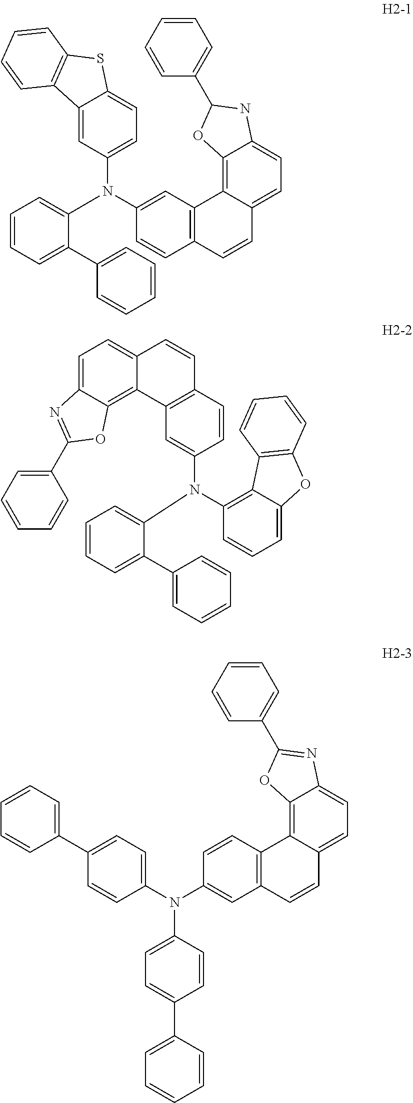

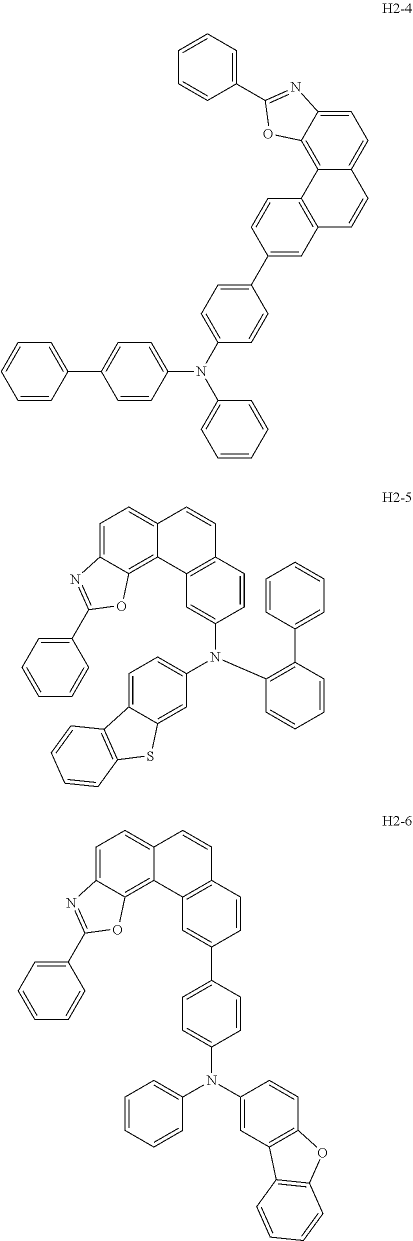 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

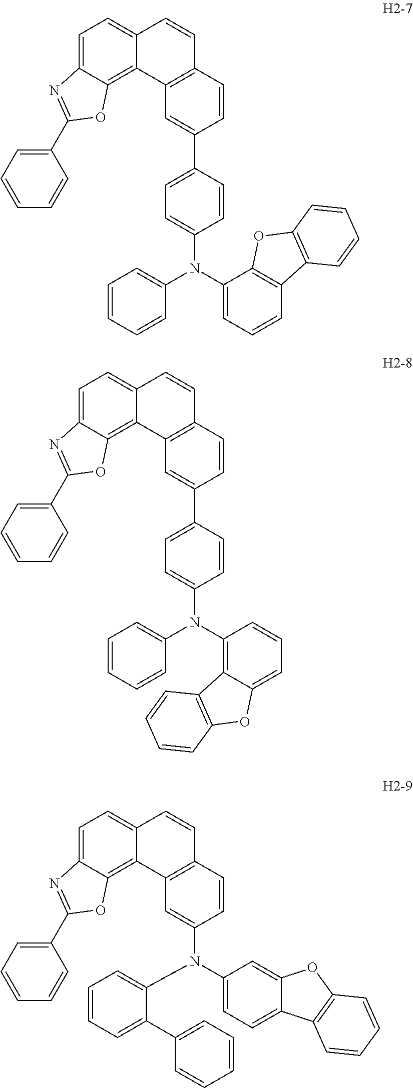 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccc7c(c6)oc6ccccc67)c6ccccc6-c6ccccc6)cc5c4c3o2)cc1]

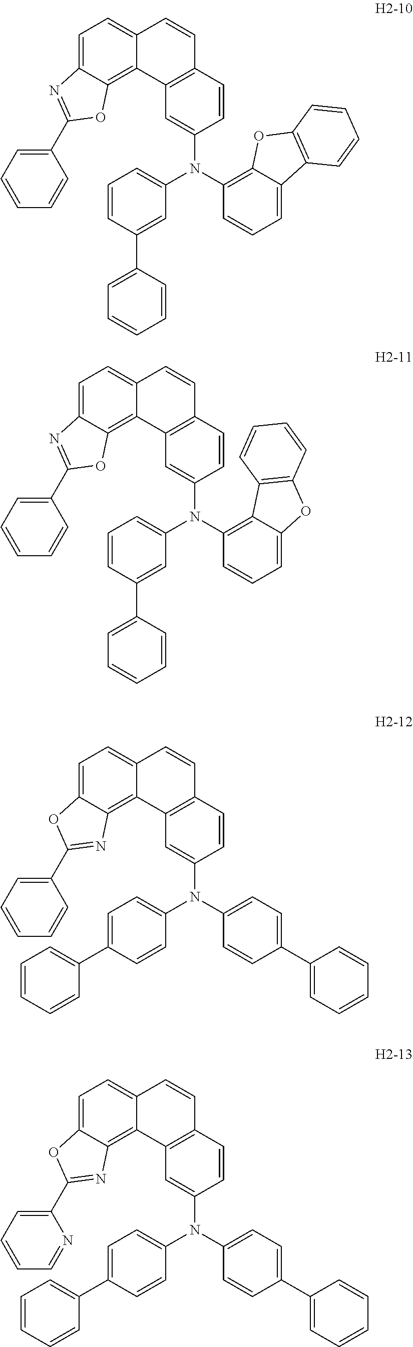

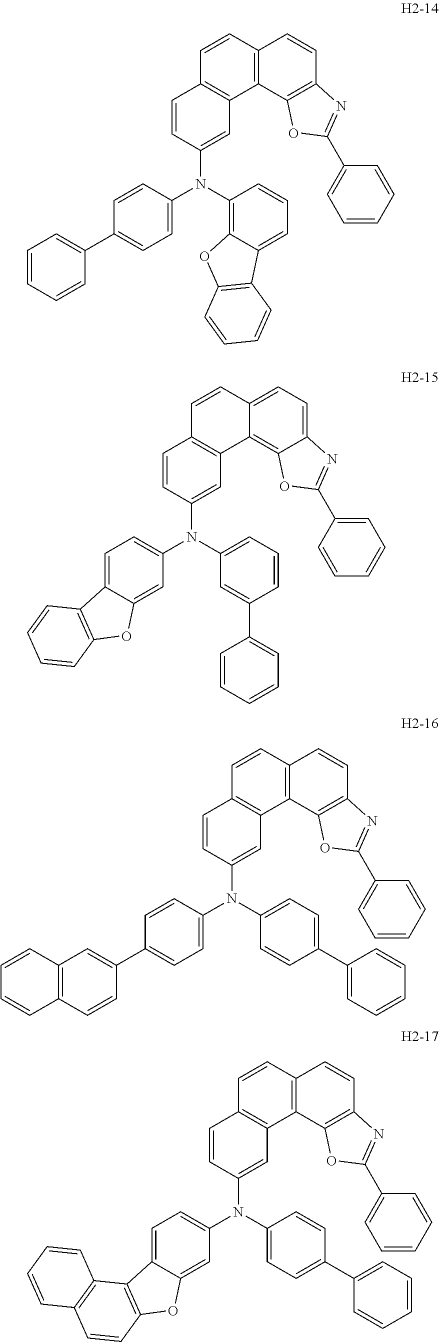

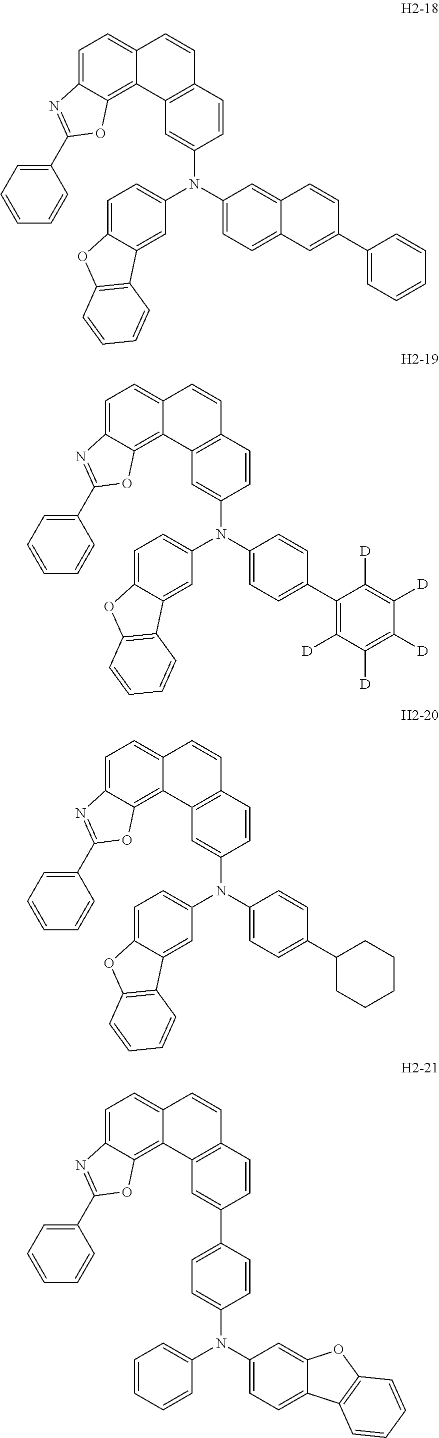

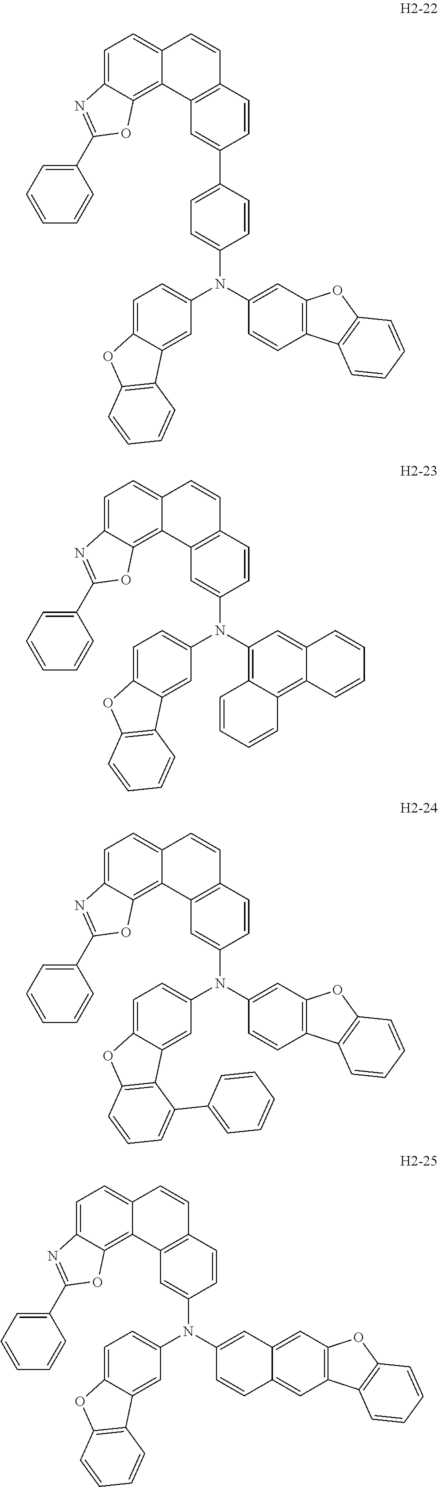

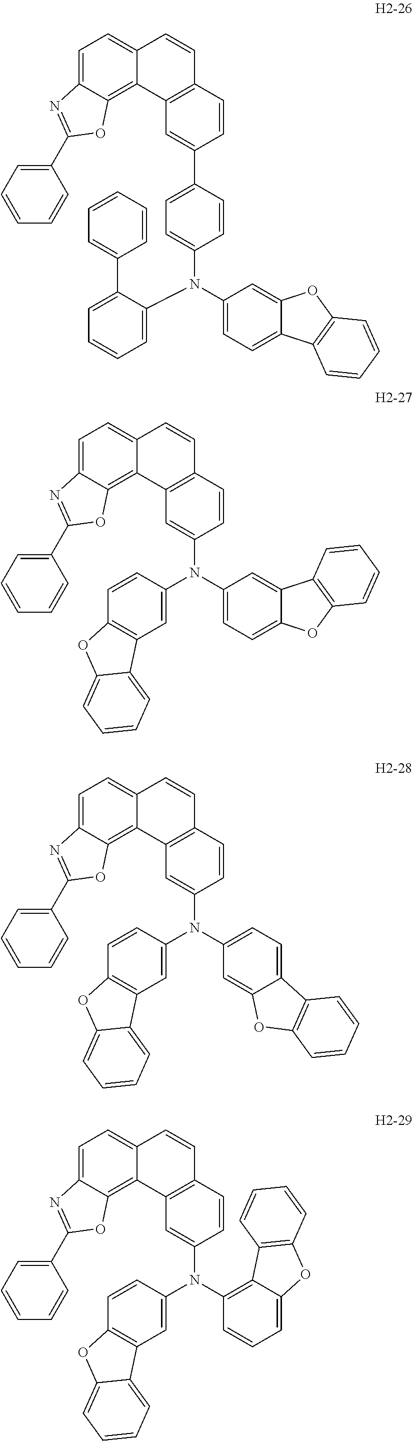

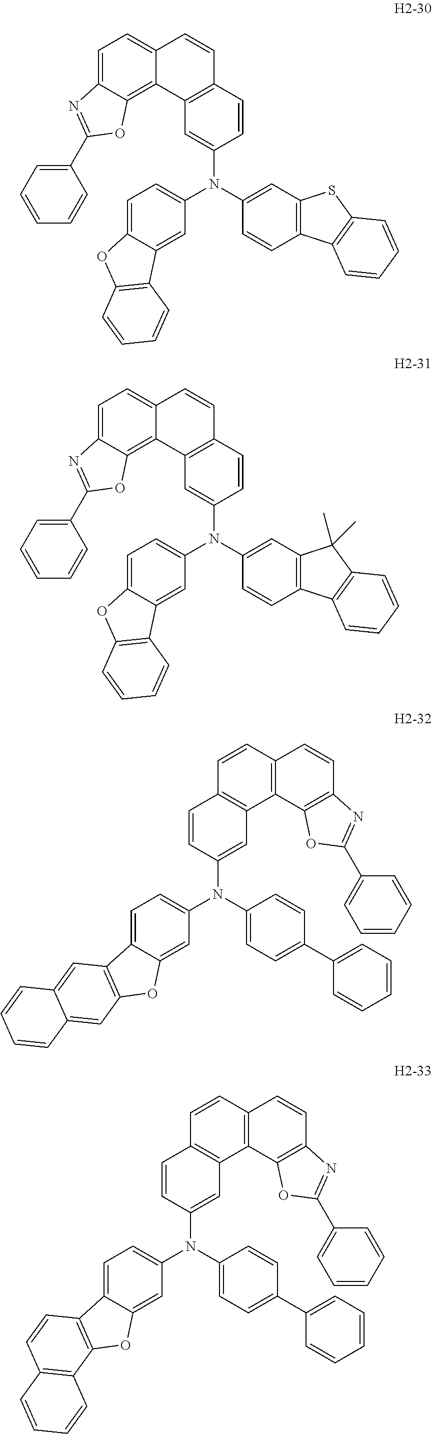

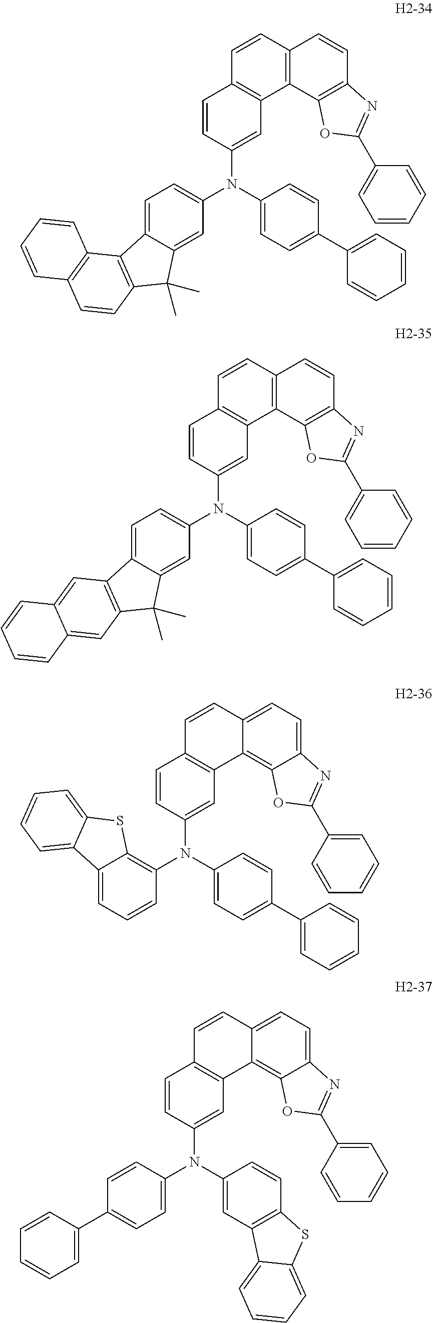 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

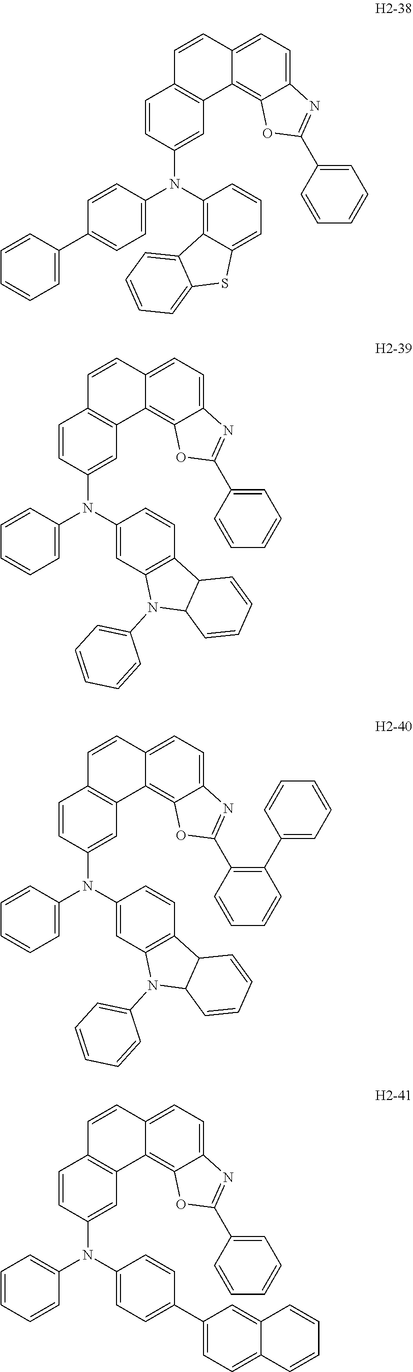

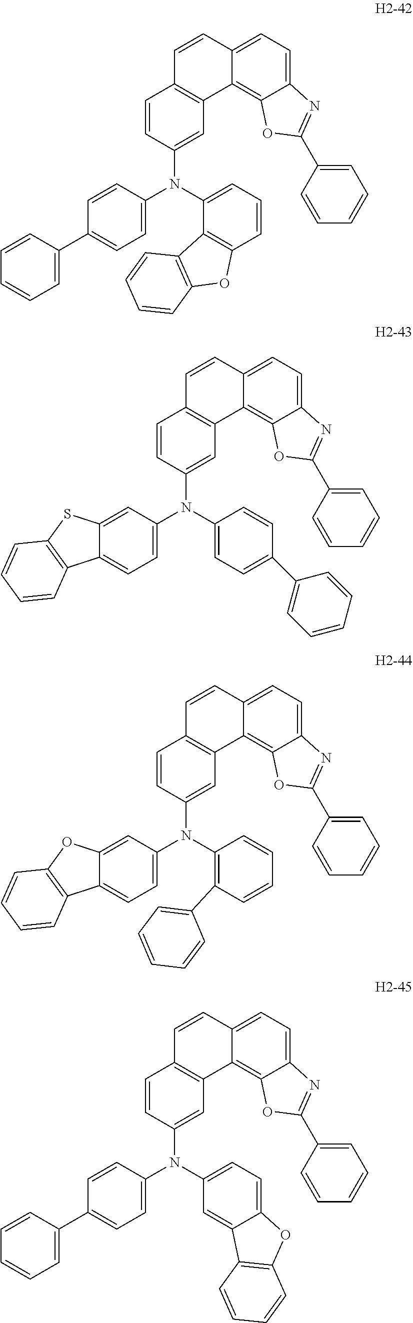 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1]

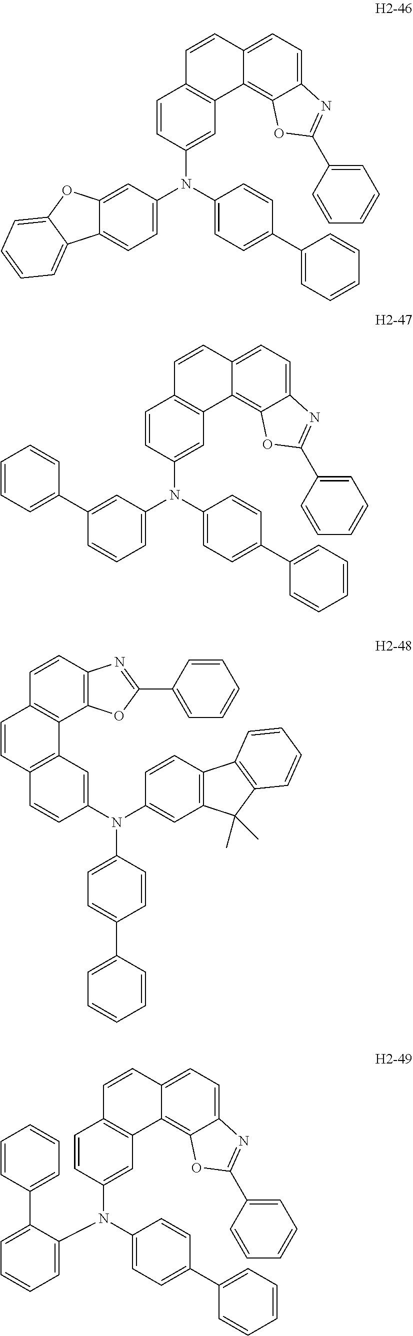 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1]

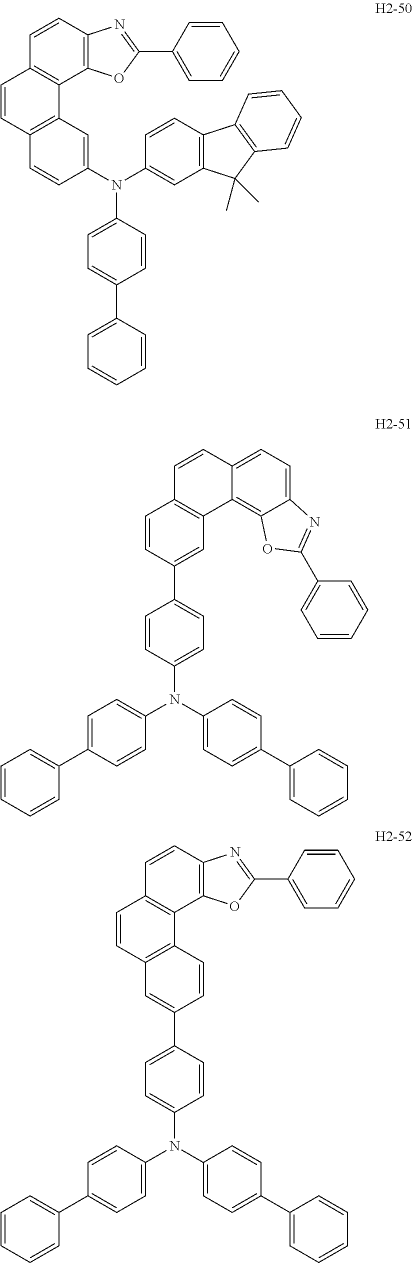

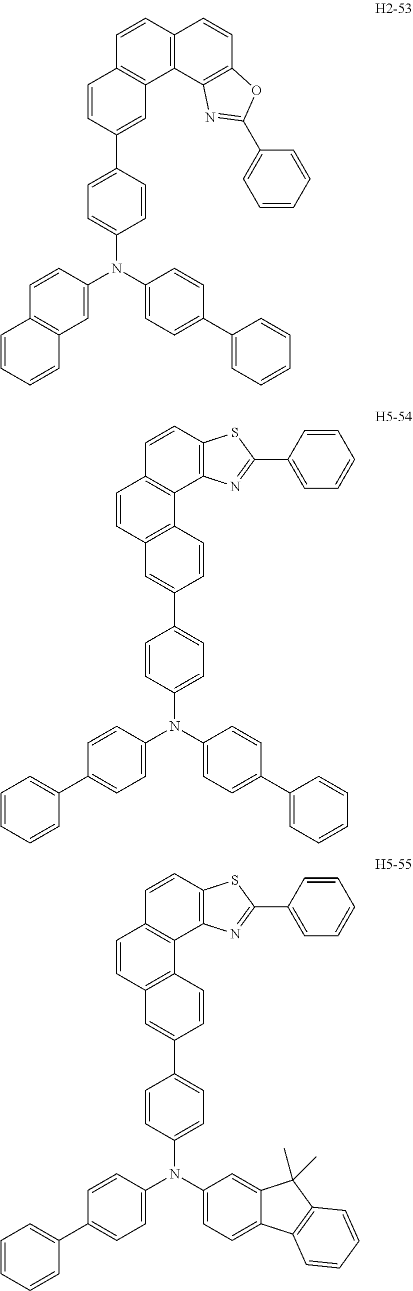

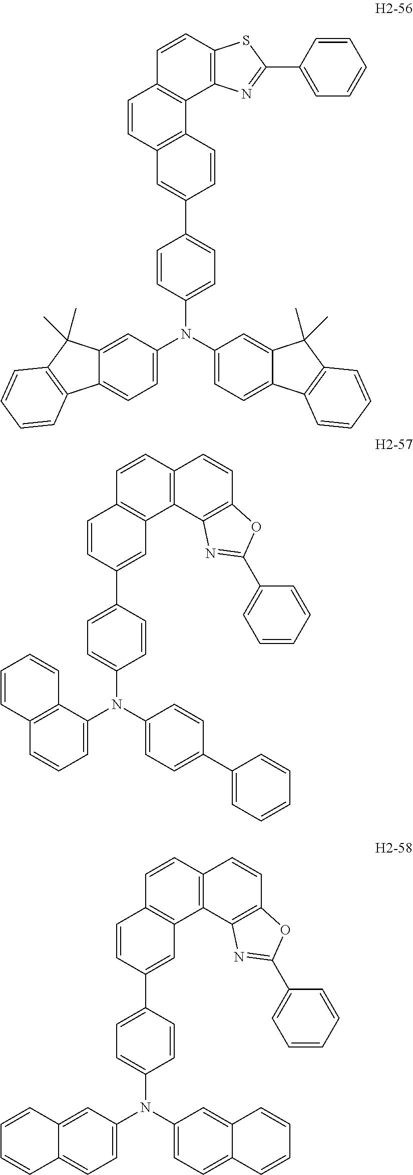 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1]

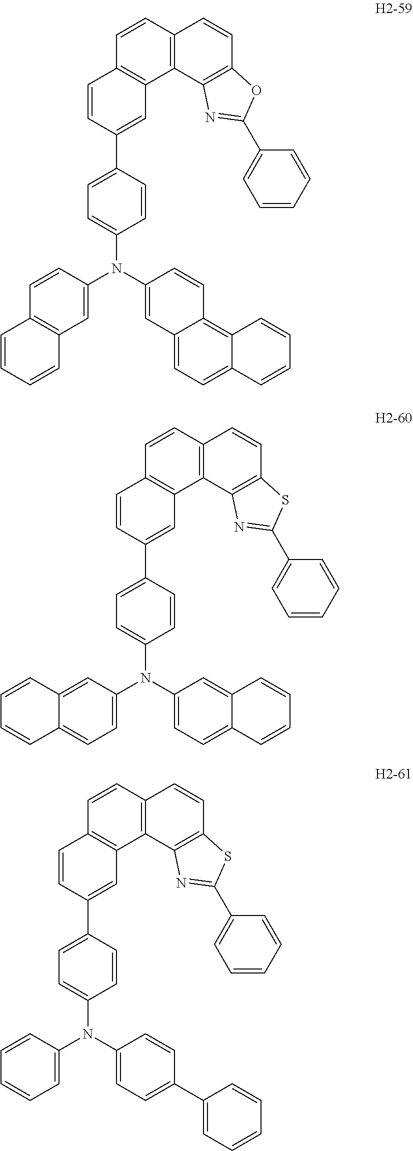 [SMILES: c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1]

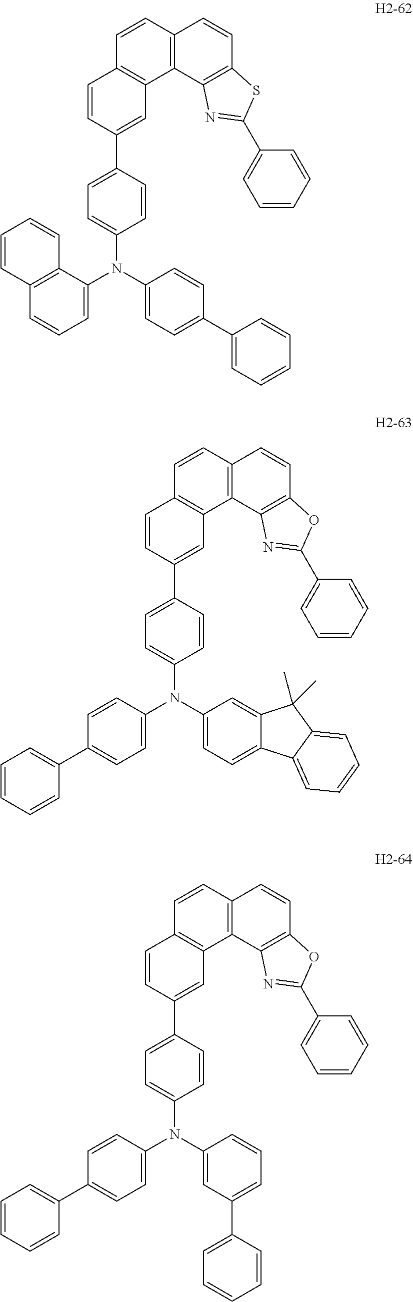 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

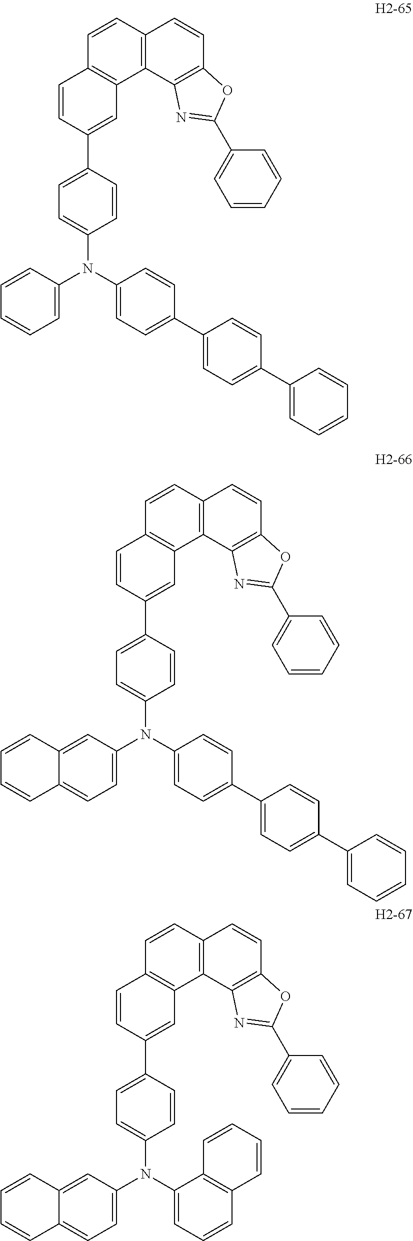 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

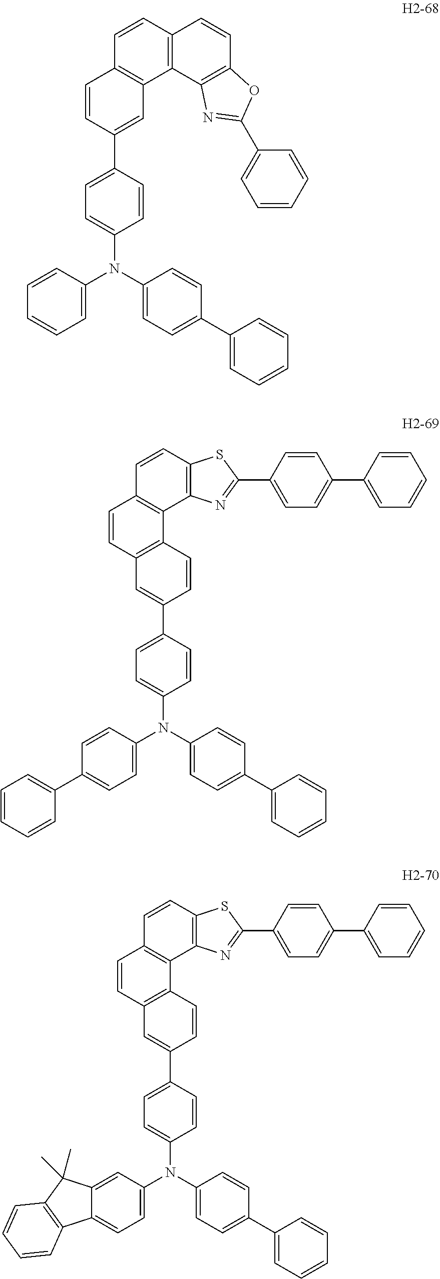 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

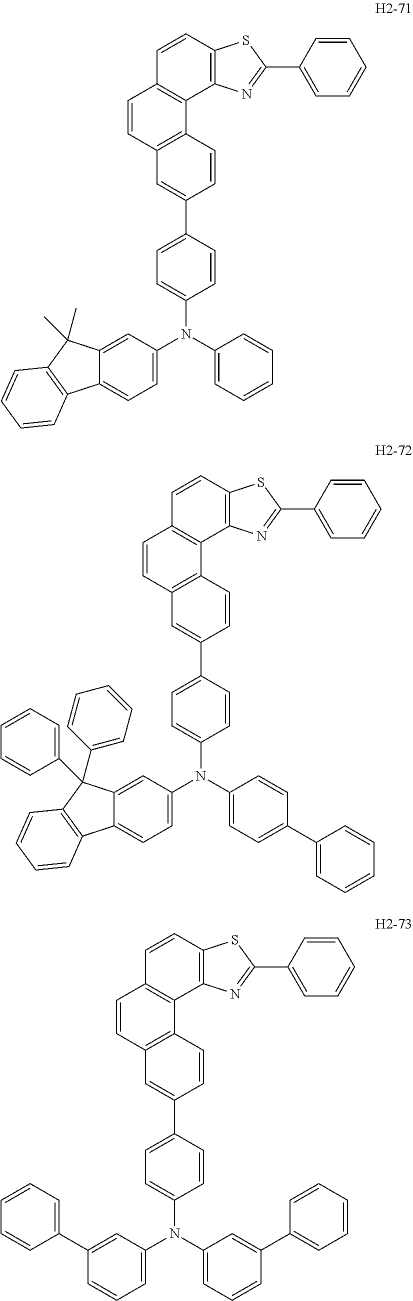

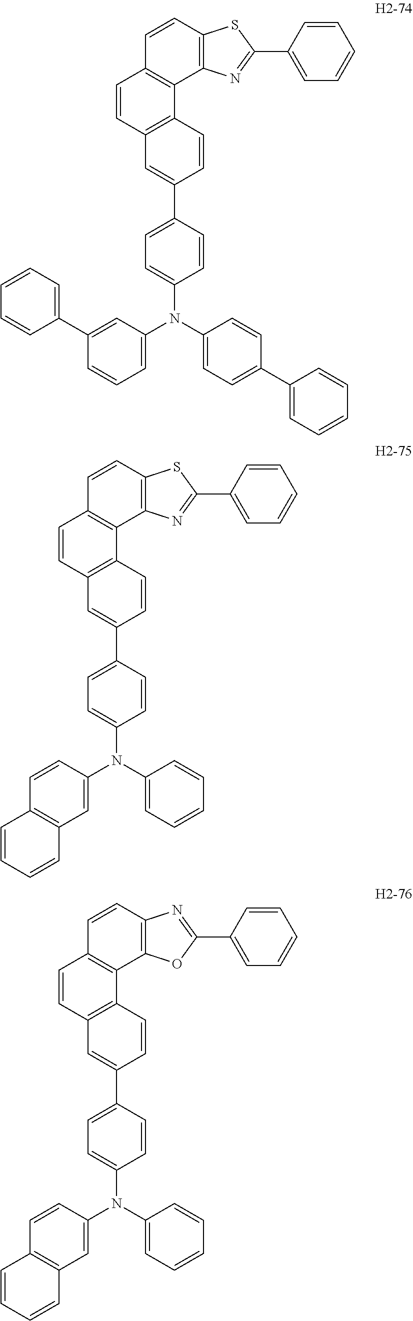 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1]

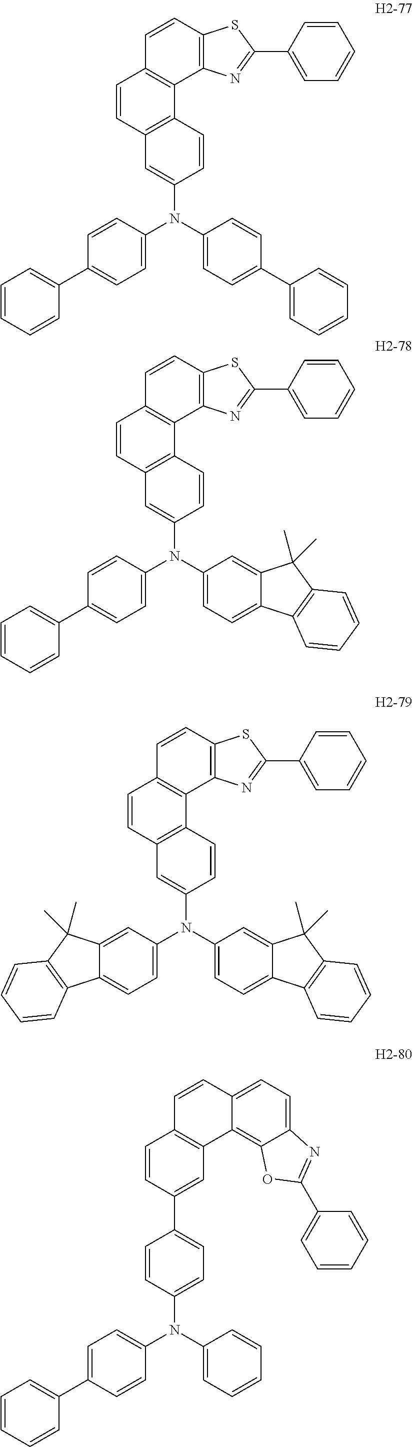

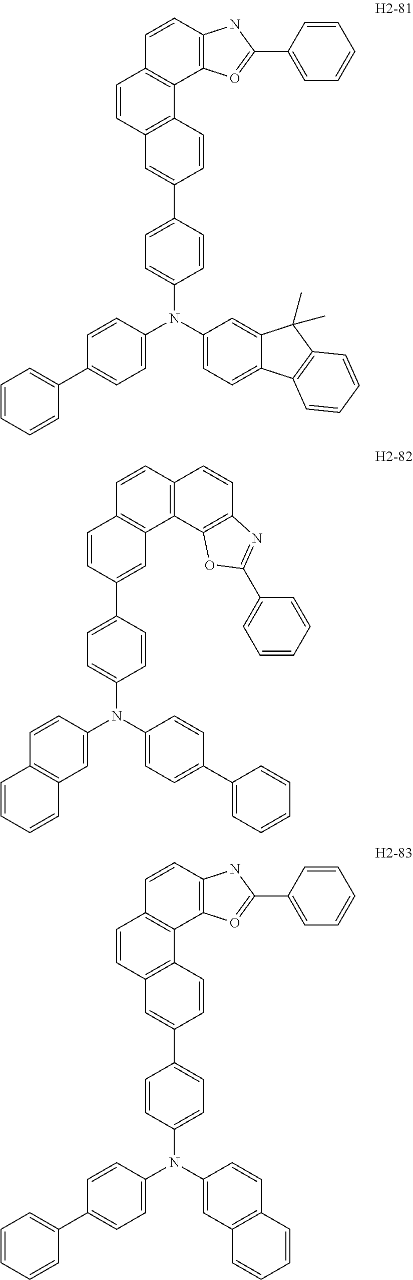

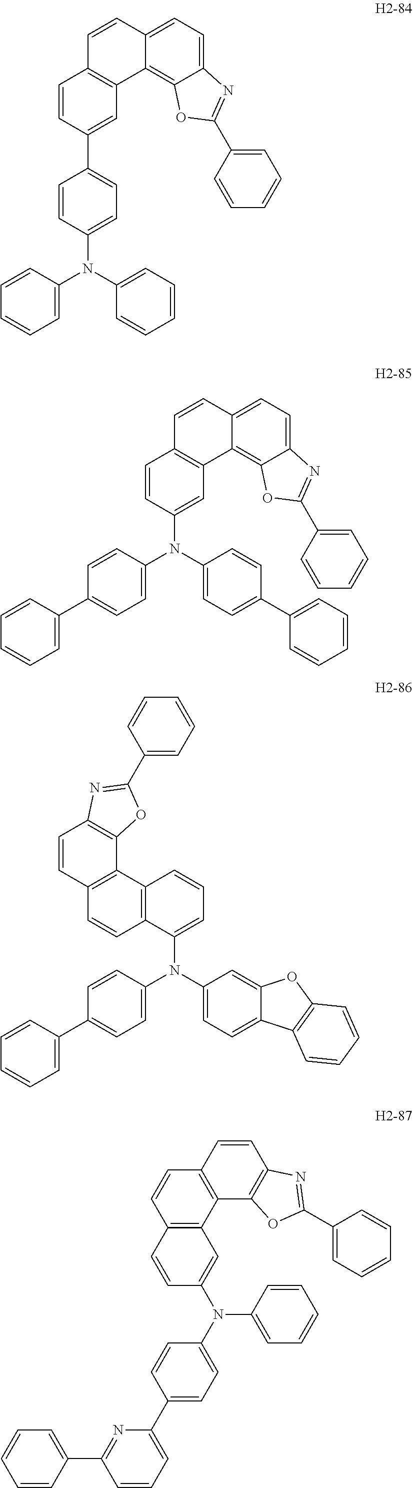 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1]

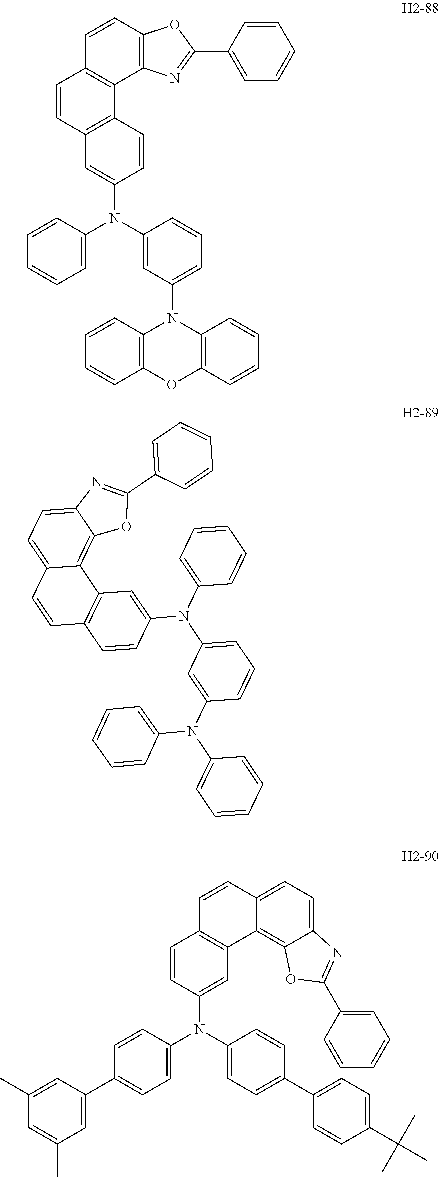

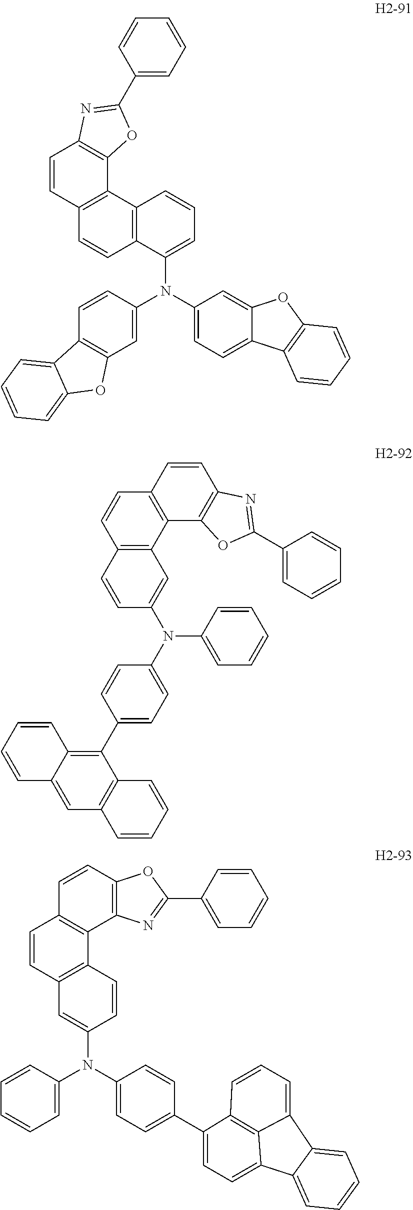

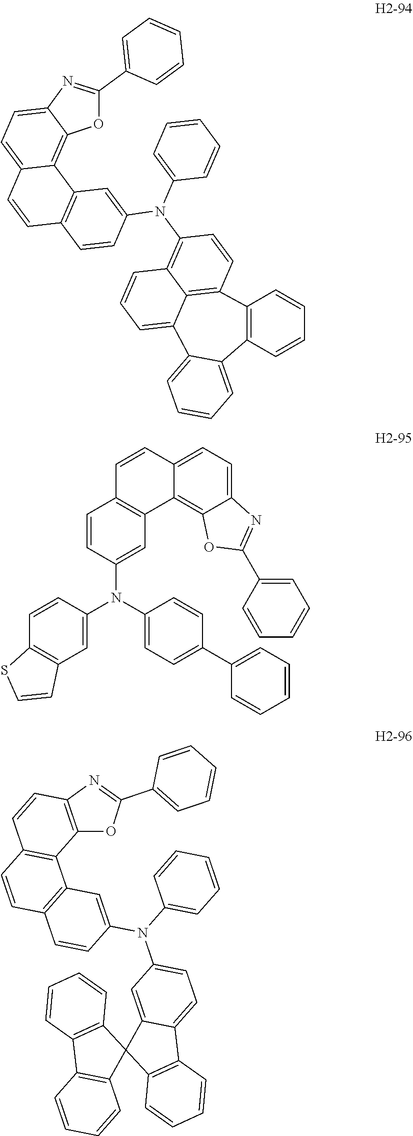

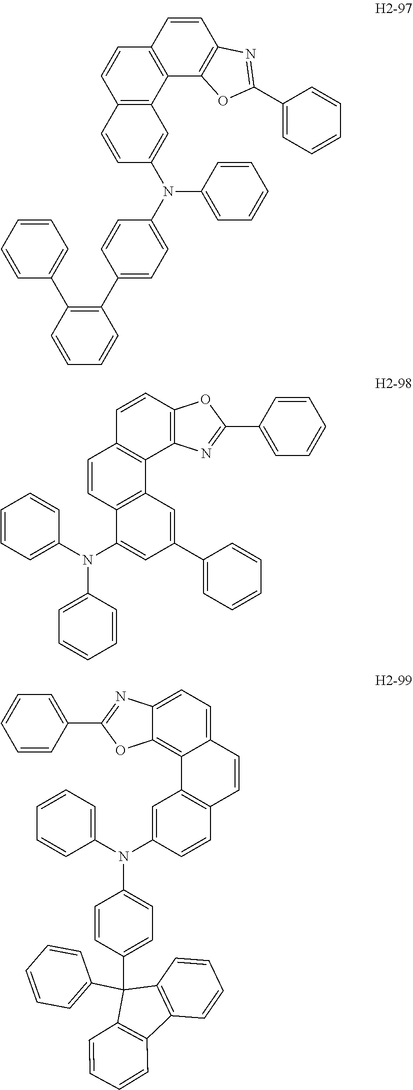 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

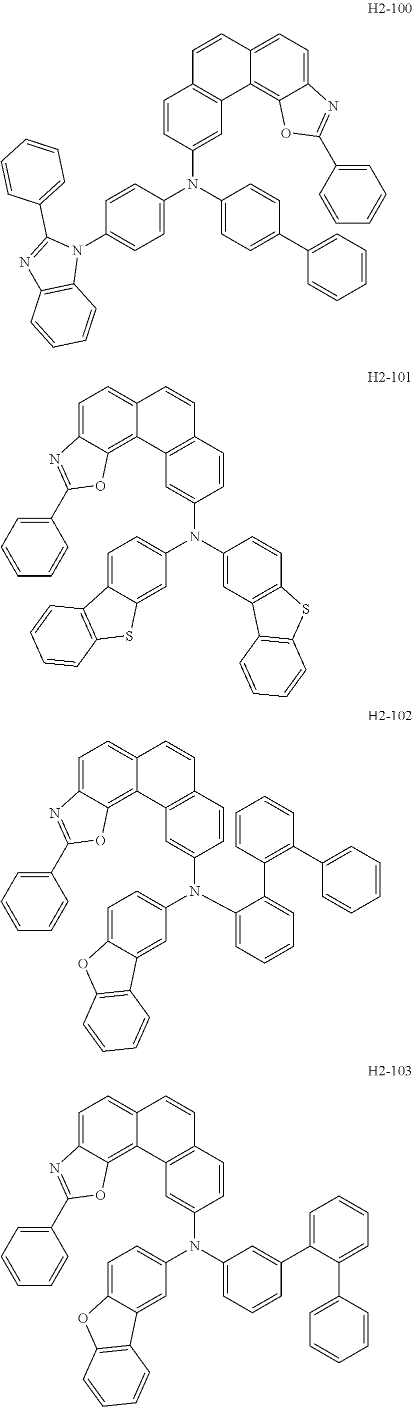

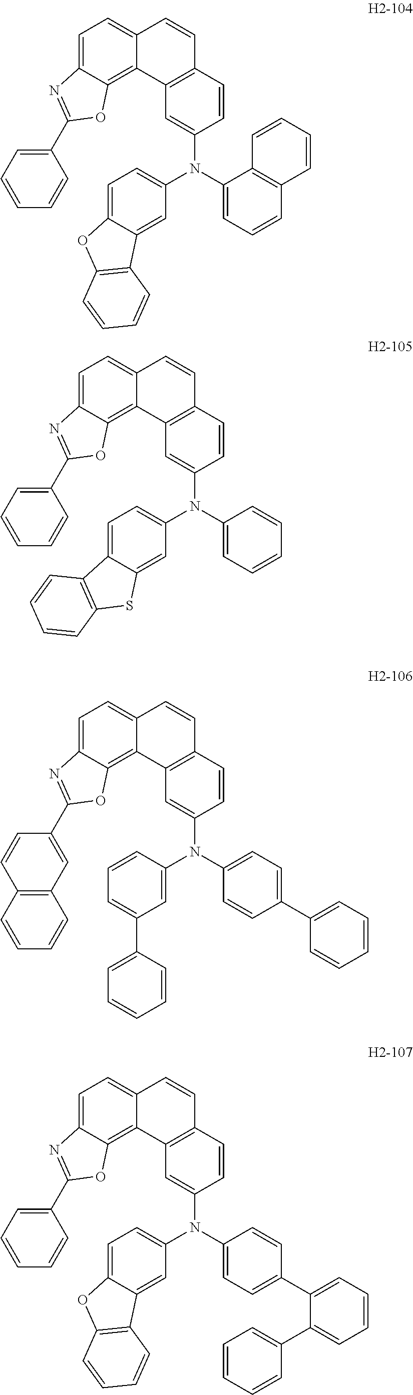

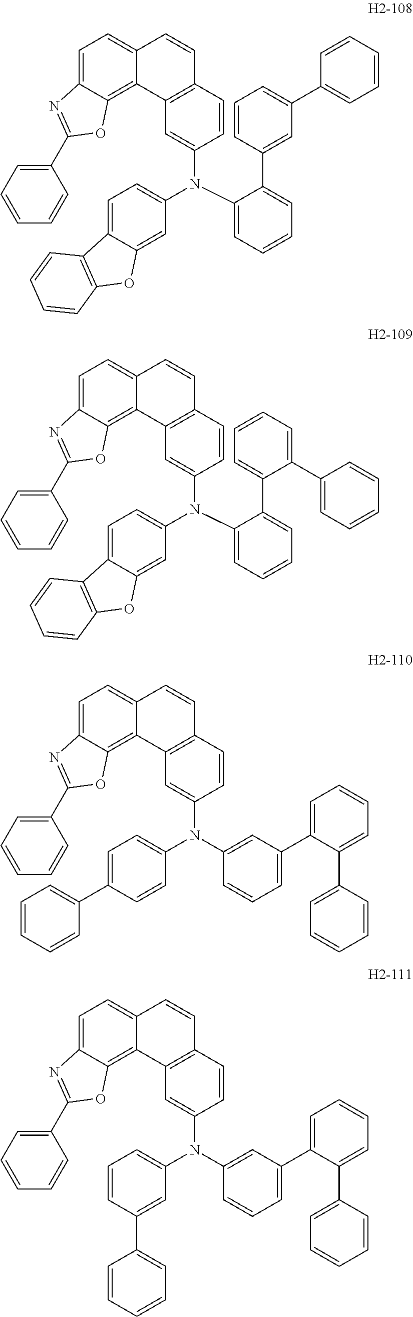 [SMILES: c1ccc(-c2cccc(-c3ccccc3N(c3ccc4c(c3)oc3ccccc34)c3ccc4ccc5ccc6nc(-c7ccccc7)oc6c5c4c3)c2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(N(c3cccc(-c4ccccc4-c4ccccc4)c3)c3ccc4ccc5ccc6nc(-c7ccccc7)oc6c5c4c3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

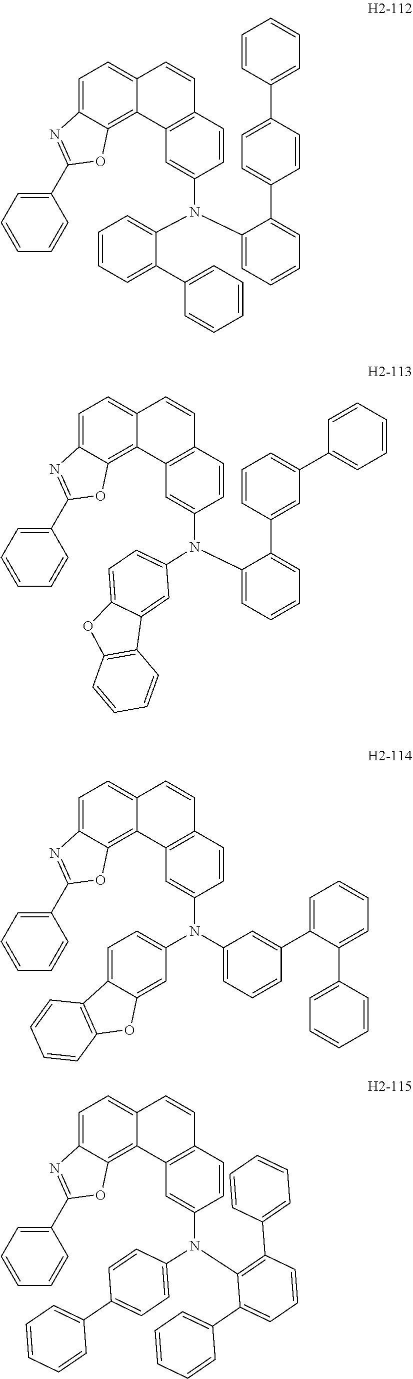

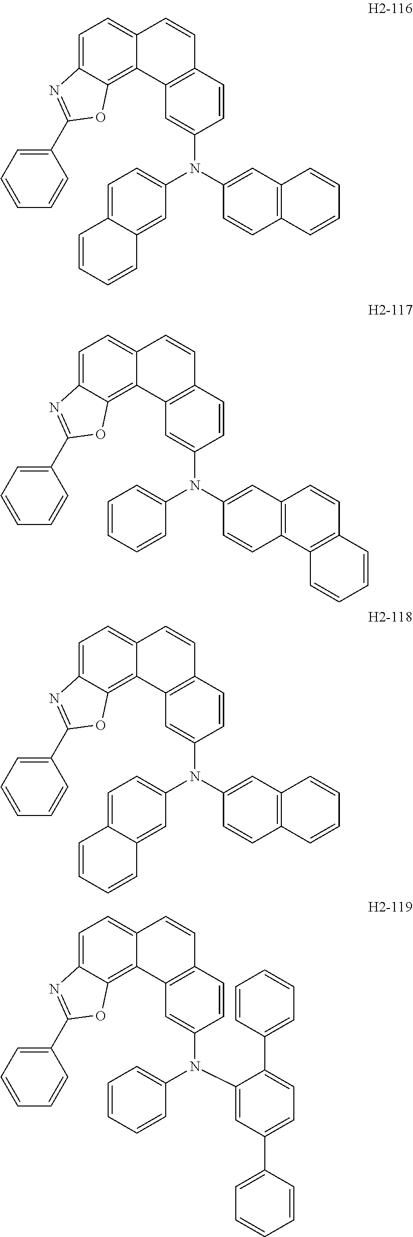

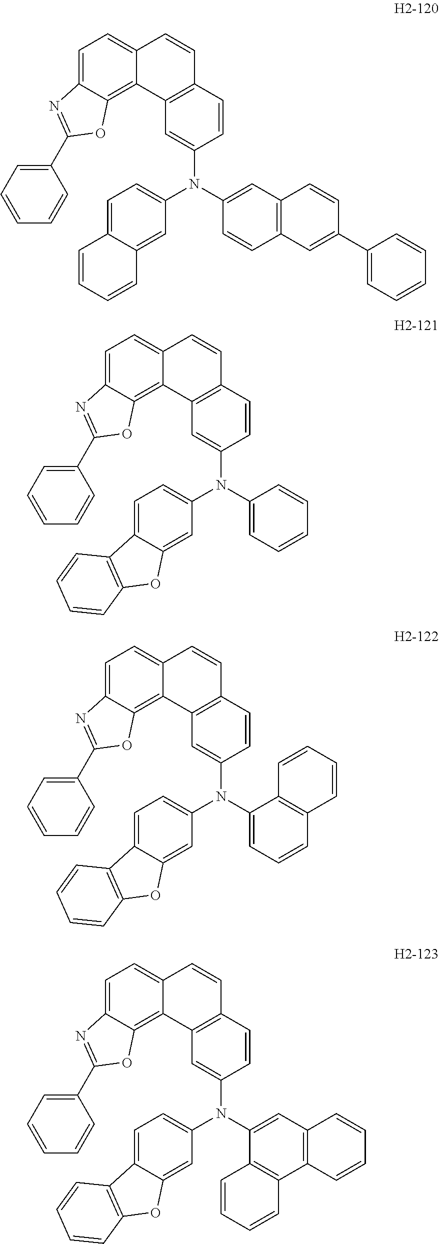

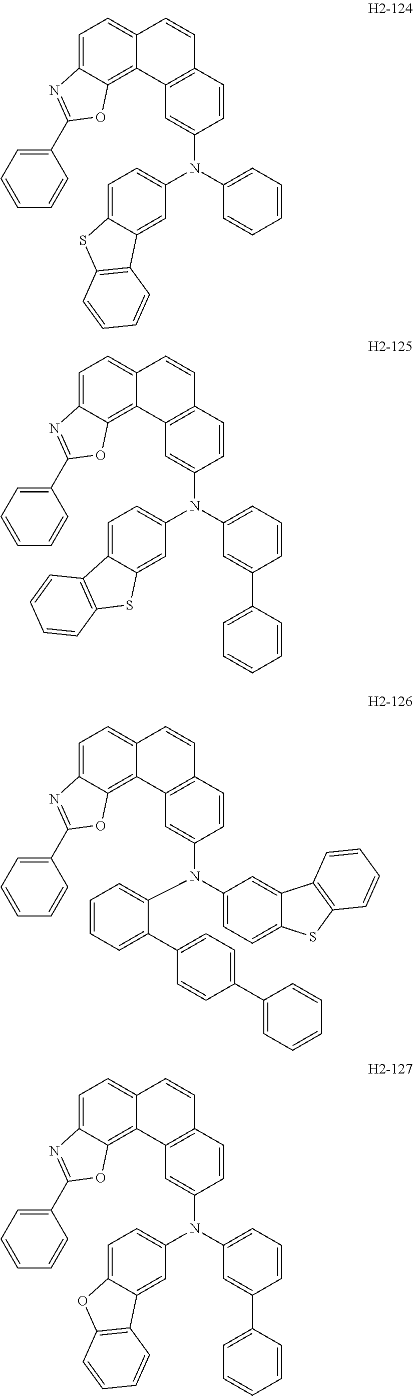

Hereinafter, the compound represented by formula 3 will be described in more detail.

In formula 3, T represents —O— or —S—.

In formula 3, L₅₁ represents a single bond, a substituted or unsubstituted (C6-C30)arylene, or a substituted or unsubstituted (3- to 30-membered)heteroarylene. According to one embodiment of the present disclosure, L₅₁ represents a single bond, or a substituted or unsubstituted (C6-C18)arylene. According to another embodiment of the present disclosure, L₅₁ represents a single bond or an unsubstituted (C6-C12)arylene. For example, L₅₁ may be a single bond, a phenylene, a biphenylene, a naphthylene, etc.

In formula 3, HAr represents a substituted or unsubstituted (3- to 30-membered)heteroaryl containing a nitrogen(s). According to one embodiment of the present disclosure, HAr represents a (3- to 20-membered)heteroaryl containing a nitrogen(s), substituted with at least one of a (C6-C30)aryl(s) and a (5- to 20-membered)heteroaryl(s). According to another embodiment of the present disclosure, HAr represents a (3- to 7-membered)heteroaryl containing a nitrogen(s), substituted with at least one of a (C6-C28)aryl(s) and a (5- to 13-membered)heteroaryl(s). For example, HAr may be a triazinyl substituted with at least one of a phenyl(s), a biphenyl(s), a naphthyl(s), a terphenyl(s), a naphthylphenyl(s), a phenylnaphthyl(s), a dimethylbenzofluorenyl(s), a chrysenyl(s), a chrysenylnaphthyl(s), a dibenzofuranyl(s), and a benzophenanthrenyl(s), etc.

In formula 3, R₇₁ and R₇₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s). According to one embodiment of the present disclosure, R₇₁ and R₇₂, each independently, represent hydrogen or a substituted or unsubstituted (C6-C18)aryl; or may be linked to an adjacent substituent(s) to form a ring(s). According to another embodiment of the present disclosure, R₇₁ and R₇₂, each independently, represent hydrogen or an unsubstituted (C6-C12)aryl; or may be linked to an adjacent substituent(s) to form a mono- or polycyclic, (6- to 12-membered) aromatic ring(s). For example, R₇₁ and R₇₂, each independently, may be hydrogen, a phenyl, a biphenyl, etc.; or may be linked to an adjacent substituent(s) to form a benzene ring(s) or a naphthalene ring(s).

In formula 3, all represents an integer of 1 to 4, and b₁₂ represents an integer of 1 to 3, where if a₁₁ and b₁₂ are each an integer of 2 or more, each of R₇₁ and each of R₇₂ may be the same or different each other.

The compound represented by formula 3 may be at least one selected from the group consisting of the following compounds, but is not limited thereto.

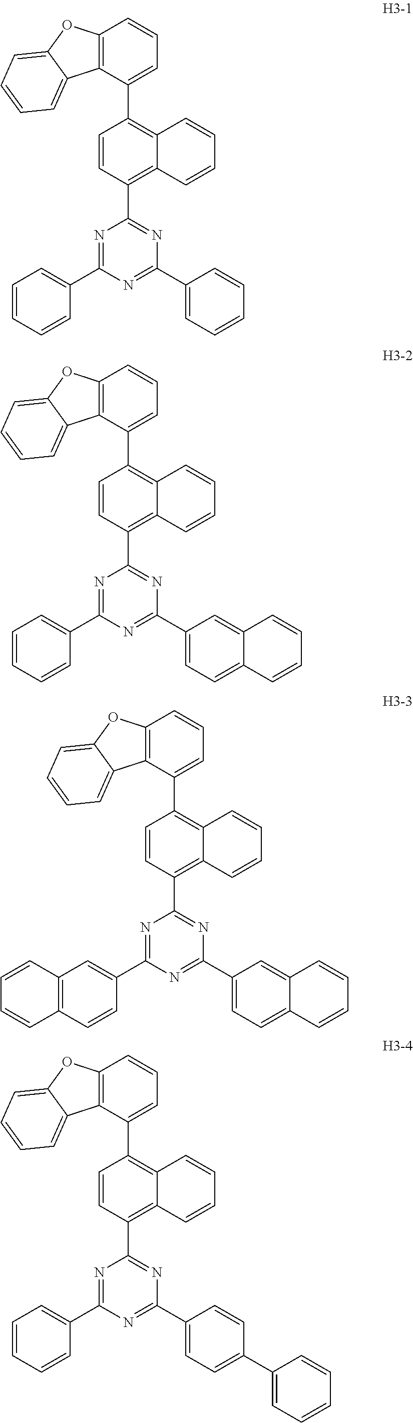

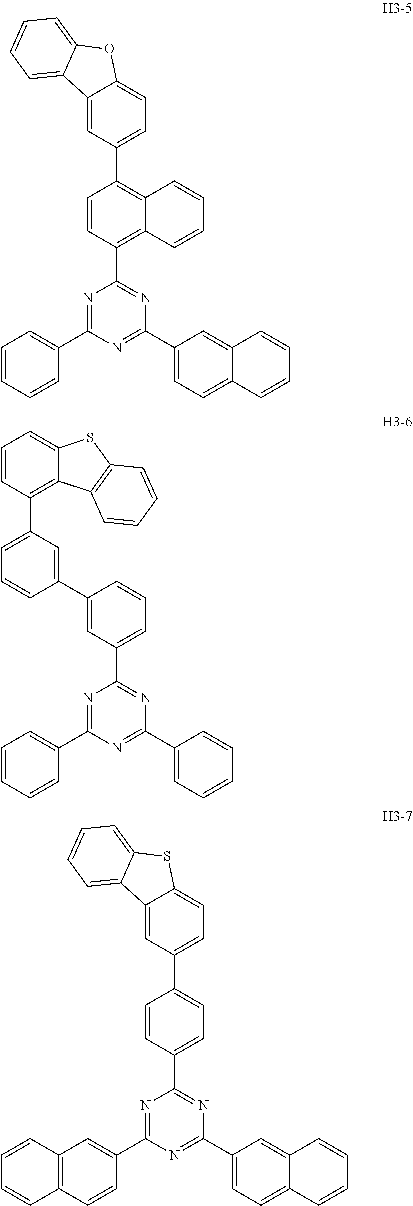

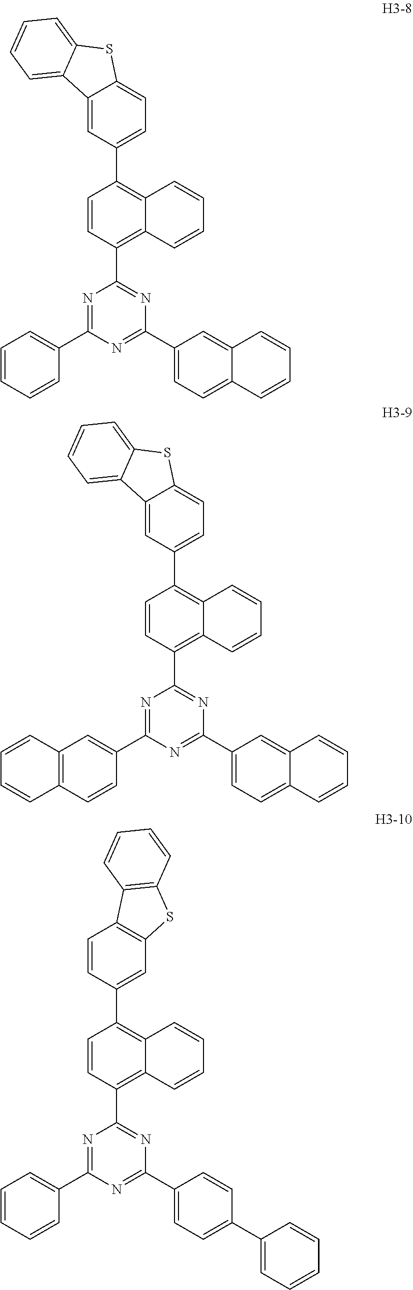

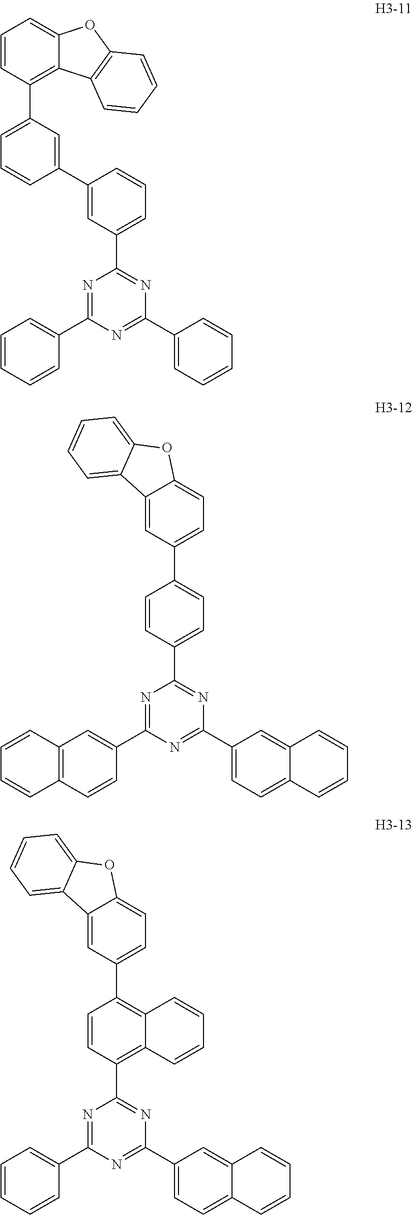

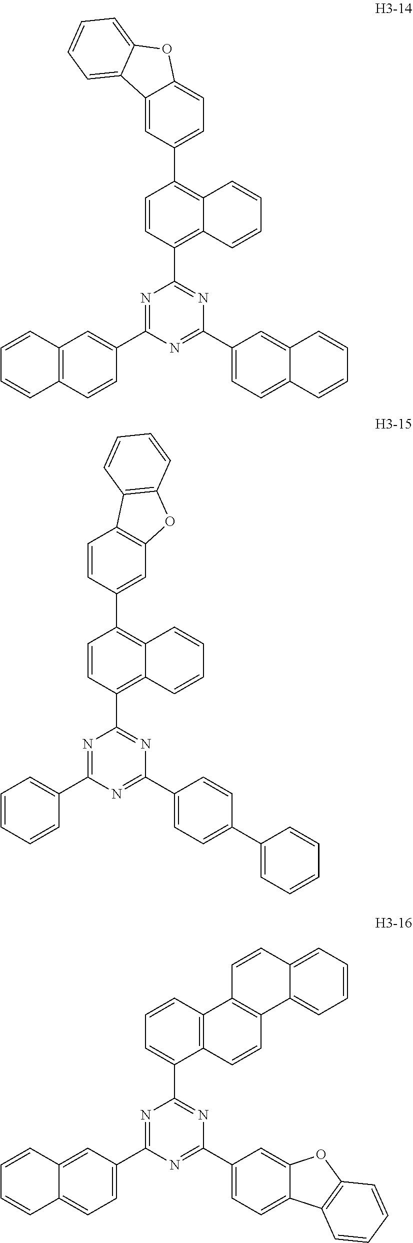 [SMILES: c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5ccc6c(c5)oc5ccccc56)c5ccccc45)n3)cc2)cc1 ; c1ccc2cc(-c3nc(-c4ccc5c(c4)oc4ccccc45)nc(-c4cccc5c4ccc4c6ccccc6ccc54)n3)ccc2c1]

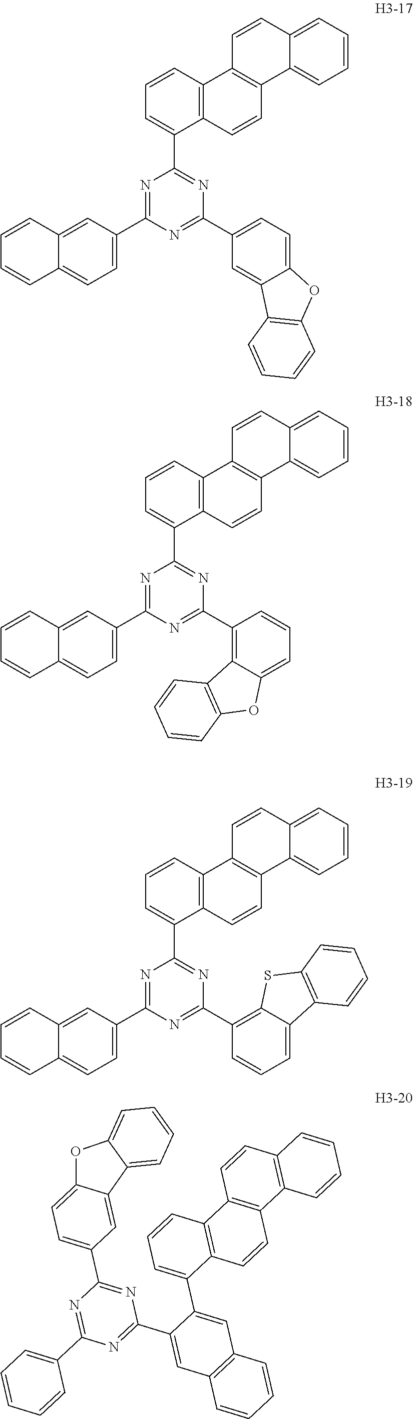

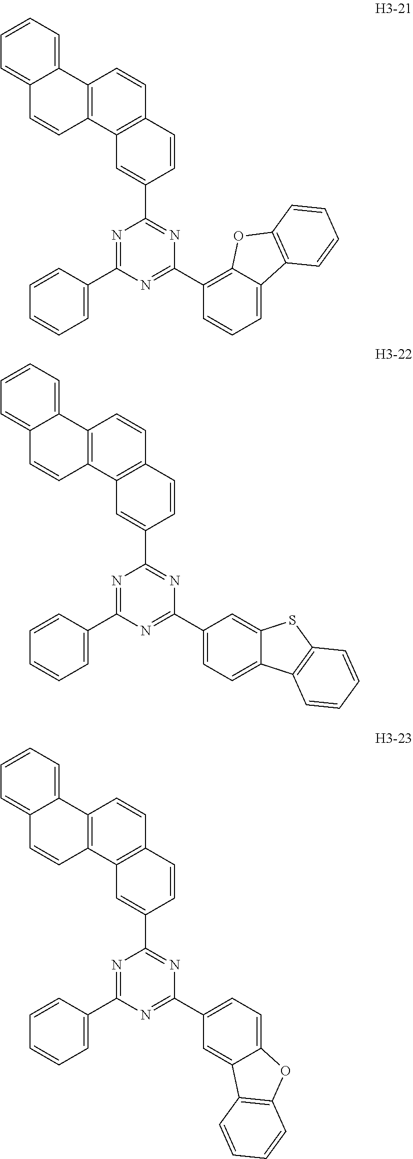

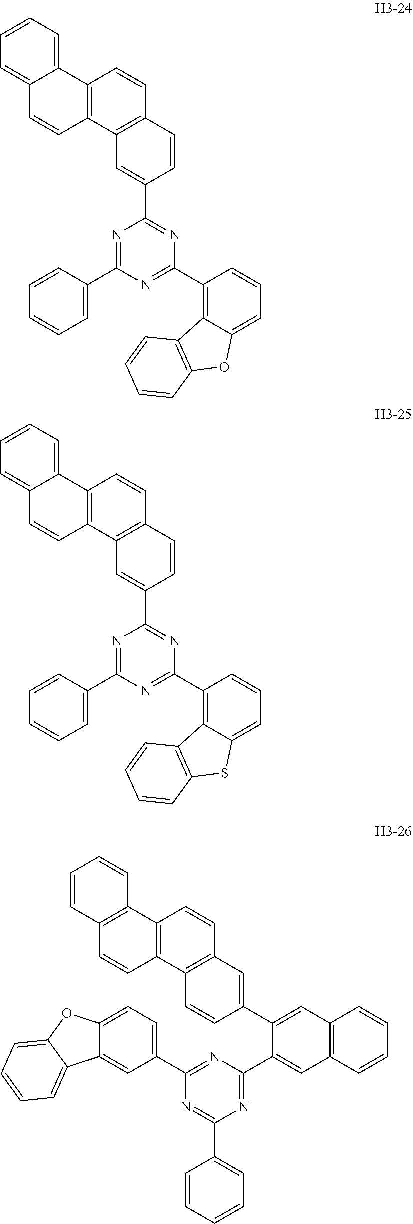 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc(-c2nc(-c3ccc4oc5ccccc5c4c3)nc(-c3cc4ccccc4cc3-c3ccc4c(ccc5c6ccccc6ccc45)c3)n2)cc1]

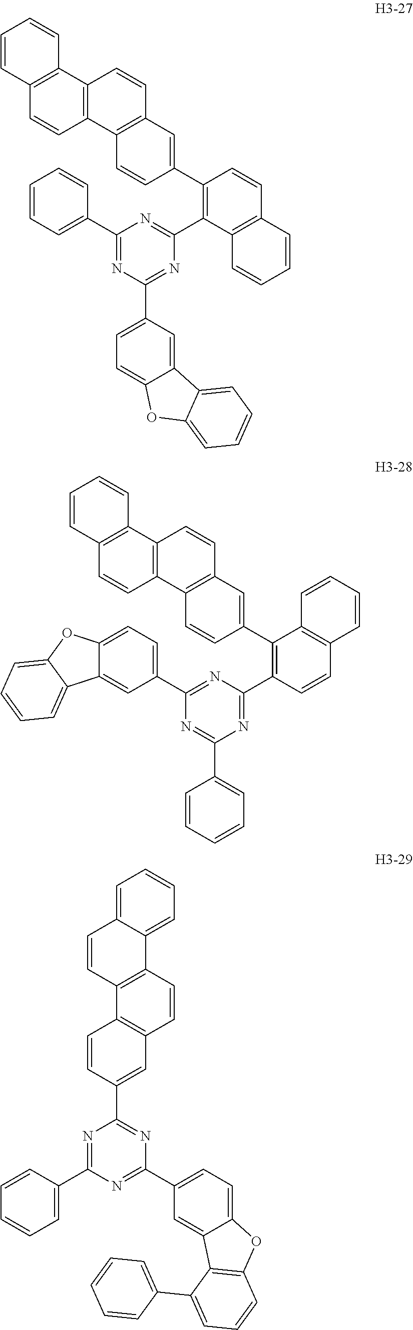 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1]

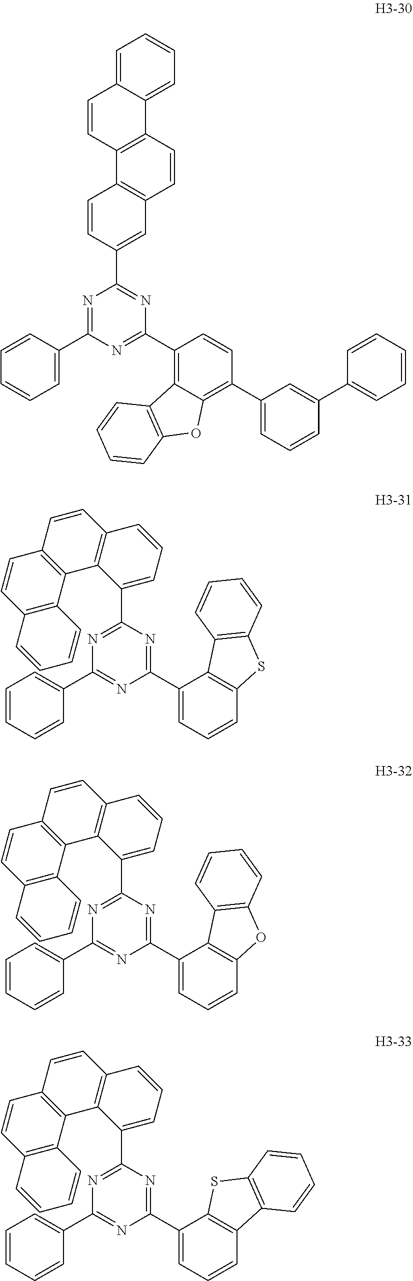 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1]

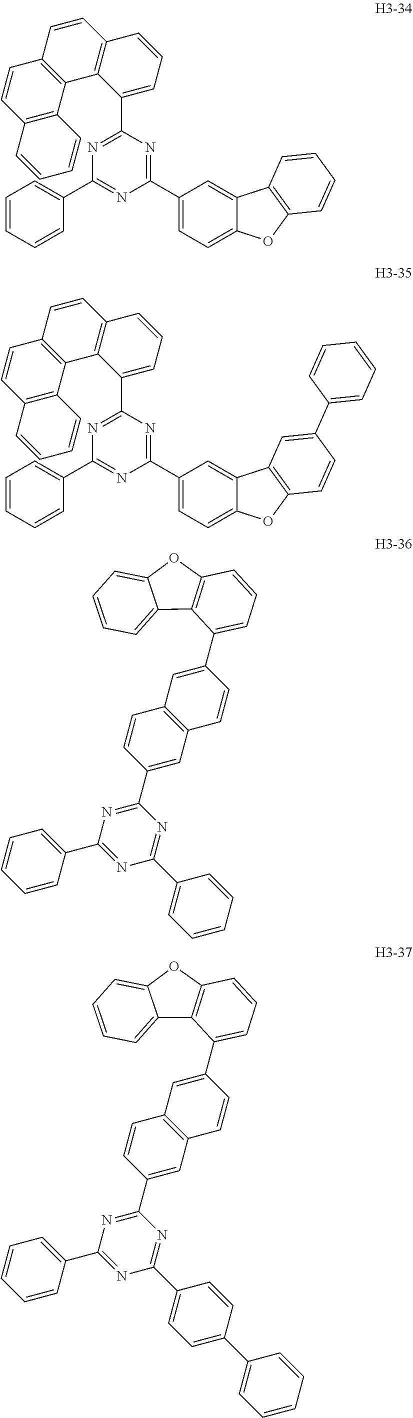

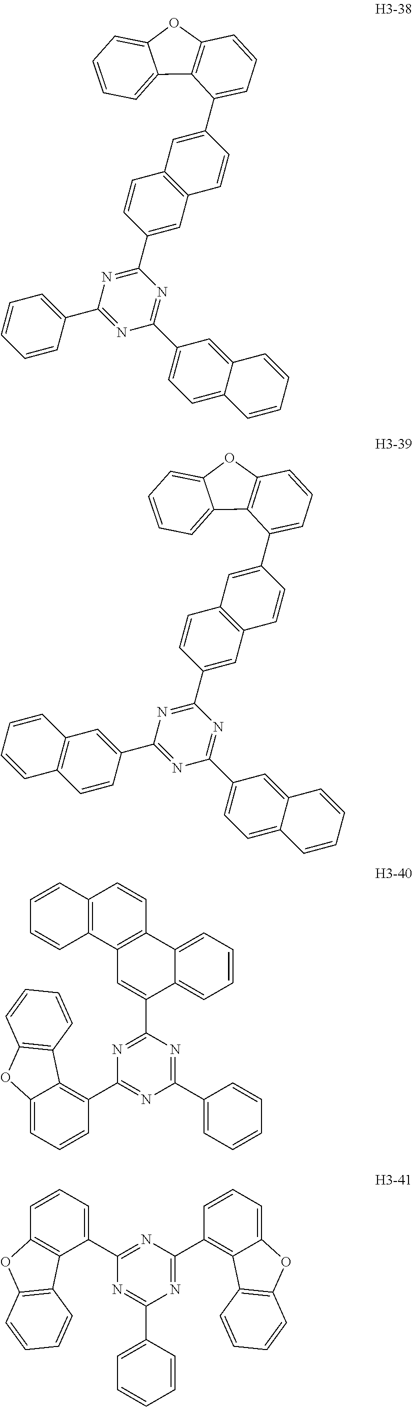

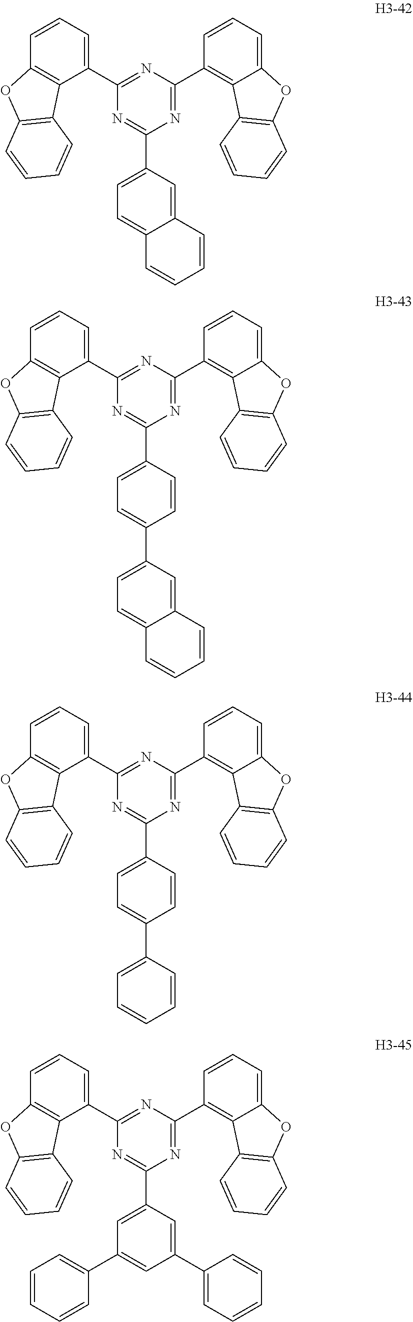 [SMILES: c1ccc2cc(-c3nc(-c4cccc5oc6ccccc6c45)nc(-c4cccc5oc6ccccc6c45)n3)ccc2c1 ; c1ccc2cc(-c3ccc(-c4nc(-c5cccc6oc7ccccc7c56)nc(-c5cccc6oc7ccccc7c56)n4)cc3)ccc2c1 ; c1ccc(-c2ccc(-c3nc(-c4cccc5oc6ccccc6c45)nc(-c4cccc5oc6ccccc6c45)n3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

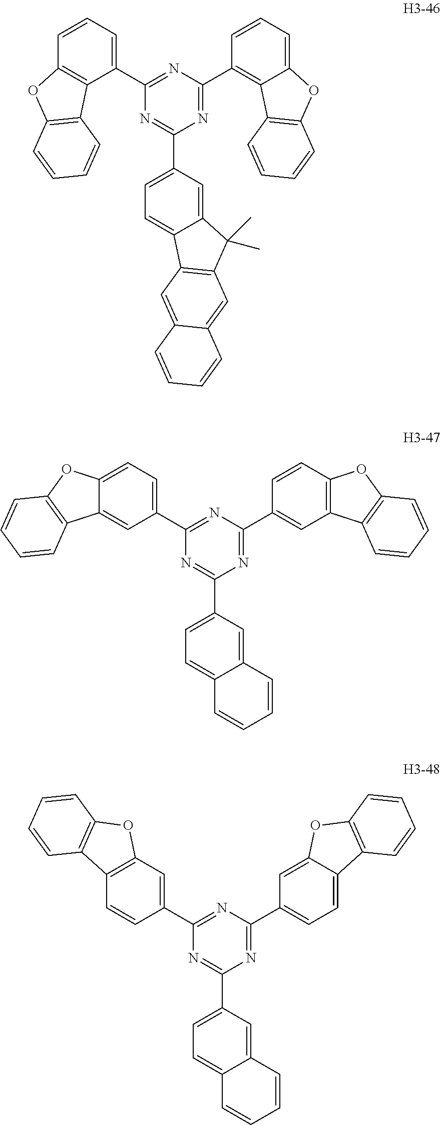 [SMILES: CC1(C)c2cc(-c3nc(-c4cccc5oc6ccccc6c45)nc(-c4cccc5oc6ccccc6c45)n3)ccc2-c2cc3ccccc3cc21 ; c1ccc2cc(-c3nc(-c4ccc5oc6ccccc6c5c4)nc(-c4ccc5oc6ccccc6c5c4)n3)ccc2c1 ; c1ccc2cc(-c3nc(-c4ccc5c(c4)oc4ccccc45)nc(-c4ccc5c(c4)oc4ccccc45)n3)ccc2c1]

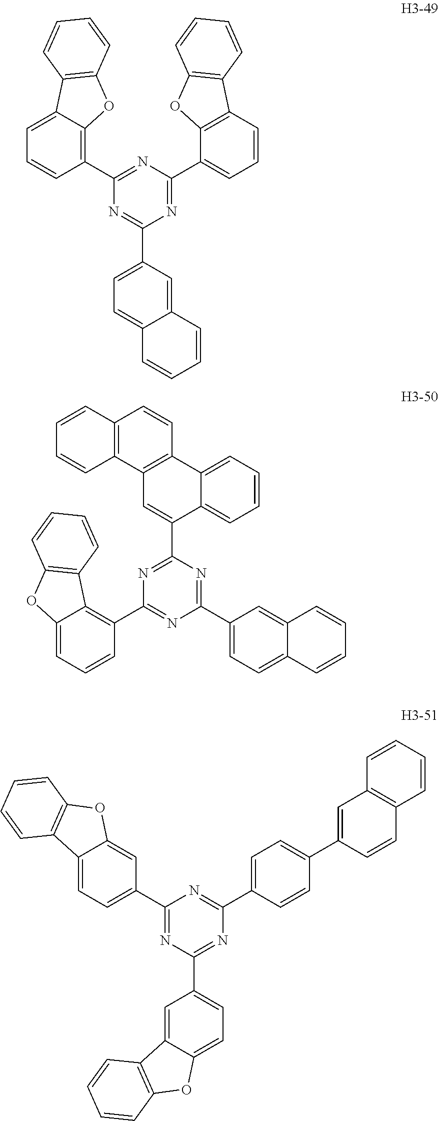

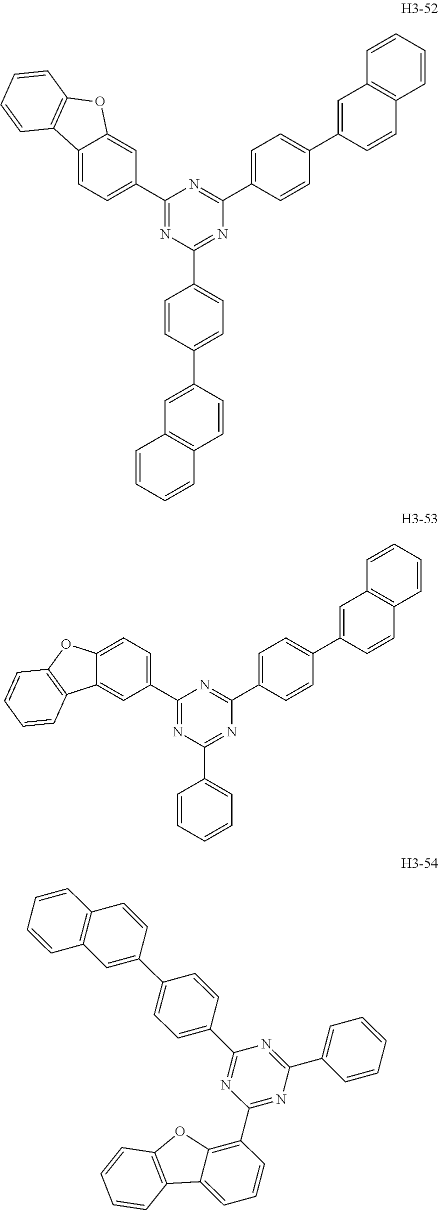 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

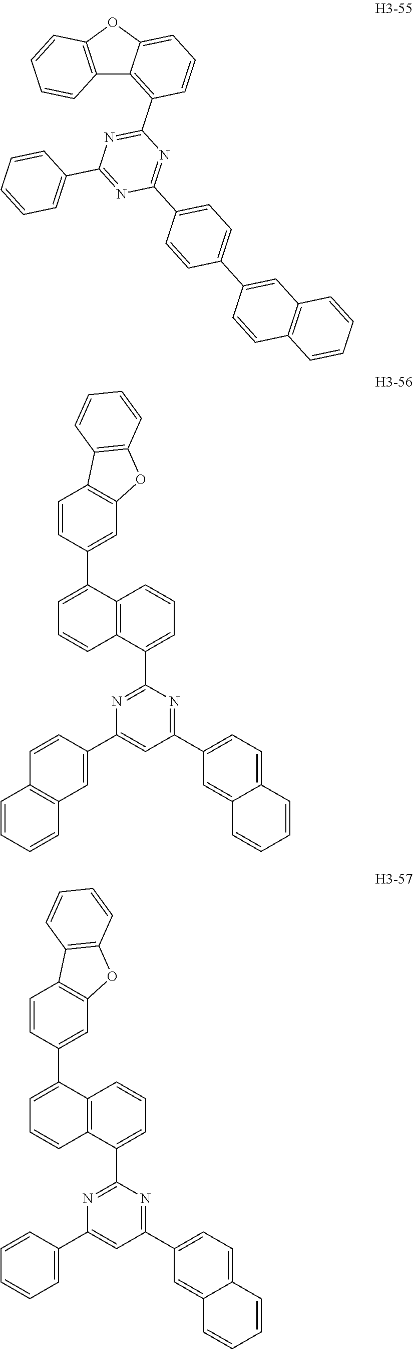 [SMILES: c1ccc(-c2nc(-c3ccc(-c4ccc5ccccc5c4)cc3)nc(-c3cccc4oc5ccccc5c34)n2)cc1 ; c1ccc2cc(-c3cc(-c4ccc5ccccc5c4)nc(-c4cccc5c(-c6ccc7c(c6)oc6ccccc67)cccc45)n3)ccc2c1 ; c1ccc(-c2cc(-c3ccc4ccccc4c3)nc(-c3cccc4c(-c5ccc6c(c5)oc5ccccc56)cccc34)n2)cc1]

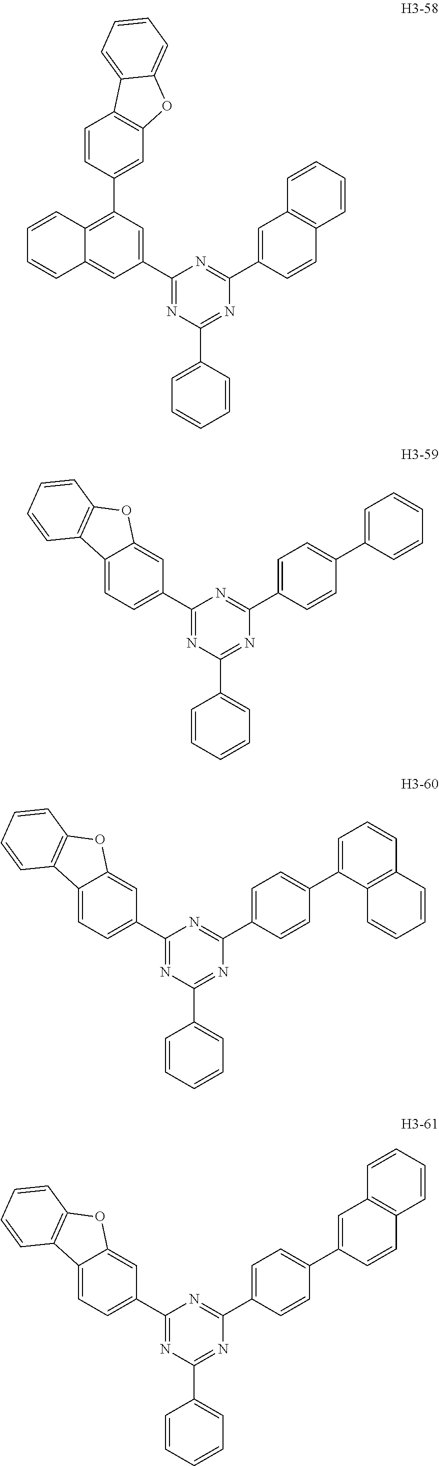 [SMILES: c1ccc(-c2nc(-c3ccc4ccccc4c3)nc(-c3cc(-c4ccc5c(c4)oc4ccccc45)c4ccccc4c3)n2)cc1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1]

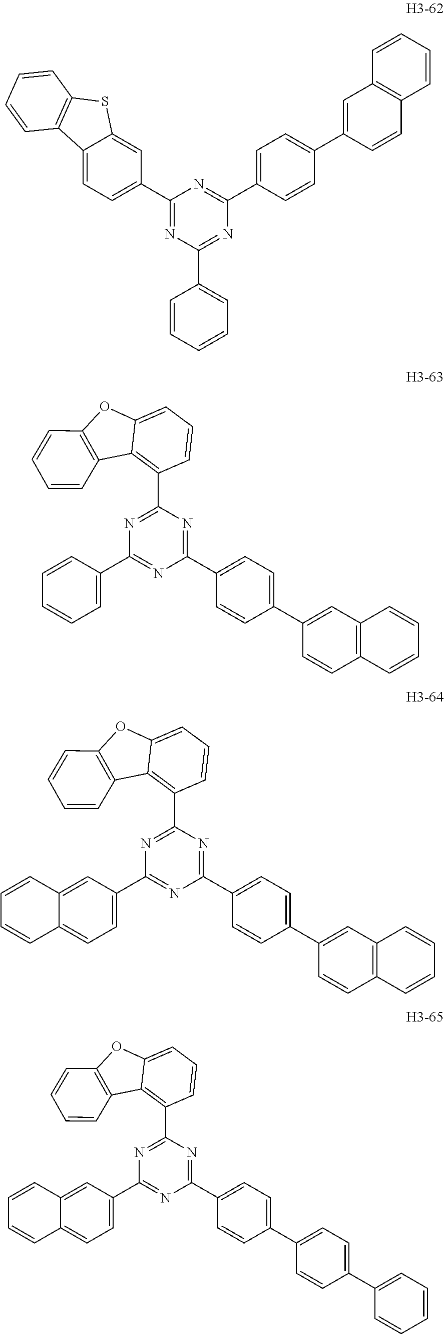 [SMILES: c1ccc(-c2nc(-c3ccc(-c4ccc5ccccc5c4)cc3)nc(-c3ccc4c(c3)sc3ccccc34)n2)cc1 ; c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc(-c4nc(-c5ccc6ccccc6c5)nc(-c5cccc6oc7ccccc7c56)n4)cc3)cc2)cc1]

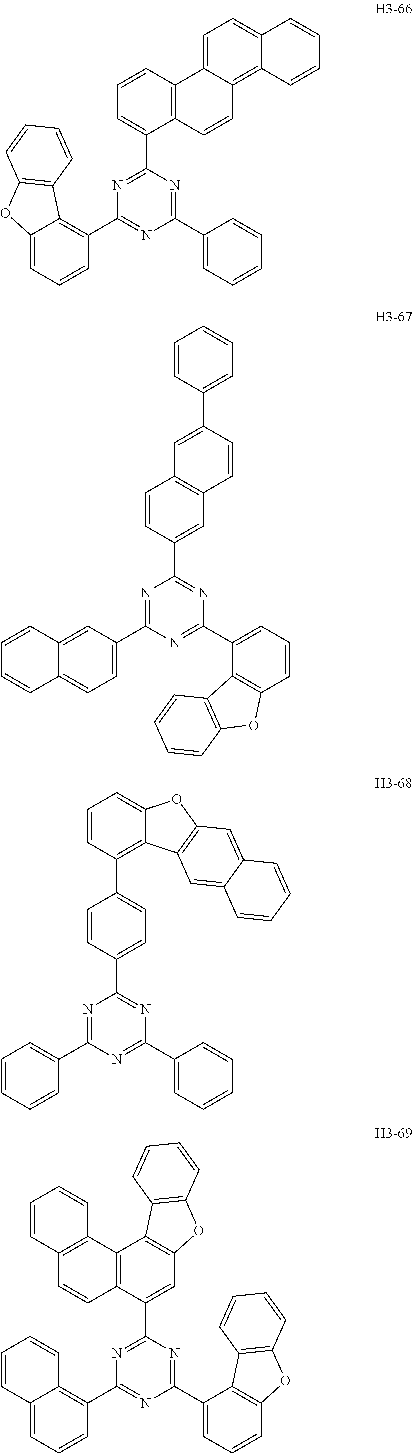

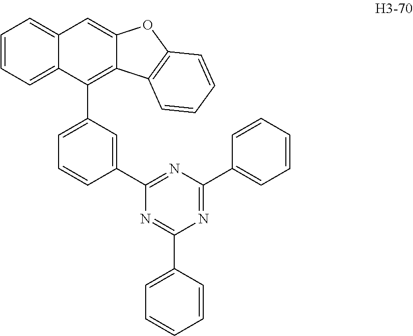 [SMILES: c1ccc(-c2nc(-c3ccccc3)nc(-c3cccc(-c4c5ccccc5cc5oc6ccccc6c45)c3)n2)cc1]

The present disclosure provides a plurality of host materials comprising at least one first host material comprising the organic electroluminescent compound represented by formula 1 and at least one second host material different from the first host material. According to one embodiment of the present disclosure, the combination of at least one of compounds H1-1 to H1-222 and C-1 to C-200 and at least one of compounds H2-1 to H2-127 may be used in an organic electroluminescent device as a plurality of host materials.

The compound represented by formula 1 according to the present disclosure may be produced as shown in the following reaction schemes 1 to 4, but is not limited thereto.

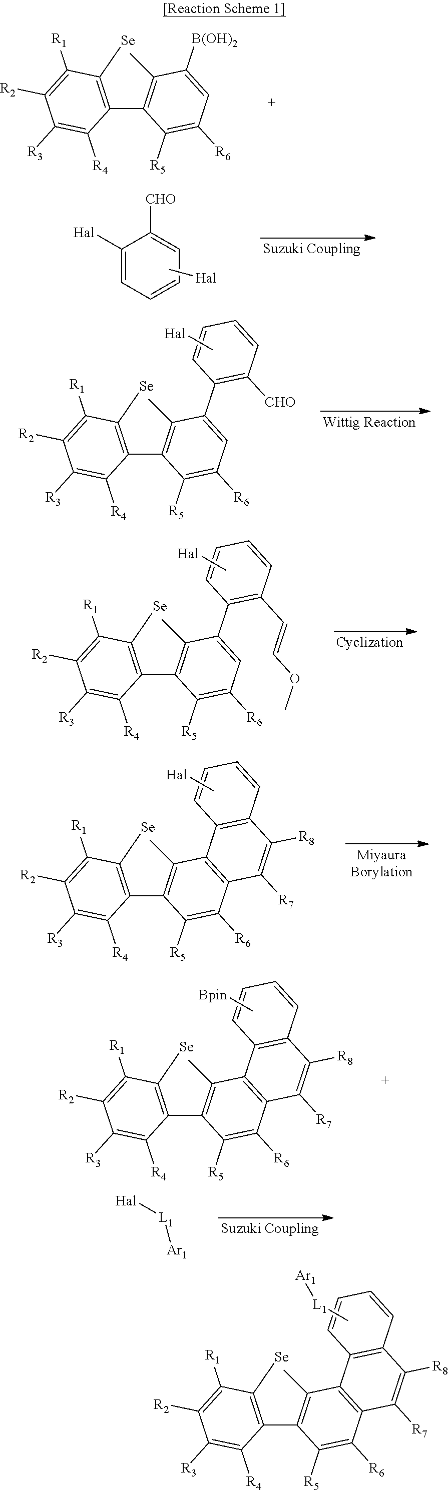

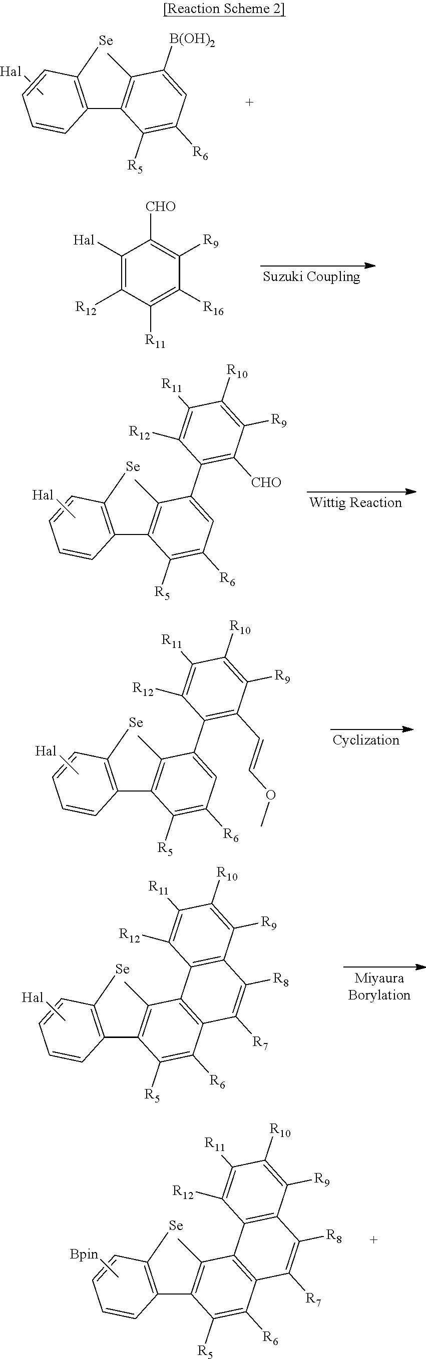

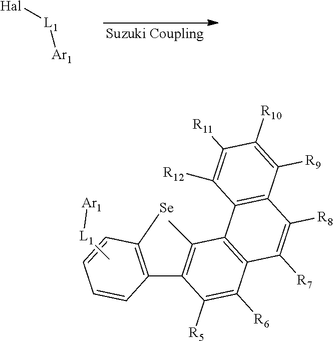

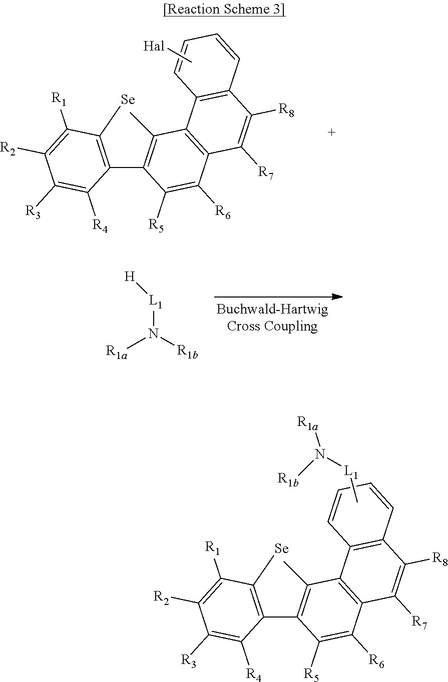

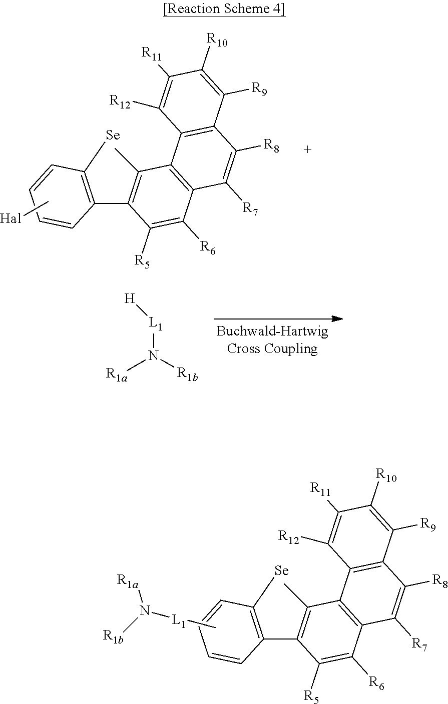

In reaction schemes 1 to 4, R₁ to R₁₂, L₁, Ar₁, Rₐ₁, and Rₐ₂ are as defined in formula 1. In reaction schemes 1 to 4, Hal represents a halogen, and Bpin represents bis(pinacolato)diboron.

Although illustrative synthesis examples of the compounds represented by formula 1 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the reactions above proceed even when substituents which are defined in formula 1 above, but are not specified in the specific synthesis examples, are bonded.

The compound represented by formula 2 according to the present disclosure may be produced by a synthetic method known to one skilled in the art, and for example, by referring to Korean Patert Application Laying-Open No. 2021-0006283 (published on Jan. 18, 2021), etc., but is not limited thereto.

The compound represented by formula 3 according to the present disclosure may be produced by a synthetic method known to one skilled in the art, and for example, by referring to Korean Patert Application Laying-Open No. 2020-0026083 (published on Mar. 10, 2020), and Korean Patert Application Laying-Open No. 2020-0011884 (published on Feb. 4, 2020), etc., but is not limited thereto.

The organic electroluminescent compound represented by formula 1 of the present disclosure may be included in at least one layer of a light-emitting layer, a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer.

An organic electroluminescent device according to the present disclosure comprises an anode, a cathode, and at least one organic layer between the anode and the cathode. The organic layer may comprise a plurality of organic electroluminescent materials comprising the compound represented by formula 1 as the first organic electroluminescent material, and a compound different from the compound represented by formula 1 as the second organic electroluminescent material, According to one embodiment of the present disclosure, an organic electroluminescent device according to the present disclosure comprises an anode, a cathode, and at least one light-emitting layer between the anode and the cathode, wherein at least one of the light-emitting layers may comprise a plurality of host materials. According to another embodiment of the present disclosure, the plurality of host materials may comprise at least one first host material comprising the compound represented by formula 1 and at least one second host material different from the first host material, and the second host material may comprise the compound represented by formula 2 or 3.

The electrodes may be a transflective or reflective electrode, and may be a top emission type, a bottom emission type, or a both-sides emission type, depending on the materials forming the electrodes. In addition, a hole injection layer may be further doped with a p-dopant, and an electron injection layer may be further doped with an n-dopant.

The light-emitting layer may include a host and a dopant. The host may include a plurality of host materials, in which the compound represented by formula 1 and a compound different from the compound represented by formula 1 may be included as the first and second host compounds, respectively, in the plurality of host materials. According to one embodiment of the present disclosure, the second host compound may be the compound represented by formula 2 or 3. The ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50. When the at least two host materials are comprised in one layer, they may be mixture-evaporated to form a layer, or separately co-evaporated at the same time to form a layer.

In the present disclosure, the light-emitting layer is a layer that emits light, which may be a single layer or a plurality of layers in which two or more layers are stacked. In the plurality of host materials of the present disclosure, the first and second host materials may be included in one layer or in different light-emitting layers, respectively. According to one embodiment of the present disclosure, the doping concentration of the dopant compound with respect to the host compound of the light-emitting layer may be less than 20 wt %.

The dopant comprised in the organic electroluminescent material of the present disclosure may be at least one phosphorescent or fluorescent dopant, and is preferably a phosphorescent dopant. The phosphorescent dopant material applied to the present disclosure is not particularly limited, but may be a complex compound of a metal atom selected from iridium (Ir), osmium (Os), copper (Cu) and platinum (Pt), and preferably ortho-metallated complex compounds of a metal atom selected from iridium (Ir), osmium (Os), copper (Cu), and platinum (Pt), and more preferably ortho-metallated iridium complex compounds.

The dopant comprised in the organic electroluminescent device of the present disclosure may comprise a compound represented by the following formula 101, but is not limited thereto.

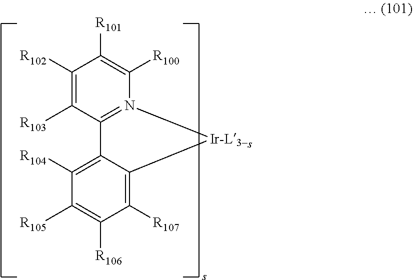

In formula 101,

L′ is selected from the following structures 1 to 3:

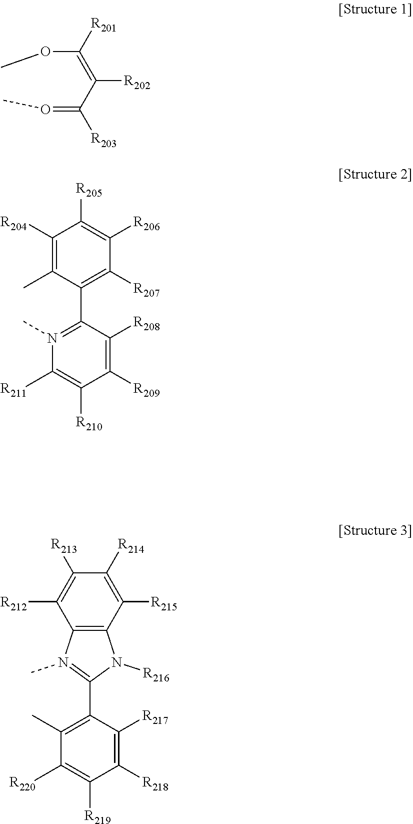

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy, or may be linked to an adjacent substituent(s) to form a ring(s), e.g., a substituted or unsubstituted quinoline, a substituted or unsubstituted isoquinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a substituted or unsubstituted ring(s), e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and

s represents an integer of 1 to 3.

The specific examples of the dopant compound are as follows, but are not limited thereto.

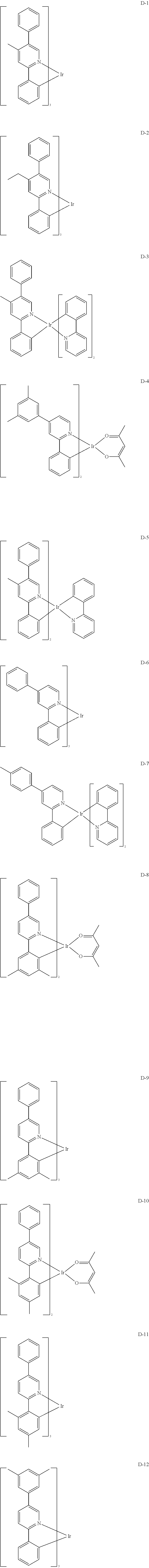

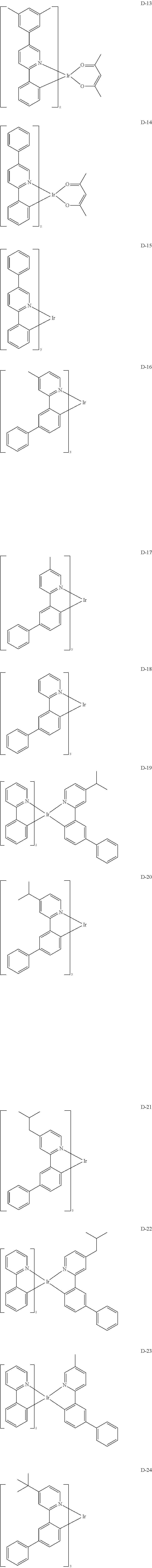

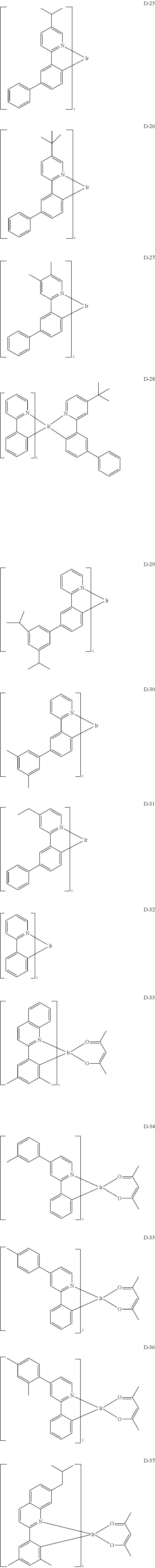

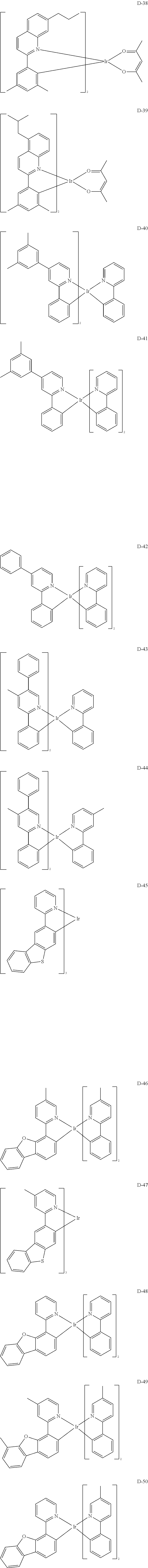

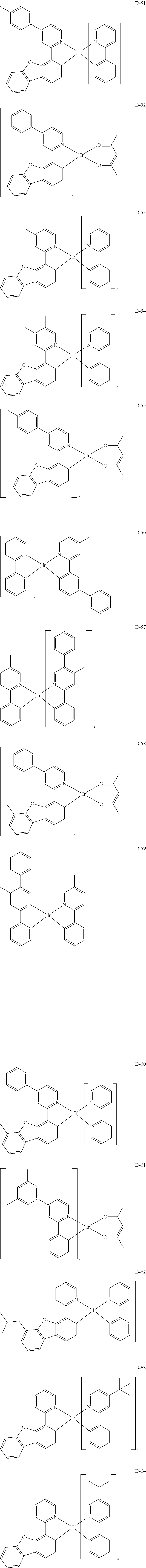

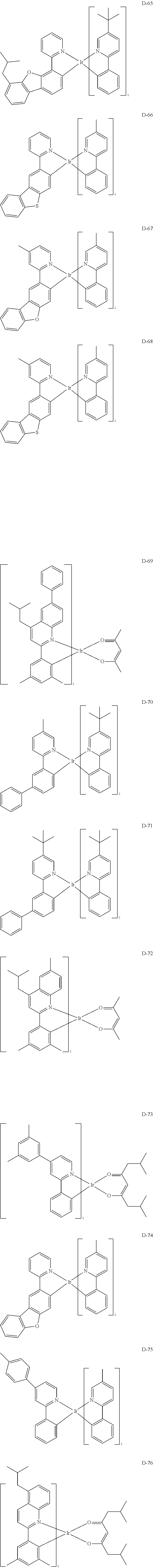

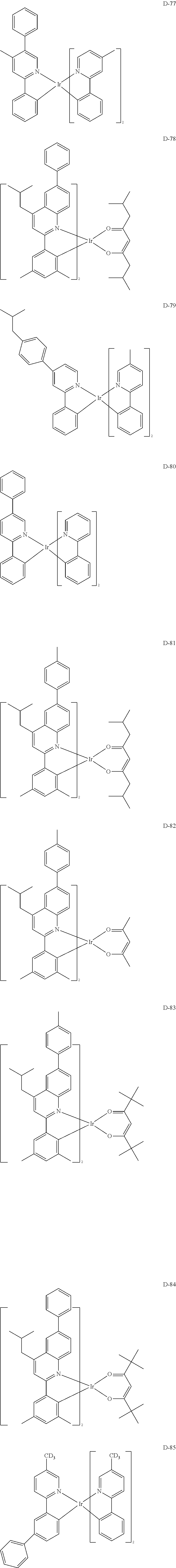

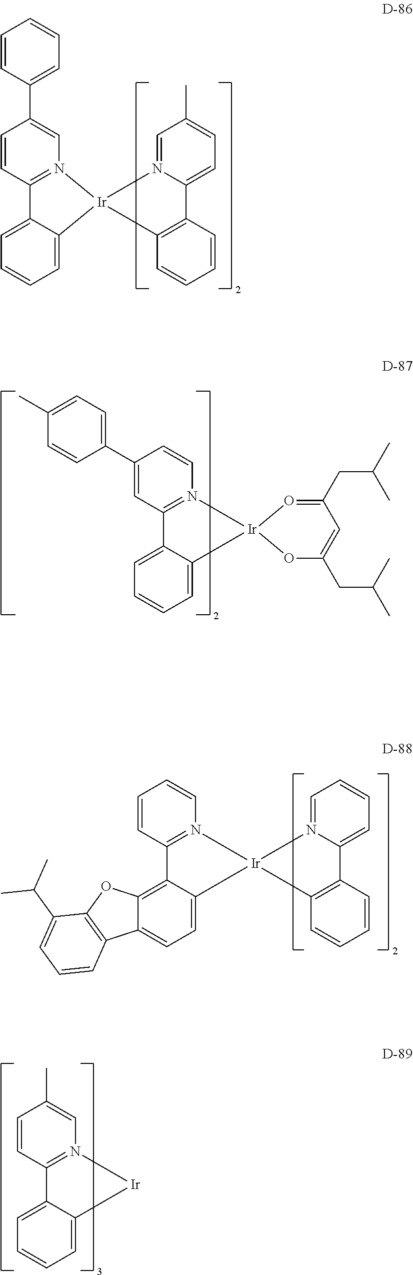

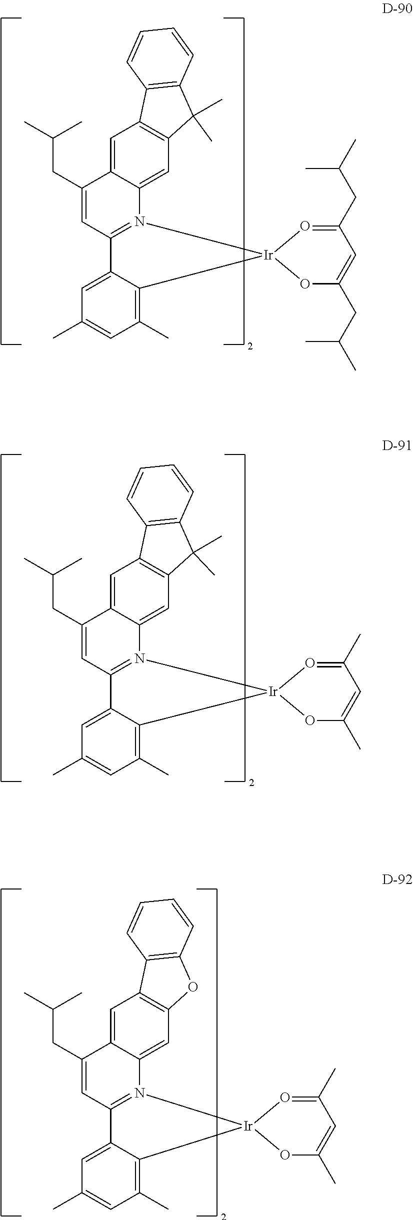

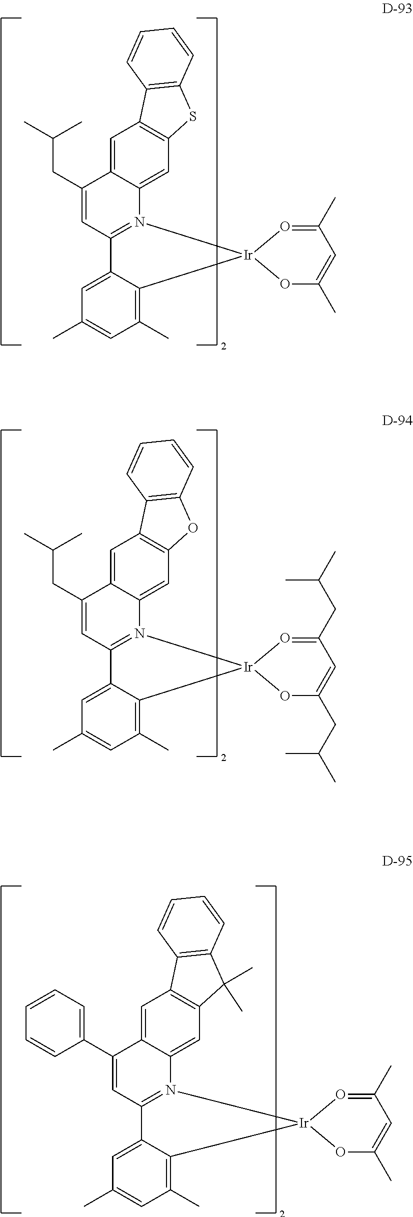

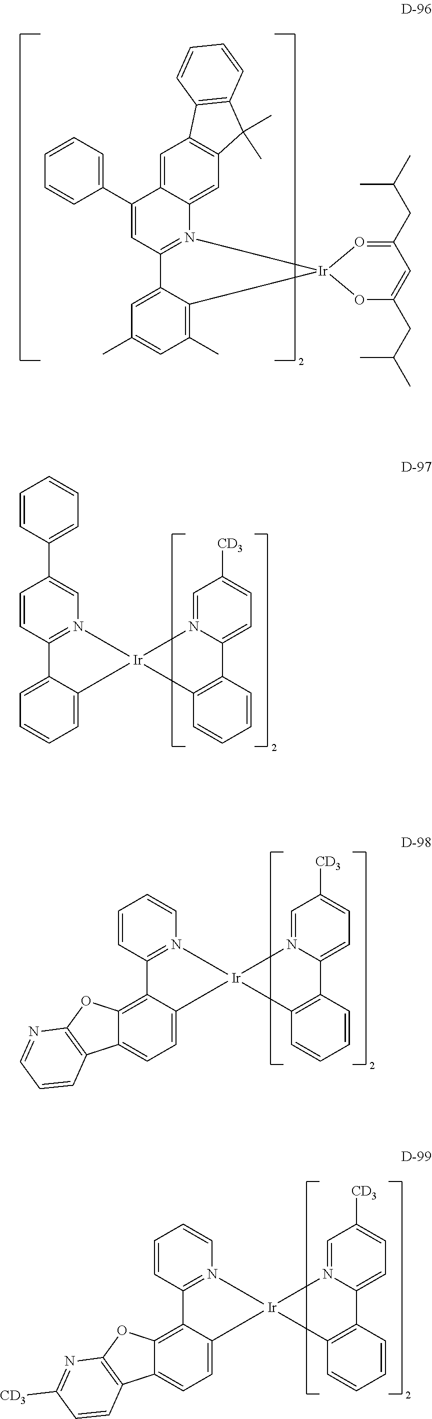

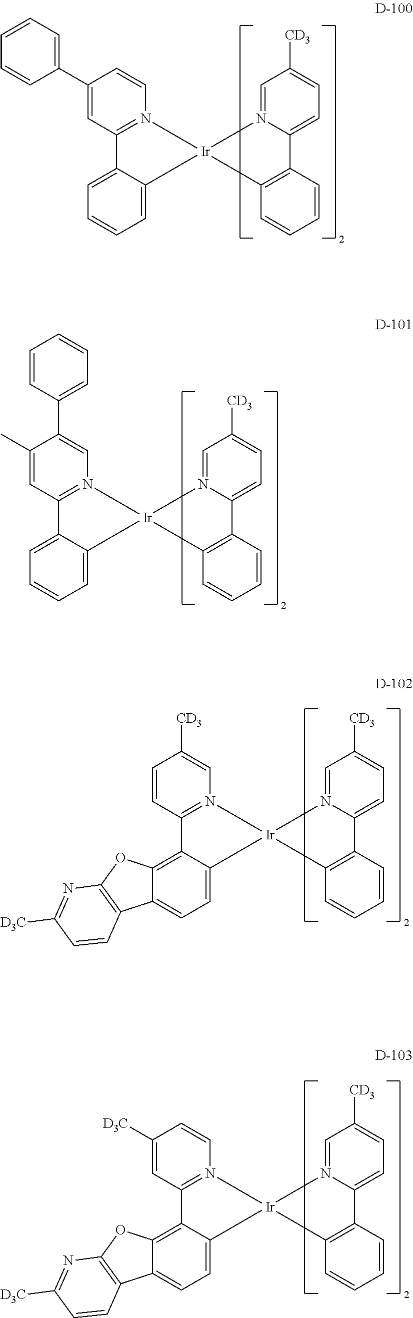

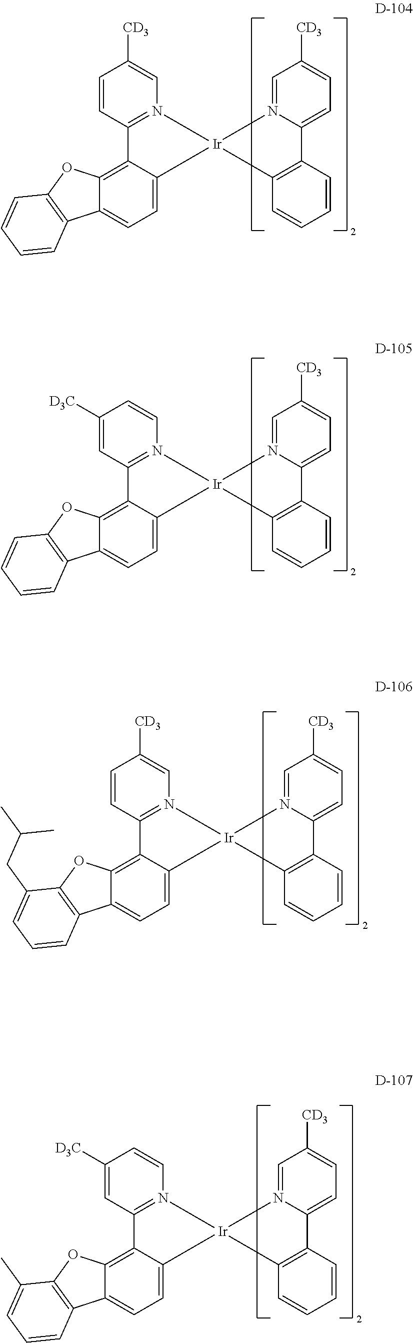

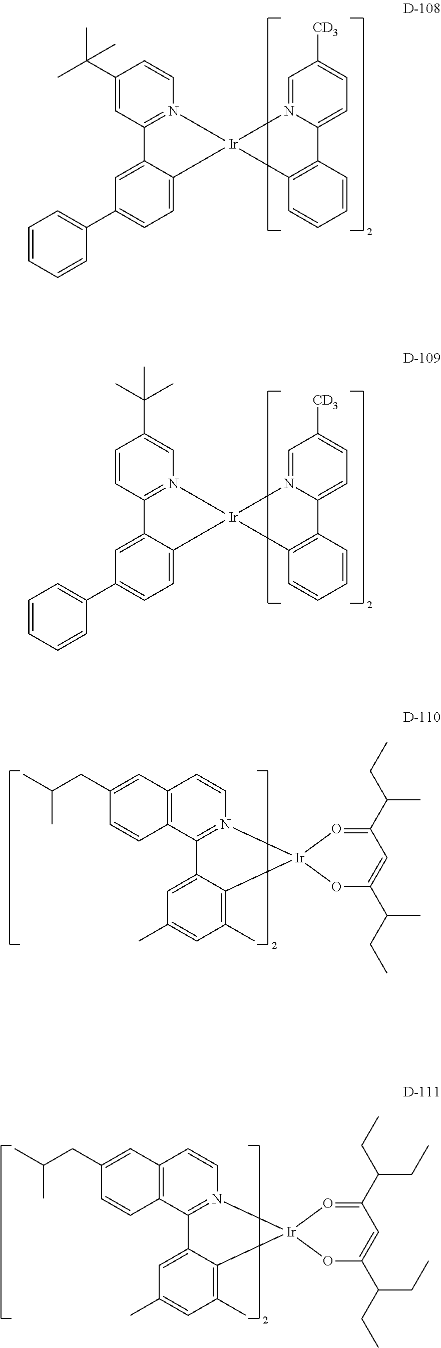

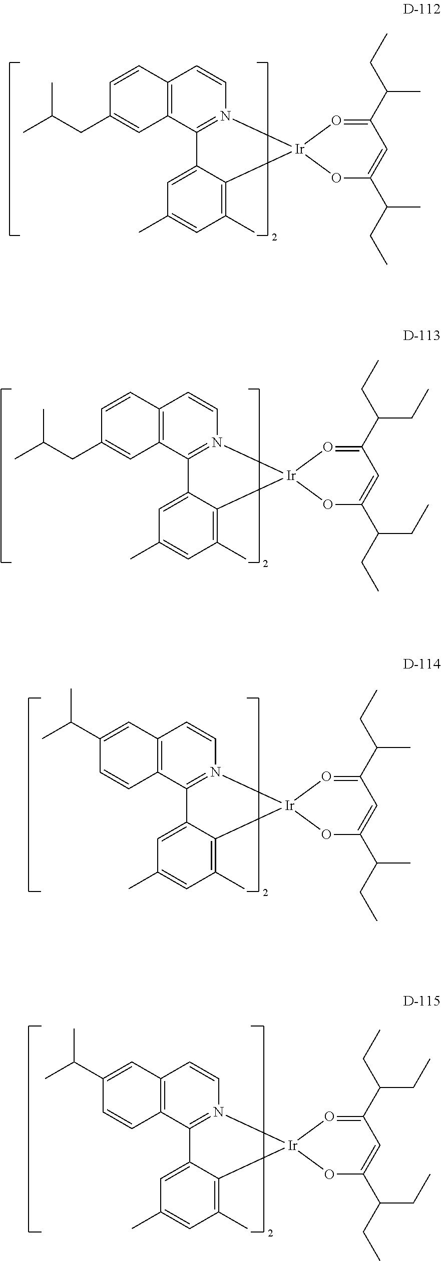

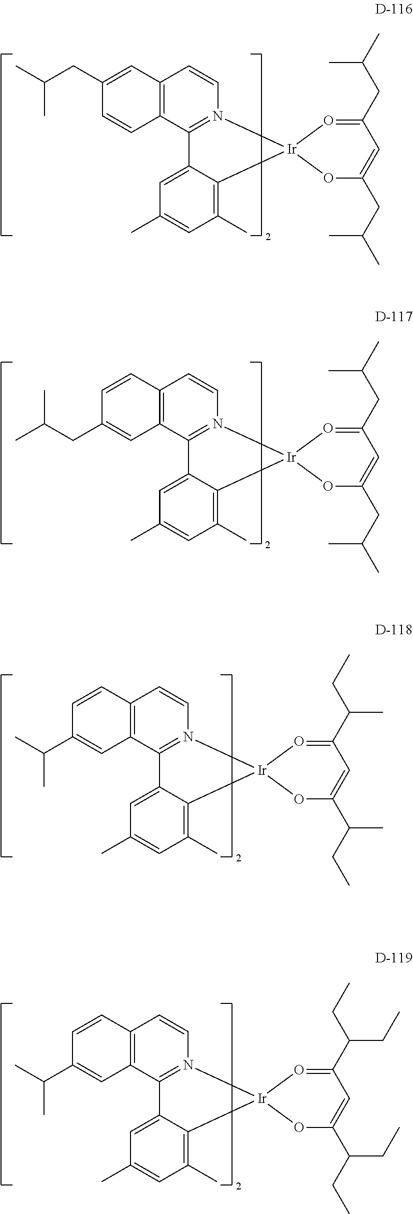

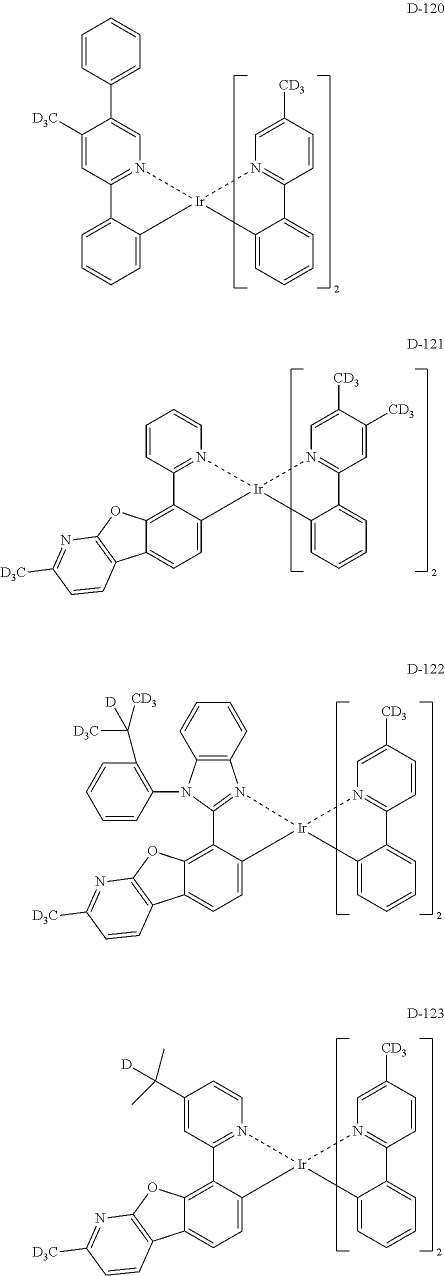

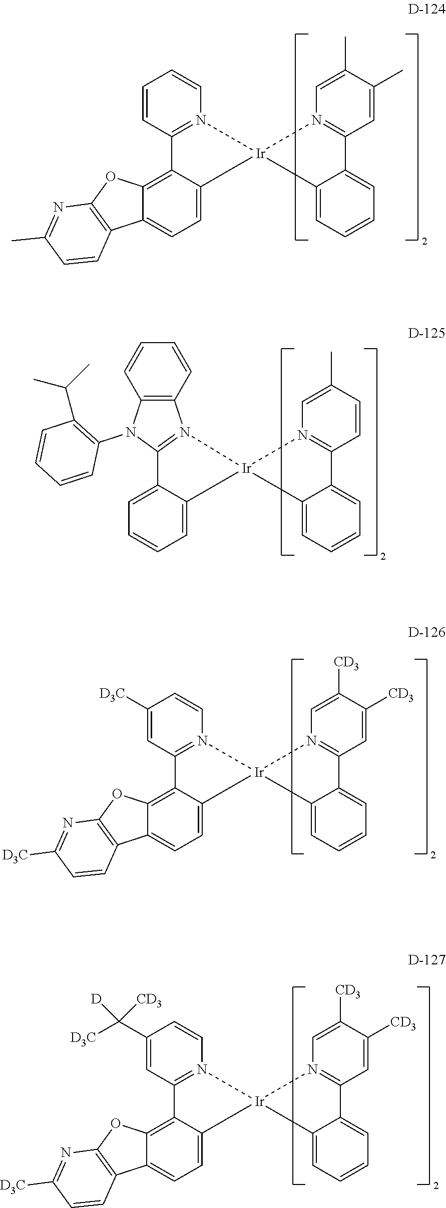

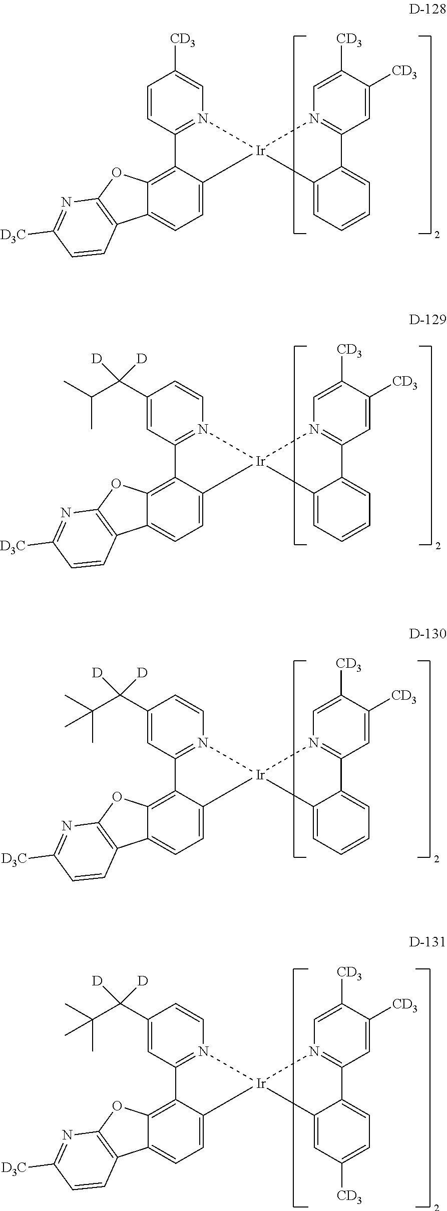

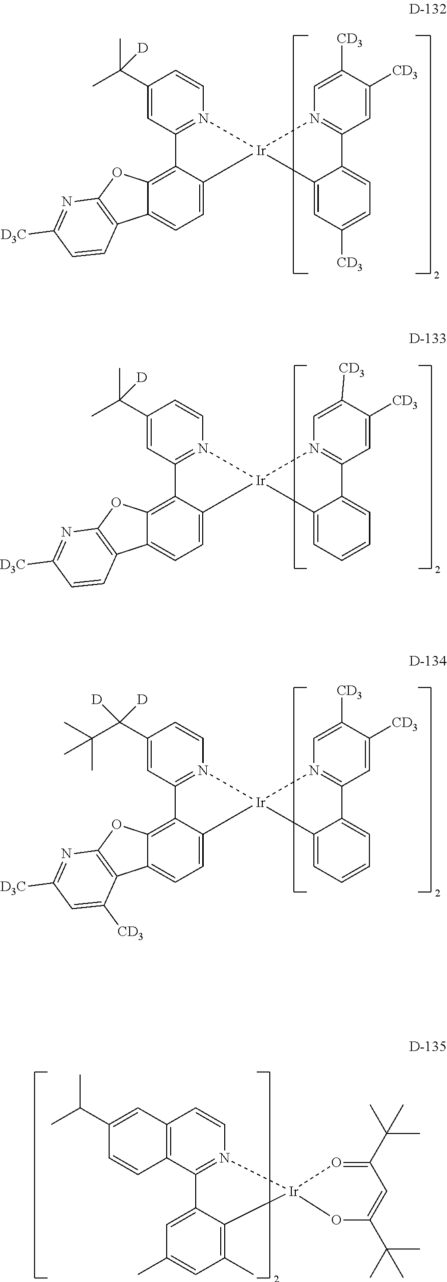

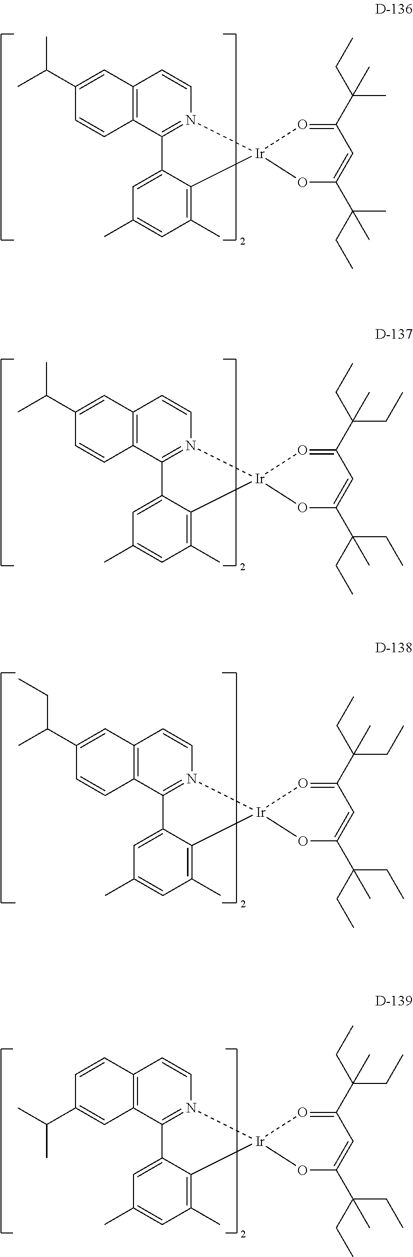

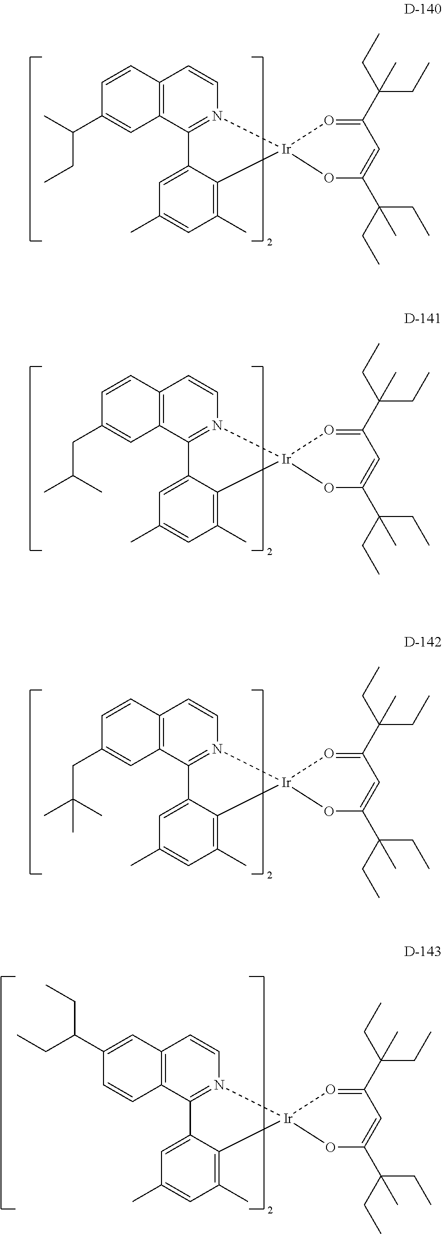

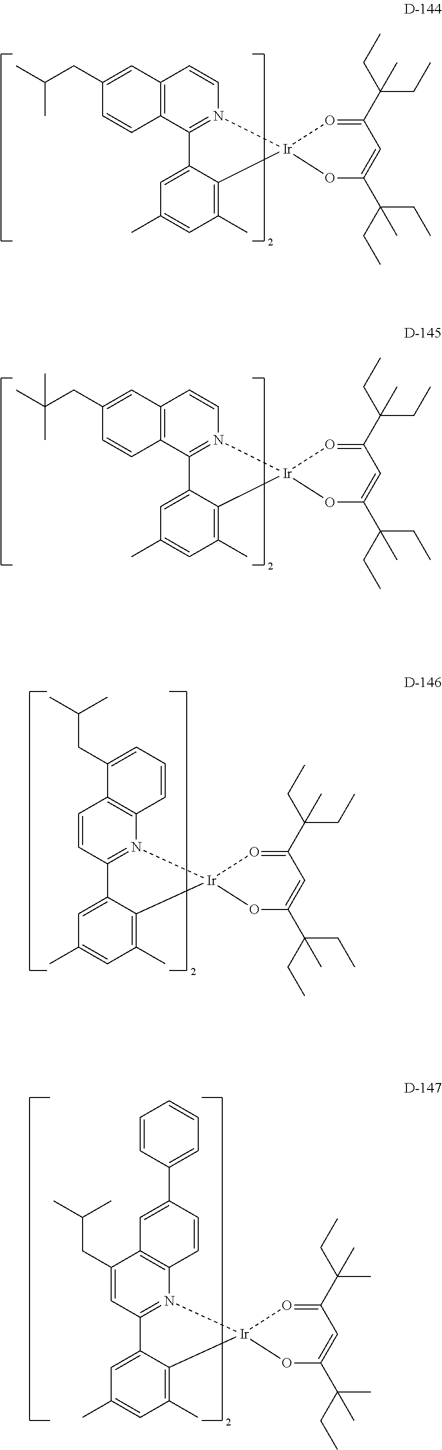

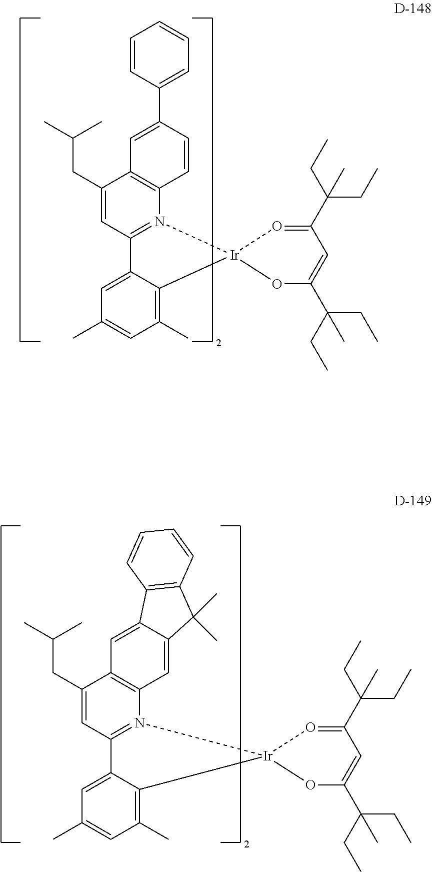

An organic electroluminescent device according to the present disclosure comprises an anode, a cathode, and at least one organic layer between the anode and the cathode. The organic layer comprises a light-emitting layer and may further comprise at least one layer selected from the group consisting of a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the layers may further consist of a plurality of layers.

The anode and the cathode may be respectively formed with a transparent conductive material, or a transflective or reflective conductive material. The organic electroluminescent device may be a top emission type, a bottom emission type, or a both-sides emission type, depending on the materials forming the anode and the cathode. In addition, the hole injection layer may be further doped with a p-dopant, and the electron injection layer may be further doped with an n-dopant.

The organic layer may further comprise at least one compound selected from the group consisting of arylamine-based compounds and styrylarylamine-based compounds. Further, the organic layer may further comprise at least one metal selected from the group consisting of metals of Group 1, metals of Group 2, transition metals of the period, transition metals of the 5^{th} period, lanthanides, and organic metals of the d-transition elements of the Periodic Table, or at least one complex compound comprising the metal.

In addition, the organic electroluminescent device of the present disclosure may emit white light by further comprising at least one light-emitting layer, which comprises a blue, a red, or a green electroluminescent compound known in the field, besides the compound of the present disclosure. If necessary, it may further comprise a yellow or an orange light-emitting layer.

In the organic electroluminescent device of the present disclosure, preferably, at least one layer selected from the group consisting of a chalcogenide layer, a metal halide layer, and a metal oxide layer (hereinafter, “a surface layer”) may be placed on an inner surface(s) of one or both electrode(s). Specifically, a chalcogenide (including oxides) layer of silicon or aluminum is preferably placed on an anode surface of an electroluminescent medium layer, and a metal halide layer or a metal oxide layer is preferably placed on a cathode surface of an electroluminescent medium layer. Such a surface layer provides operation stability for the organic electroluminescent device. Preferably, the chalcogenide includes SiO_{X}(1≤X≤2), AlO_{X}(1≤X≤5), SiON, SiAlON, etc.; the metal halide includes LF, MgF₂, CaF₂, a rare earth metal fluoride, etc.; and the metal oxide includes Cs₂O , Li₂O, MgO, SrO, BaO, CaO, etc.

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. The hole transport layer or the electron blocking layer may also be multi-layers.

An electron buffer layer, a hole blocking layer, an electron transport layer, an electron injection layer, or a combination thereof can be used between the light-emitting layer and the cathode; The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or hole transport, or for preventing the overflow of electrons. When the light-emitting auxiliary layer is placed between the cathode and the light-emitting layer, it can be used for promoting the electron injection and/or electron transport, or for preventing the overflow of holes. Also, the hole auxiliary layer may be placed between the hole transport layer (or hole injection layer) and the light-emitting layer, and may be effective to promote or block the hole transport rate (or hole injection rate), thereby enabling the charge balance to be controlled. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When an organic electroluminescent device includes two or more hole transport layers, the hole transport layer, which is further included, may be used as a hole auxiliary layer or an electron blocking layer. The light-emitting auxiliary layer, the hole auxiliary layer or the electron blocking layer may have an effect of improving the efficiency and/or the lifetime of the organic electroluminescent device.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to the light-emitting medium. Furthermore, the hole transport compound is oxidized to a cation, and thus it becomes easier to inject and transport holes from the mixed region to the light-emitting medium. Preferably, the oxidative dopant includes various Lewis acids and acceptor compounds; and the reductive dopant includes alkali metals, alkali metal compounds, alkaline earth metals, rare-earth metals, and mixtures thereof. The reductive dopant layer may be employed as a charge-generating layer to produce an organic electroluminescent device having two or more light-emitting layers and emitting white light.

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, the compound represented by formula 1, and a compound different from the compound represented by formula 1, for example, the compound represented by formula 2 or 3, may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to one cell to evaporate the materials. In addition, if the first host compound and the second host compound are present in the same layer or different layers in an organic electroluminescent device, the two host compounds may individually form films. For example, the second host compound may be deposited after depositing the first host compound.

Further, the organic electroluminescent material according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The present disclosure may also be applied to such a white organic light-emitting device. In addition, the organic electroluminescent material according to the present disclosure may also be used in an organic electroluminescent device comprising a quantum dot (QD).

A display system may be provided by using the host material comprising the compound represented by formula 1, or the plurality of host materials comprising at least one first host material including the compound represented by formula 1 and at least second host material different from the first host material, That is, it is possible to produce a display system or a lighting system by using the organic electroluminescent device of the present disclosure. Specifically, it is possible to produce a display system, for example, a display system for smart phones, tablets, notebooks, PCs, TVs, or cars; or a lighting system, for example an outdoor or indoor lighting system, by using the organic electroluminescent device of the present disclosure.

Hereinafter, the preparation method of the compounds according to the present disclosure and the properties thereof, and the properties of an organic electroluminescent device (OLED) comprising a plurality of host materials according to the present disclosure will be explained in detail with reference to the representative compounds of the present disclosure. However, the present disclosure is not limited by the following examples.

### Example 1: Preparation of Compound H1-1

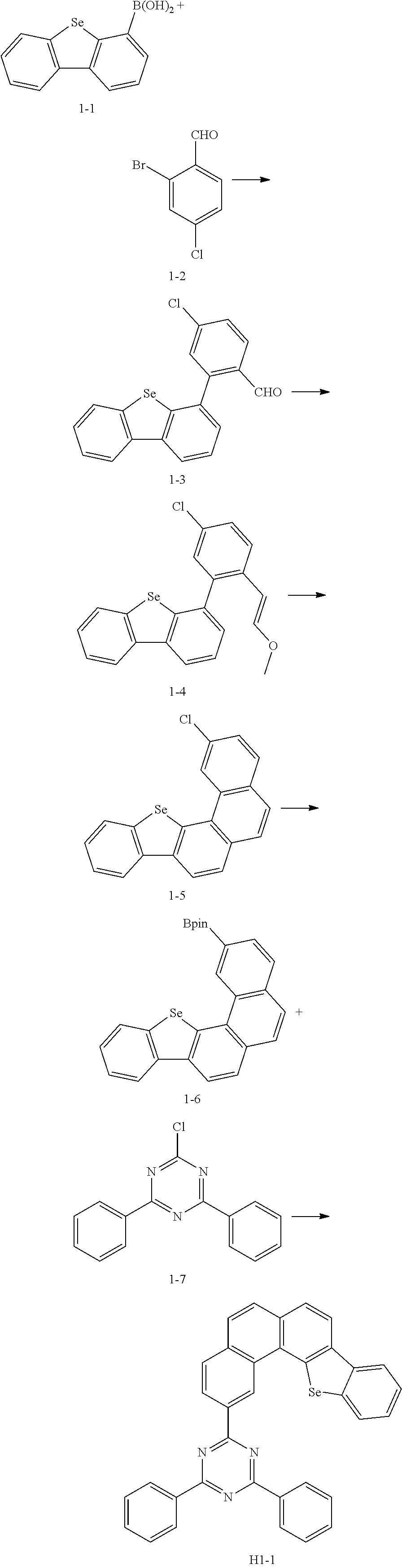

Synthesis of Compound 1-3

Compound 1-1 (3.1 g, 11.2 mmol), compound 1-2 (2.4 g, 11.2 mmol), tetrakis(triphenylphosphine)palladium(0) (C65 g, 0.56 mmol), potassium carbonate (3.9 g, 28.1 mmol), 60 mL of toluene, 15 mL of ethanol, and 15 mL of distilled water were added to a reaction vessel, and stirred under reflux for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 1-3 (3.9 g, yield: 94%).

Synthesis of Compound 1-4

Compound 1-3 (3,9 g, 10.6 mmol), (methoxymethyl)triphenylphosphonium chloride (4.7 g, 13.8 mmol), potassium tert-butoxide (1M in THE) (16 mL, 15.9 mmol), and 55 mL of tetrahydrofuran were added to a reaction vessel, and stirred for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 1-4 (3.6 g, yield: 86%).

Synthesis of Compound 1-5

Compound 1-4 (1.8 g, 4.52 mmol), boron trifluoride diethyl ether complex (1.2 mL, 9.04 mmol), and 45 mL of methylene chloride were added to a reaction vessel, and stirred for hours. After completion of the reaction, the mixture was washed with distilled water, and the organic layer was extracted with ethyl acetate. The residual moisture was removed with magnesium sulfate, and the organic layer was dried and separated by column chromatography to obtain compound 1-5 (1.2 g, yield: 70%).

Synthesis of Compound 1-6

Compound 1-5 (5 g, 13.6 mmol), bis(pinacolato)diboron (4.2 g, 16.4 mmol), tris(dibenzylideneacetone)dipalladium (0.37 g, 0.41 mmol), 2-dichlorohexylphosphino-2′,6′-dimethoxybiphenyl (0.56 g, 1.36 mmol), potassium acetate (3.3 g, 34.1 mmol), and 70 mL of 1,4-dioxane were added to a reaction vessel, and stirred under reflux for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 1-6 (3,8 g, yield: 65%).

Synthesis of Compound H1-1

Compound 1-6 (2.1 g, 4.593 mmol), compound 1-7 (1.4 g, 5.052 mmol), tetrakis(triphenylphosphine)palladium(0) (0,26 g, 0,229 mmol), potassium carbonate (1.9 g, 13.77 mmol), 7 mL of distilled water, 7 mL of ethanol, and 28 mL of toluene were added to a flask, dissolved, and then stirred under reflux for 2 hours, After completion of the reaction, the mixture was cooled to room temperature, and methanol was added dropwise thereto. The resulting product was filtered, dissolved in chlorobenzene, and then silica-filtered to obtain compound H1-1 (1.2 g, yield: 46%).

| | MW | M.P. |
|---|---|---|
| H1-1 | 562.5 | 319.2° C. |

### Example 2: Preparation of Compound H1-3

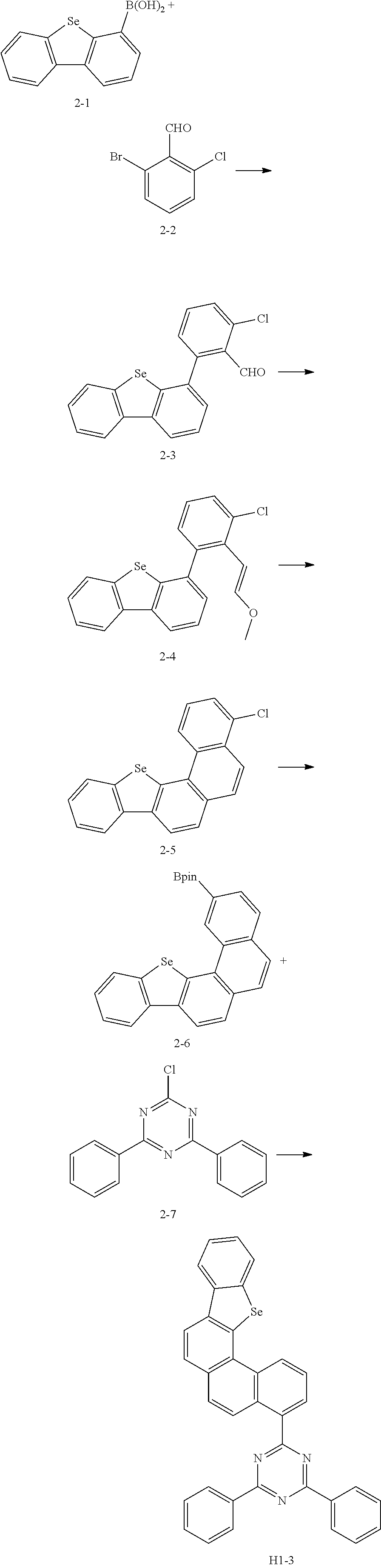

Synthesis of Compound 2-3

Compound 2-1 (20 g, 72.7 mmol), compound 2-2 (15.9 g, 72.7 mmol), tetrakis(triphenylphosphine)palladium(0) (4.2 g, 3.63 mmol), potassium carbonate (25.1 g, 181 mmol), 360 mL of toluene, 90 mL of ethanol, and 90 mL of distilled water were added to a reaction vessel, and stirred under reflux for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 2-3 (24 g, yield: 91%).

Synthesis of Compound 2-4

Compound 2-3 (24 g, 64.9 mmol), (methoxymethyl)triphenylphosphonium chloride (28.9 g, 84.4 mmol), potassium tert-butoxide (1M in THF) (98 mL, 15.9 mmol), and 320 mL of tetrahydrofuran were added to a reaction vessel, and stirred for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 2-4 (26.7 g).

Synthesis of compound 2-5 Compound 2-4 (26.7 g), boron trifluoride diethyl ether complex (16 mL, 128 mmol), and 640 mL of methylene chloride were added to a reaction vessel, and stirred for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 2-5 (18 g, 2 step yield: 76%).

Synthesis of Compound 2-6

Compound 2-5 (8 g, 21.8 mmol), bis(pinacolato)diboron (6.7 g, 26.2 mmol), tris(dibenzylideneacetone)dipalladium (0.6 g, 0.65 mmol), 2-dichlorohexylphosphino-2′,6-dimethoxybiphenyl (0.89 g, 2.18 mmol), potassium acetate (5.3 g, 54.6 mmol), and 110 mL of 1,4-dioxane were added to a reaction vessel, and stirred under reflux for 2 hours. After completion of the reaction, the mixture was washed with distilled water, and the organic layer was extracted with ethyl acetate. The residual moisture was removed with magnesium sulfate, and the organic layer was dried and separated by column chromatography to obtain compound 2-6 (6.9 g, yield: 72%).

Synthesis of Compound H1-3

Compound 2-6 (6 g, 13.2 mmol), compound 2-7 (3.5 g, 13.2 mmol), tetrakis(triphenylphosphine)palladium(0) (0.76 g, 0.66 mmol), potassium carbonate (4.5 g, 33.1 mmol), 18 mL of distilled water, 18 mL of ethanol, and 70 mL of toluene were added to a flask, dissolved, and then stirred under reflux for 4 hours. After completion of the reaction, the mixture was cooled to room temperature, and methanol was added dropwise thereto. The resulting product was filtered, dissolved in chlorobenzene, and then silica-filtered to obtain compound H1-3 g, yield: 55%).

| | MW | M.P. |
|---|---|---|
| H1-3 | 562.52 | 295° C. |

### Example 3: Preparation of Compound H1-21

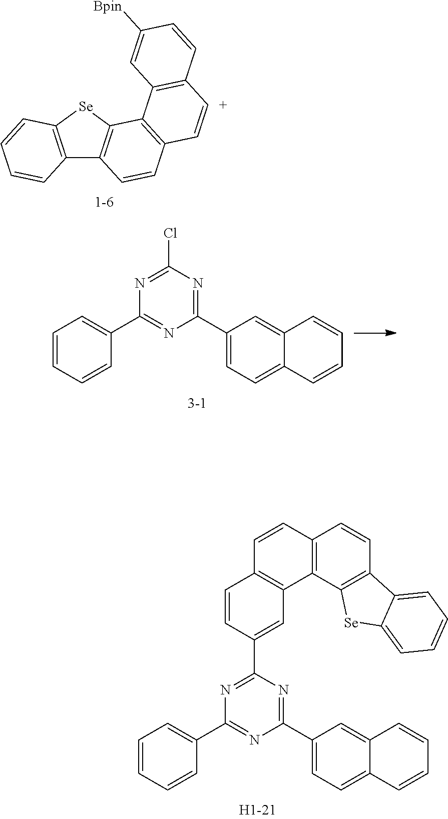

Compound 1-6 (1.9 g, 4,155 mmol), compound 3-1 (1.6 g, 4.986 mmol), tetrakis(triphenylphosphine)palladium(0) (0,24 g, 0,207 mmol), potassium carbonate (1.7 g, 12.46 mmol), 6 mL of distilled water, 6 mL of ethanol, and 24 mL of toluene were added to a flask, dissolved, and then stirred under reflux for 2 hours, After completion of the reaction, the mixture was cooled to room temperature, and methanol was added dropwise thereto. The resulting product was filtered, dissolved in chlorobenzene, and then silica-filtered to obtain compound H1-21 (1.3 g, yield: 51%).

| | MW | M.P. |
|---|---|---|
| H1-21 | 612.5 | 265.4° C. |

### Example 4: Preparation of Compound H1-81

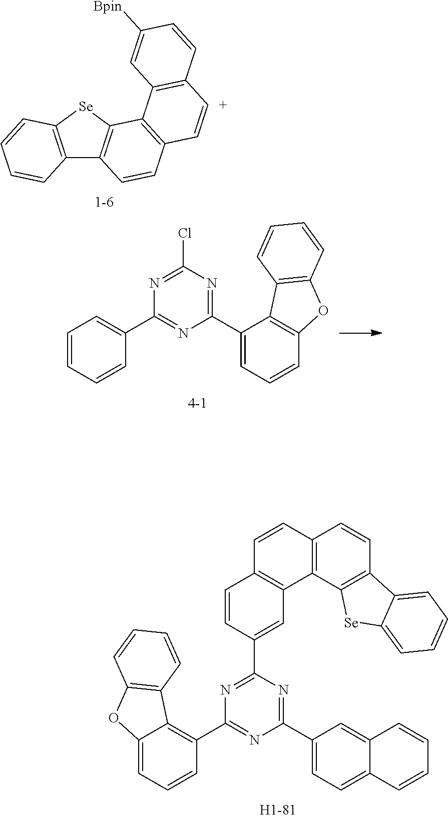

Compound 1-6 (3.8 g, 8.4 mmol), compound 4-1 (3 g, 8.4 mmol), tetrakis(triphenylphosphine)palladium(0) (0.48 g, 0.42 mmol), potassium carbonate (2.9 g, 21 mmol), 10 mL of distilled water, 10 mL of ethanol, and 40 mL of toluene were added to a flask, dissolved, and then stirred under reflux for 2 hours. After completion of the reaction, the mixture was cooled to room temperature, and methanol was added dropwise thereto. The resulting product was filtered, dissolved in chlorobenzene, and then silica-filtered to obtain compound H1-81 (4,9 g, yield: 93%).

| | MW | M.P. |
|---|---|---|
| H1-81 | 652.62 | 320° C. |

### Example 5: Preparation of Compound C-3

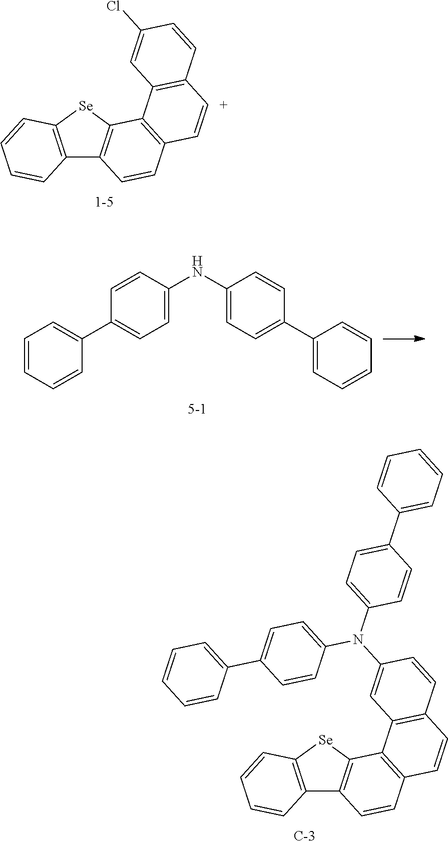

Compound 1-5 (5.5 g, 14.94 mmol), compound 5-1 (4 g. 12.45 mmol), tris(dibenzylideneacetone)dipalladium(0) (0.6 g, 0.622 mmol), sodium tert-butoxide (1.8 g, 18.68 mmol), tri-tert-butylphosphine (50%) (0.6 mL, 0.622 mmol), and 65 mL of toluene were added to a flask, dissolved, and then stirred under reflux for 2 hours. After completion of the reaction, the mixture was cooled to room temperature and washed with distilied water. The organic layer was extracted with ethyl acetate, distilled under reduced pressure, and then separated by column chromatography to obtain compound C-3 (1.1 g, yield: 13%).

| | MW | M.P. |
|---|---|---|
| C-3 | 650.6 | 237.8° C. |

### Example 6: Preparation of Compound C-24

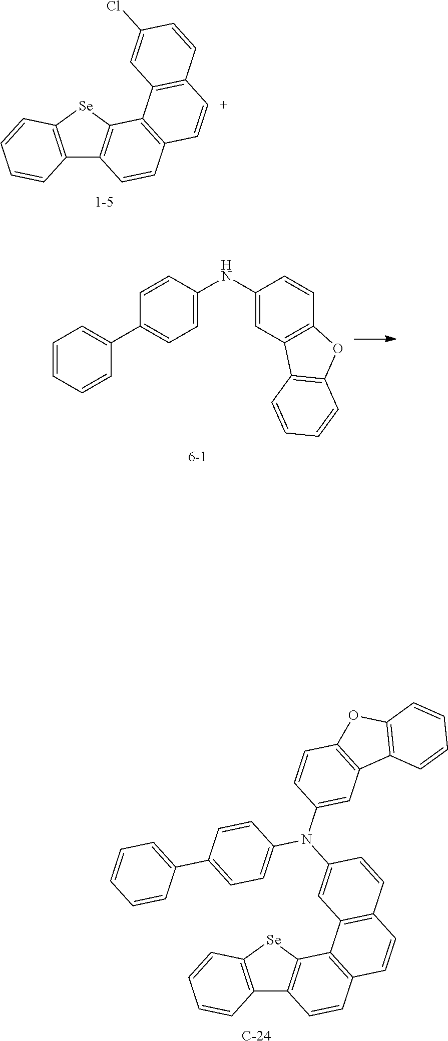

Compound 1-5 (5 g, 13.67 mmol), compound 6-1 (4.6 g, 13.94 mmol), tris(dibenzylideneacetone)dipalladium(0) (0.62 g, 0.68 mmol), 2-dichlorohexylphosphino-2′,6′-dimethoxybiphenyl (0.56 g, 1.36 mmol), sodium test-butoxide (3.3 g, 34.1 mmol), and 70 mL of toluene were added to a flask, dissolved, and then stirred under reflux for 3 hours. After completion of the reaction, the mixture was cooled to room temperature, silica-filtered, dried, and then separated by column chromatography to obtain compound C-24 (8.3 g, yield: 91%),

| | MW | M.P. |
|---|---|---|
| C-24 | 664.6 | 242° C. |

### Example 7: Preparation of Compound C-42

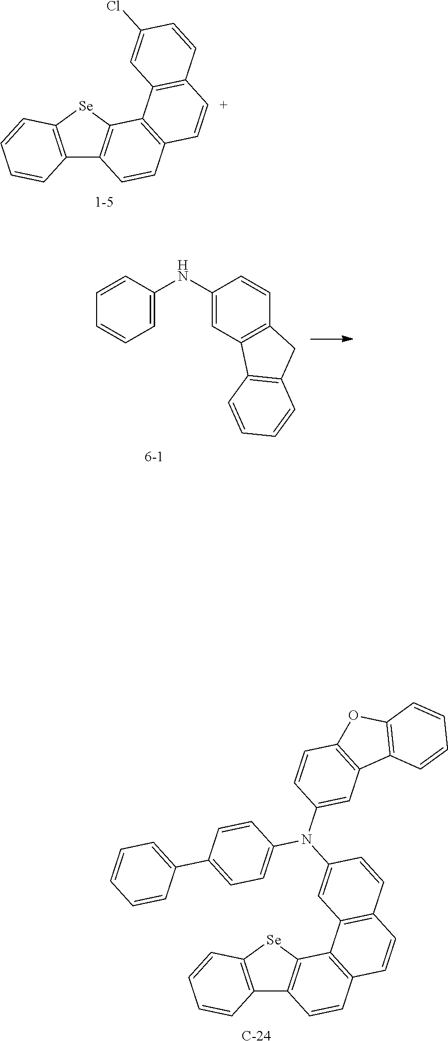

Compound 1-5 (5 g, 13.67 mmol), compound 7-1 (3.9 g, 13.81 mmol), tris(dibenzylideneacetone)dipalladium(0) (0.62 g, 0.68 mmol). 2-dichiorohexylphosphino-2″,6′-dimethoxybiphenyl (0.56 g, 1.36 mmol), sodium tert-butoxide (3.3 g, 34.1 mmol), and 70 mL of toluene were added to a flask, dissolved, and then stirred under reflux for 4 hours. After completion of the reaction, the mixture was cooled to room temperature, silica-filtered, dried, and then separated by column chromatography to obtain compound C-42 (8.2 g, yield: 97%).

| | MW | M.P. |
|---|---|---|
| C-42 | 614.6 | 215° C. |

### Example 8: Preparation of Compound C-96

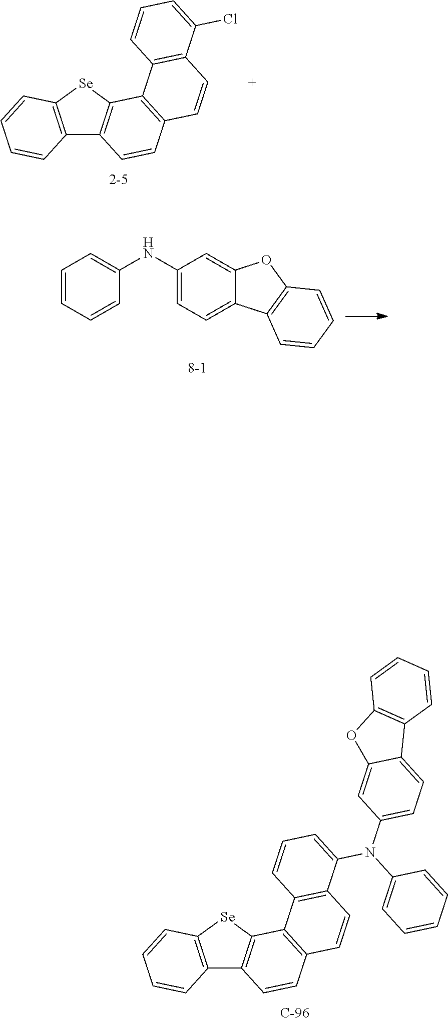

Compound 2-5 (5 g, 13.67 mmol), compound 8-1 (3.58 g, 13.81 mmol), tris(dibenzylideneacetone)dipalladium(0) (0.62 g, 0.68 mmol), 2-dichlorohexylphosphino-2′,6′-dimethoxybiphenyl (0.56 g, 1.36 mmol), sodium tert-butoxide (3.3 g, 34.1 mmol), and 70 mL of toluene were added to a flask, dissolved, and then stirred under reflux for 4 hours. After completion of the reaction, the mixture was cooled to room temperature, silica-filtered, dried, and then separated by column chromatography to obtain compound C-96 (6.6 g, yield: 83%).

| | MW | M.P. |
|---|---|---|
| C-96 | 588.5 | 228° C. |

### Example 9: Preparation of Compound C-35

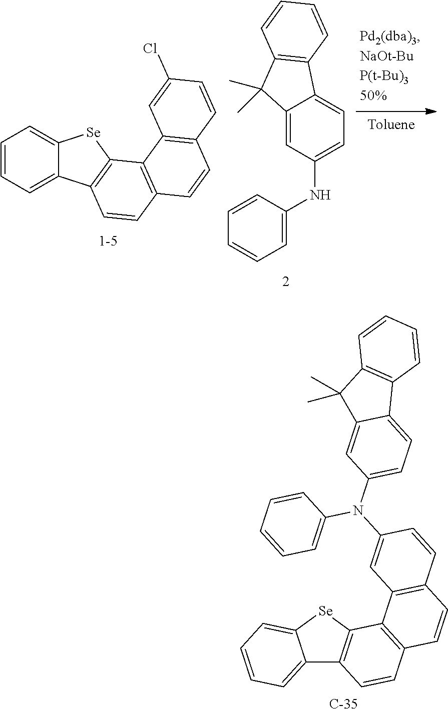

Compound 1-5 (7.7 g, 21.03 mmol), compound 2 (5 g, 17.52 mmol), tris(dibenzylideneacetone)dipalladium(0) (0.8 g, 0.876 mmol), sodium tert-butoxide (2.5 g, 26.28 mmol), and tri-tert-butylphosphine (50%) (0.86 mL, 1,752 mmol) were added to 90 mL of toluene, and stirred under reflux for 2 hours, After completion of the reaction, the mixture was cooled to room temperature, and methanol was added dropwise thereto. The resulting product was filtered and recrystallized with toluene to obtain compound C-35 (7.3 g, yield: 61%).

| | MW | M.P. |
|---|---|---|
| C-35 | 614.6 | 296.3° C. |

### Example 10: Preparation of Compound C-48

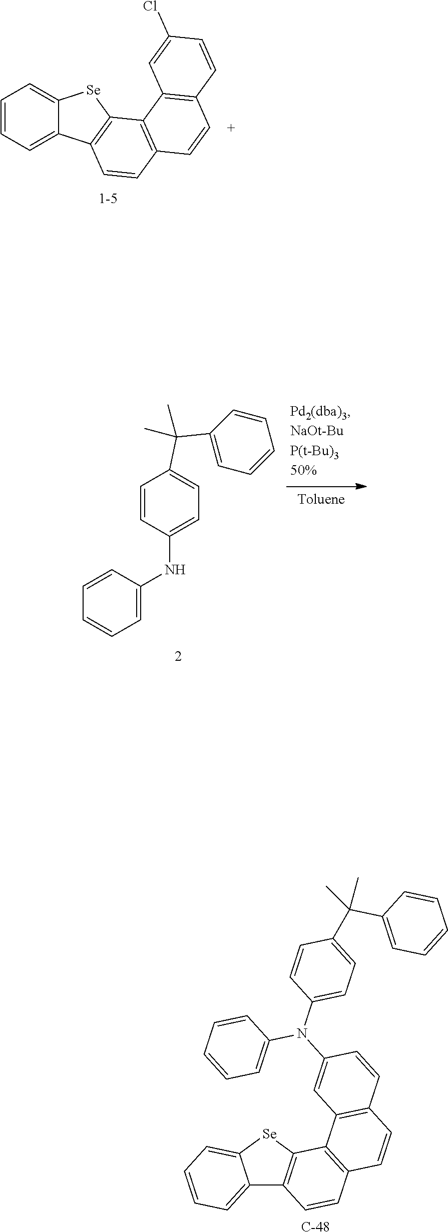

Compound 1-5 (5.1 g, 17.74 mmol), compound 2 (7.8 g, 21.29 mmol), tris(dibenzylideneacetone)dipalladium(0) (0.81 g, 0.887 mmol), sodium tert-butoxide (2.5 g, 26.61 mmol), and tri-tert-butylphosphine (50%) (0.9 mL, 1.774 mmol) were added to 90 mL of toluene, and stirred under reflux for 2 hours. After completion of the reaction, the mixture was cooled to room temperature, cellite-filtered, distilled under reduced pressure, and then separated by column chromatography to obtain compound C-48 (4.6 g, yield: 42%).

| | MW | M.P. |
|---|---|---|
| C-48 | 616.6 | 159.5° C. |

### Example 11: Preparation of Compound C-43

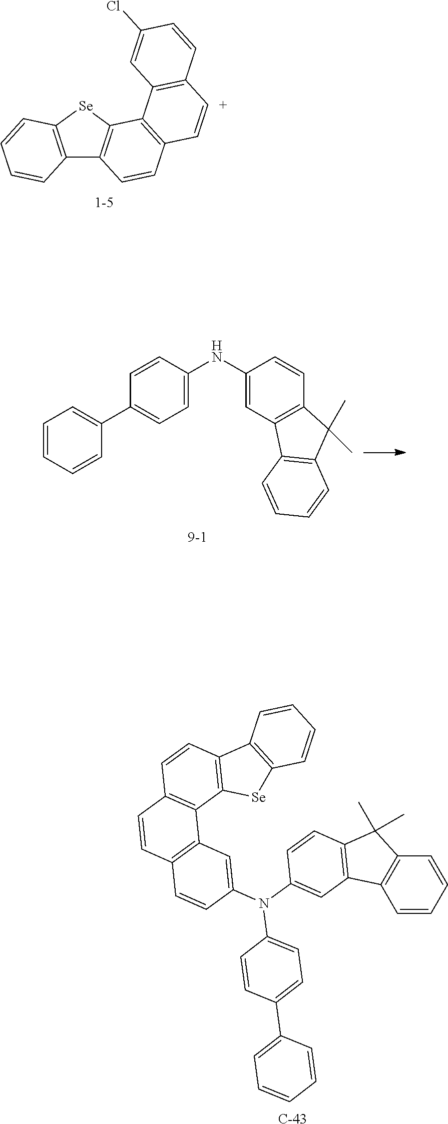

Compound 1-5 (7 g, 19.14 mmol), compound 9-1 (6.7 g, 19.14 mmol), tris(dibenzylideneacetone)dipalladium(0) (0.876 g, 0.95 mmol), sodium tert-butoxide (5.5 g, 57.42 mmol), tri-tert-butylphosphine (50%) (0.774 mL, 1.91 mmol), and 96 mL of toluene were added to a flask, dissolved, and then stirred under reflux for 2 hours. After completion of the reaction, the mixture was cooled to room temperature, silica-filtered, dried, and then separated by column chromatography to obtain compound C-43 (6.7 g, yield: 51%).

| | MW | M.P. |
|---|---|---|
| C-43 | 691.1 | 245.6° C. |

### Example 12: Preparation of Compound C-44

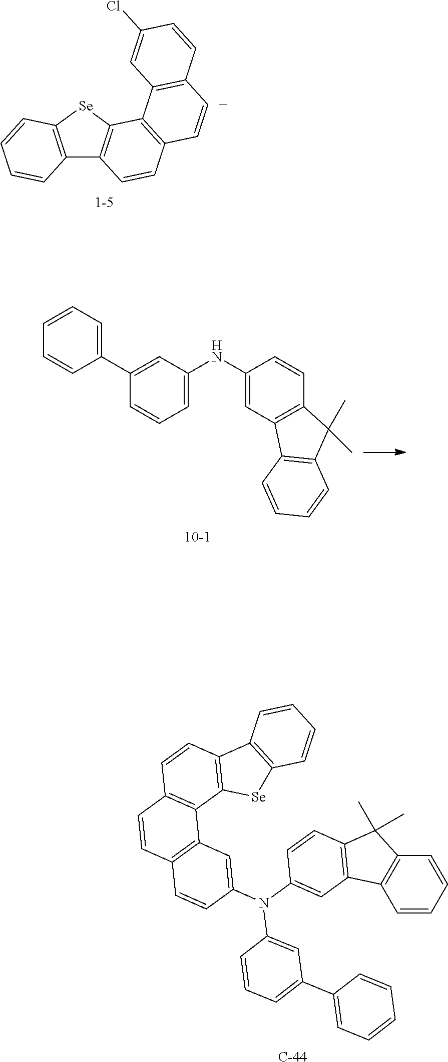

Compound 1-5 (5.5 g, 15.04 mmol), compound 10-1 (5 g, 13.83 mmol), tris(dibenzylideneacetone)dipalladium(0) (0.633 g, 0.69 mmol), sodium tert-butoxide (3.9 g, 41.49 mmol), tri-tart-butylphosphine (50%) (0.560 mL, 1,38 mmol), and 70 mL of toluene were added to a flask, dissolved, and then stirred under reflux for 2 hours. After completion of the reaction, the mixture was cooled to room temperature, silica-filtered, dried, and then separated by column chromatography to obtain compound C-44 (3.3 g, yield: 35%).

| | MW | M.P. |
|---|---|---|
| C-44 | 691.1 | 275.5° C. |

Device Examples 1 to 13: Producing an OLED Co-Deposited with the First Host Compound and the Second Host Compound According to the Present Disclosure

An OLED according to the present disclosure was produced. A transparent electrode indium tin oxide (ITO) thin film (10 n/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 2 below was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm. Next, compound HT-1 was deposited on the hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: the first host compound and second host compound shown in Table 1 below were introduced into two cells of the vacuum vapor deposition apparatus as hosts, and compound D-39 was introduced into another cell as a dopant. The two host materials were evaporated at a rate of 1:1 and the dopant material was simultaneously evaporated at a different rate, and the dopant was deposited in a doping amount of 3 wt % based on the total amount of the host and the dopant to form a light-emitting layer having a thickness of 40 nm on the second hole transport layer. Next, compound ETL-1 and compound EIL-1 were deposited in a weight ratio of 50:50 as an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

Comparative Examples 1 and 2: Producing an OLED Comprising a Single Host Compound

OLEDs were produced in the same manner as in Device Example 1, except that the second host compound shown in Table 1 below was solely used as a host of the light-emitting layer.

The driving voltage; luminous efficiency, and light-emitting color at a luminance of 1,000 nit, and the time taken for luminance to decrease from 100% to 95% at a luminance of 10000 nit (lifetime; T95) of the OLEDs produced in Device Examples 1 to 13 and Comparative Examples 1 and 2 are provided in Table 1 below.

TABLE 1

| | | | Driving | Luminous | Light- | |
|---|---|---|---|---|---|---|
| | First | Second | Voltage | Efficiency | Emitting | Lifetime |
| | Host | Host | (V) | (cd/A) | Color | [T95(hr)] |
| Device | H1-81 | H2-45 | 3.4 | 33.1 | Red | 289 |
| Example 1 | | | | | | |
| Device | H1-21 | H2-45 | 3.5 | 32.6 | Red | 248 |
| Example 2 | | | | | | |
| Device | C-3 | H3-24 | 3.6 | 31.8 | Red | 411 |
| Example 3 | | | | | | |
| Device | C-24 | H3-24 | 3.5 | 33.7 | Red | 387 |
| Example 4 | | | | | | |
| Device | H1-1 | H2-127 | 3.7 | 34.0 | Red | 197 |
| Example 5 | | | | | | |
| Device | C-35 | H3-66 | 3.1 | 34.9 | Red | 306 |
| Example 6 | | | | | | |
| Device | C-48 | H3-66 | 3.2 | 34.8 | Red | 91 |
| Example 7 | | | | | | |
| Device | C-43 | H3-66 | 2.9 | 36.3 | Red | 280 |
| Example 8 | | | | | | |
| Device | C-44 | H3-66 | 2.9 | 36.2 | Red | 294 |
| Example 9 | | | | | | |
| Device | C-42 | H3-66 | 2.9 | 36.5 | Red | 242 |
| Example 10 | | | | | | |
| Device | C-61 | H3-66 | 2.9 | 35.5 | Red | 326 |
| Example 11 | | | | | | |
| Device | C-96 | H3-66 | 3.7 | 35.6 | Red | 103 |
| Example 12 | | | | | | |
| Device | H1-3 | H2-127 | 3.0 | 36.0 | Red | 152 |
| Example 13 | | | | | | |
| Comparative | | H2-45 | 3.8 | 8.6 | Red | 2 |
| Example 1 | | | | | | |
| Comparative | — | H3-66 | 3.4 | 32.6 | Red | 25 |
| Example 2 | | | | | | |

From Table 1 above, it can be seen that the OLEDs comprising the plurality of host materials according to the present disclosure exhibit lower driving voltage, higher luminous efficiency, and excellent lifetime properties, compared to the OLEDs comprising a single host material.

The compounds used in the Device Examples 1 to 13, and the Comparative Examples 1 and 2 are shown in Table 2 below.

TABLE 2

| | |
|---|---|
| Hole Injection Layer/ Hole Transport Layer | 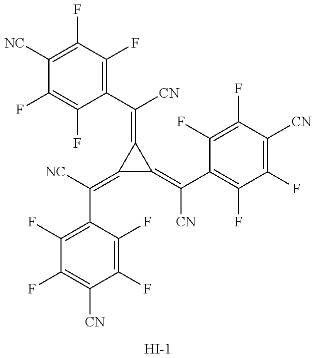 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 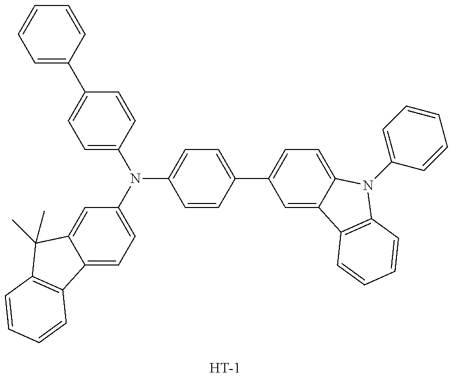 |
| | 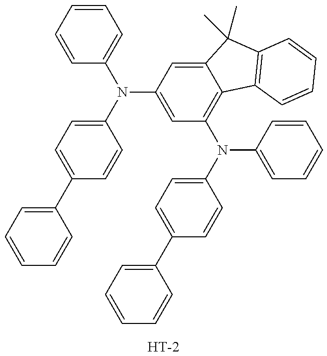 |
| Light- Emitting Layer/ Electron Buffer Layer | 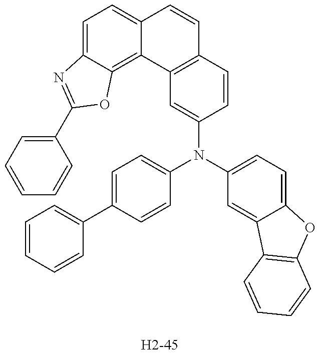 [SMILES: c1ccc(-c2ccc(N(c3ccc4oc5ccccc5c4c3)c3ccc4ccc5ccc6nc(-c7ccccc7)oc6c5c4c3)cc2)cc1] |
| | 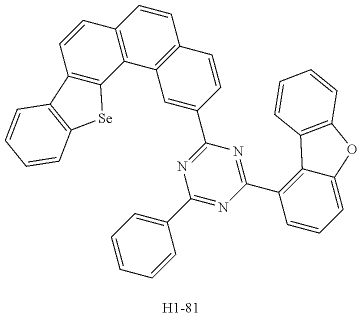 [SMILES: c1ccc(-c2nc(-c3ccc4ccc5ccc6c7ccccc7[se]c6c5c4c3)nc(-c3cccc4oc5ccccc5c34)n2)cc1] |
| | 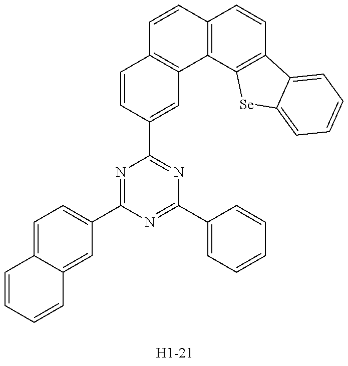 [SMILES: c1ccc(-c2nc(-c3ccc4ccccc4c3)nc(-c3ccc4ccc5ccc6c7ccccc7[se]c6c5c4c3)n2)cc1] |
| | 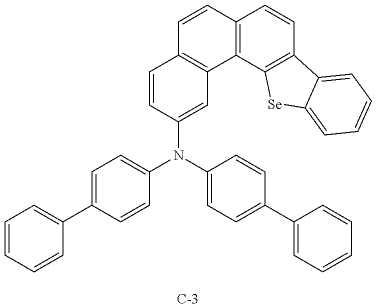 |
| | 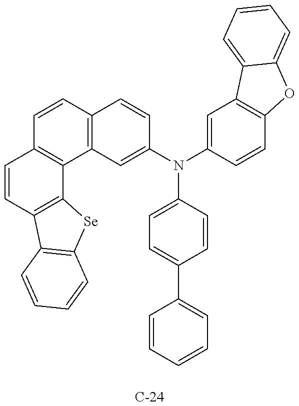 |
| | 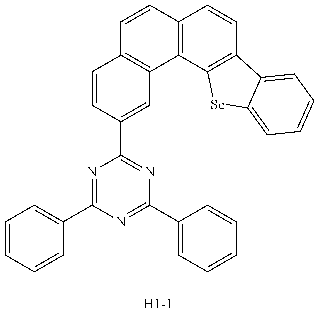 [SMILES: c1ccc(-c2nc(-c3ccccc3)nc(-c3ccc4ccc5ccc6c7ccccc7[se]c6c5c4c3)n2)cc1 ; "H1-1"] |
| | 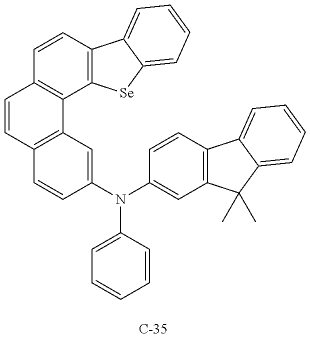 |
| | 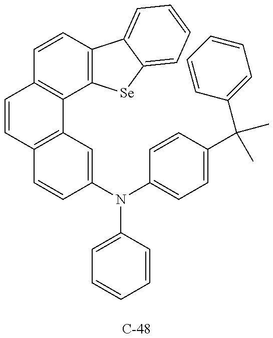 |
| | 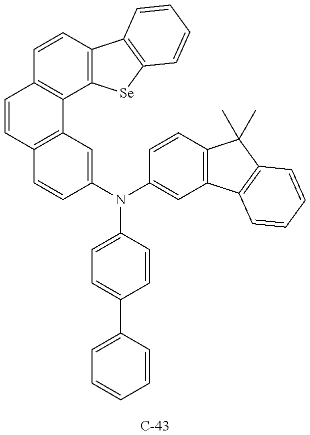 |
| | 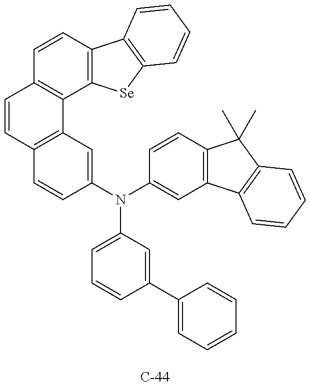 |
| | 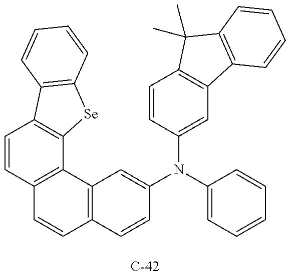 |
| | 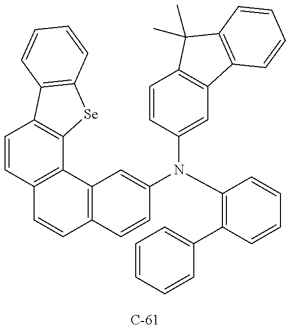 |
| | 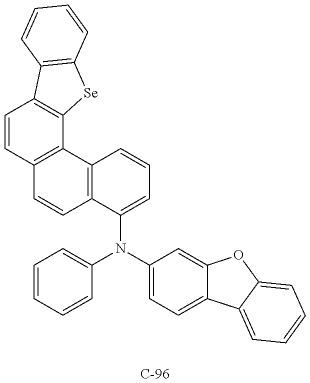 |
| | 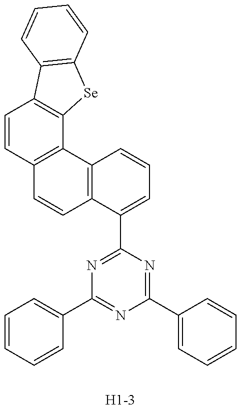 [SMILES: c1ccc(-c2nc(-c3ccccc3)nc(-c3cccc4c3ccc3ccc5c6ccccc6[se]c5c34)n2)cc1] |
| | 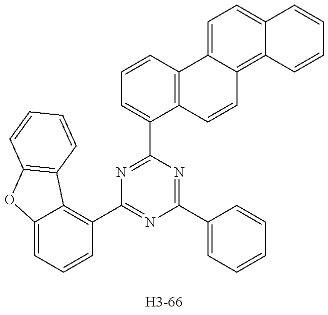 [SMILES: c1ccc(-c2nc(-c3cccc4c3ccc3c5ccccc5ccc43)nc(-c3cccc4oc5ccccc5c34)n2)cc1] |
| | 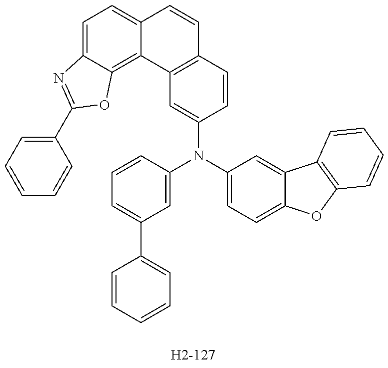 [SMILES: c1ccc(-c2cccc(N(c3ccc4oc5ccccc5c4c3)c3ccc4ccc5ccc6nc(-c7ccccc7)oc6c5c4c3)c2)cc1] |
| | 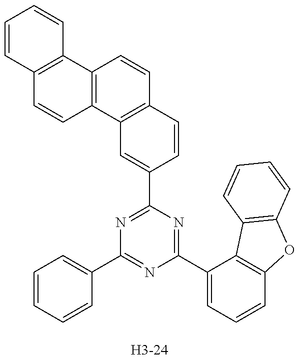 [SMILES: c1ccc(-c2nc(-c3ccc4ccc5c6ccccc6ccc5c4c3)nc(-c3cccc4oc5ccccc5c34)n2)cc1] |
| | 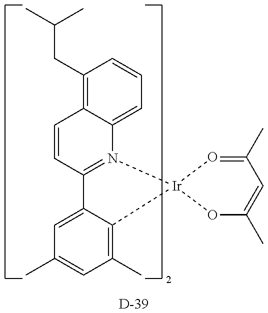 |
| Electron Transport Layer/ Electron Injection Layer | 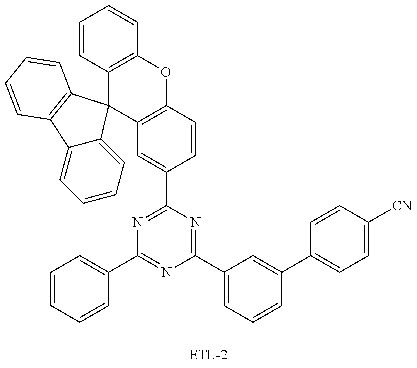 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | 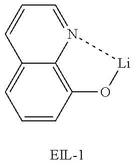 |

Device Examples 14 and 15: Producing an OLED Comprising the Compound According to the Present Disclosure in the Second Hole Transport Layer

An OLED according to the present disclosure was produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 shown in Table 4 below was introduced into a cell of the vacuum vapor deposition apparatus, and compound HT-1 was introduced into another cell of the vacuum vapor deposition apparatus. The two materials were evaporated at different rates, and compound HI-1 was deposited in a doping amount of 3 wt % based on the total amount of compound HI-1 and compound HT-1 to form a hole injection layer having a thickness of 10 nm. Next, compound HT-1 was deposited on the hole injection layer to form a first hole transport layer having a thickness of 90 nm. The compound shown in Table 3 below was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: compounds Y-1 were introduced into one cell of two cells of the vacuum vapor deposition apparatus as a host compound, and compound D-39 was introduced into other cell as a dopant. The host material and the dopant material were evaporated at a different rate simultaneously, and the dopant was deposited in a doping amount of 2 wt % based on the total amount of the host and the dopant to form a light-emitting layer having a thickness of 40 nm on the second hole transport layer. Next, compound HBL was deposited as a hole blocking layer having a thickness of 5 nm on the light-emitting layer. In addition, compound ETL-2 and compound EIL-1 were deposited in a weight ratio of 50:50 as an electron transport layer having a thickness of 30 nm on the hole blocking layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

### Comparative Example 3: Producing an OLED Comprising the Compound not

according to the present disclosure in the second hole transport layer.

OLEDs were produced in the same manner as in Device Example 3, except that the compound shown in Table 3 below was used in a second hole transport layer.

The driving voltage, luminous efficiency, and light-emitting color at a luminance of 1,000 nit of the OLEDs produced in Device Examples 14, 15, and Comparative Example 3 are provided in Table 3 below.

TABLE 3

| | Second Host | Driving | Luminous | Light- |
|---|---|---|---|---|
| | Transport | Voltage | Efficiency | Emitting |
| | Layer | (V) | (cd/A) | Color |
| Device | C-24 | 3.7 | 24.6 | Red |
| Example 14 | | | | |
| Device | C-35 | 2.8 | 31.5 | Red |
| Example 15 | | | | |
| Comparative | T-1 | 4.2 | 21.6 | Red |
| Example 3 | | | | |

From Table 3 above, it can be seen that the OLEDs comprising the compound according to the present disclosure as a hole transport layer material exhibit lower driving voltage and higher luminous efficiency, compared to the OLEDs comprising the conventional compound as a hole transport layer material.

The compounds used in the Device Examples 14, 15, and the Comparative Example 3 are shown in Table 4 below.

TABLE 4

| | |
|---|---|
| Hole Injection Layer/ Hole Transport Layer | 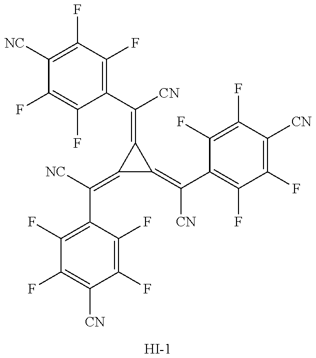 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 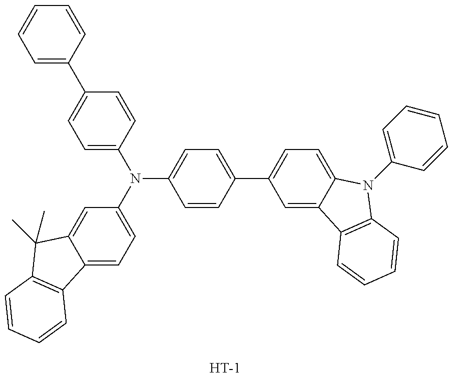 |
| | 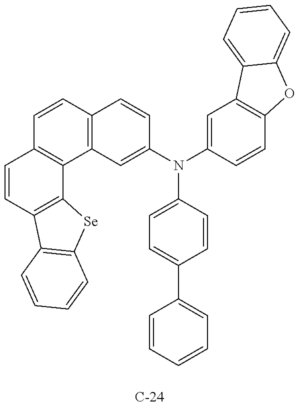 |
| | 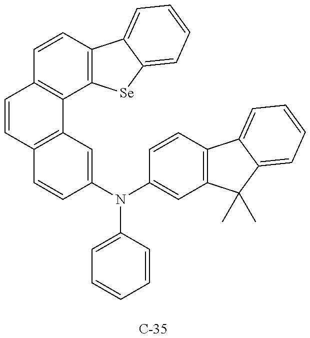 |
| | 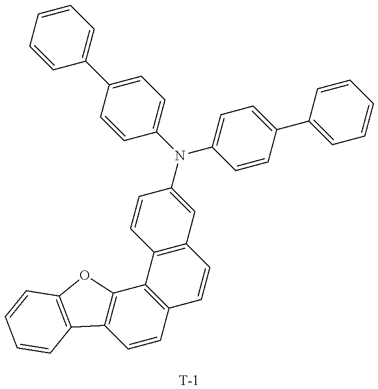 |
| Light- Emitting Layer/ Electron Buffer Layer | 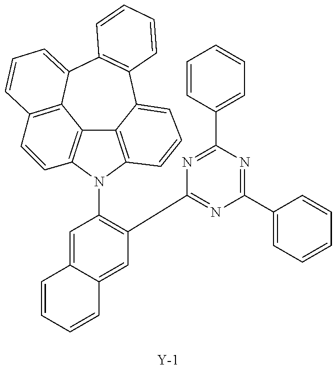 |
| | 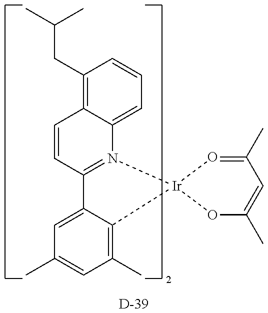 |
| Hole Blocking Layer | 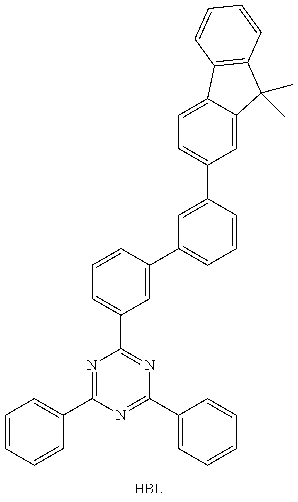 [SMILES: CC1(C)c2ccccc2-c2ccc(-c3cccc(-c4cccc(-c5nc(-c6ccccc6)nc(-c6ccccc6)n5)c4)c3)cc21] |
| Electron Transport Layer/ Electron Injection Layer | 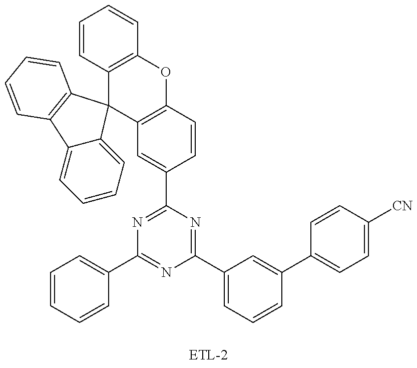 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | 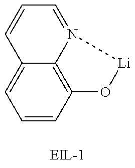 |

1. An organic electroluminescent compound represented by the following formula 1: 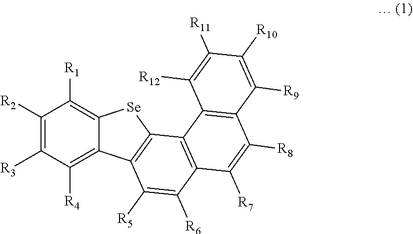
in formula 1,
R₁ to R₁₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or *—L₁—Ar₁;
with the proviso that at least one of R₁ to R₁₂ represents *—L₁—Ar₁;
L₁, each independently, represents a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;
Ar₁, each independently, represents a halogen, a cyano, a substituted or unsubstituted (C1-C30)alkyl, a substituted or unsubstituted (C6-C30)aryl, a substituted or unsubstituted (3-to 30-membered)heteroaryl, a substituted or unsubstituted (C3-C30)cycloalkyl, or a substituted or unsubstituted (C1-C30)alkoxy, or is represented by the following formula a: 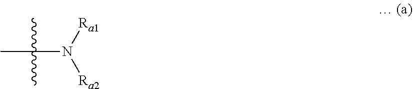
in formula a,
Rₐ₁ and Rₐ₂, each independently, represent a substituted or unsubstituted (C6-C30)aryl, a substituted or unsubstituted (3- to 30-membered)heteroaryl, or a substituted or unsubstituted (C3-C30)cycloalkyl; and [symbol]
represents a bonding position with L₁.

2. The organic electroluminescent compound according to claim 1, wherein the substituent(s) of the substituted alkyl, the substituted alkylene, the substituted cycloalkyl, the substituted cycloalkylene, the substituted alkoxy, the substituted trialkylsilyl, the substituted dialkylarylsilyl, the substituted alkyldiarylsilyl, the substituted triarylsilyl, the substituted aryl, the substituted arylene, the substituted heteroaryl, and the substituted heteroarylene in R₁ to R₁₂, L₁, Ar₁, Rₐ₁, and Rₐ₂, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with at least one of a (C6-C30)aryl(s) and a (3- to 30-membered)heteroaryl(s); a (C6-C30)aryl unsubstituted or substituted with at least one of deuterium, a (C1-C30)alkyl(s), a (C6-C30)aryl(s), and a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di- (C2-C30)alkenylamino; a mono- or di- (C6-C30)arylamino unsubstituted or substituted with a (C1-C30)alkyl(s); a mono- or di- (3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino unsubstituted or substituted with a (C6-C30)aryl(s); a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl: a (C6-C30)aryl(C1-C30)alkyl; and a (C1-C30)alkyl(C6-C30)aryl.

3. The organic electroluminescent compound according to claim 1, wherein the compound represented by formula 1 is selected from the group consisting of the following compounds:

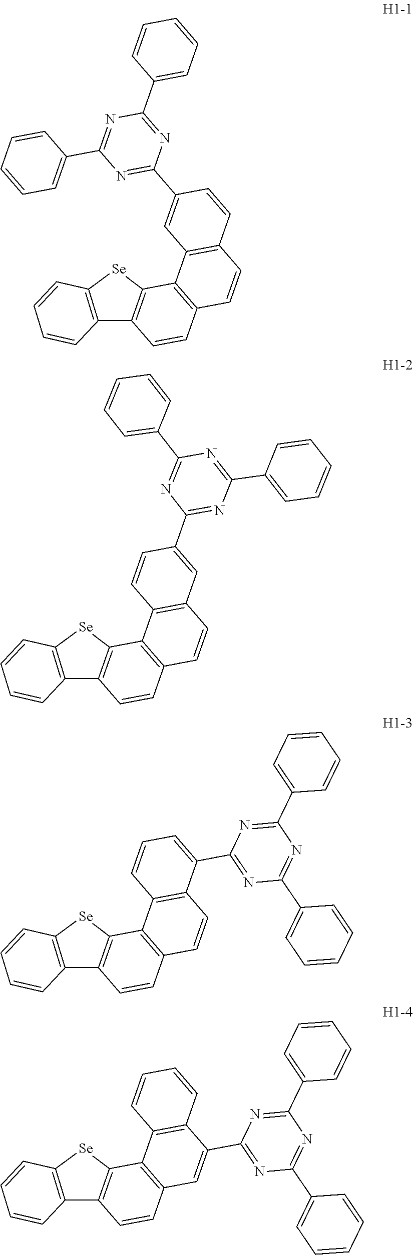

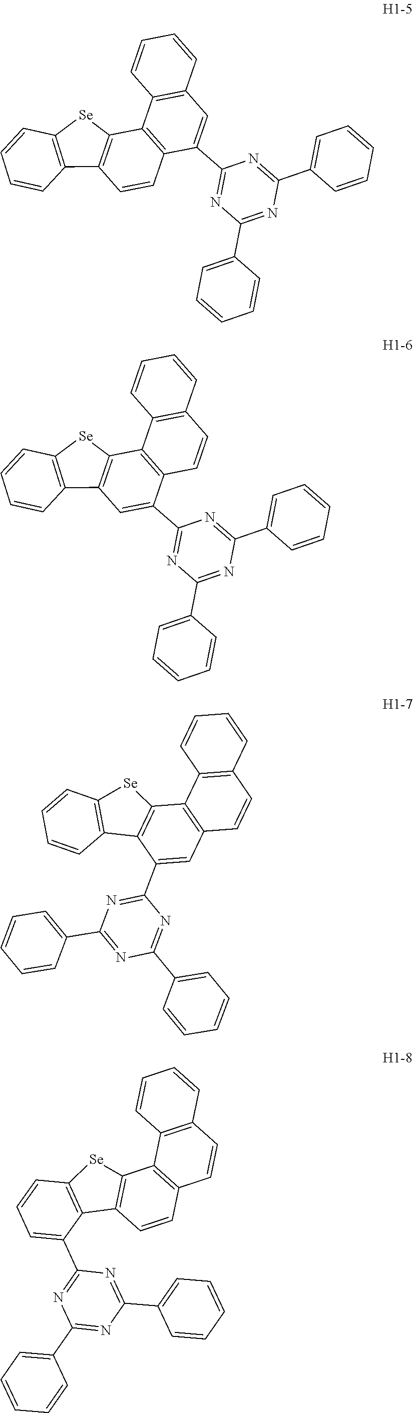

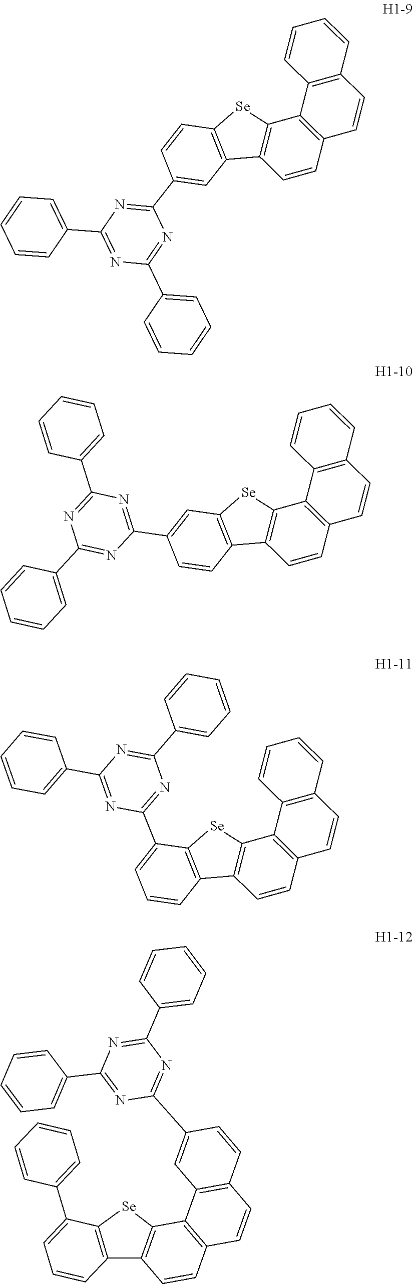

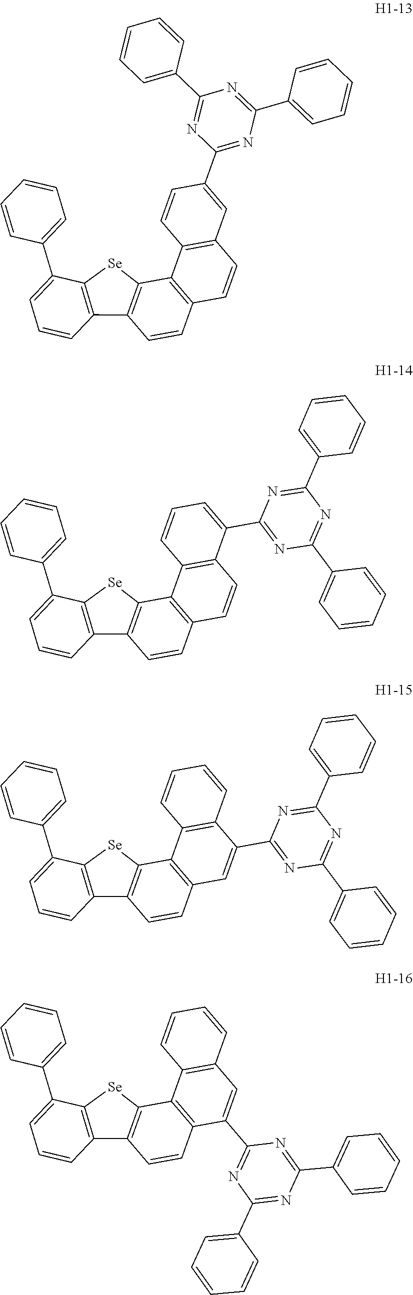 [SMILES: c1ccc(-c2nc(-c3ccccc3)nc(-c3ccc4c(ccc5ccc6c7cccc(-c8ccccc8)c7[se]c6c54)c3)n2)cc1 ; c1ccc(-c2nc(-c3ccccc3)nc(-c3cccc4c3ccc3ccc5c6cccc(-c7ccccc7)c6[se]c5c34)n2)cc1 ; c1ccc(-c2nc(-c3ccccc3)nc(-c3cc4ccc5c6cccc(-c7ccccc7)c6[se]c5c4c4ccccc34)n2)cc1 ; c1ccc(-c2nc(-c3ccccc3)nc(-c3cc4ccccc4c4c3ccc3c5cccc(-c6ccccc6)c5[se]c34)n2)cc1]

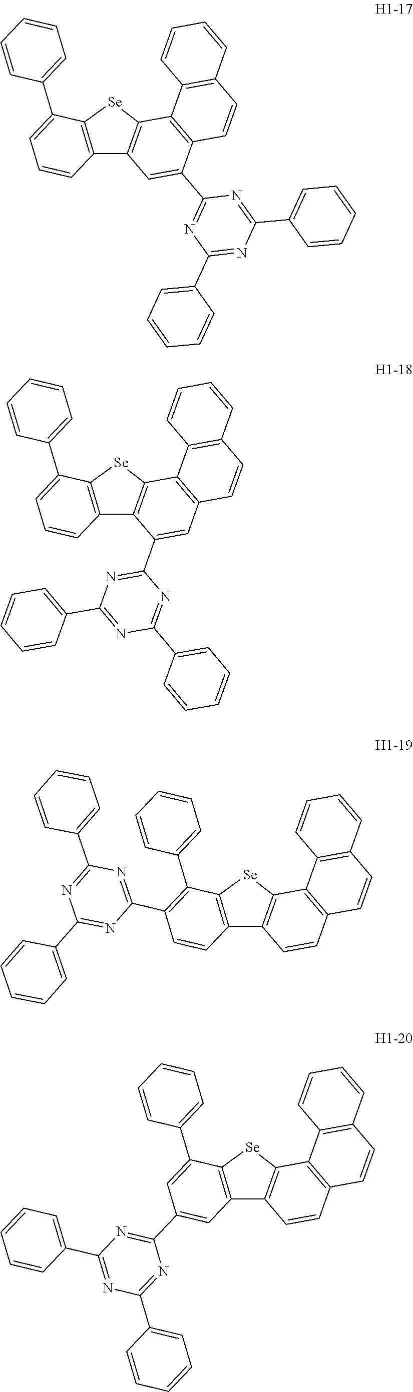

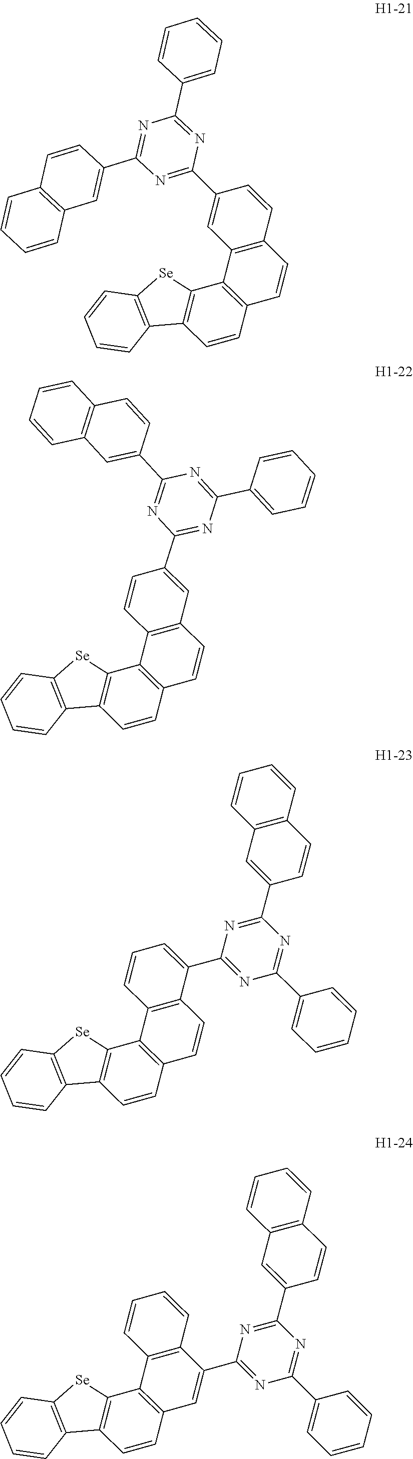

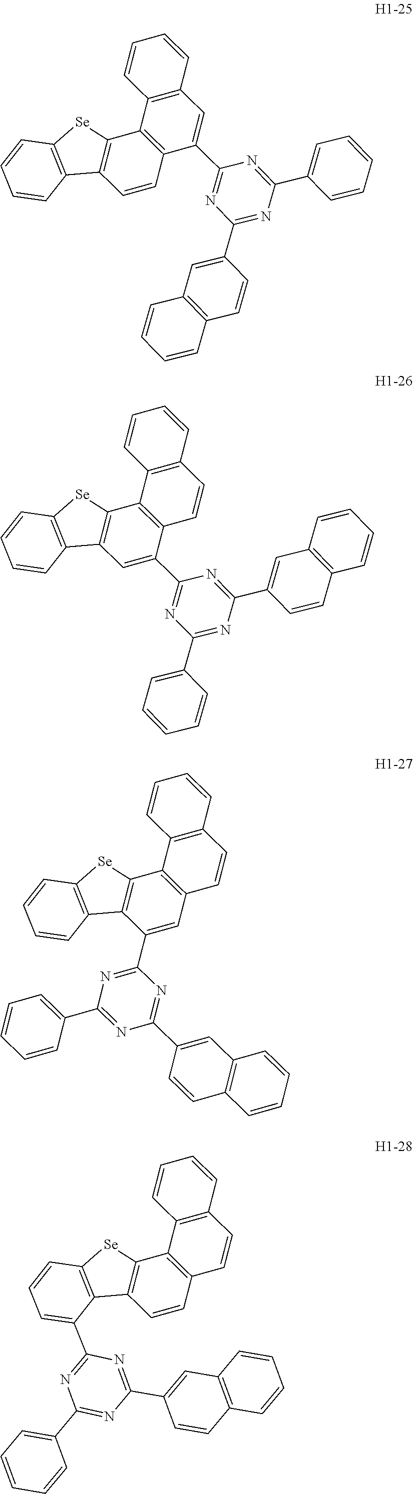

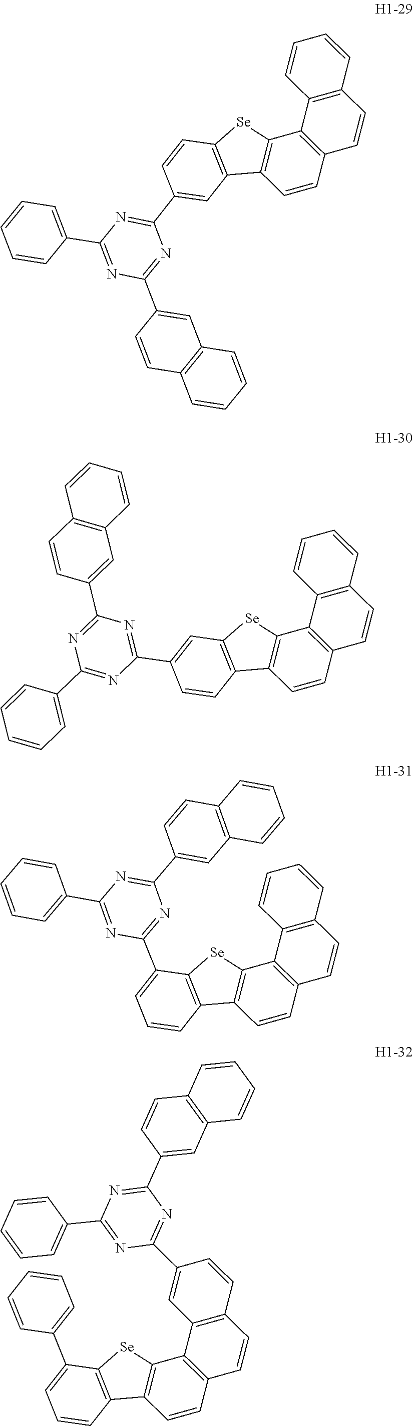

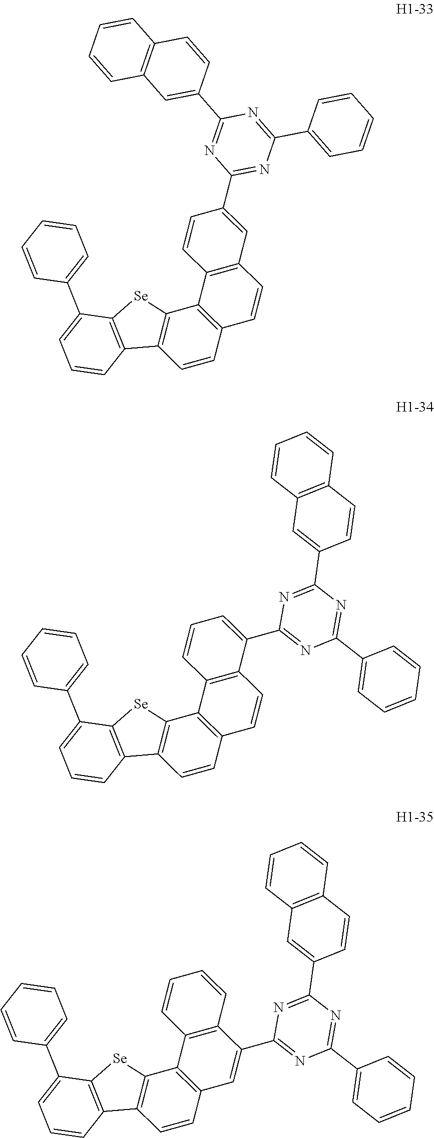 [SMILES: c1ccc(-c2nc(-c3ccc4ccccc4c3)nc(-c3ccc4c(ccc5ccc6c7cccc(-c8ccccc8)c7[se]c6c54)c3)n2)cc1 ; c1ccc(-c2nc(-c3ccc4ccccc4c3)nc(-c3cccc4c3ccc3ccc5c6cccc(-c7ccccc7)c6[se]c5c34)n2)cc1 ; c1ccc(-c2nc(-c3ccc4ccccc4c3)nc(-c3cc4ccc5c6cccc(-c7ccccc7)c6[se]c5c4c4ccccc34)n2)cc1]

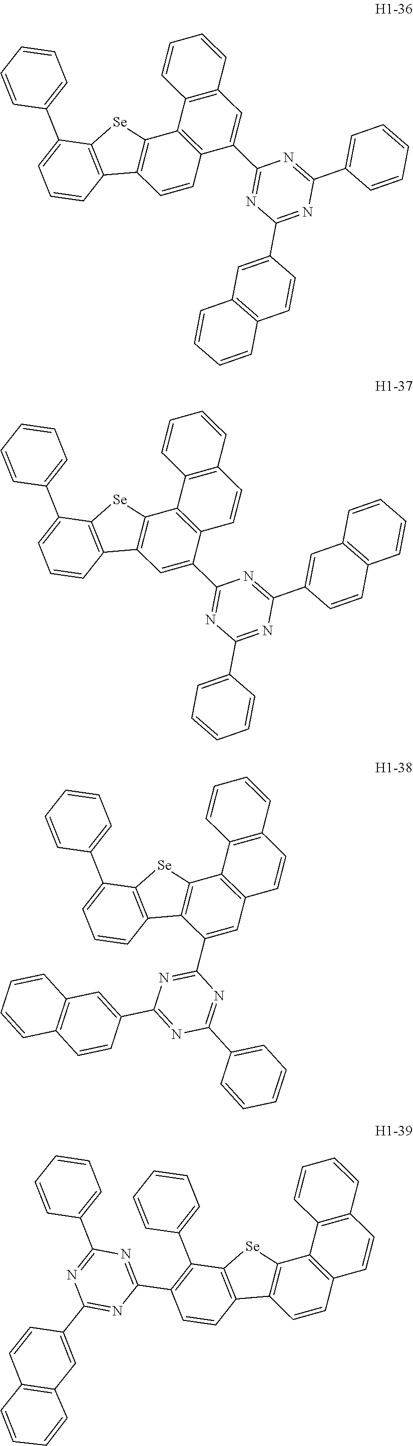

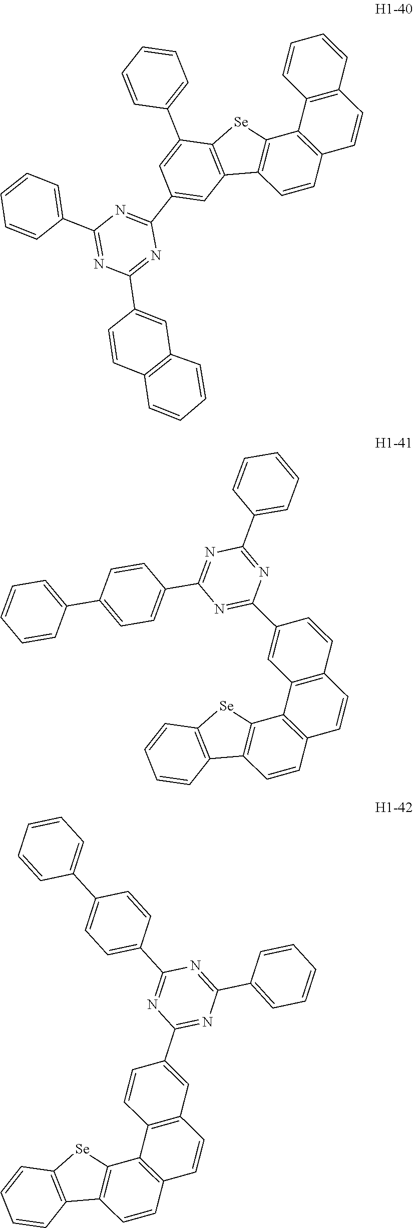 [SMILES: c1ccc(-c2nc(-c3ccc4ccccc4c3)nc(-c3cc(-c4ccccc4)c4[se]c5c(ccc6ccc7ccccc7c65)c4c3)n2)cc1 ; c1ccc(-c2ccc(-c3nc(-c4ccccc4)nc(-c4ccc5ccc6ccc7c8ccccc8[se]c7c6c5c4)n3)cc2)cc1 ; c1ccc(-c2ccc(-c3nc(-c4ccccc4)nc(-c4ccc5c(ccc6ccc7c8ccccc8[se]c7c65)c4)n3)cc2)cc1]

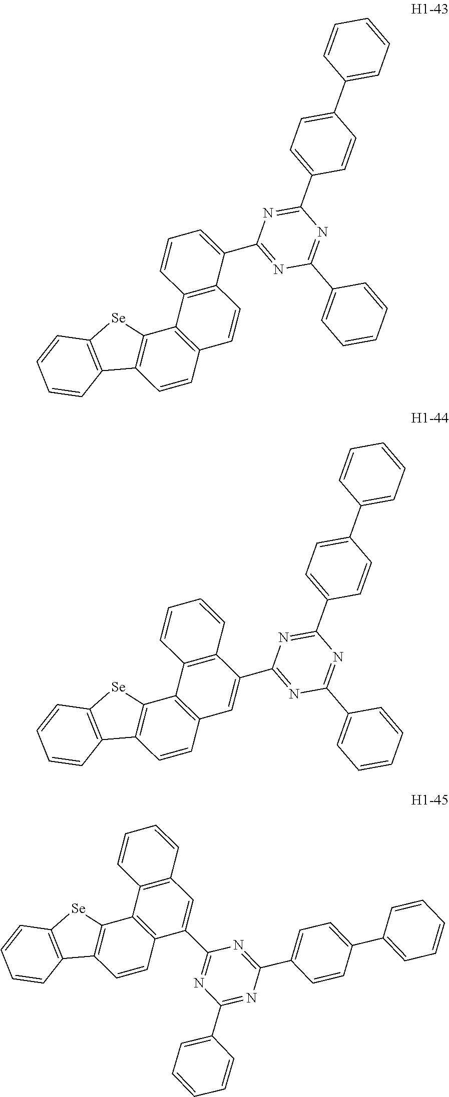 [SMILES: c1ccc(-c2ccc(-c3nc(-c4ccccc4)nc(-c4cccc5c4ccc4ccc6c7ccccc7[se]c6c45)n3)cc2)cc1 ; c1ccc(-c2ccc(-c3nc(-c4ccccc4)nc(-c4cc5ccc6c7ccccc7[se]c6c5c5ccccc45)n3)cc2)cc1 ; c1ccc(-c2ccc(-c3nc(-c4ccccc4)nc(-c4cc5ccccc5c5c4ccc4c6ccccc6[se]c45)n3)cc2)cc1]

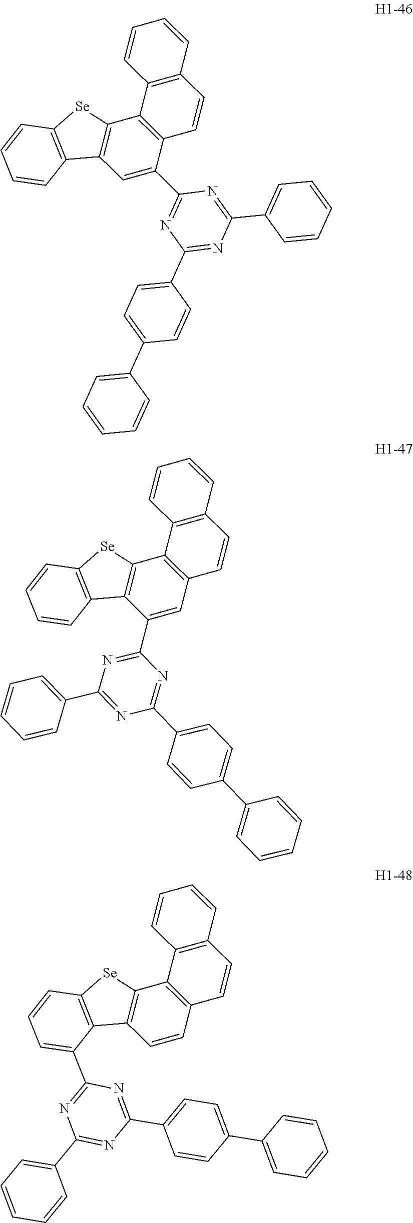

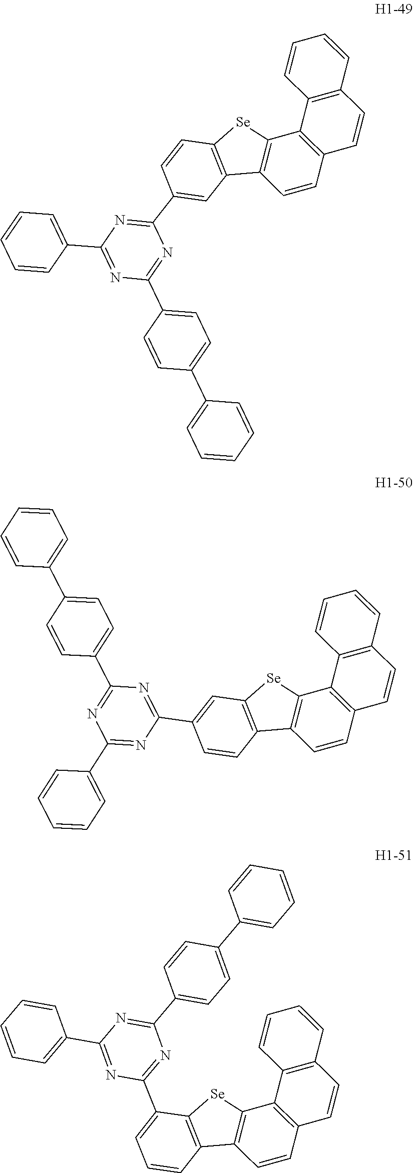 [SMILES: c1ccc(-c2ccc(-c3nc(-c4ccccc4)nc(-c4ccc5[se]c6c(ccc7ccc8ccccc8c76)c5c4)n3)cc2)cc1 ; c1ccc(-c2ccc(-c3nc(-c4ccccc4)nc(-c4ccc5c(c4)[se]c4c5ccc5ccc6ccccc6c54)n3)cc2)cc1 ; c1ccc(-c2ccc(-c3nc(-c4ccccc4)nc(-c4cccc5c4[se]c4c5ccc5ccc6ccccc6c54)n3)cc2)cc1]

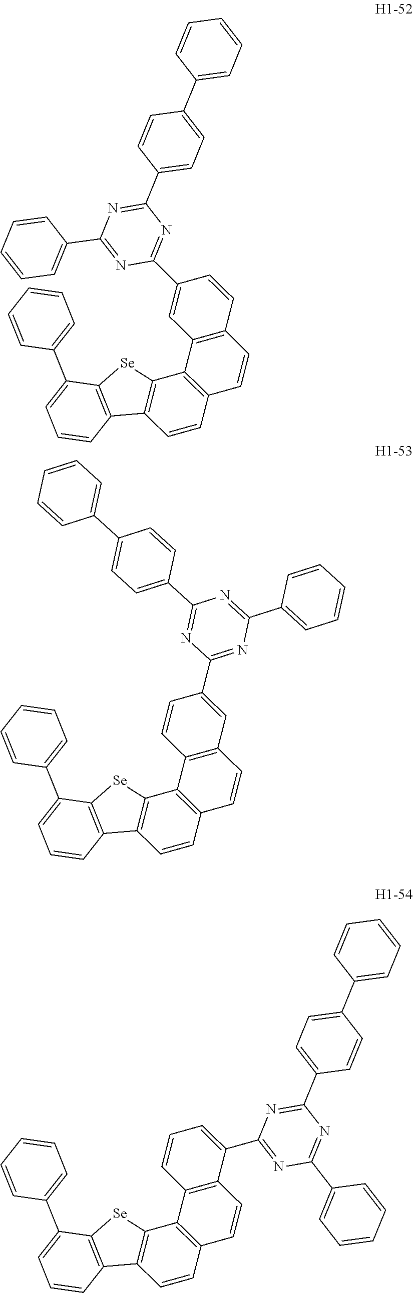

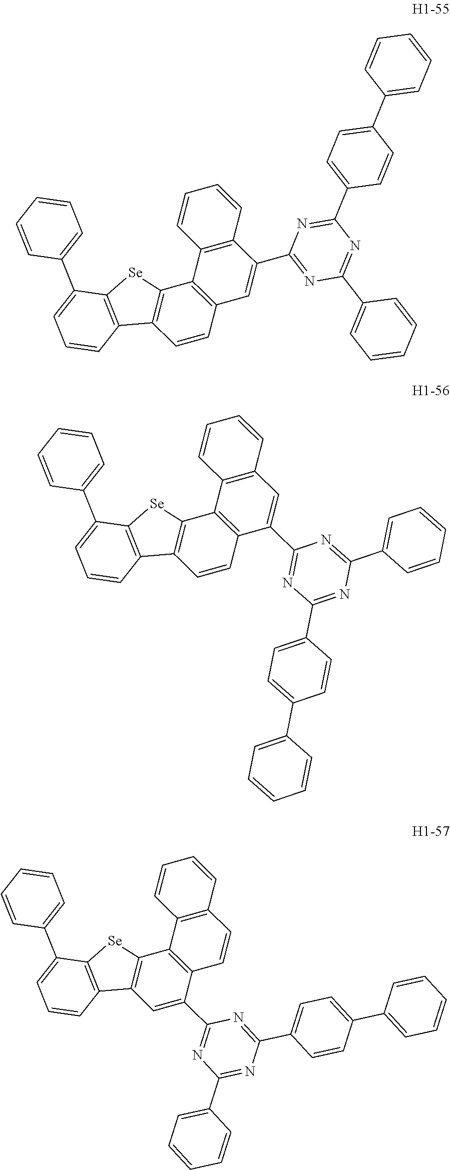 [SMILES: c1ccc(-c2ccc(-c3nc(-c4ccccc4)nc(-c4cc5ccc6c7cccc(-c8ccccc8)c7[se]c6c5c5ccccc45)n3)cc2)cc1 ; c1ccc(-c2ccc(-c3nc(-c4ccccc4)nc(-c4cc5ccccc5c5c4ccc4c6cccc(-c7ccccc7)c6[se]c45)n3)cc2)cc1 ; c1ccc(-c2ccc(-c3nc(-c4ccccc4)nc(-c4cc5c6cccc(-c7ccccc7)c6[se]c5c5c4ccc4ccccc45)n3)cc2)cc1]

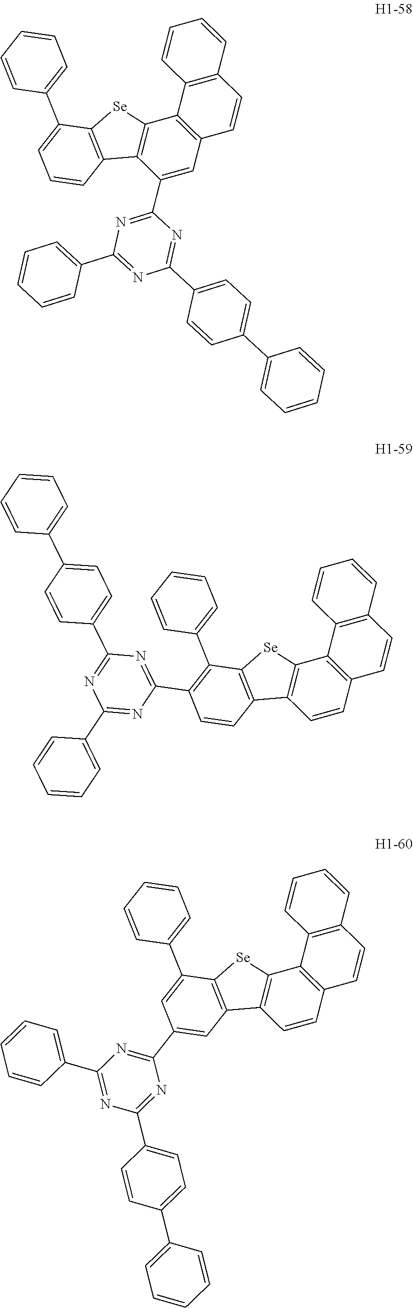 [SMILES: c1ccc(-c2ccc(-c3nc(-c4ccccc4)nc(-c4cc5ccc6ccccc6c5c5[se]c6c(-c7ccccc7)cccc6c45)n3)cc2)cc1 ; c1ccc(-c2ccc(-c3nc(-c4ccccc4)nc(-c4ccc5c([se]c6c5ccc5ccc7ccccc7c56)c4-c4ccccc4)n3)cc2)cc1 ; c1ccc(-c2ccc(-c3nc(-c4ccccc4)nc(-c4cc(-c5ccccc5)c5[se]c6c(ccc7ccc8ccccc8c76)c5c4)n3)cc2)cc1]

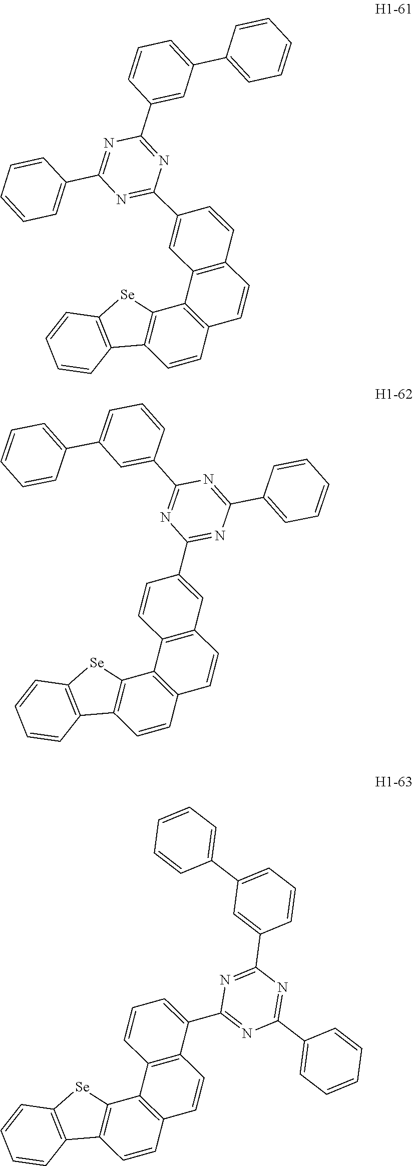

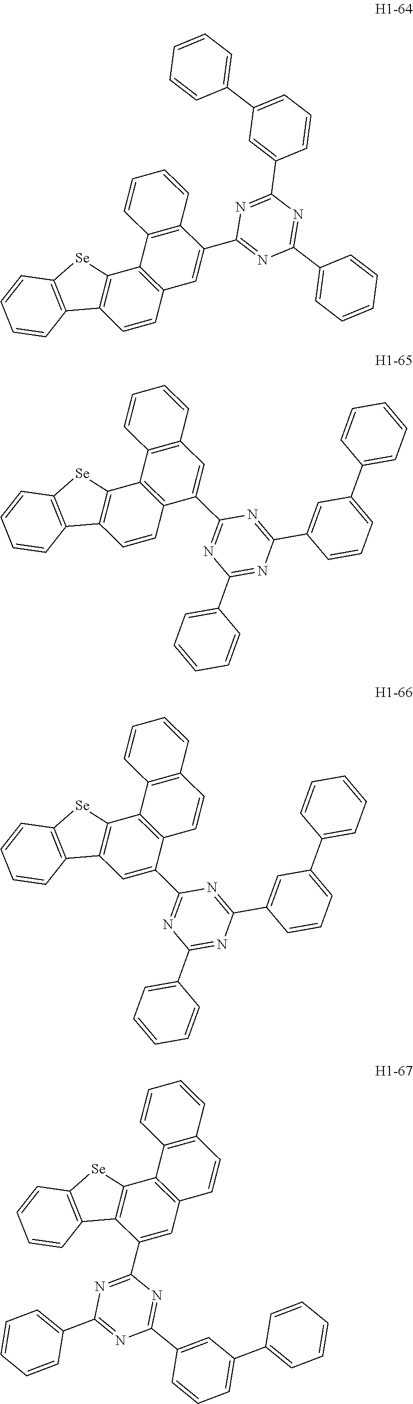

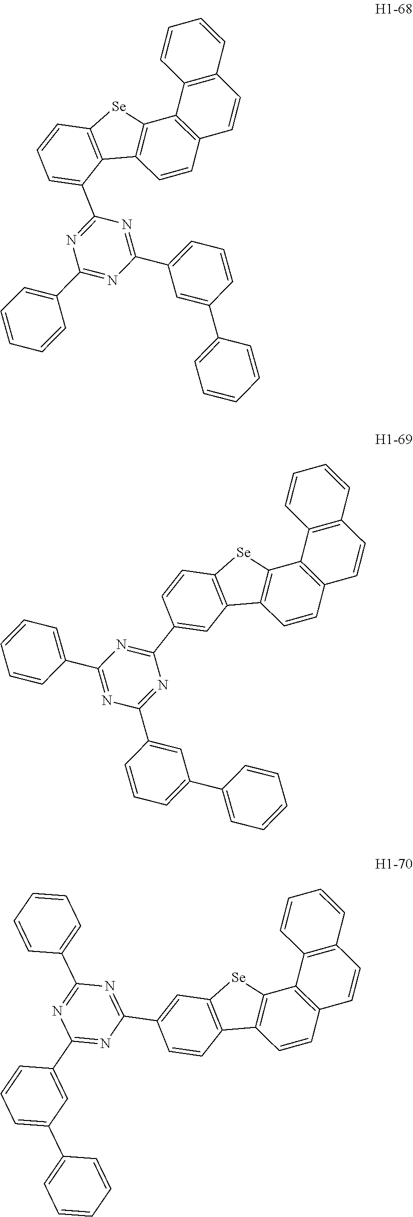 [SMILES: c1ccc(-c2cccc(-c3nc(-c4ccccc4)nc(-c4cccc5[se]c6c(ccc7ccc8ccccc8c76)c45)n3)c2)cc1 ; c1ccc(-c2cccc(-c3nc(-c4ccccc4)nc(-c4ccc5[se]c6c(ccc7ccc8ccccc8c76)c5c4)n3)c2)cc1 ; c1ccc(-c2cccc(-c3nc(-c4ccccc4)nc(-c4ccc5c(c4)[se]c4c5ccc5ccc6ccccc6c54)n3)c2)cc1]

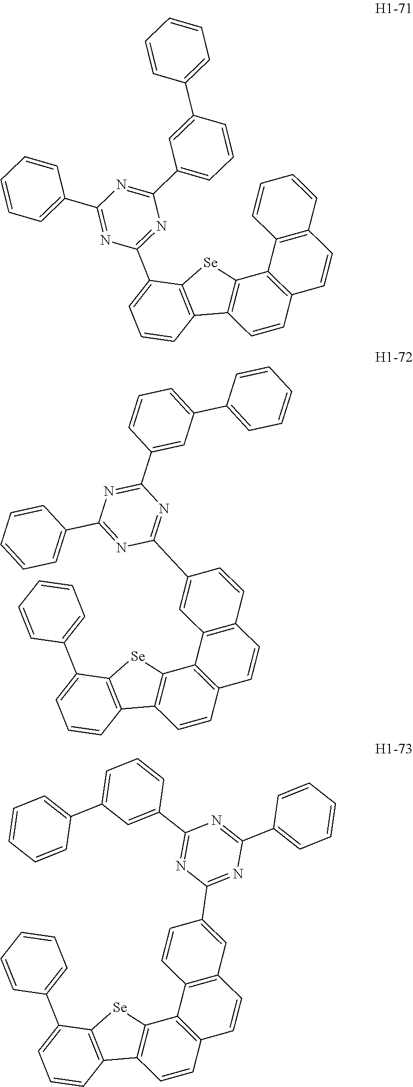

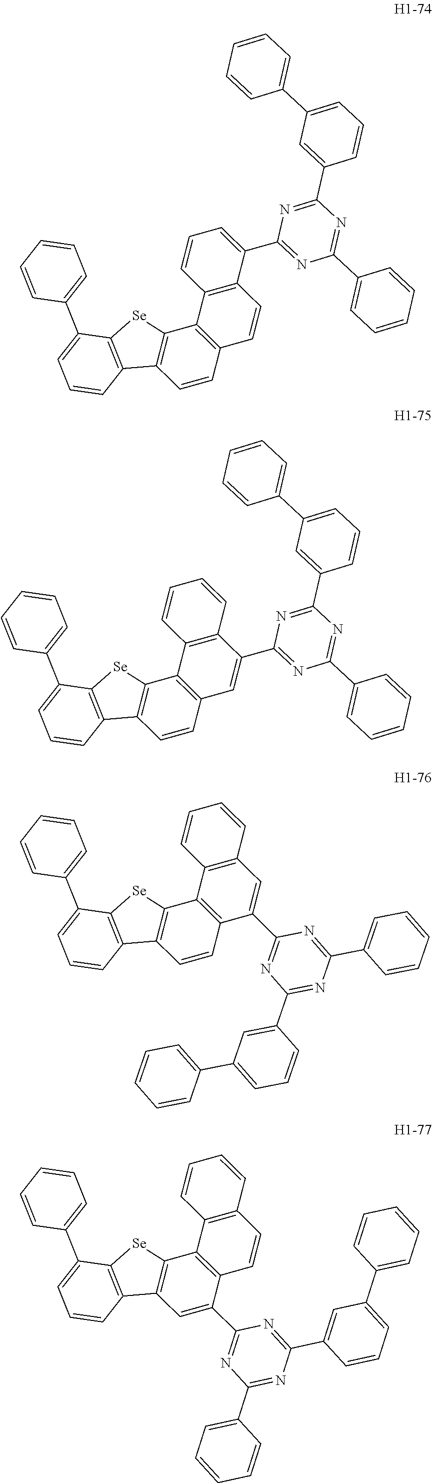 [SMILES: c1ccc(-c2cccc(-c3nc(-c4ccccc4)nc(-c4cccc5c4ccc4ccc6c7cccc(-c8ccccc8)c7[se]c6c45)n3)c2)cc1 ; c1ccc(-c2cccc(-c3nc(-c4ccccc4)nc(-c4cc5ccc6c7cccc(-c8ccccc8)c7[se]c6c5c5ccccc45)n3)c2)cc1 ; c1ccc(-c2cccc(-c3nc(-c4ccccc4)nc(-c4cc5ccccc5c5c4ccc4c6cccc(-c7ccccc7)c6[se]c45)n3)c2)cc1 ; c1ccc(-c2cccc(-c3nc(-c4ccccc4)nc(-c4cc5c6cccc(-c7ccccc7)c6[se]c5c5c4ccc4ccccc45)n3)c2)cc1]

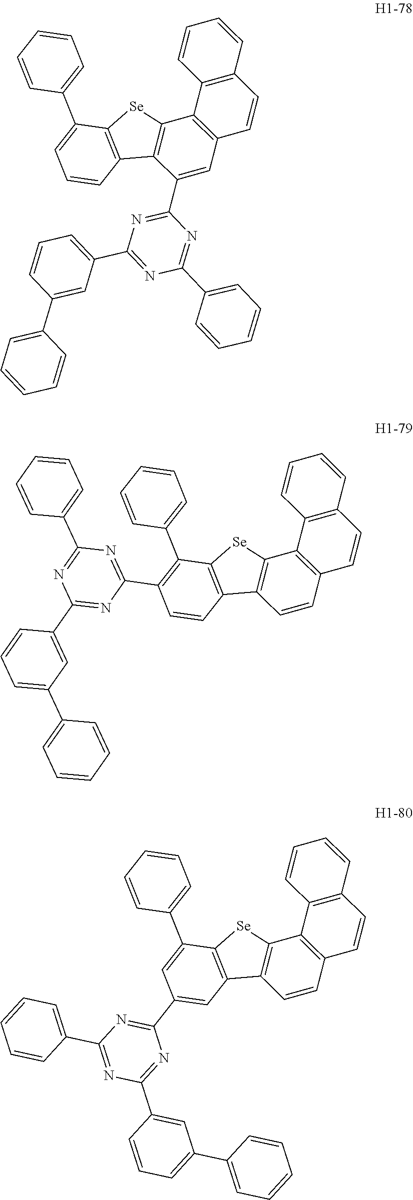 [SMILES: c1ccc(-c2cccc(-c3nc(-c4ccccc4)nc(-c4cc5ccc6ccccc6c5c5[se]c6c(-c7ccccc7)cccc6c45)n3)c2)cc1 ; c1ccc(-c2cccc(-c3nc(-c4ccccc4)nc(-c4ccc5c([se]c6c5ccc5ccc7ccccc7c56)c4-c4ccccc4)n3)c2)cc1 ; c1ccc(-c2cccc(-c3nc(-c4ccccc4)nc(-c4cc(-c5ccccc5)c5[se]c6c(ccc7ccc8ccccc8c76)c5c4)n3)c2)cc1]

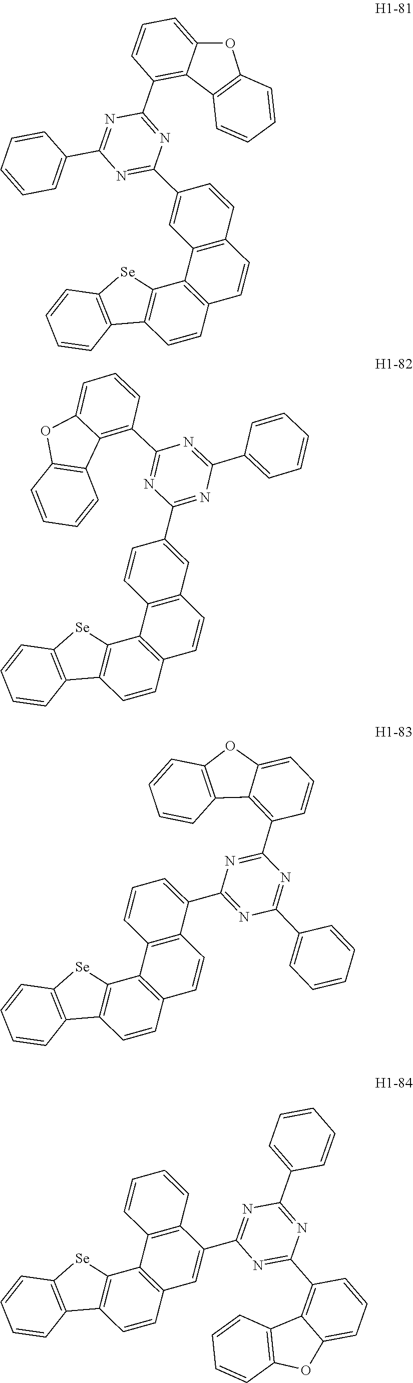

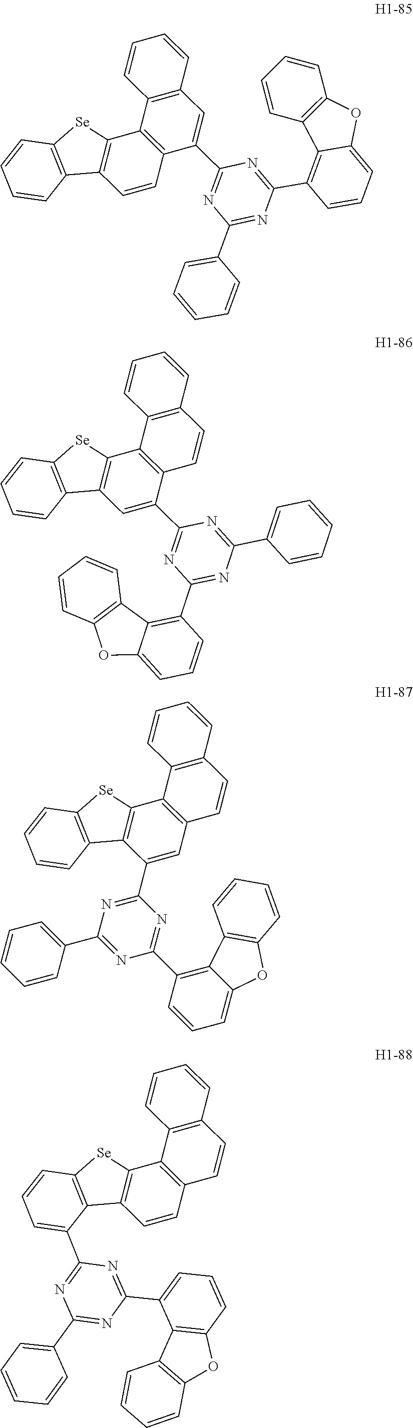

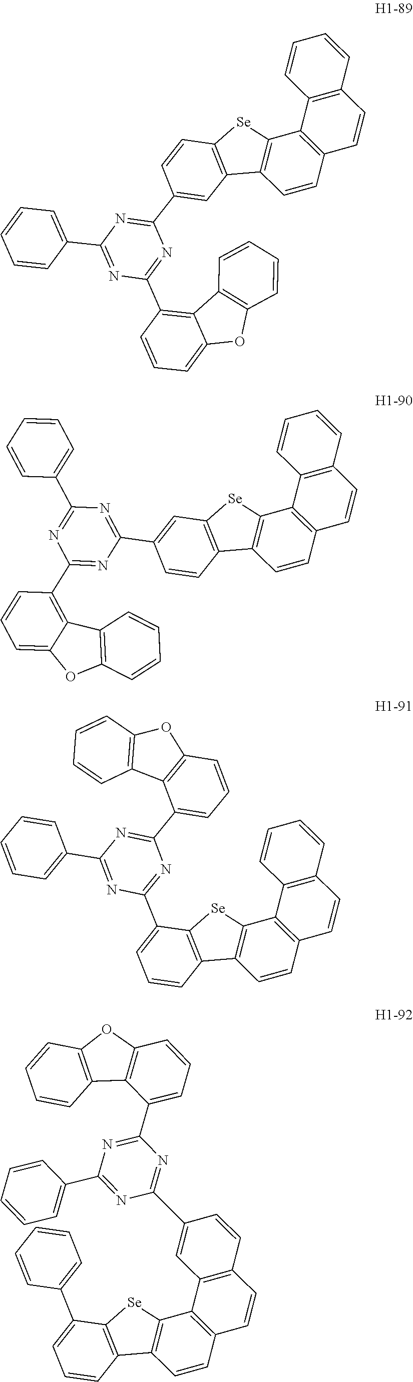

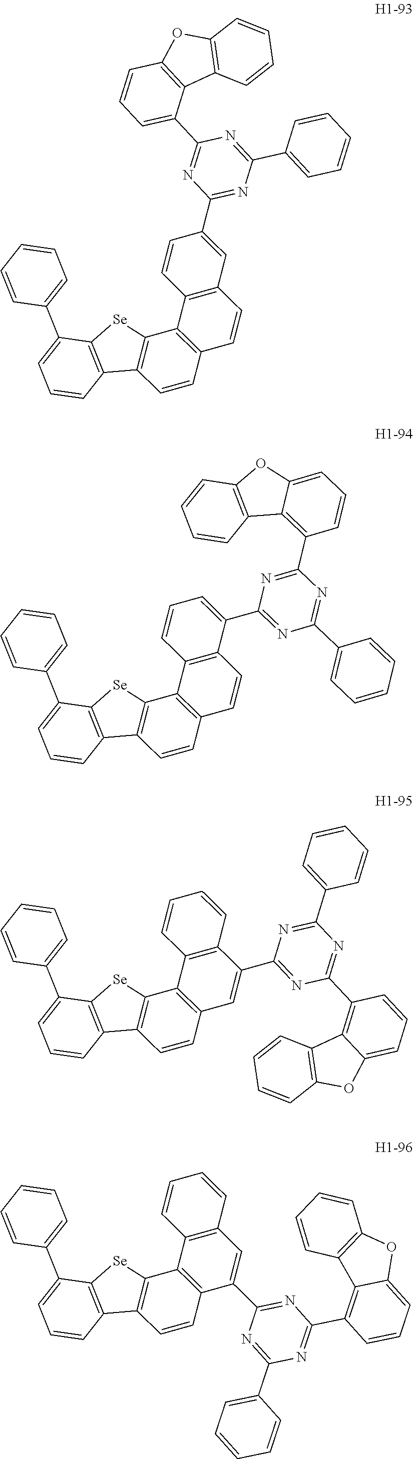 [SMILES: c1ccc(-c2nc(-c3ccc4c(ccc5ccc6c7cccc(-c8ccccc8)c7[se]c6c54)c3)nc(-c3cccc4oc5ccccc5c34)n2)cc1 ; c1ccc(-c2nc(-c3cccc4c3ccc3ccc5c6cccc(-c7ccccc7)c6[se]c5c34)nc(-c3cccc4oc5ccccc5c34)n2)cc1 ; c1ccc(-c2nc(-c3cc4ccc5c6cccc(-c7ccccc7)c6[se]c5c4c4ccccc34)nc(-c3cccc4oc5ccccc5c34)n2)cc1 ; c1ccc(-c2nc(-c3cc4ccccc4c4c3ccc3c5cccc(-c6ccccc6)c5[se]c34)nc(-c3cccc4oc5ccccc5c34)n2)cc1]

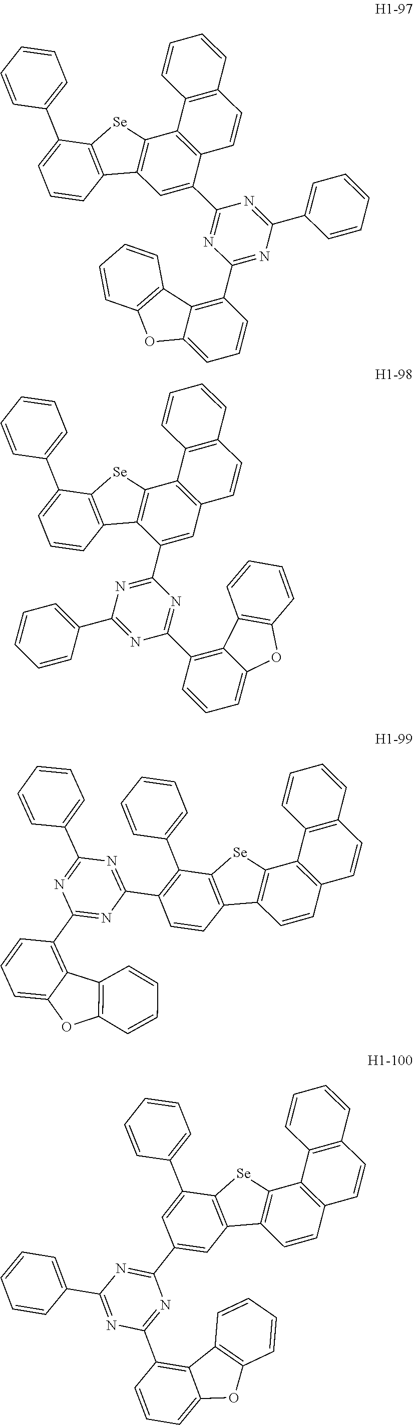

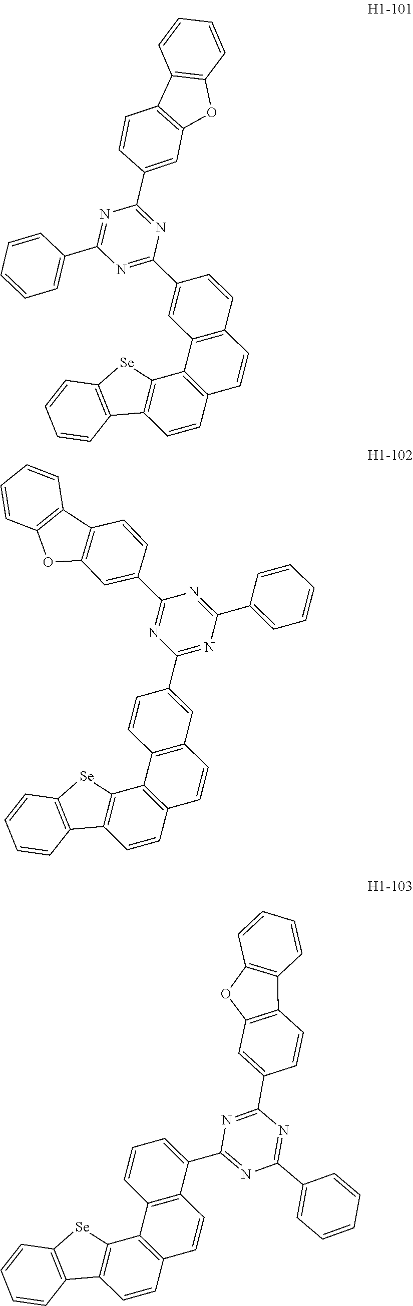

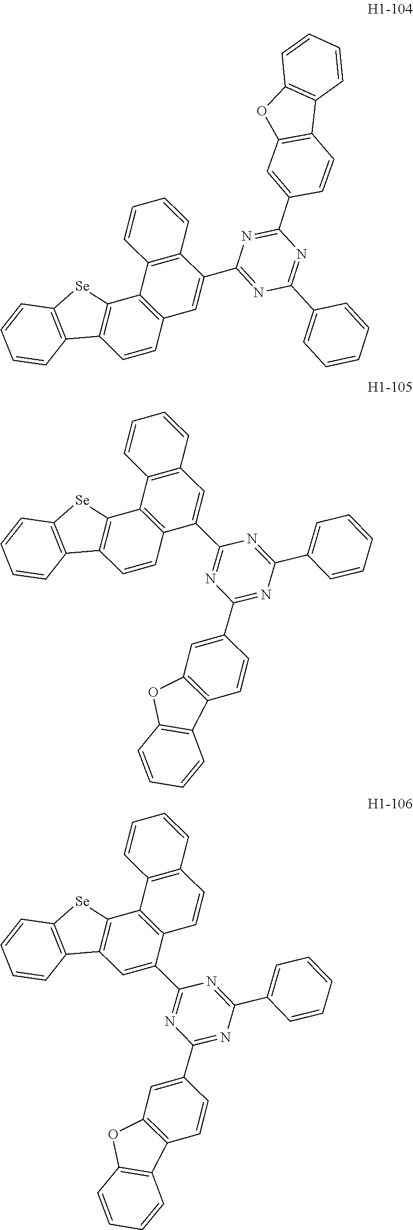

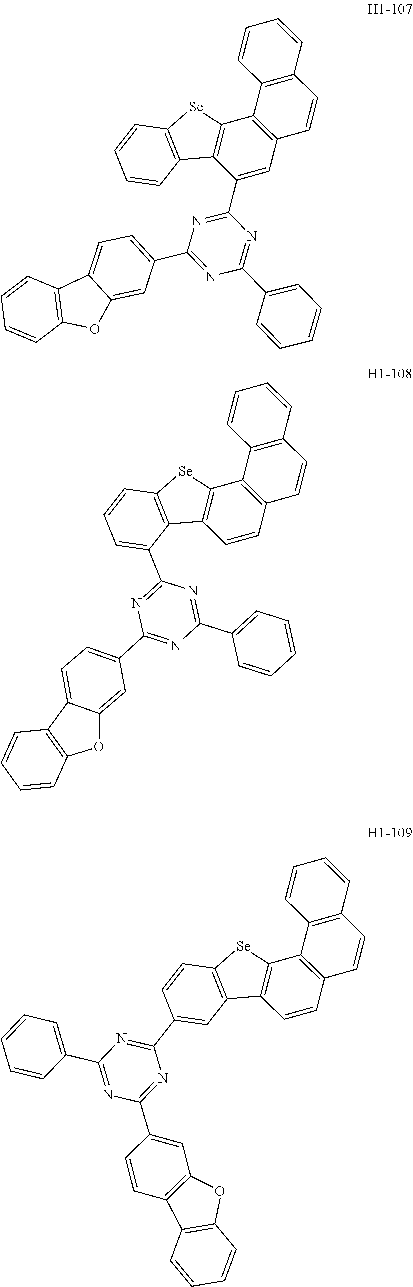

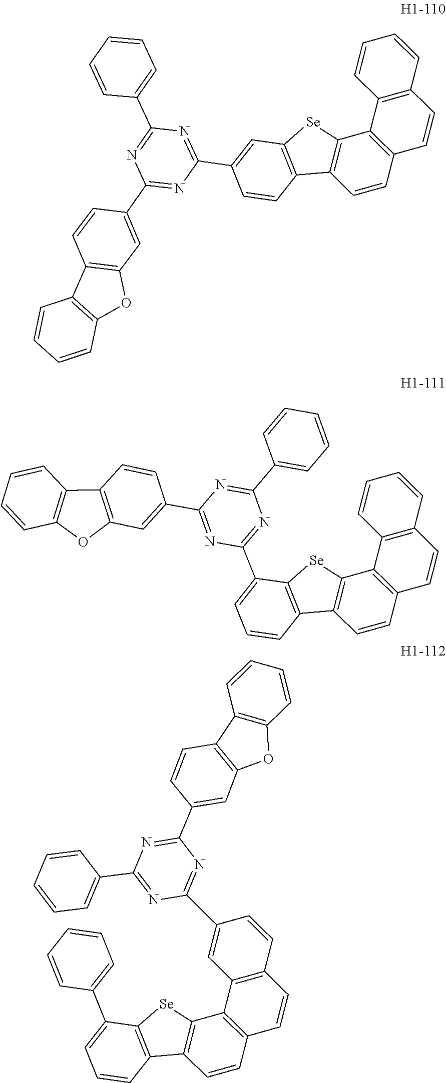

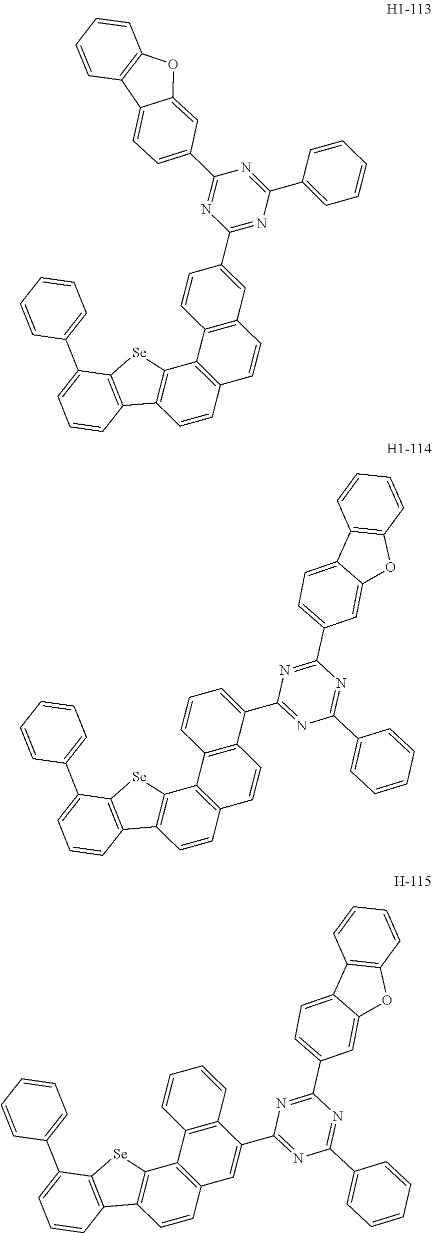 [SMILES: c1ccc(-c2nc(-c3ccc4c(ccc5ccc6c7cccc(-c8ccccc8)c7[se]c6c54)c3)nc(-c3ccc4c(c3)oc3ccccc34)n2)cc1 ; c1ccc(-c2nc(-c3ccc4c(c3)oc3ccccc34)nc(-c3cccc4c3ccc3ccc5c6cccc(-c7ccccc7)c6[se]c5c34)n2)cc1 ; c1ccc(-c2nc(-c3ccc4c(c3)oc3ccccc34)nc(-c3cc4ccc5c6cccc(-c7ccccc7)c6[se]c5c4c4ccccc34)n2)cc1]

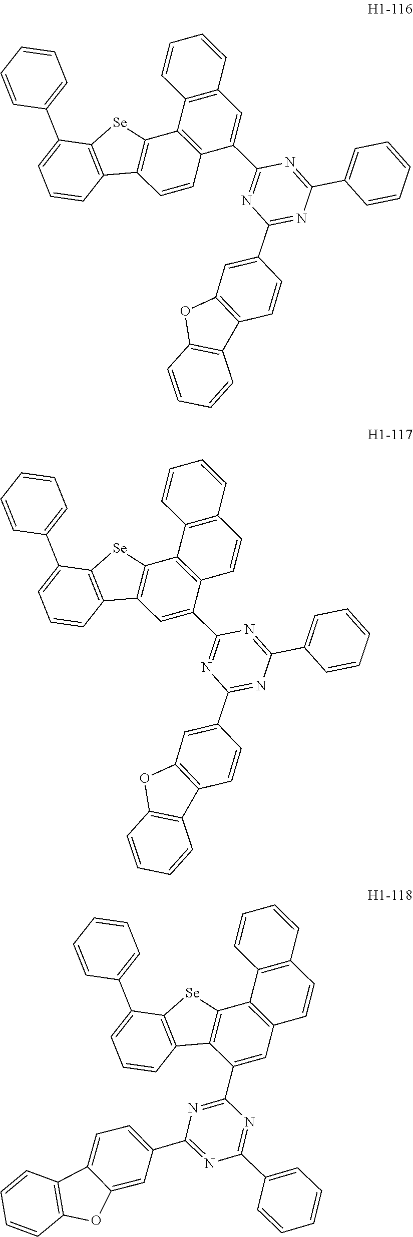 [SMILES: c1ccc(-c2nc(-c3ccc4c(c3)oc3ccccc34)nc(-c3cc4ccccc4c4c3ccc3c5cccc(-c6ccccc6)c5[se]c34)n2)cc1 ; c1ccc(-c2nc(-c3ccc4c(c3)oc3ccccc34)nc(-c3cc4c5cccc(-c6ccccc6)c5[se]c4c4c3ccc3ccccc34)n2)cc1 ; c1ccc(-c2nc(-c3ccc4c(c3)oc3ccccc34)nc(-c3cc4ccc5ccccc5c4c4[se]c5c(-c6ccccc6)cccc5c34)n2)cc1]

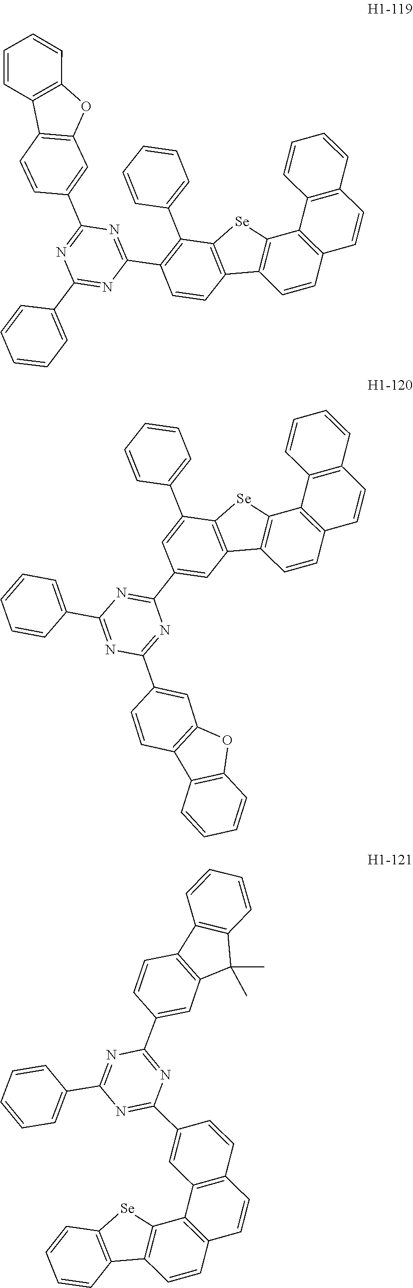

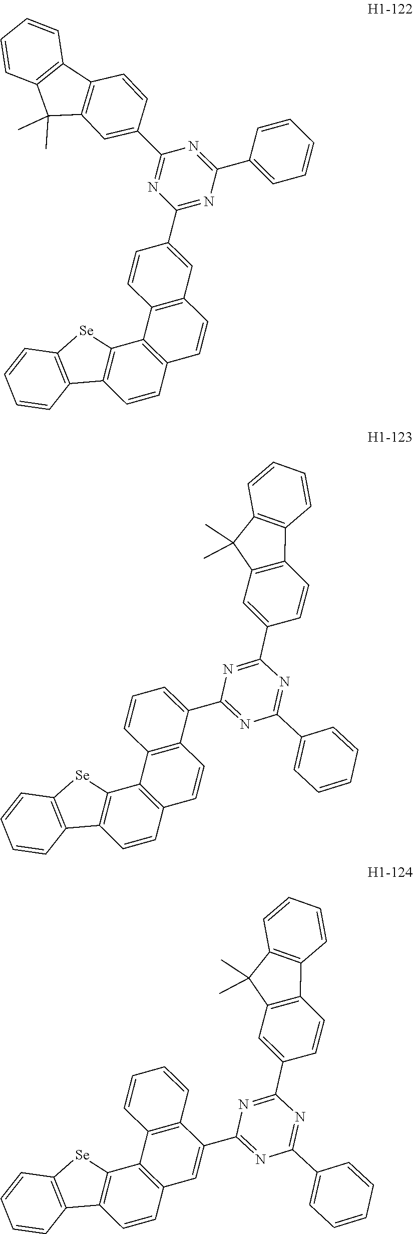 [SMILES: CC1(C)c2ccccc2-c2ccc(-c3nc(-c4ccccc4)nc(-c4ccc5c(ccc6ccc7c8ccccc8[se]c7c65)c4)n3)cc21 ; CC1(C)c2ccccc2-c2ccc(-c3nc(-c4ccccc4)nc(-c4cccc5c4ccc4ccc6c7ccccc7[se]c6c45)n3)cc21 ; CC1(C)c2ccccc2-c2ccc(-c3nc(-c4ccccc4)nc(-c4cc5ccc6c7ccccc7[se]c6c5c5ccccc45)n3)cc21]

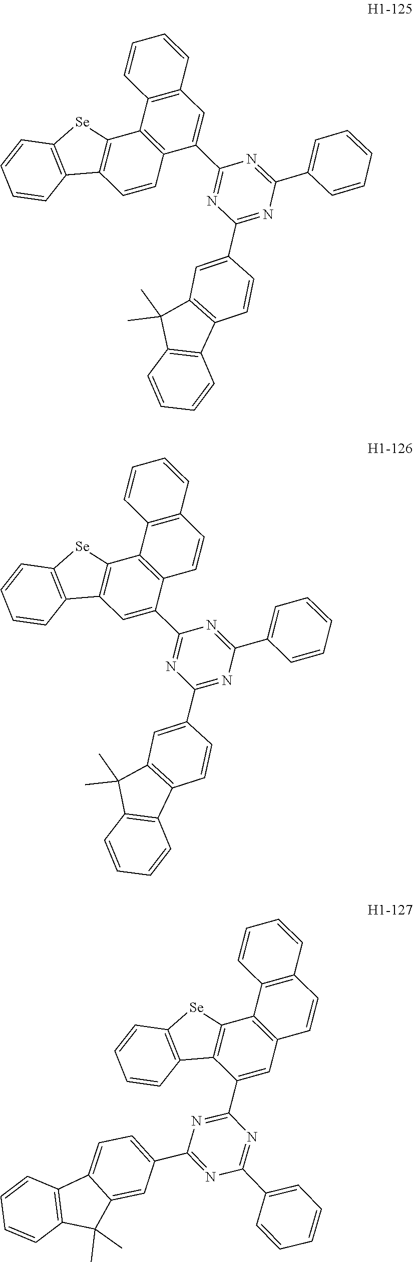 [SMILES: CC1(C)c2ccccc2-c2ccc(-c3nc(-c4ccccc4)nc(-c4cc5ccccc5c5c4ccc4c6ccccc6[se]c45)n3)cc21 ; CC1(C)c2ccccc2-c2ccc(-c3nc(-c4ccccc4)nc(-c4cc5c6ccccc6[se]c5c5c4ccc4ccccc45)n3)cc21 ; CC1(C)c2ccccc2-c2ccc(-c3nc(-c4ccccc4)nc(-c4cc5ccc6ccccc6c5c5[se]c6ccccc6c45)n3)cc21]

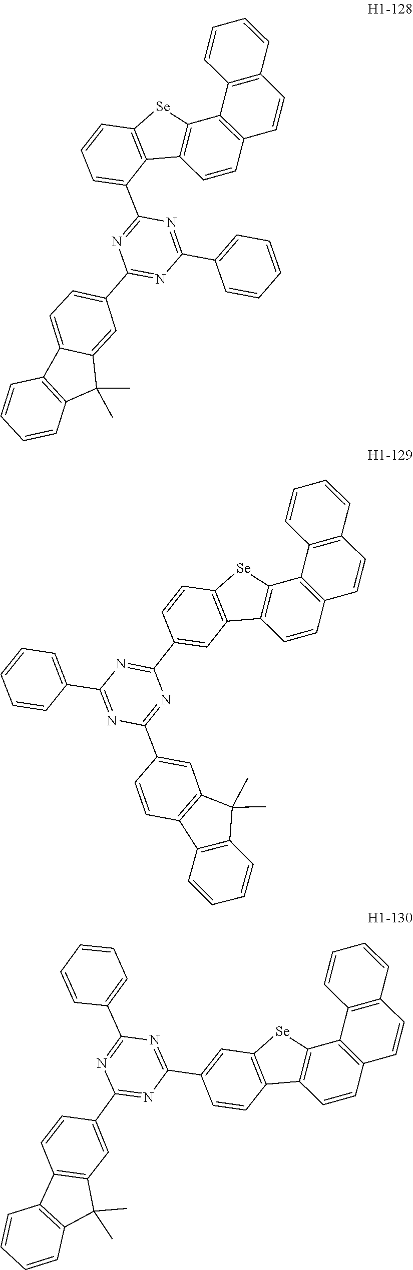 [SMILES: CC1(C)c2ccccc2-c2ccc(-c3nc(-c4ccccc4)nc(-c4cccc5[se]c6c(ccc7ccc8ccccc8c76)c45)n3)cc21 ; CC1(C)c2ccccc2-c2ccc(-c3nc(-c4ccccc4)nc(-c4ccc5[se]c6c(ccc7ccc8ccccc8c76)c5c4)n3)cc21 ; CC1(C)c2ccccc2-c2ccc(-c3nc(-c4ccccc4)nc(-c4ccc5c(c4)[se]c4c5ccc5ccc6ccccc6c54)n3)cc21]

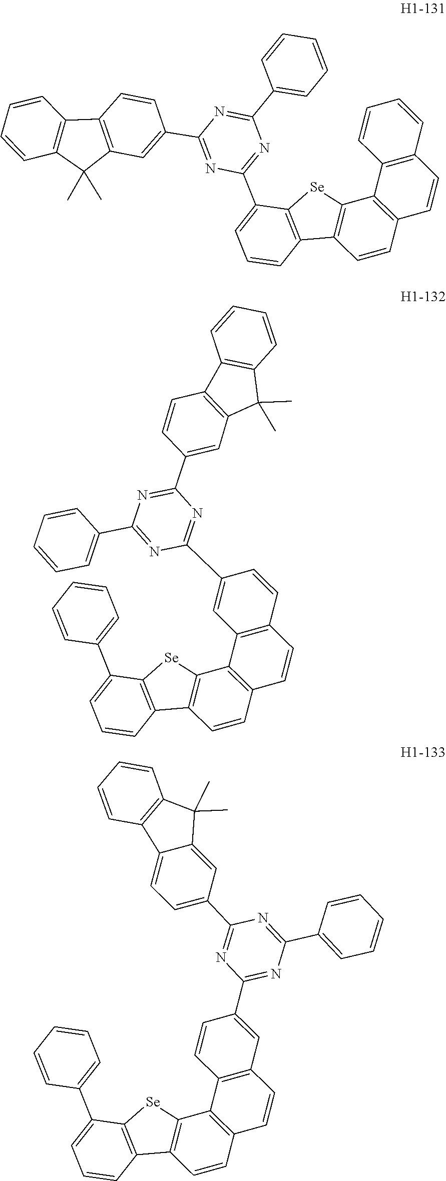

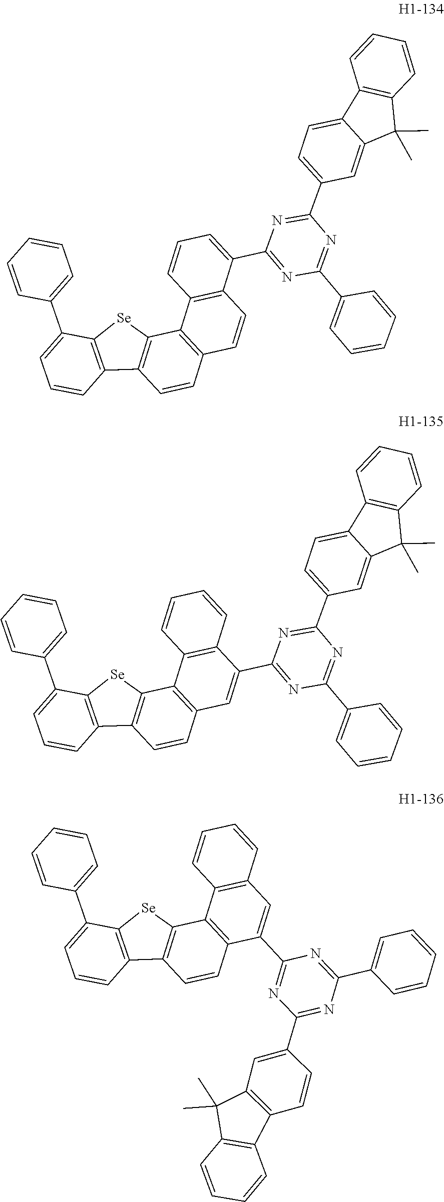 [SMILES: CC1(C)c2ccccc2-c2ccc(-c3nc(-c4ccccc4)nc(-c4cccc5c4ccc4ccc6c7cccc(-c8ccccc8)c7[se]c6c45)n3)cc21 ; CC1(C)c2ccccc2-c2ccc(-c3nc(-c4ccccc4)nc(-c4cc5ccc6c7cccc(-c8ccccc8)c7[se]c6c5c5ccccc45)n3)cc21 ; CC1(C)c2ccccc2-c2ccc(-c3nc(-c4ccccc4)nc(-c4cc5ccccc5c5c4ccc4c6cccc(-c7ccccc7)c6[se]c45)n3)cc21]

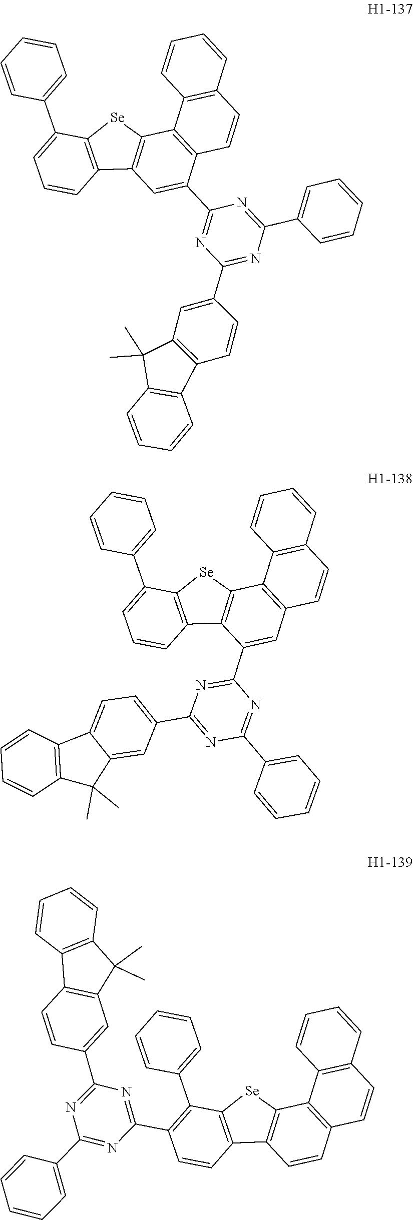 [SMILES: CC1(C)c2ccccc2-c2ccc(-c3nc(-c4ccccc4)nc(-c4cc5c6cccc(-c7ccccc7)c6[se]c5c5c4ccc4ccccc45)n3)cc21 ; CC1(C)c2ccccc2-c2ccc(-c3nc(-c4ccccc4)nc(-c4cc5ccc6ccccc6c5c5[se]c6c(-c7ccccc7)cccc6c45)n3)cc21 ; CC1(C)c2ccccc2-c2ccc(-c3nc(-c4ccccc4)nc(-c4ccc5c([se]c6c5ccc5ccc7ccccc7c56)c4-c4ccccc4)n3)cc21]

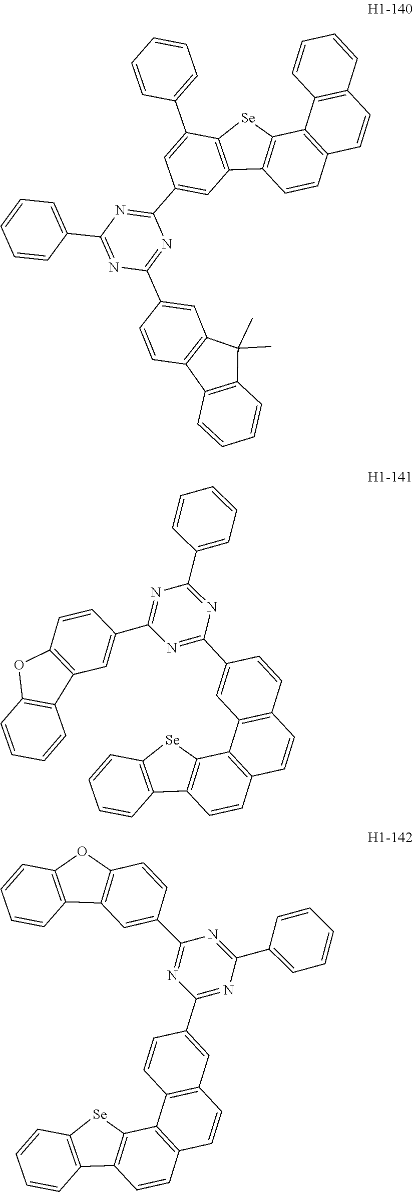

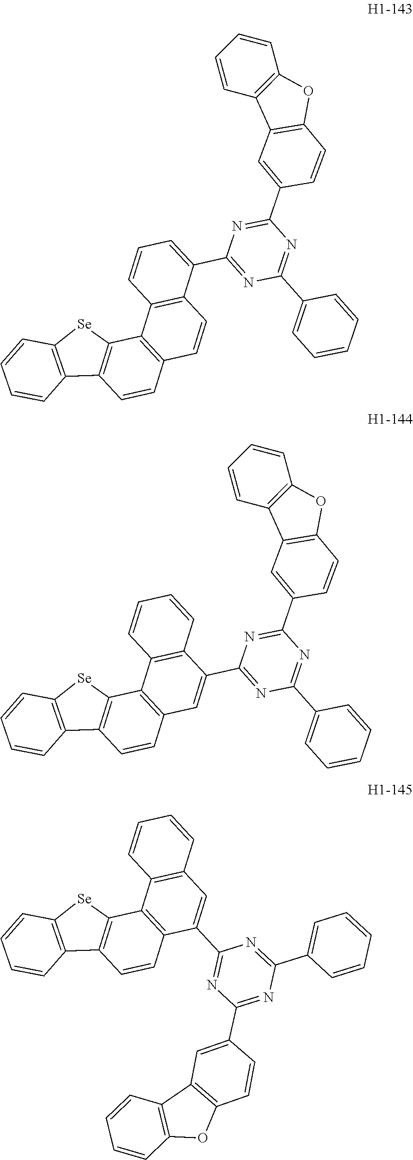 [SMILES: c1ccc(-c2nc(-c3ccc4oc5ccccc5c4c3)nc(-c3cccc4c3ccc3ccc5c6ccccc6[se]c5c34)n2)cc1 ; c1ccc(-c2nc(-c3ccc4oc5ccccc5c4c3)nc(-c3cc4ccc5c6ccccc6[se]c5c4c4ccccc34)n2)cc1 ; c1ccc(-c2nc(-c3ccc4oc5ccccc5c4c3)nc(-c3cc4ccccc4c4c3ccc3c5ccccc5[se]c34)n2)cc1]

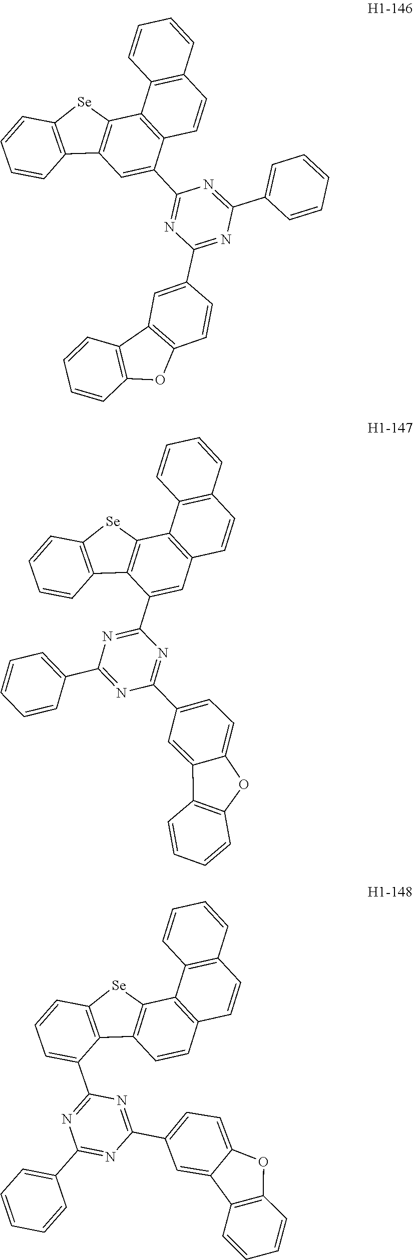

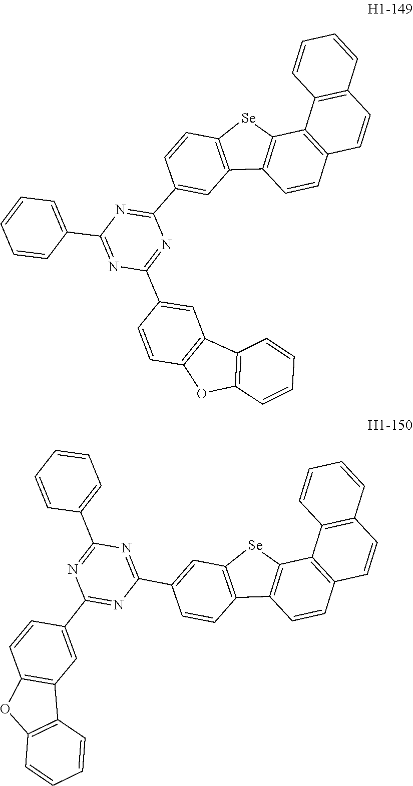 [SMILES: c1ccc(-c2nc(-c3ccc4oc5ccccc5c4c3)nc(-c3ccc4[se]c5c(ccc6ccc7ccccc7c65)c4c3)n2)cc1 ; c1ccc(-c2nc(-c3ccc4c(c3)[se]c3c4ccc4ccc5ccccc5c43)nc(-c3ccc4oc5ccccc5c4c3)n2)cc1]

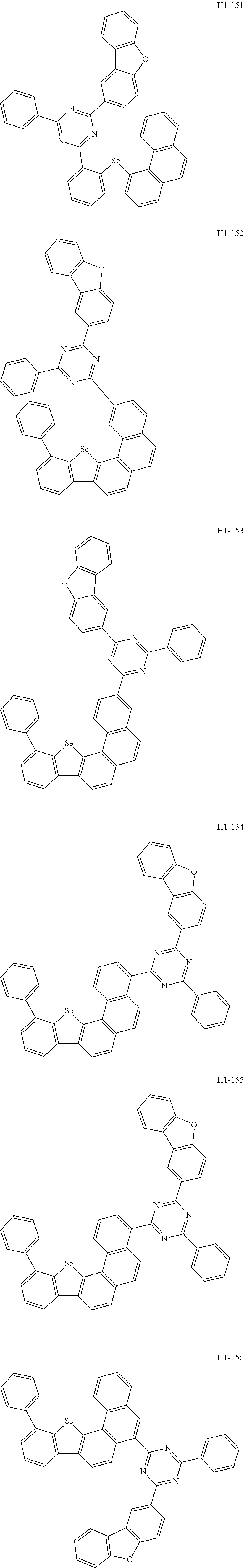

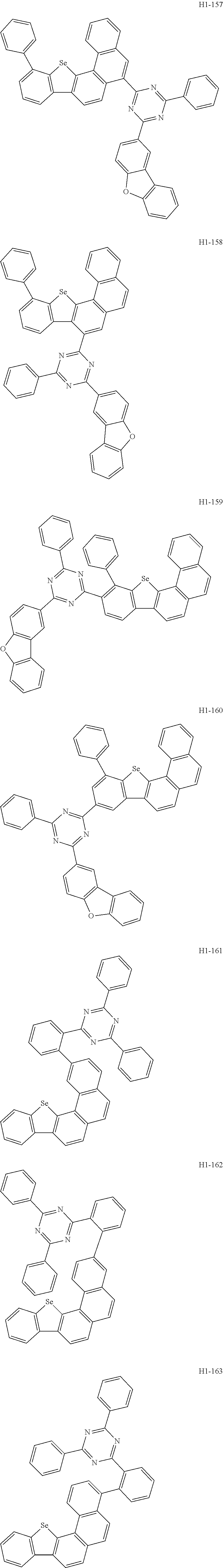

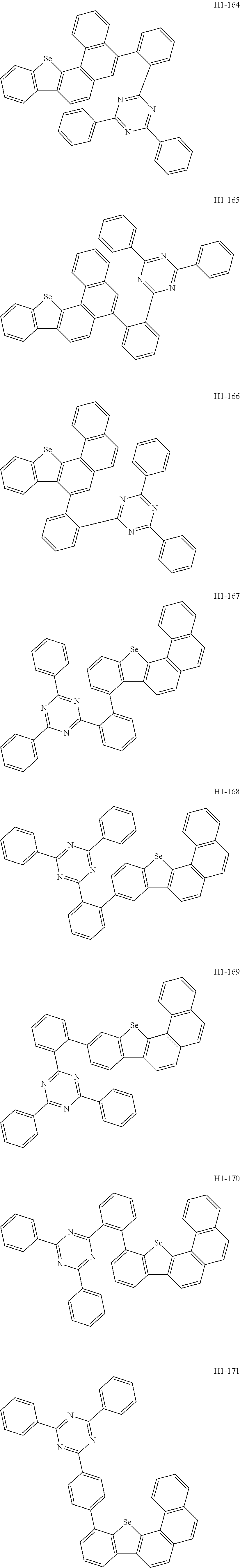

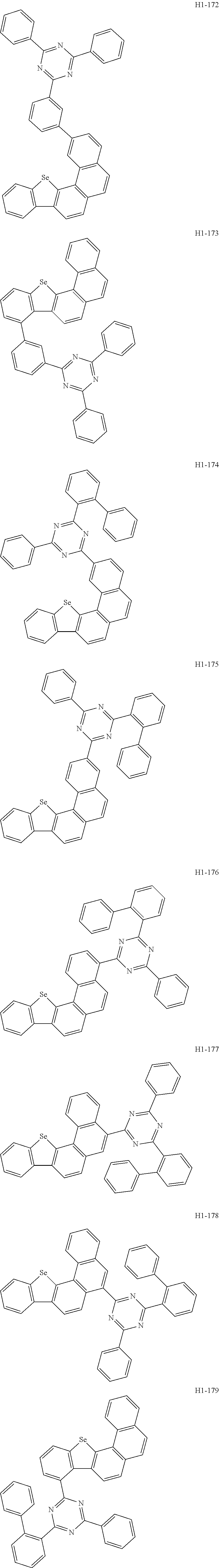

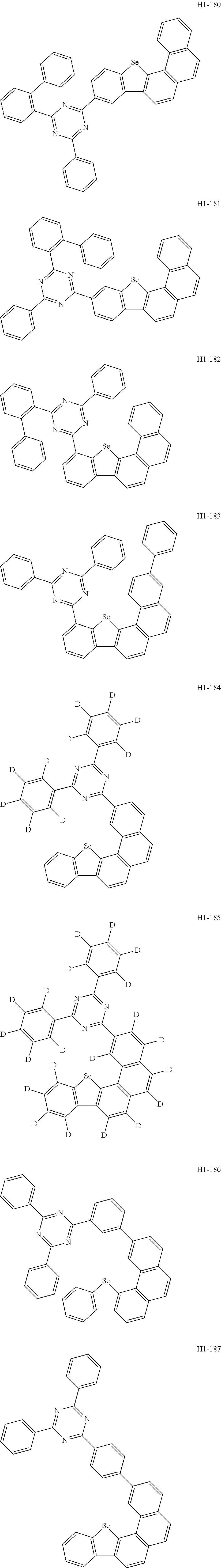

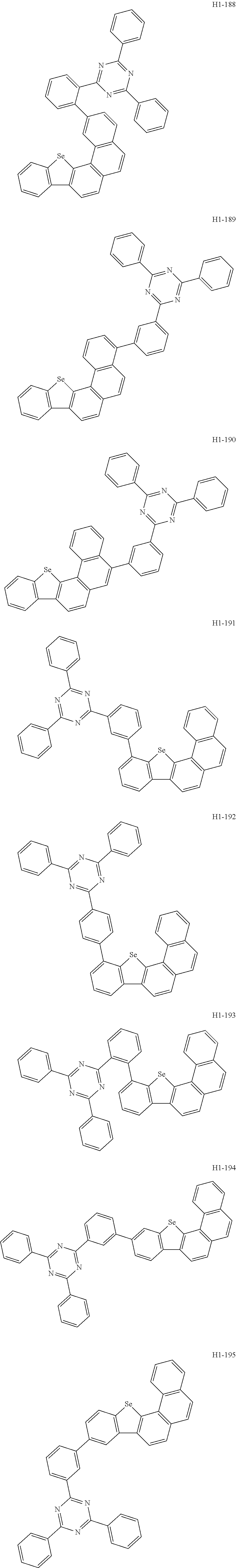

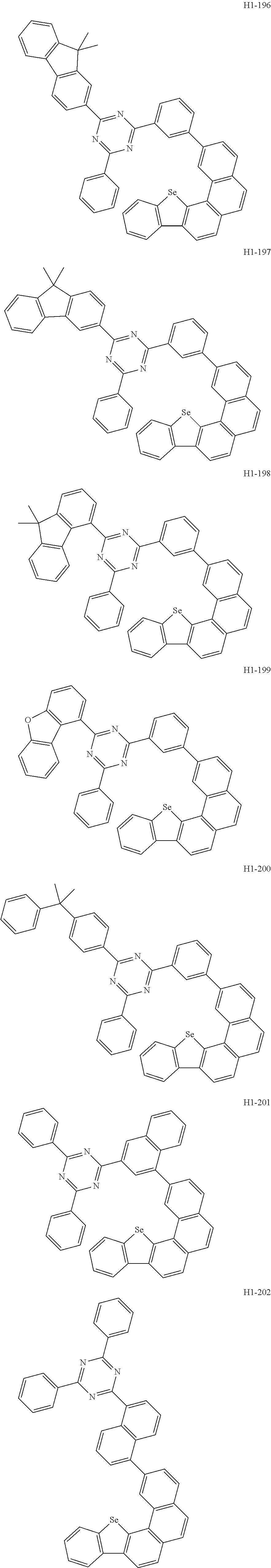

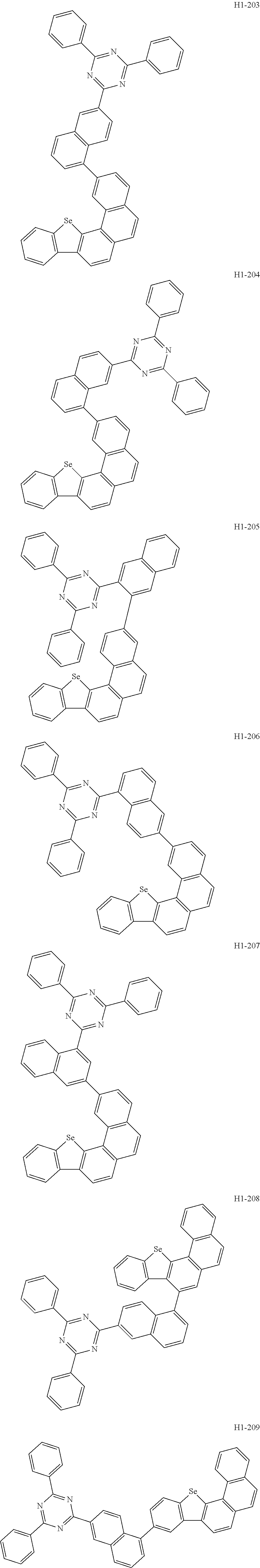

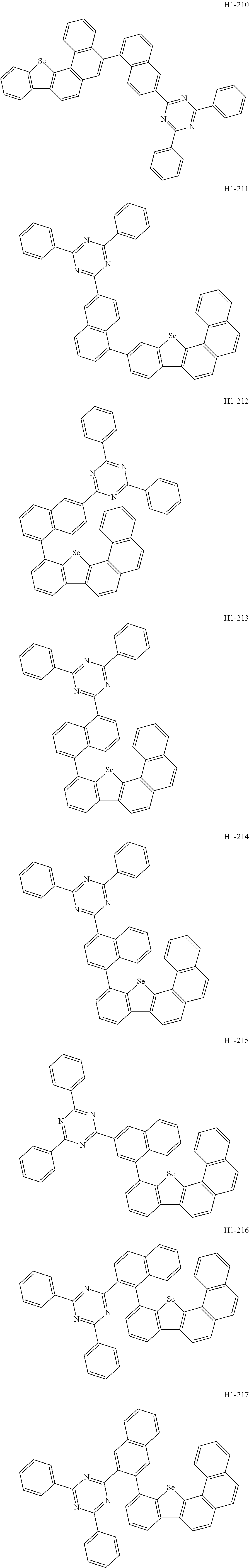

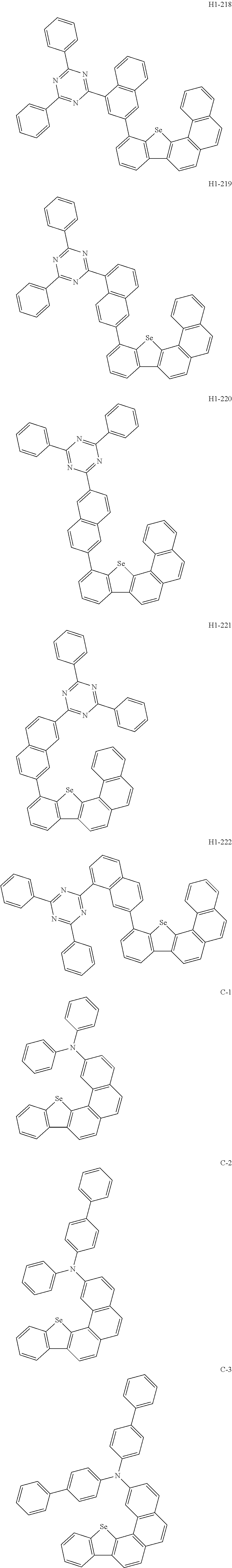

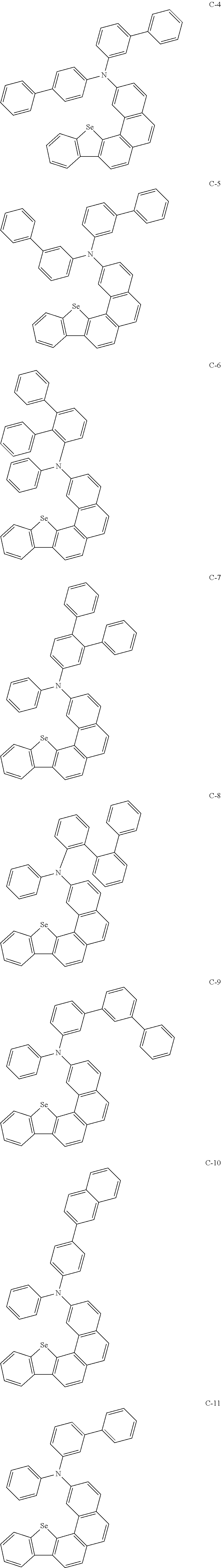

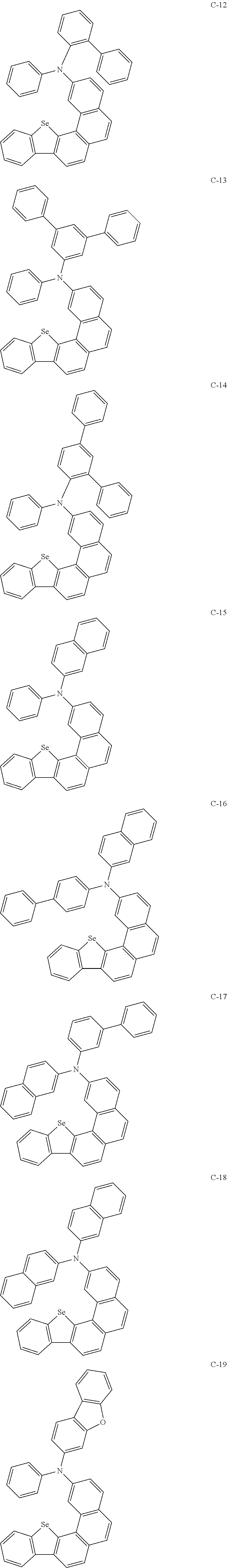

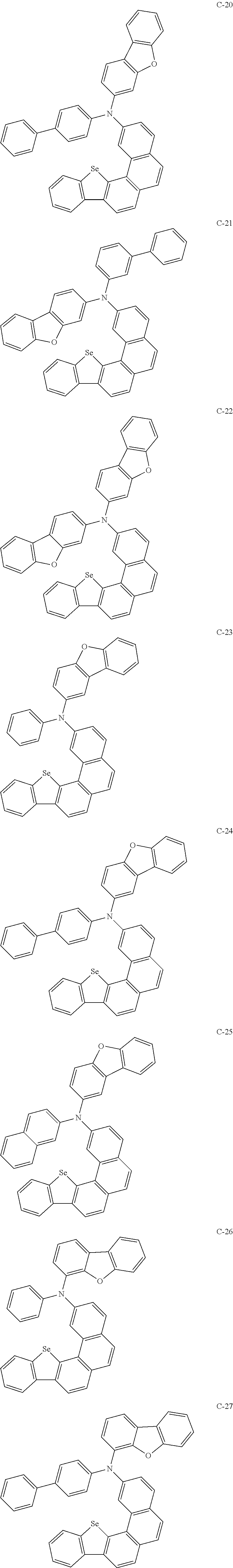

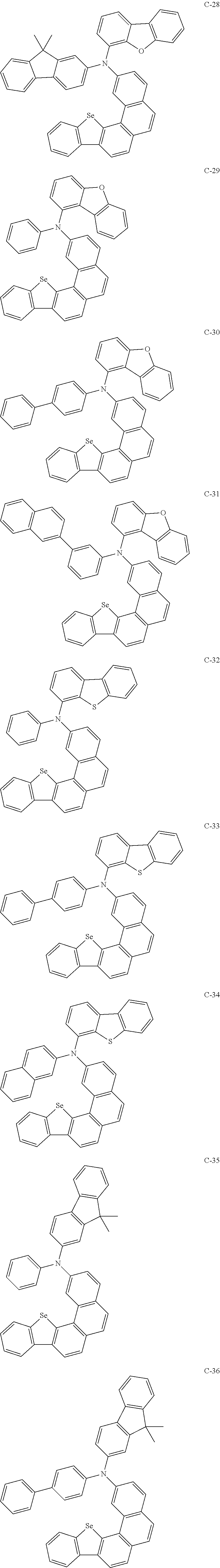

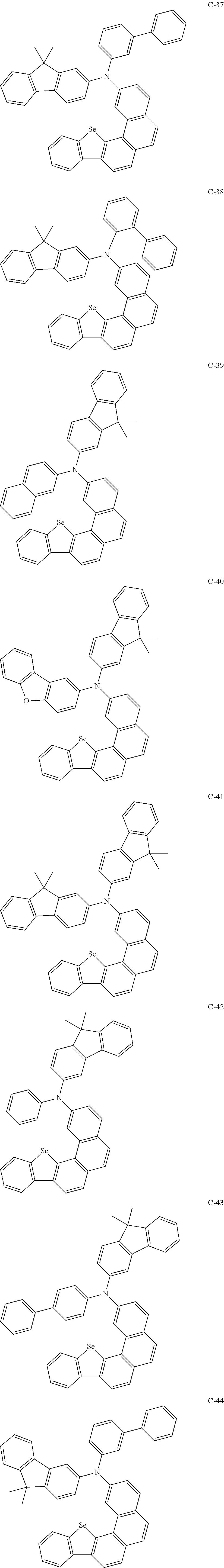

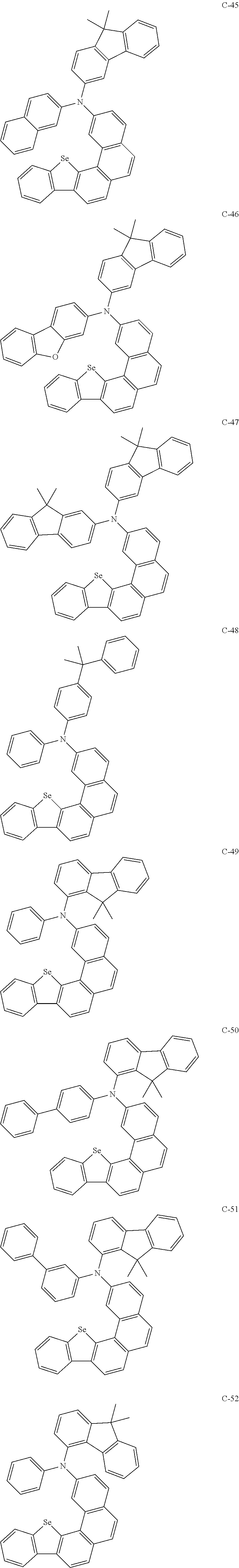

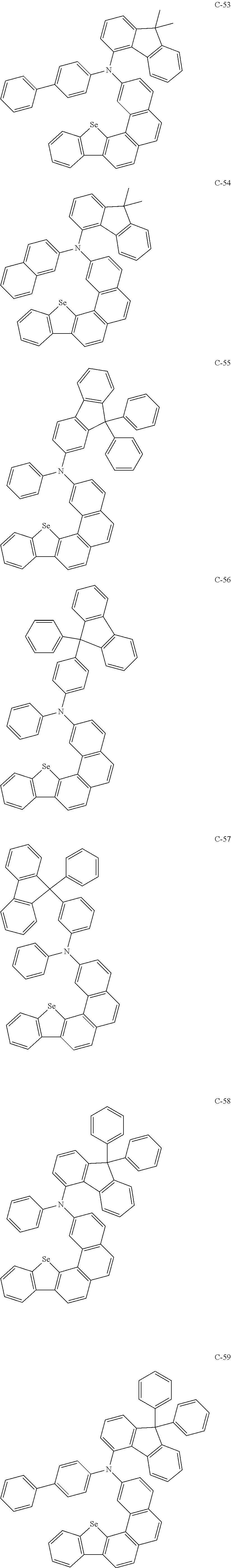

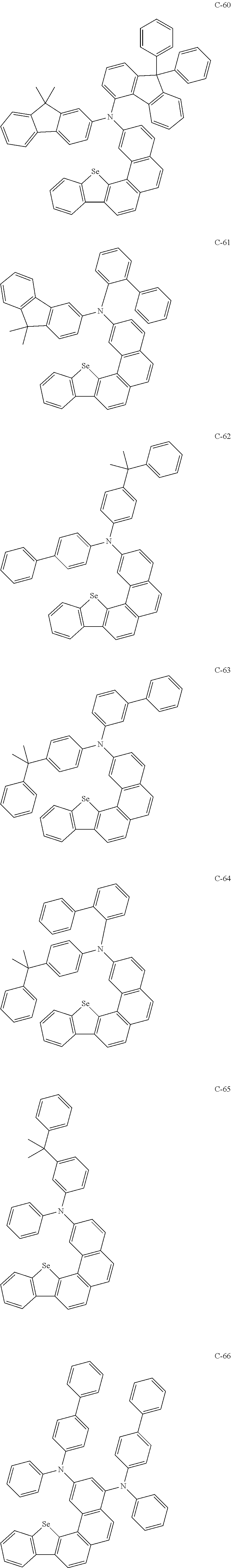

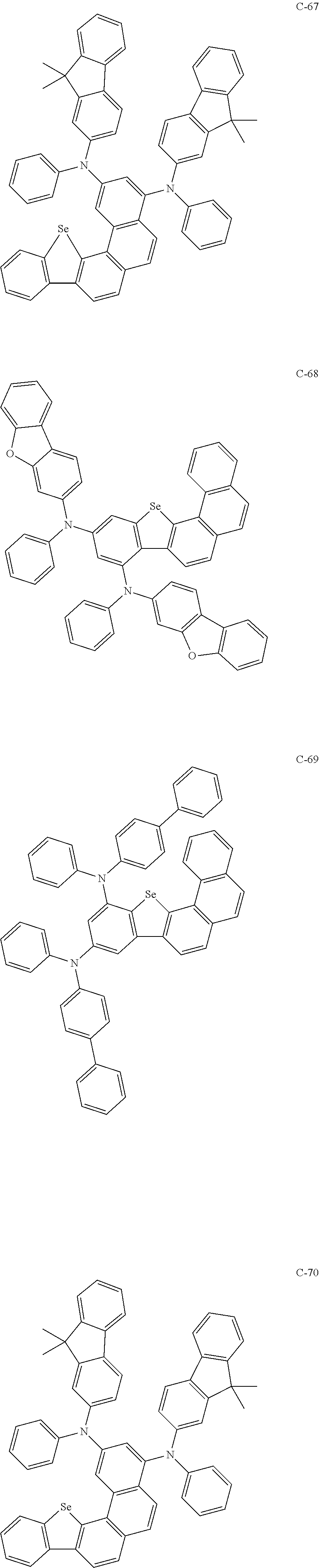 [SMILES: CC1(C)c2ccccc2-c2ccc(N(c3ccccc3)c3cc(N(c4ccccc4)c4ccc5c(c4)C(C)(C)c4ccccc4-5)c4ccc5ccc6c7ccccc7[se]c6c5c4c3)cc21 ; c1ccc(N(c2ccc3c(c2)oc2ccccc23)c2cc(N(c3ccccc3)c3ccc4c(c3)oc3ccccc34)c3c(c2)[se]c2c3ccc3ccc4ccccc4c32)cc1 ; c1ccc(-c2ccc(N(c3ccccc3)c3cc(N(c4ccccc4)c4ccc(-c5ccccc5)cc4)c4[se]c5c(ccc6ccc7ccccc7c65)c4c3)cc2)cc1 ; CC1(C)c2ccccc2-c2ccc(N(c3ccccc3)c3cc(N(c4ccccc4)c4ccc5c(c4)C(C)(C)c4ccccc4-5)c4ccc5ccc6c7ccccc7[se]c6c5c4c3)cc21]

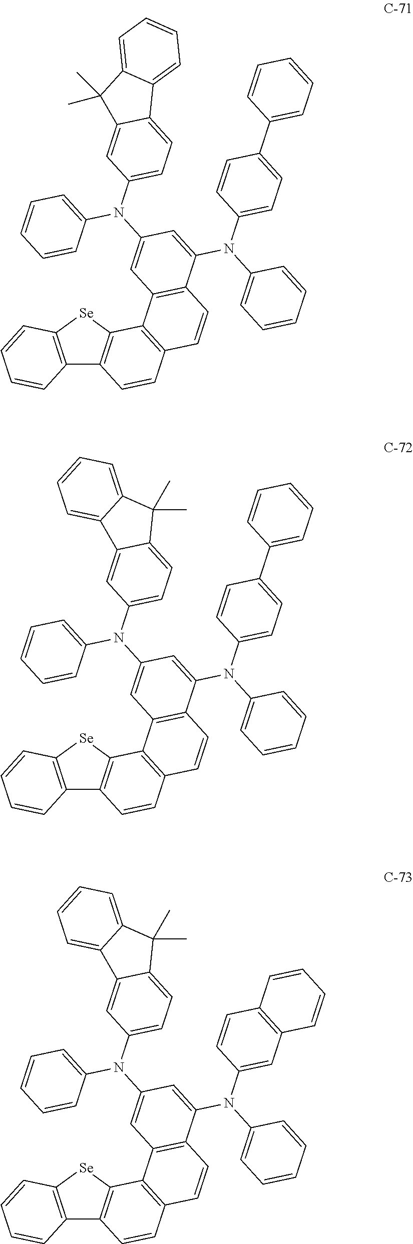 [SMILES: CC1(C)c2ccccc2-c2ccc(N(c3ccccc3)c3cc(N(c4ccccc4)c4ccc(-c5ccccc5)cc4)c4ccc5ccc6c7ccccc7[se]c6c5c4c3)cc21 ; CC1(C)c2ccccc2-c2cc(N(c3ccccc3)c3cc(N(c4ccccc4)c4ccc(-c5ccccc5)cc4)c4ccc5ccc6c7ccccc7[se]c6c5c4c3)ccc21 ; CC1(C)c2ccccc2-c2cc(N(c3ccccc3)c3cc(N(c4ccccc4)c4ccc5ccccc5c4)c4ccc5ccc6c7ccccc7[se]c6c5c4c3)ccc21]

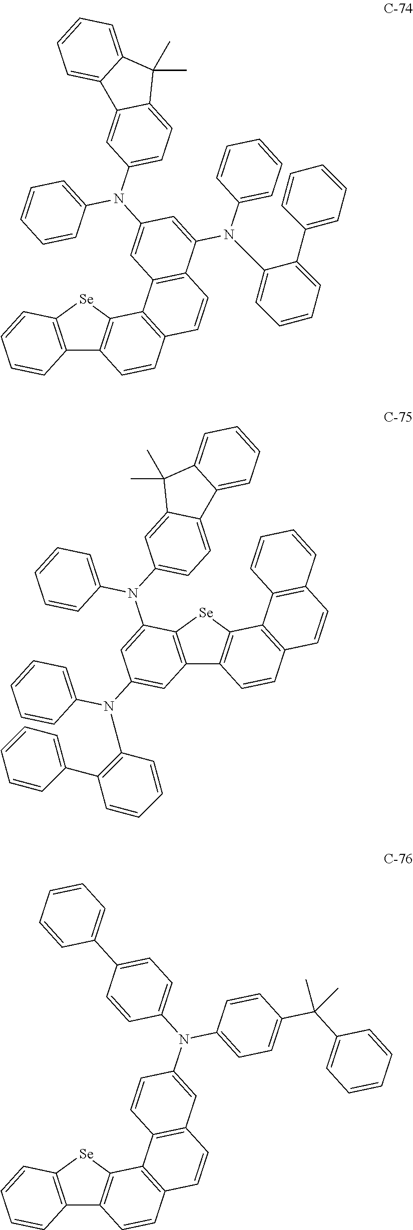 [SMILES: CC1(C)c2ccccc2-c2cc(N(c3ccccc3)c3cc(N(c4ccccc4)c4ccccc4-c4ccccc4)c4ccc5ccc6c7ccccc7[se]c6c5c4c3)ccc21 ; CC1(C)c2ccccc2-c2ccc(N(c3ccccc3)c3cc(N(c4ccccc4)c4ccccc4-c4ccccc4)cc4c3[se]c3c4ccc4ccc5ccccc5c43)cc21 ; CC(C)(c1ccccc1)c1ccc(N(c2ccc(-c3ccccc3)cc2)c2ccc3c(ccc4ccc5c6ccccc6[se]c5c43)c2)cc1]

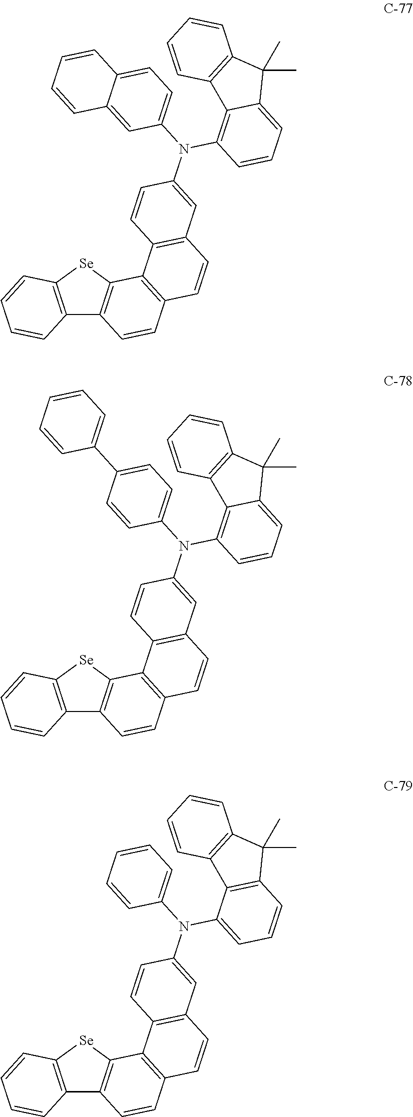 [SMILES: CC1(C)c2ccccc2-c2c(N(c3ccc4ccccc4c3)c3ccc4c(ccc5ccc6c7ccccc7[se]c6c54)c3)cccc21 ; CC1(C)c2ccccc2-c2c(N(c3ccc(-c4ccccc4)cc3)c3ccc4c(ccc5ccc6c7ccccc7[se]c6c54)c3)cccc21 ; CC1(C)c2ccccc2-c2c(N(c3ccccc3)c3ccc4c(ccc5ccc6c7ccccc7[se]c6c54)c3)cccc21]

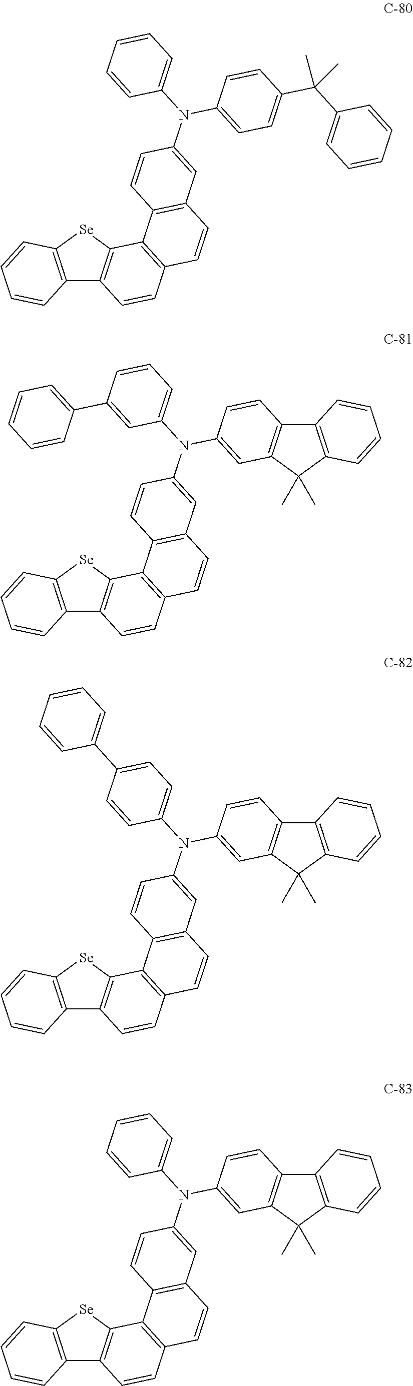 [SMILES: CC(C)(c1ccccc1)c1ccc(N(c2ccccc2)c2ccc3c(ccc4ccc5c6ccccc6[se]c5c43)c2)cc1 ; CC1(C)c2ccccc2-c2ccc(N(c3cccc(-c4ccccc4)c3)c3ccc4c(ccc5ccc6c7ccccc7[se]c6c54)c3)cc21 ; CC1(C)c2ccccc2-c2ccc(N(c3ccc(-c4ccccc4)cc3)c3ccc4c(ccc5ccc6c7ccccc7[se]c6c54)c3)cc21 ; CC1(C)c2ccccc2-c2ccc(N(c3ccccc3)c3ccc4c(ccc5ccc6c7ccccc7[se]c6c54)c3)cc21]

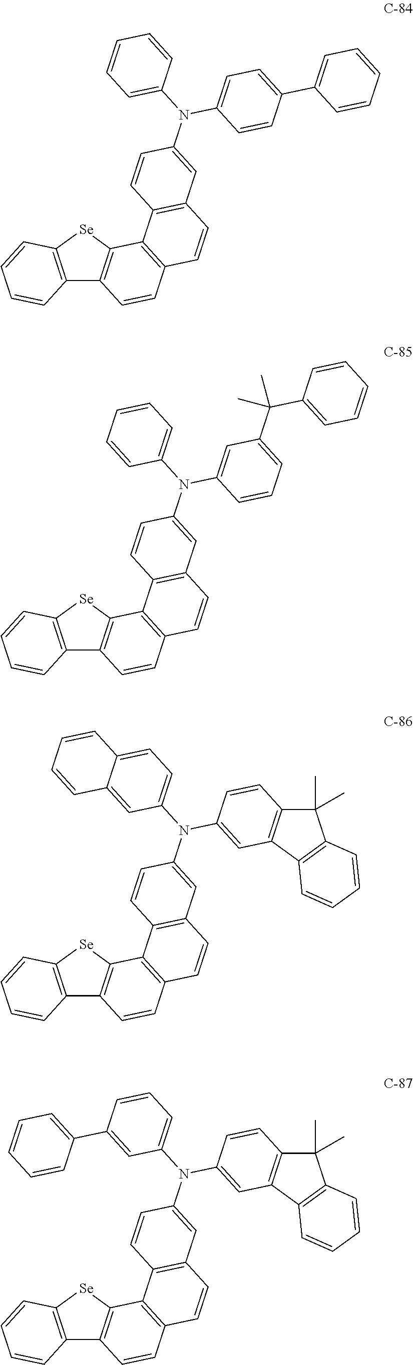 [SMILES: c1ccc(-c2ccc(N(c3ccccc3)c3ccc4c(ccc5ccc6c7ccccc7[se]c6c54)c3)cc2)cc1 ; CC(C)(c1ccccc1)c1cccc(N(c2ccccc2)c2ccc3c(ccc4ccc5c6ccccc6[se]c5c43)c2)c1 ; CC1(C)c2ccccc2-c2cc(N(c3ccc4ccccc4c3)c3ccc4c(ccc5ccc6c7ccccc7[se]c6c54)c3)ccc21 ; CC1(C)c2ccccc2-c2cc(N(c3cccc(-c4ccccc4)c3)c3ccc4c(ccc5ccc6c7ccccc7[se]c6c54)c3)ccc21]

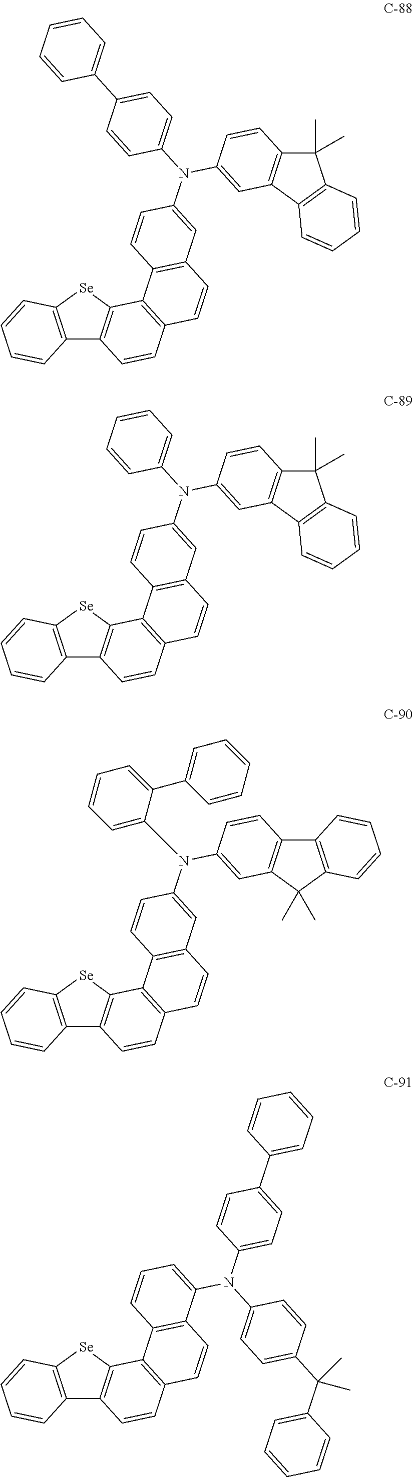 [SMILES: CC1(C)c2ccccc2-c2cc(N(c3ccc(-c4ccccc4)cc3)c3ccc4c(ccc5ccc6c7ccccc7[se]c6c54)c3)ccc21 ; CC1(C)c2ccccc2-c2cc(N(c3ccccc3)c3ccc4c(ccc5ccc6c7ccccc7[se]c6c54)c3)ccc21 ; CC1(C)c2ccccc2-c2ccc(N(c3ccc4c(ccc5ccc6c7ccccc7[se]c6c54)c3)c3ccccc3-c3ccccc3)cc21 ; CC(C)(c1ccccc1)c1ccc(N(c2ccc(-c3ccccc3)cc2)c2cccc3c2ccc2ccc4c5ccccc5[se]c4c23)cc1]

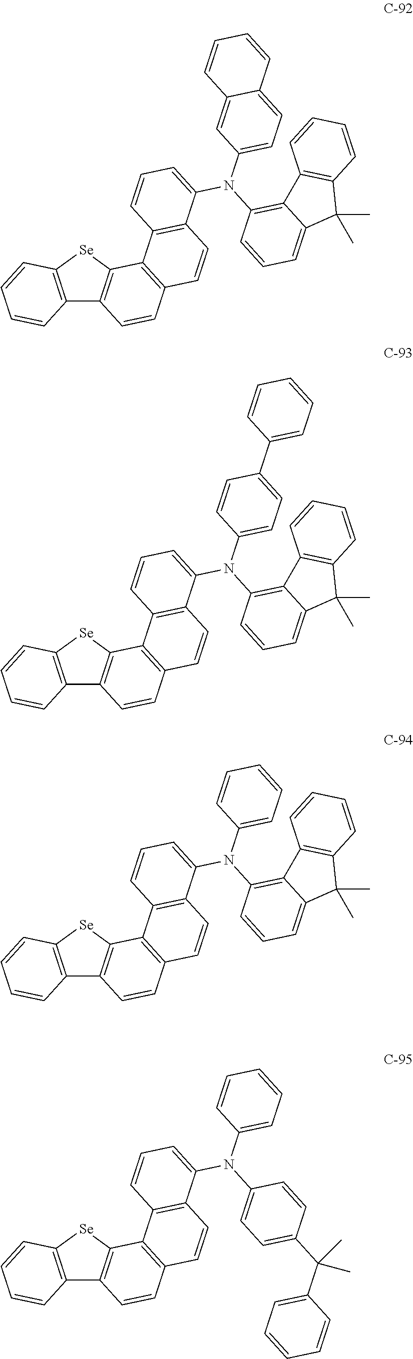 [SMILES: CC1(C)c2ccccc2-c2c(N(c3ccc4ccccc4c3)c3cccc4c3ccc3ccc5c6ccccc6[se]c5c34)cccc21 ; CC1(C)c2ccccc2-c2c(N(c3ccc(-c4ccccc4)cc3)c3cccc4c3ccc3ccc5c6ccccc6[se]c5c34)cccc21 ; CC1(C)c2ccccc2-c2c(N(c3ccccc3)c3cccc4c3ccc3ccc5c6ccccc6[se]c5c34)cccc21 ; CC(C)(c1ccccc1)c1ccc(N(c2ccccc2)c2cccc3c2ccc2ccc4c5ccccc5[se]c4c23)cc1]

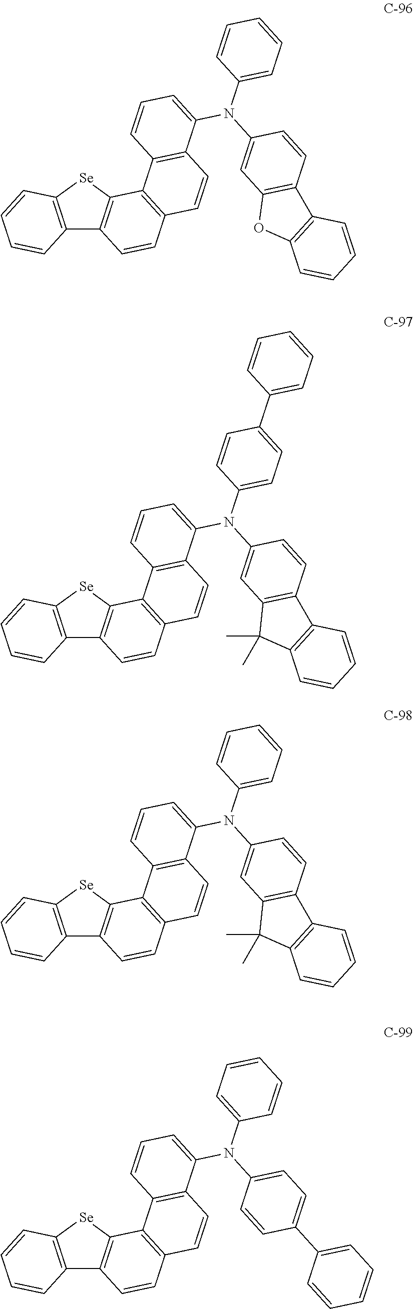 [SMILES: c1ccc(N(c2ccc3c(c2)oc2ccccc23)c2cccc3c2ccc2ccc4c5ccccc5[se]c4c23)cc1 ; CC1(C)c2ccccc2-c2ccc(N(c3ccc(-c4ccccc4)cc3)c3cccc4c3ccc3ccc5c6ccccc6[se]c5c34)cc21 ; CC1(C)c2ccccc2-c2ccc(N(c3ccccc3)c3cccc4c3ccc3ccc5c6ccccc6[se]c5c34)cc21 ; c1ccc(-c2ccc(N(c3ccccc3)c3cccc4c3ccc3ccc5c6ccccc6[se]c5c34)cc2)cc1]

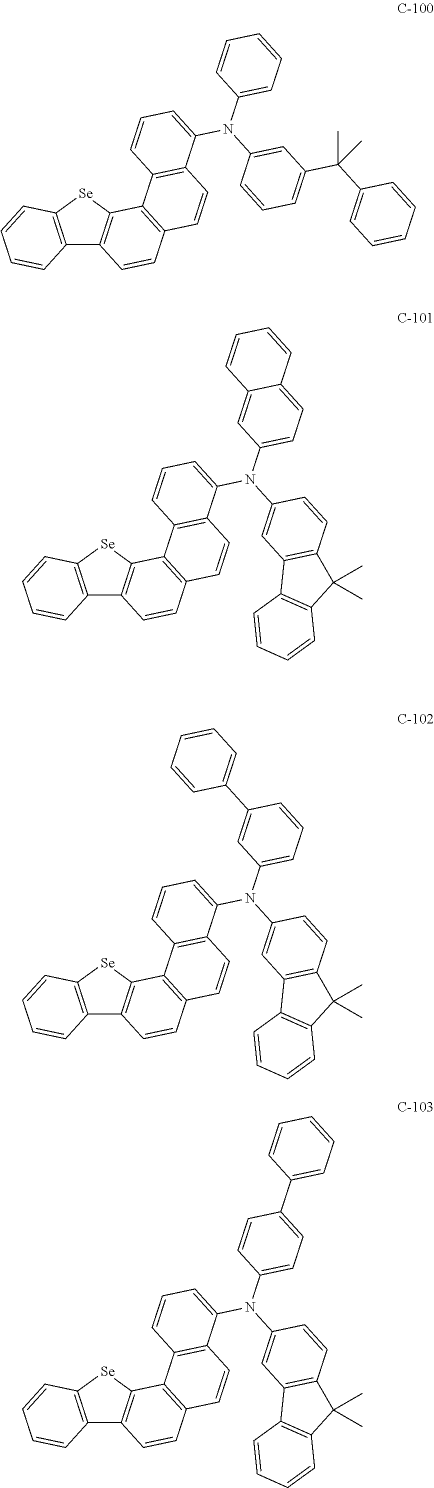

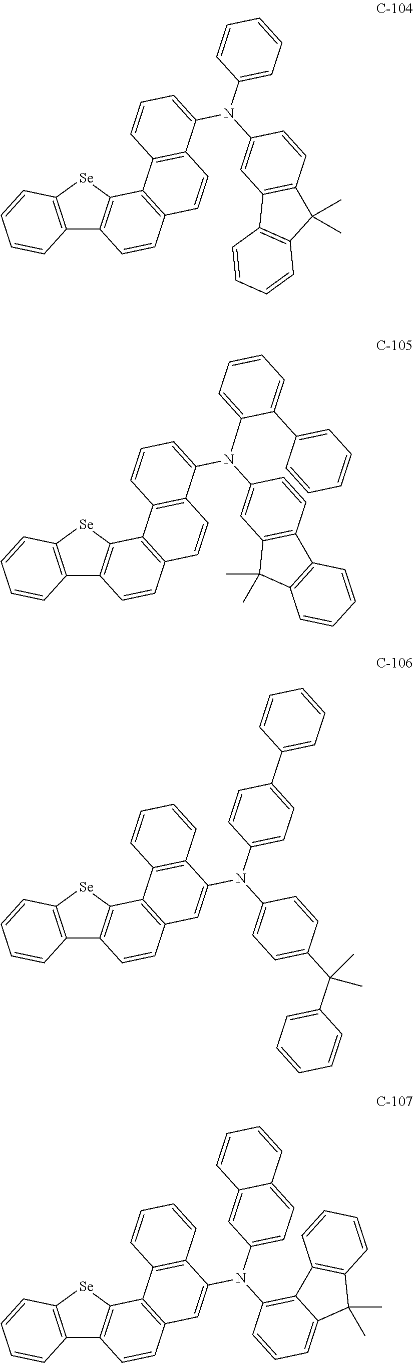 [SMILES: CC1(C)c2ccccc2-c2cc(N(c3ccccc3)c3cccc4c3ccc3ccc5c6ccccc6[se]c5c34)ccc21 ; CC1(C)c2ccccc2-c2ccc(N(c3ccccc3-c3ccccc3)c3cccc4c3ccc3ccc5c6ccccc6[se]c5c34)cc21 ; CC(C)(c1ccccc1)c1ccc(N(c2ccc(-c3ccccc3)cc2)c2cc3ccc4c5ccccc5[se]c4c3c3ccccc23)cc1 ; CC1(C)c2ccccc2-c2c(N(c3ccc4ccccc4c3)c3cc4ccc5c6ccccc6[se]c5c4c4ccccc34)cccc21]

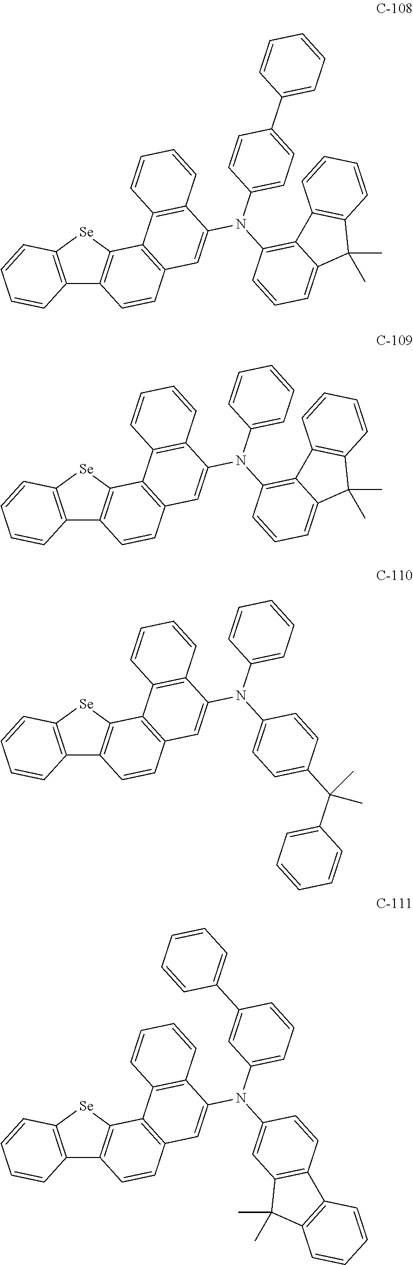 [SMILES: CC1(C)c2ccccc2-c2c(N(c3ccc(-c4ccccc4)cc3)c3cc4ccc5c6ccccc6[se]c5c4c4ccccc34)cccc21 ; CC1(C)c2ccccc2-c2c(N(c3ccccc3)c3cc4ccc5c6ccccc6[se]c5c4c4ccccc34)cccc21 ; CC(C)(c1ccccc1)c1ccc(N(c2ccccc2)c2cc3ccc4c5ccccc5[se]c4c3c3ccccc23)cc1 ; CC1(C)c2ccccc2-c2ccc(N(c3cccc(-c4ccccc4)c3)c3cc4ccc5c6ccccc6[se]c5c4c4ccccc34)cc21]

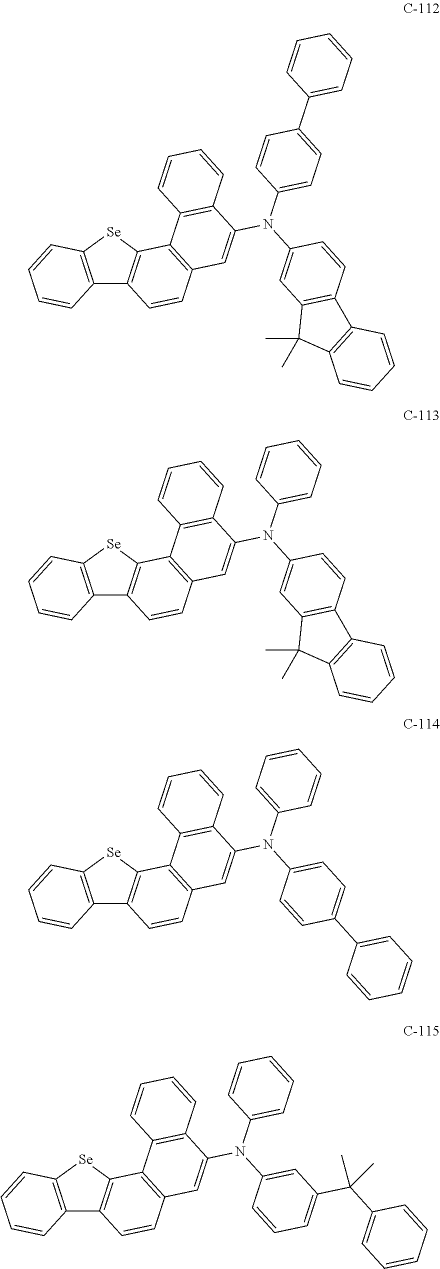 [SMILES: CC1(C)c2ccccc2-c2ccc(N(c3ccc(-c4ccccc4)cc3)c3cc4ccc5c6ccccc6[se]c5c4c4ccccc34)cc21 ; CC1(C)c2ccccc2-c2ccc(N(c3ccccc3)c3cc4ccc5c6ccccc6[se]c5c4c4ccccc34)cc21 ; c1ccc(-c2ccc(N(c3ccccc3)c3cc4ccc5c6ccccc6[se]c5c4c4ccccc34)cc2)cc1 ; CC(C)(c1ccccc1)c1cccc(N(c2ccccc2)c2cc3ccc4c5ccccc5[se]c4c3c3ccccc23)c1]

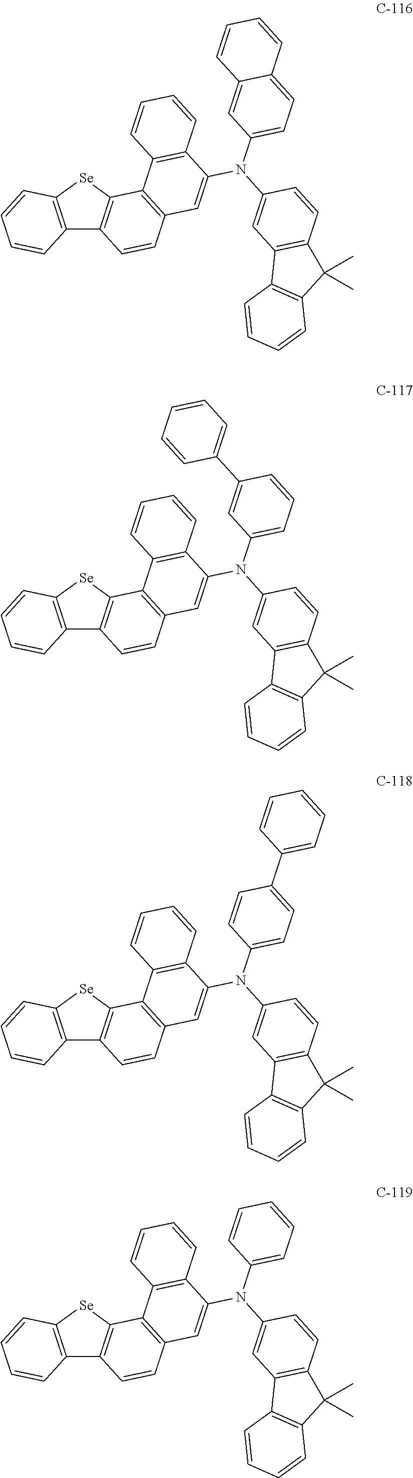

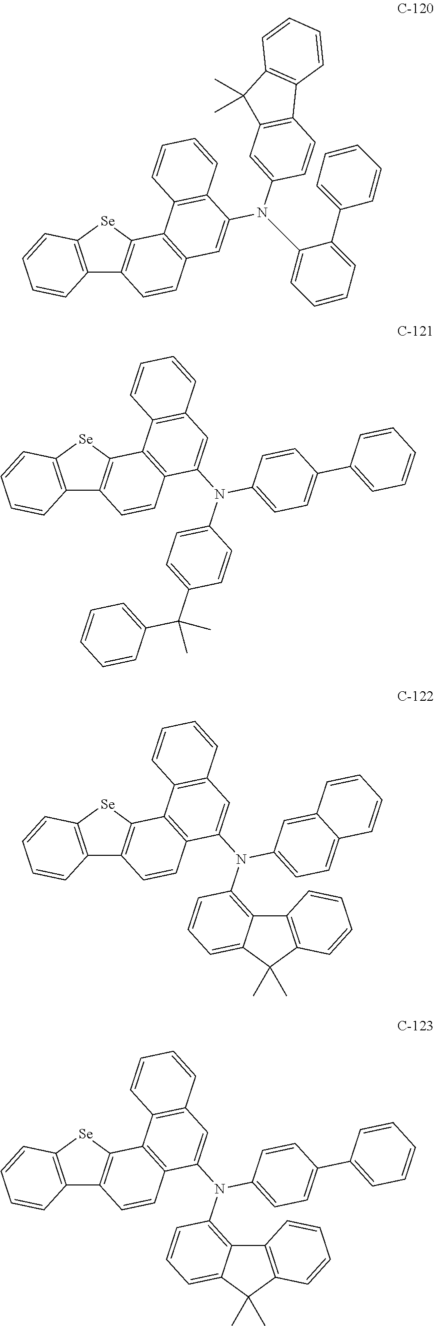 [SMILES: CC1(C)c2ccccc2-c2ccc(N(c3ccccc3-c3ccccc3)c3cc4ccc5c6ccccc6[se]c5c4c4ccccc34)cc21 ; CC(C)(c1ccccc1)c1ccc(N(c2ccc(-c3ccccc3)cc2)c2cc3ccccc3c3c2ccc2c4ccccc4[se]c23)cc1 ; CC1(C)c2ccccc2-c2c(N(c3ccc4ccccc4c3)c3cc4ccccc4c4c3ccc3c5ccccc5[se]c34)cccc21 ; CC1(C)c2ccccc2-c2c(N(c3ccc(-c4ccccc4)cc3)c3cc4ccccc4c4c3ccc3c5ccccc5[se]c34)cccc21]

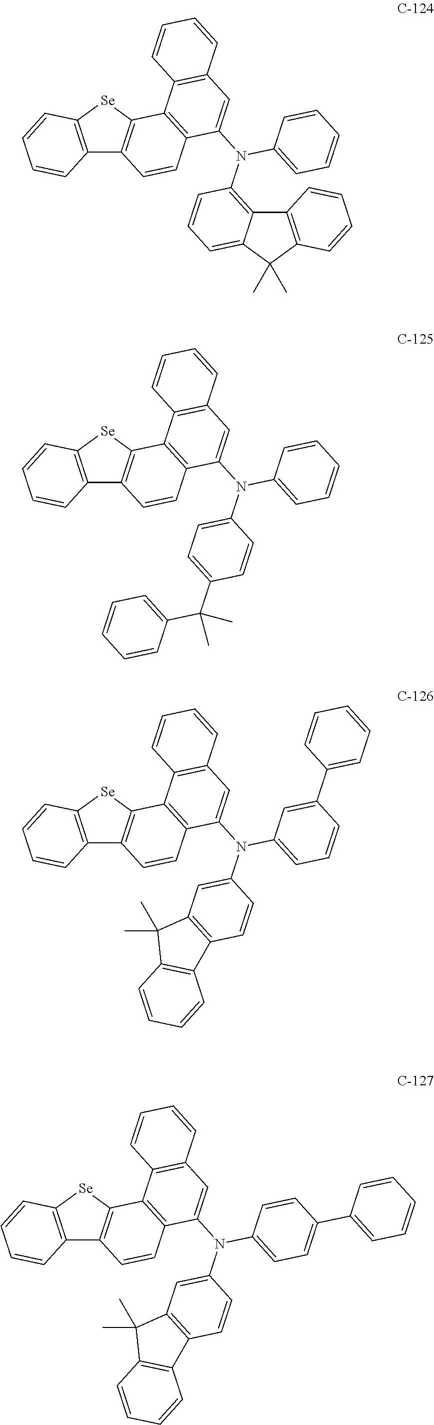 [SMILES: CC1(C)c2ccccc2-c2c(N(c3ccccc3)c3cc4ccccc4c4c3ccc3c5ccccc5[se]c34)cccc21 ; CC(C)(c1ccccc1)c1ccc(N(c2ccccc2)c2cc3ccccc3c3c2ccc2c4ccccc4[se]c23)cc1 ; CC1(C)c2ccccc2-c2ccc(N(c3cccc(-c4ccccc4)c3)c3cc4ccccc4c4c3ccc3c5ccccc5[se]c34)cc21 ; CC1(C)c2ccccc2-c2ccc(N(c3ccc(-c4ccccc4)cc3)c3cc4ccccc4c4c3ccc3c5ccccc5[se]c34)cc21]

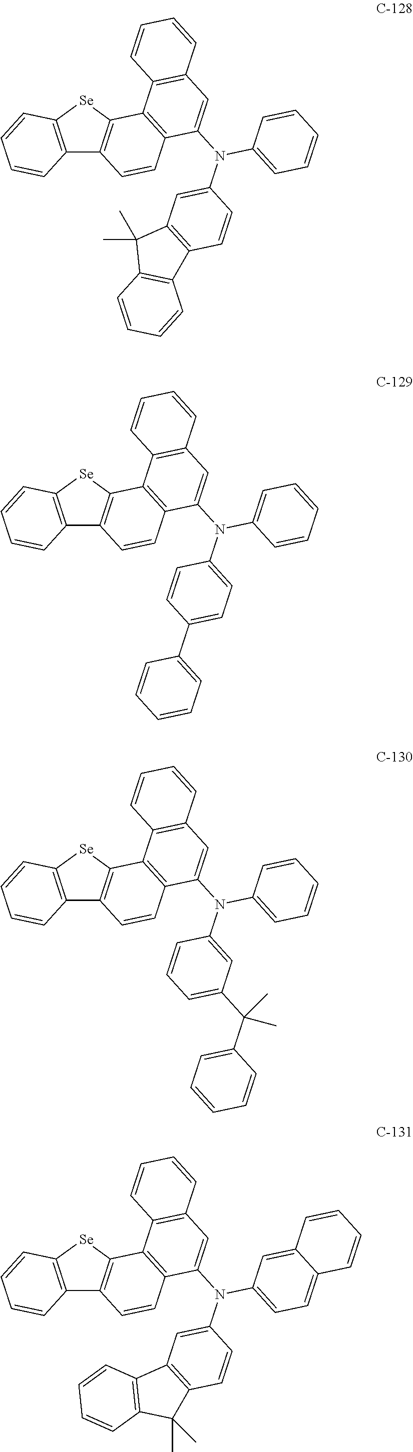 [SMILES: CC1(C)c2ccccc2-c2ccc(N(c3ccccc3)c3cc4ccccc4c4c3ccc3c5ccccc5[se]c34)cc21 ; c1ccc(-c2ccc(N(c3ccccc3)c3cc4ccccc4c4c3ccc3c5ccccc5[se]c34)cc2)cc1 ; CC(C)(c1ccccc1)c1cccc(N(c2ccccc2)c2cc3ccccc3c3c2ccc2c4ccccc4[se]c23)c1 ; CC1(C)c2ccccc2-c2cc(N(c3ccc4ccccc4c3)c3cc4ccccc4c4c3ccc3c5ccccc5[se]c34)ccc21]

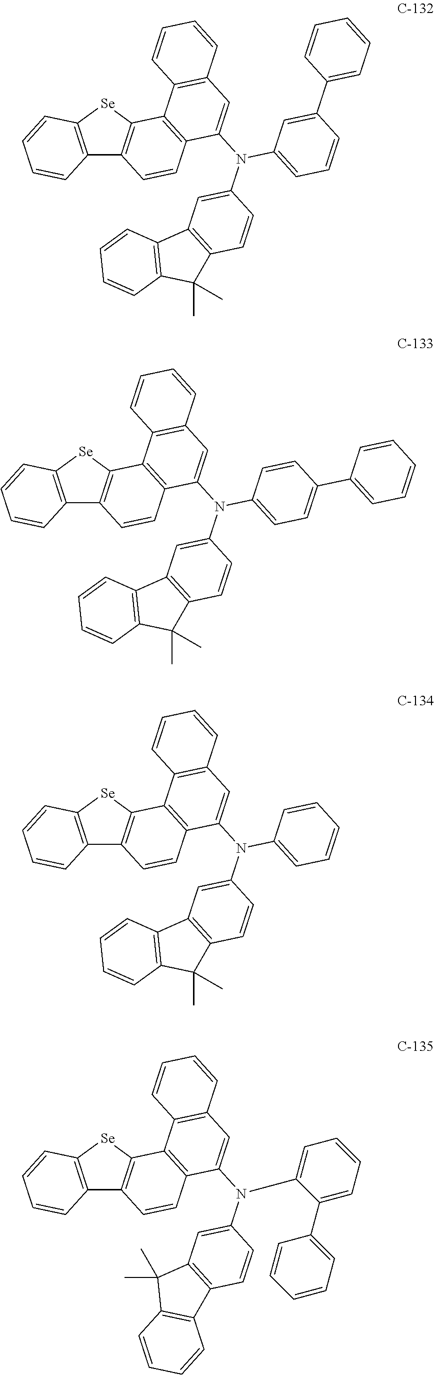 [SMILES: CC1(C)c2ccccc2-c2cc(N(c3cccc(-c4ccccc4)c3)c3cc4ccccc4c4c3ccc3c5ccccc5[se]c34)ccc21 ; CC1(C)c2ccccc2-c2cc(N(c3ccc(-c4ccccc4)cc3)c3cc4ccccc4c4c3ccc3c5ccccc5[se]c34)ccc21 ; CC1(C)c2ccccc2-c2cc(N(c3ccccc3)c3cc4ccccc4c4c3ccc3c5ccccc5[se]c34)ccc21 ; CC1(C)c2ccccc2-c2ccc(N(c3ccccc3-c3ccccc3)c3cc4ccccc4c4c3ccc3c5ccccc5[se]c34)cc21]

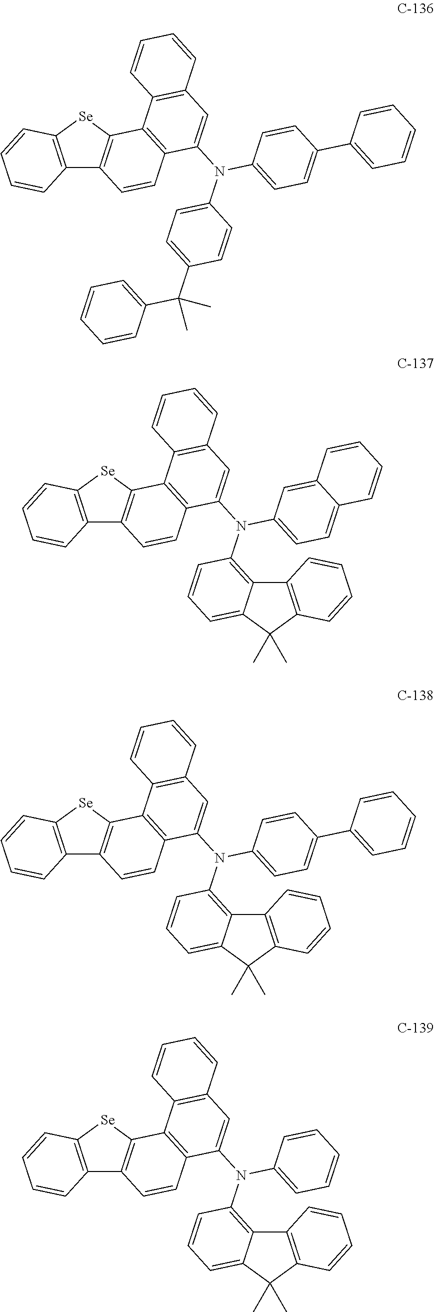 [SMILES: CC(C)(c1ccccc1)c1ccc(N(c2ccc(-c3ccccc3)cc2)c2cc3ccccc3c3c2ccc2c4ccccc4[se]c23)cc1 ; CC1(C)c2ccccc2-c2c(N(c3ccc4ccccc4c3)c3cc4ccccc4c4c3ccc3c5ccccc5[se]c34)cccc21 ; CC1(C)c2ccccc2-c2c(N(c3ccc(-c4ccccc4)cc3)c3cc4ccccc4c4c3ccc3c5ccccc5[se]c34)cccc21 ; CC1(C)c2ccccc2-c2c(N(c3ccccc3)c3cc4ccccc4c4c3ccc3c5ccccc5[se]c34)cccc21]

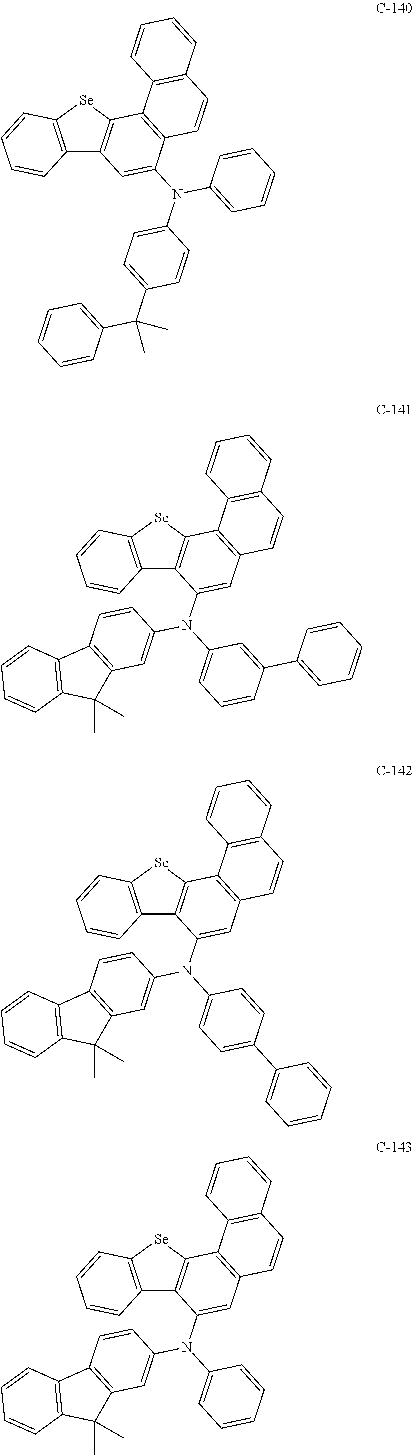 [SMILES: CC(C)(c1ccccc1)c1ccc(N(c2ccccc2)c2cc3c4ccccc4[se]c3c3c2ccc2ccccc23)cc1 ; CC1(C)c2ccccc2-c2ccc(N(c3cccc(-c4ccccc4)c3)c3cc4ccc5ccccc5c4c4[se]c5ccccc5c34)cc21 ; CC1(C)c2ccccc2-c2ccc(N(c3ccc(-c4ccccc4)cc3)c3cc4ccc5ccccc5c4c4[se]c5ccccc5c34)cc21 ; CC1(C)c2ccccc2-c2ccc(N(c3ccccc3)c3cc4ccc5ccccc5c4c4[se]c5ccccc5c34)cc21]

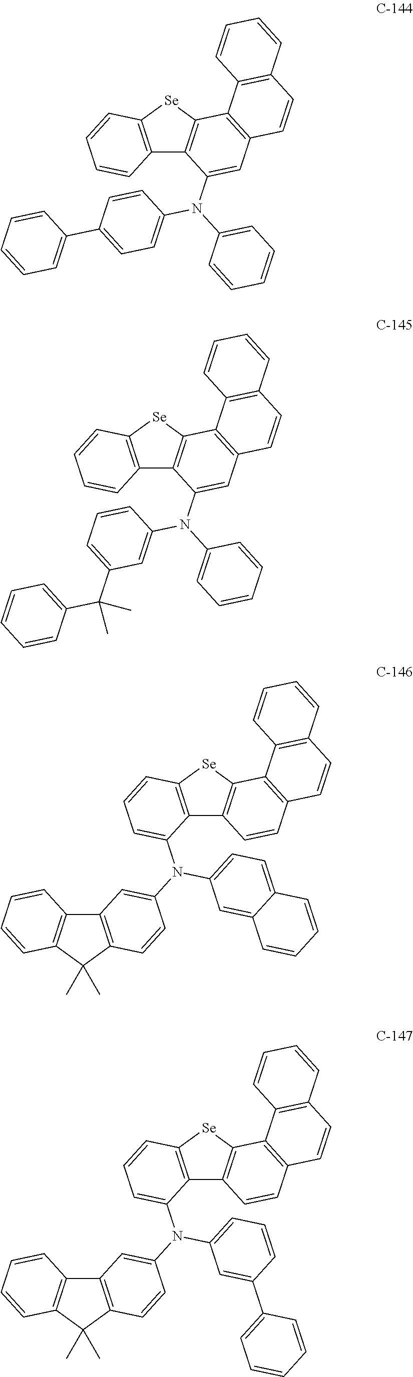 [SMILES: c1ccc(-c2ccc(N(c3ccccc3)c3cc4ccc5ccccc5c4c4[se]c5ccccc5c34)cc2)cc1 ; CC(C)(c1ccccc1)c1cccc(N(c2ccccc2)c2cc3ccc4ccccc4c3c3[se]c4ccccc4c23)c1 ; CC1(C)c2ccccc2-c2cc(N(c3ccc4ccccc4c3)c3cccc4[se]c5c(ccc6ccc7ccccc7c65)c34)ccc21 ; CC1(C)c2ccccc2-c2cc(N(c3cccc(-c4ccccc4)c3)c3cccc4[se]c5c(ccc6ccc7ccccc7c65)c34)ccc21]

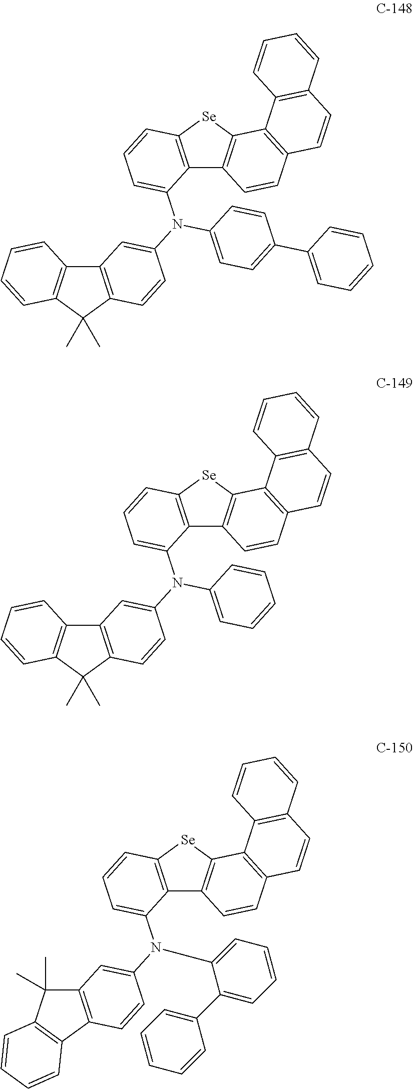 [SMILES: CC1(C)c2ccccc2-c2cc(N(c3ccc(-c4ccccc4)cc3)c3cccc4[se]c5c(ccc6ccc7ccccc7c65)c34)ccc21 ; CC1(C)c2ccccc2-c2cc(N(c3ccccc3)c3cccc4[se]c5c(ccc6ccc7ccccc7c65)c34)ccc21 ; CC1(C)c2ccccc2-c2ccc(N(c3ccccc3-c3ccccc3)c3cccc4[se]c5c(ccc6ccc7ccccc7c65)c34)cc21]

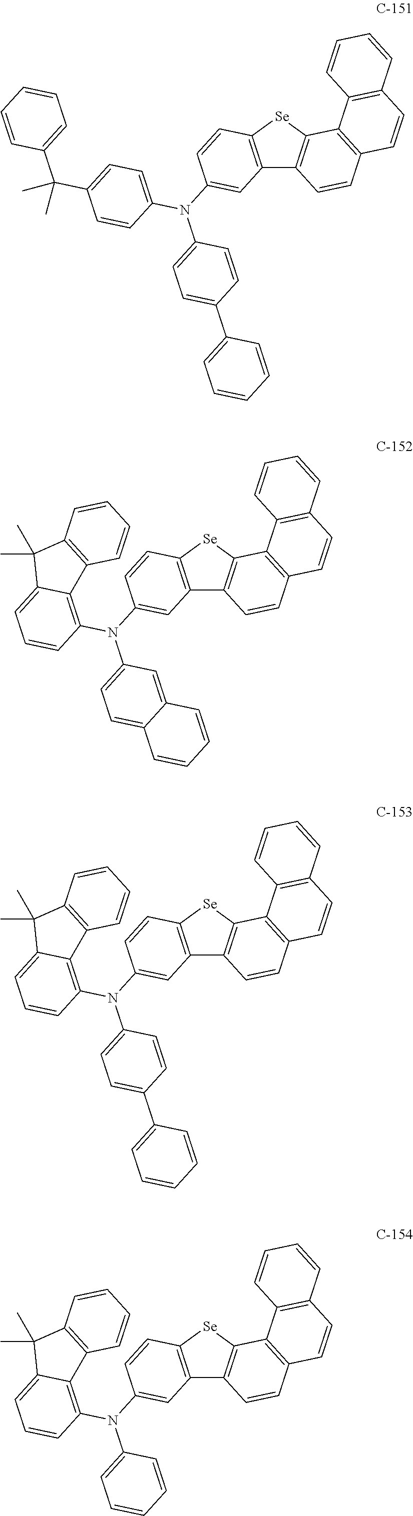 [SMILES: CC(C)(c1ccccc1)c1ccc(N(c2ccc(-c3ccccc3)cc2)c2ccc3[se]c4c(ccc5ccc6ccccc6c54)c3c2)cc1 ; CC1(C)c2ccccc2-c2c(N(c3ccc4ccccc4c3)c3ccc4[se]c5c(ccc6ccc7ccccc7c65)c4c3)cccc21 ; CC1(C)c2ccccc2-c2c(N(c3ccc(-c4ccccc4)cc3)c3ccc4[se]c5c(ccc6ccc7ccccc7c65)c4c3)cccc21 ; CC1(C)c2ccccc2-c2c(N(c3ccccc3)c3ccc4[se]c5c(ccc6ccc7ccccc7c65)c4c3)cccc21]

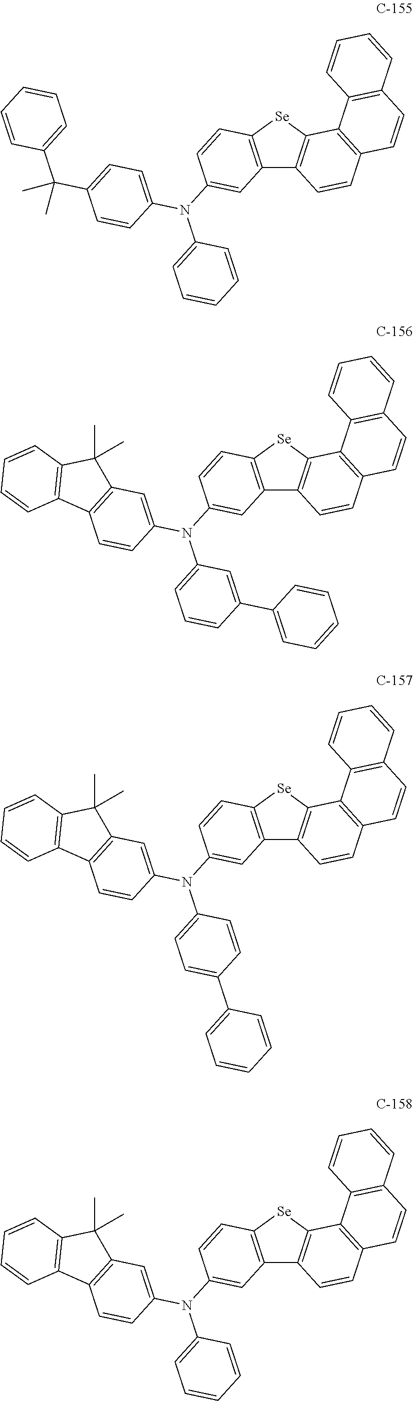 [SMILES: CC(C)(c1ccccc1)c1ccc(N(c2ccccc2)c2ccc3[se]c4c(ccc5ccc6ccccc6c54)c3c2)cc1 ; CC1(C)c2ccccc2-c2ccc(N(c3cccc(-c4ccccc4)c3)c3ccc4[se]c5c(ccc6ccc7ccccc7c65)c4c3)cc21 ; CC1(C)c2ccccc2-c2ccc(N(c3ccc(-c4ccccc4)cc3)c3ccc4[se]c5c(ccc6ccc7ccccc7c65)c4c3)cc21 ; CC1(C)c2ccccc2-c2ccc(N(c3ccccc3)c3ccc4[se]c5c(ccc6ccc7ccccc7c65)c4c3)cc21]

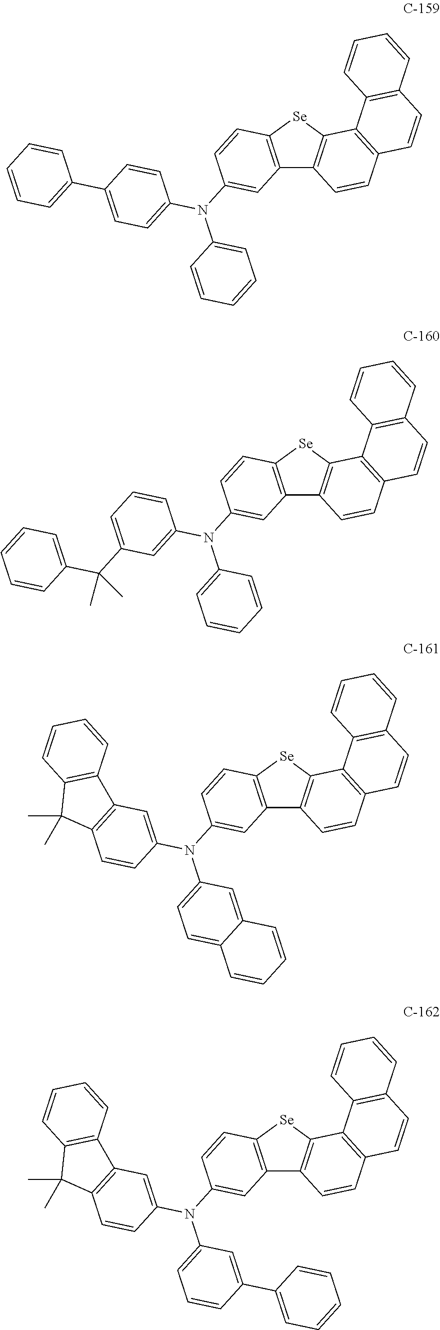 [SMILES: c1ccc(-c2ccc(N(c3ccccc3)c3ccc4[se]c5c(ccc6ccc7ccccc7c65)c4c3)cc2)cc1 ; CC(C)(c1ccccc1)c1cccc(N(c2ccccc2)c2ccc3[se]c4c(ccc5ccc6ccccc6c54)c3c2)c1 ; CC1(C)c2ccccc2-c2cc(N(c3ccc4ccccc4c3)c3ccc4[se]c5c(ccc6ccc7ccccc7c65)c4c3)ccc21 ; CC1(C)c2ccccc2-c2cc(N(c3cccc(-c4ccccc4)c3)c3ccc4[se]c5c(ccc6ccc7ccccc7c65)c4c3)ccc21]

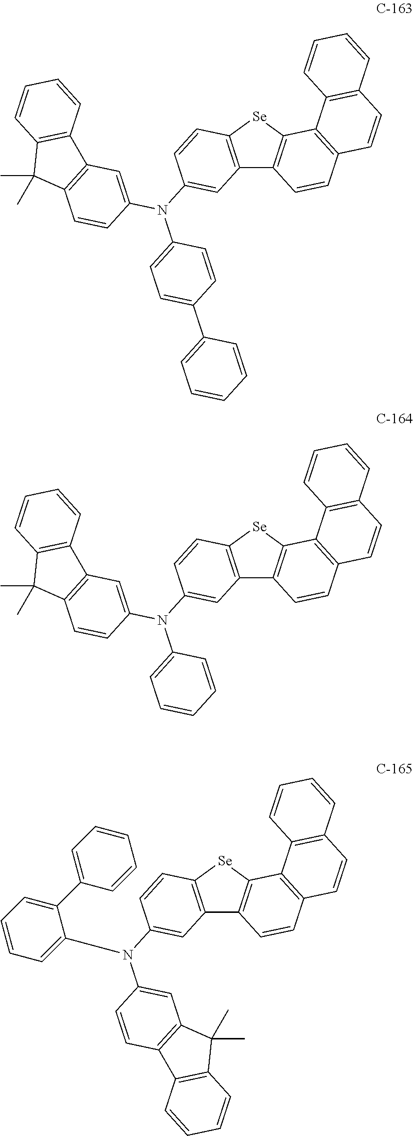 [SMILES: CC1(C)c2ccccc2-c2cc(N(c3ccc(-c4ccccc4)cc3)c3ccc4[se]c5c(ccc6ccc7ccccc7c65)c4c3)ccc21 ; CC1(C)c2ccccc2-c2cc(N(c3ccccc3)c3ccc4[se]c5c(ccc6ccc7ccccc7c65)c4c3)ccc21 ; CC1(C)c2ccccc2-c2ccc(N(c3ccc4[se]c5c(ccc6ccc7ccccc7c65)c4c3)c3ccccc3-c3ccccc3)cc21]

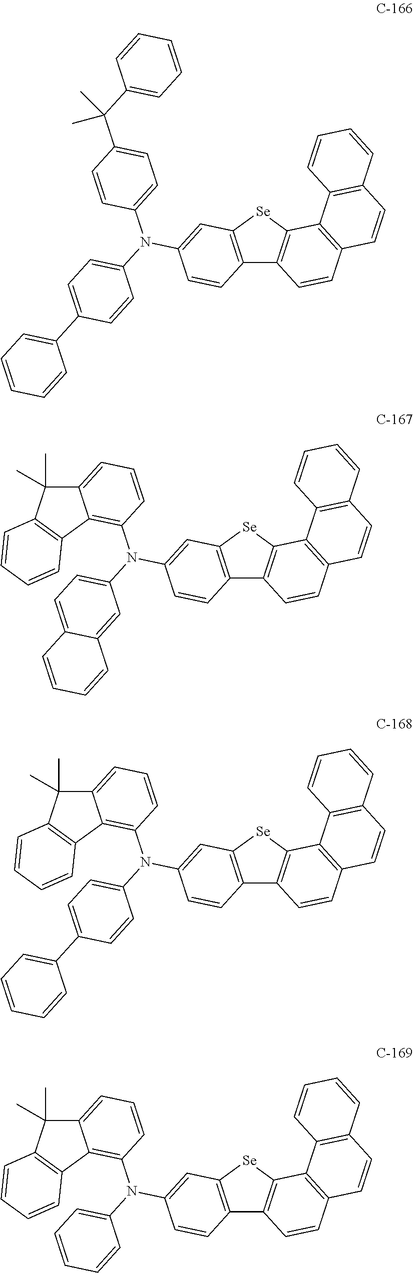 [SMILES: CC(C)(c1ccccc1)c1ccc(N(c2ccc(-c3ccccc3)cc2)c2ccc3c(c2)[se]c2c3ccc3ccc4ccccc4c32)cc1 ; CC1(C)c2ccccc2-c2c(N(c3ccc4ccccc4c3)c3ccc4c(c3)[se]c3c4ccc4ccc5ccccc5c43)cccc21 ; CC1(C)c2ccccc2-c2c(N(c3ccc(-c4ccccc4)cc3)c3ccc4c(c3)[se]c3c4ccc4ccc5ccccc5c43)cccc21 ; CC1(C)c2ccccc2-c2c(N(c3ccccc3)c3ccc4c(c3)[se]c3c4ccc4ccc5ccccc5c43)cccc21]

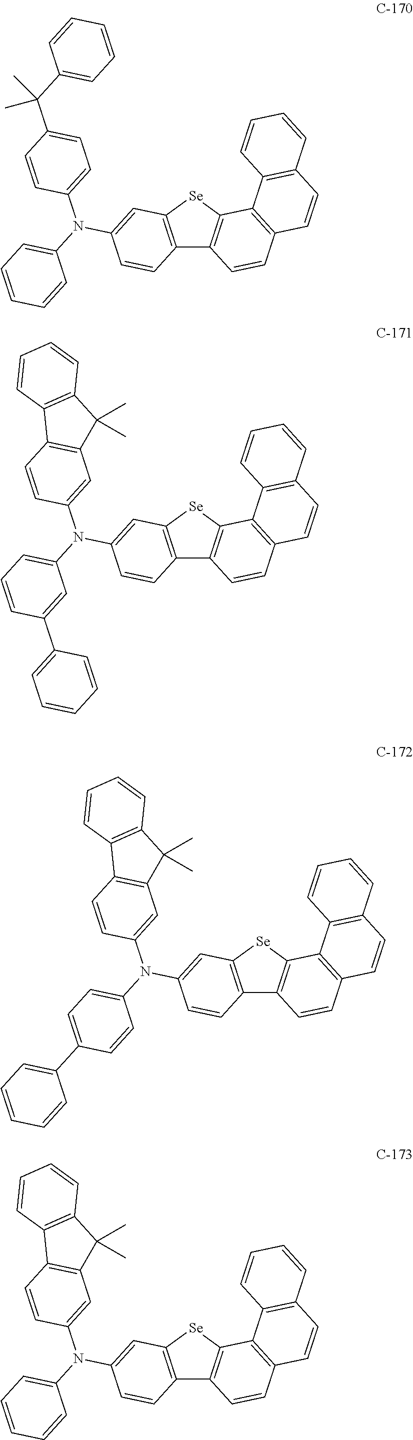

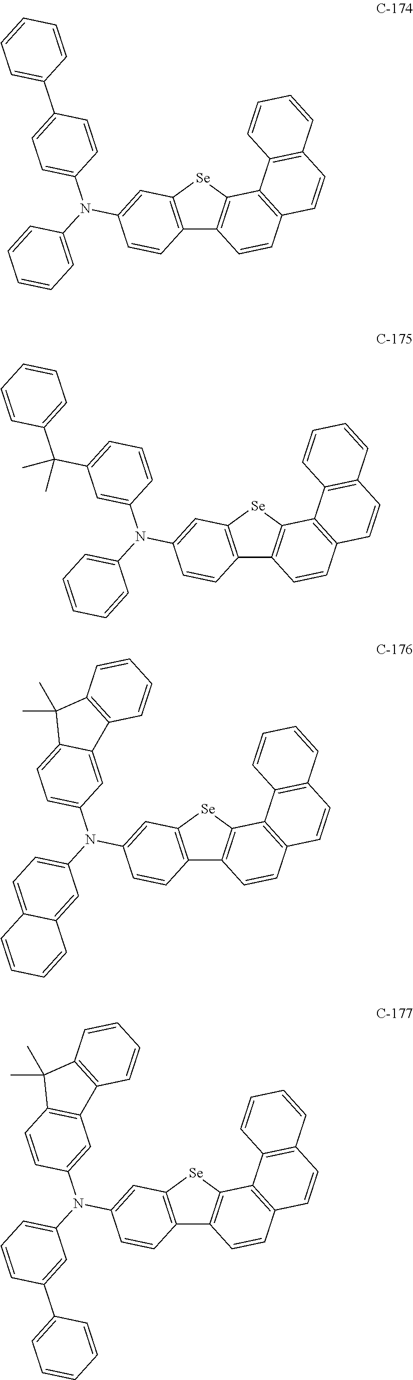 [SMILES: c1ccc(-c2ccc(N(c3ccccc3)c3ccc4c(c3)[se]c3c4ccc4ccc5ccccc5c43)cc2)cc1 ; CC(C)(c1ccccc1)c1cccc(N(c2ccccc2)c2ccc3c(c2)[se]c2c3ccc3ccc4ccccc4c32)c1 ; CC1(C)c2ccccc2-c2cc(N(c3ccc4ccccc4c3)c3ccc4c(c3)[se]c3c4ccc4ccc5ccccc5c43)ccc21 ; CC1(C)c2ccccc2-c2cc(N(c3cccc(-c4ccccc4)c3)c3ccc4c(c3)[se]c3c4ccc4ccc5ccccc5c43)ccc21]

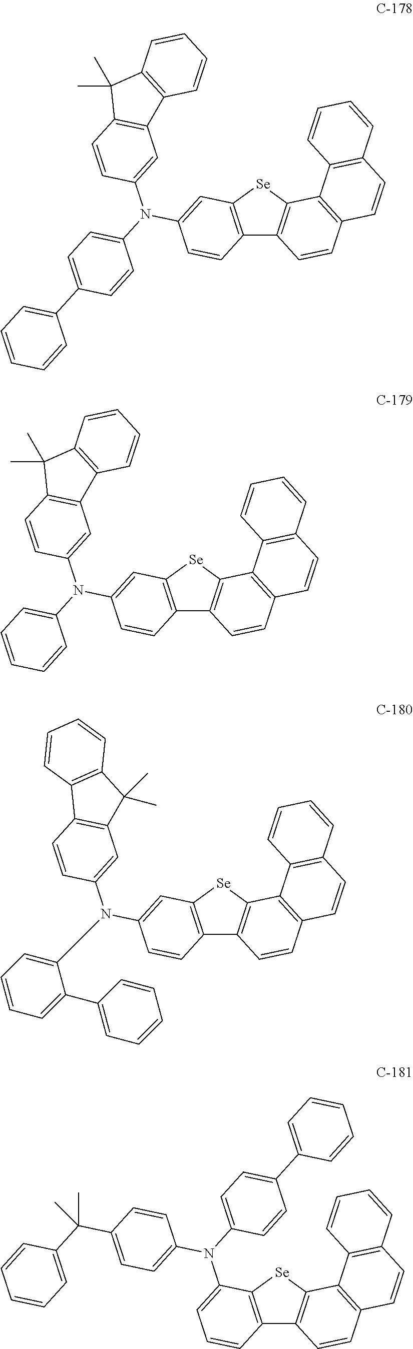 [SMILES: CC1(C)c2ccccc2-c2cc(N(c3ccc(-c4ccccc4)cc3)c3ccc4c(c3)[se]c3c4ccc4ccc5ccccc5c43)ccc21 ; CC1(C)c2ccccc2-c2cc(N(c3ccccc3)c3ccc4c(c3)[se]c3c4ccc4ccc5ccccc5c43)ccc21 ; CC1(C)c2ccccc2-c2ccc(N(c3ccc4c(c3)[se]c3c4ccc4ccc5ccccc5c43)c3ccccc3-c3ccccc3)cc21 ; CC(C)(c1ccccc1)c1ccc(N(c2ccc(-c3ccccc3)cc2)c2cccc3c2[se]c2c3ccc3ccc4ccccc4c32)cc1]

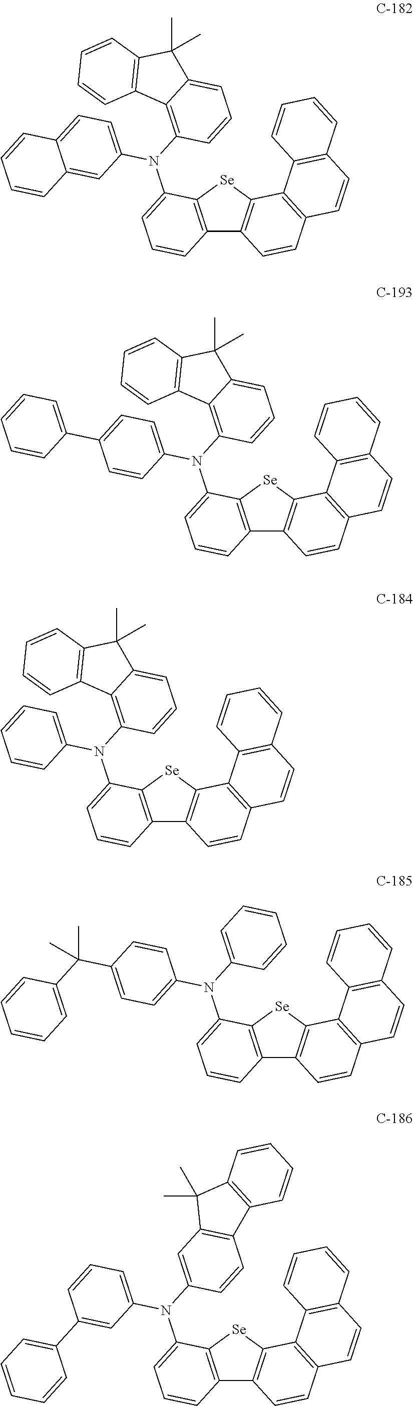 [SMILES: CC1(C)c2ccccc2-c2c(N(c3ccc4ccccc4c3)c3cccc4c3[se]c3c4ccc4ccc5ccccc5c43)cccc21 ; CC1(C)c2ccccc2-c2c(N(c3ccc(-c4ccccc4)cc3)c3cccc4c3[se]c3c4ccc4ccc5ccccc5c43)cccc21 ; CC1(C)c2ccccc2-c2c(N(c3ccccc3)c3cccc4c3[se]c3c4ccc4ccc5ccccc5c43)cccc21 ; CC(C)(c1ccccc1)c1ccc(N(c2ccccc2)c2cccc3c2[se]c2c3ccc3ccc4ccccc4c32)cc1 ; CC1(C)c2ccccc2-c2ccc(N(c3cccc(-c4ccccc4)c3)c3cccc4c3[se]c3c4ccc4ccc5ccccc5c43)cc21]

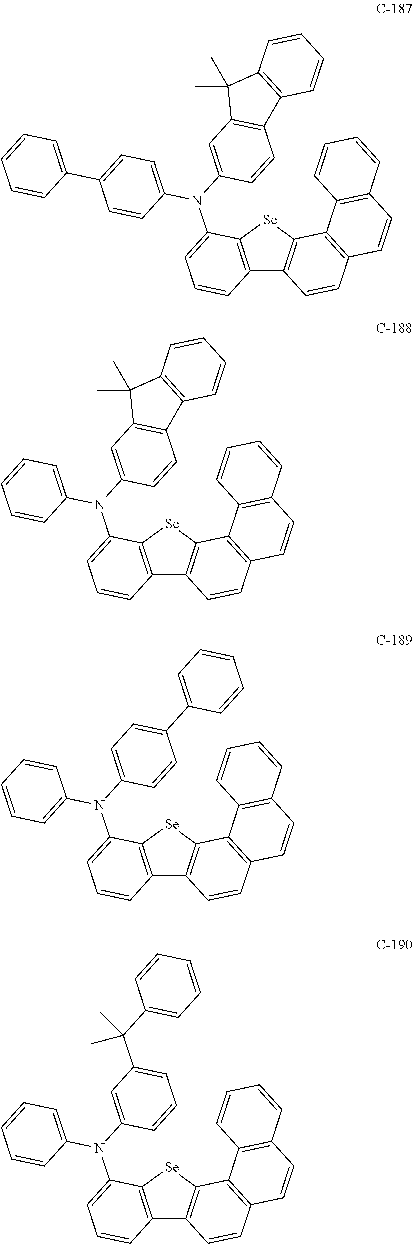 [SMILES: CC1(C)c2ccccc2-c2ccc(N(c3ccc(-c4ccccc4)cc3)c3cccc4c3[se]c3c4ccc4ccc5ccccc5c43)cc21 ; CC1(C)c2ccccc2-c2ccc(N(c3ccccc3)c3cccc4c3[se]c3c4ccc4ccc5ccccc5c43)cc21 ; c1ccc(-c2ccc(N(c3ccccc3)c3cccc4c3[se]c3c4ccc4ccc5ccccc5c43)cc2)cc1 ; CC(C)(c1ccccc1)c1cccc(N(c2ccccc2)c2cccc3c2[se]c2c3ccc3ccc4ccccc4c32)c1]

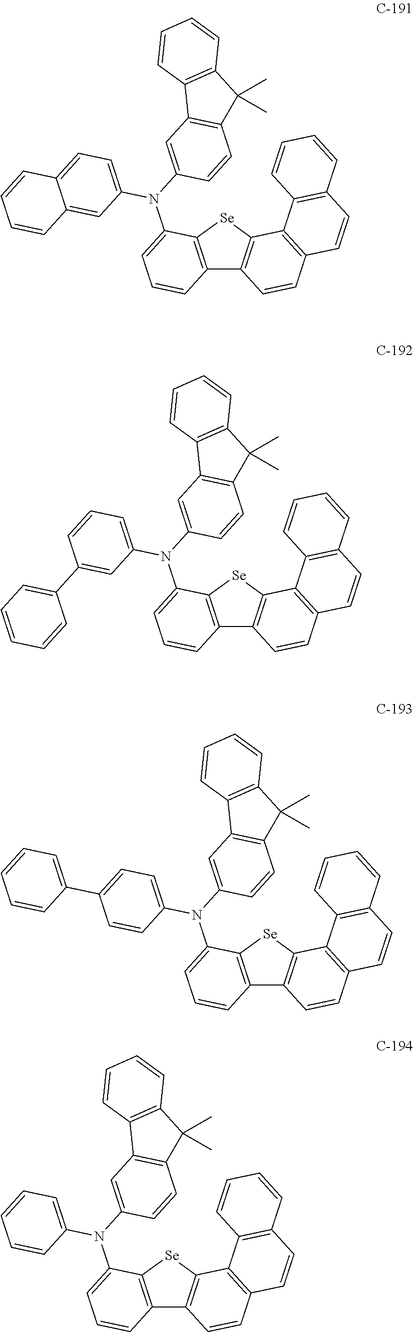 [SMILES: CC1(C)c2ccccc2-c2cc(N(c3ccc4ccccc4c3)c3cccc4c3[se]c3c4ccc4ccc5ccccc5c43)ccc21 ; CC1(C)c2ccccc2-c2cc(N(c3cccc(-c4ccccc4)c3)c3cccc4c3[se]c3c4ccc4ccc5ccccc5c43)ccc21 ; CC1(C)c2ccccc2-c2cc(N(c3ccc(-c4ccccc4)cc3)c3cccc4c3[se]c3c4ccc4ccc5ccccc5c43)ccc21 ; CC1(C)c2ccccc2-c2cc(N(c3ccccc3)c3cccc4c3[se]c3c4ccc4ccc5ccccc5c43)ccc21]

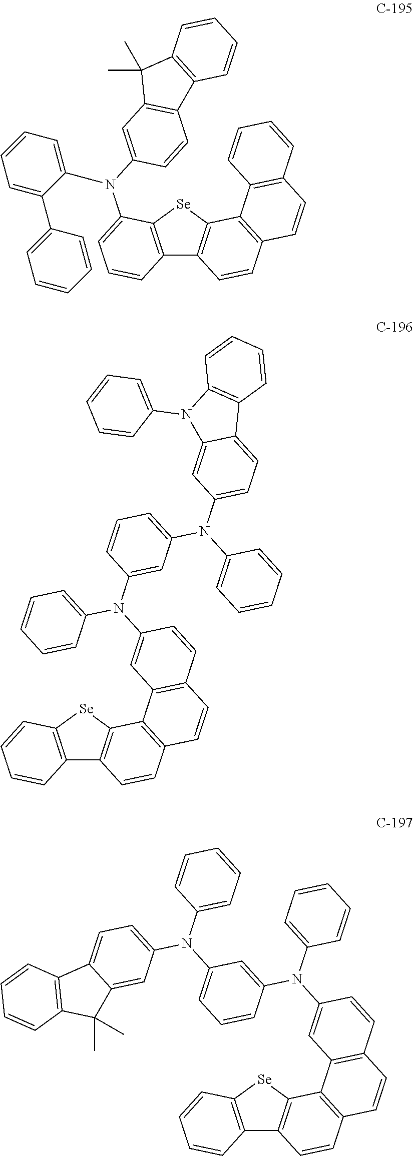 [SMILES: CC1(C)c2ccccc2-c2ccc(N(c3ccccc3-c3ccccc3)c3cccc4c3[se]c3c4ccc4ccc5ccccc5c43)cc21 ; c1ccc(N(c2cccc(N(c3ccccc3)c3ccc4c5ccccc5n(-c5ccccc5)c4c3)c2)c2ccc3ccc4ccc5c6ccccc6[se]c5c4c3c2)cc1 ; CC1(C)c2ccccc2-c2ccc(N(c3ccccc3)c3cccc(N(c4ccccc4)c4ccc5ccc6ccc7c8ccccc8[se]c7c6c5c4)c3)cc21]

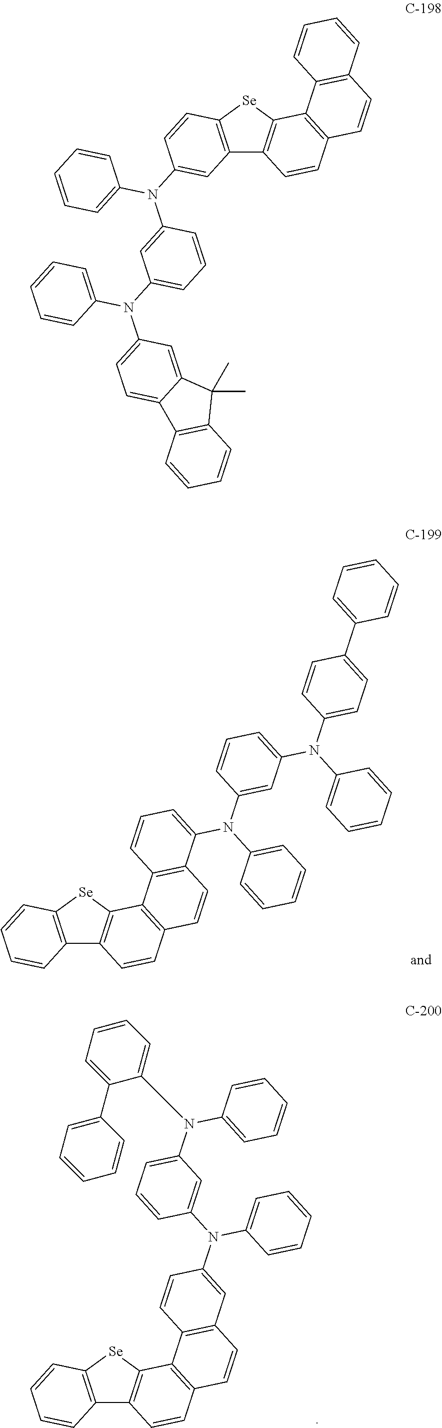 [SMILES: CC1(C)c2ccccc2-c2ccc(N(c3ccccc3)c3cccc(N(c4ccccc4)c4ccc5[se]c6c(ccc7ccc8ccccc8c76)c5c4)c3)cc21 ; c1ccc(-c2ccc(N(c3ccccc3)c3cccc(N(c4ccccc4)c4cccc5c4ccc4ccc6c7ccccc7[se]c6c45)c3)cc2)cc1 ; c1ccc(-c2ccccc2N(c2ccccc2)c2cccc(N(c3ccccc3)c3ccc4c(ccc5ccc6c7ccccc7[se]c6c54)c3)c2)cc1]

4. An organic electroluminescent device comprising the organic electroluminescent compound according to claim 1.

5. A plurality of host materials comprising the organic electroluminescent compound according to claim 1, wherein the plurality of host materials comprise at least one first host compound represented by the following formula 1′ and at least one second host compound represented by the following formula 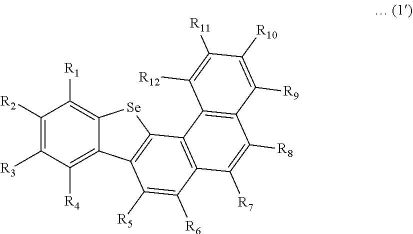
in formula 1′,
R₁ to R₁₂, L₁, Rₐ₁, and Rₐ₂ are as defined in claim 1;
Ar₁ in the first host compound is represented by 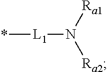
and
Ar₁ in the second host compound represents a substituted or unsubstituted (3- to 30-membered)heteroaryl.

6. A plurality of host materials comprising at least one first host material comprising the organic electroluminescent compound according to claim 1 and at least one second host material different from the first host material.

7. The plurality of host materials according to claim 6, wherein the second host material comprises an organic electroluminescent compound represented by the following formula 2: 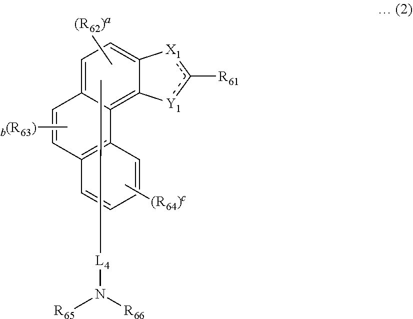
in formula 2,
X₁ and Y₁, each independently, represent —N═, —O— or —S—, with the proviso that any one of X₁ and Y₁ represents —N═, and the other one represents —NR₆₇—, —O— or -3-;
R₆₁ represents a substituted or unsubstituted (C6-C30)aryl, or a substituted or unsubstituted (3- to 30-membered)heteroaryl;
R₆₂ to R₆₄ and R₆₇, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring group of a (C3-C30) aliphatic ring(s) and a (C6-C30) aromatic ring(s), or -L₃″—N(Ar₃″)(Ar₄″); or may be linked to an adjacent substituent(s) to form a ring(s);
L₃″, each independently, represents a single bond, a substituted or unsubstituted (C6-C30)arylene, or a substituted or unsubstituted (3- to 30-membered)heteroarylene;
Ar₃″ and Ar₄″, each independently, represent hydrogen, a substituted or unsubstituted (C1-C30)alkyl, a substituted or unsubstituted (C2-C30)alkenyl, a substituted or unsubstituted fused ring group of a (C3-C30) aliphatic ring(s) and a (C6-C30) aromatic ring(s), a substituted or unsubstituted (C6-C30)aryl, or a substituted or unsubstituted (3- to 30-membered)heteroaryl;
R₆₅ and R₆₆, each independently, represent a substituted or unsubstituted (C6-C30)aryl, or a substituted or unsubstituted (3- to 30-membered)heteroaryl;
L₄ represents a single bond, a substituted or unsubstituted (C6-C30)arylene, or a substituted or unsubstituted (3- to 30-membered)heteroarylene; and
a and b, each independently, represent an integer of 1 or 2, and c represents an integer of 1 to 4, where if a to c are each an integer of 2 or more, each of R₆₂ to each of R₆₄ may be the same or different from each other.

8. The plurality of host materials according to claim 7, wherein the compound represented by formula 2 is at least one selected from the group consisting of the following compounds:

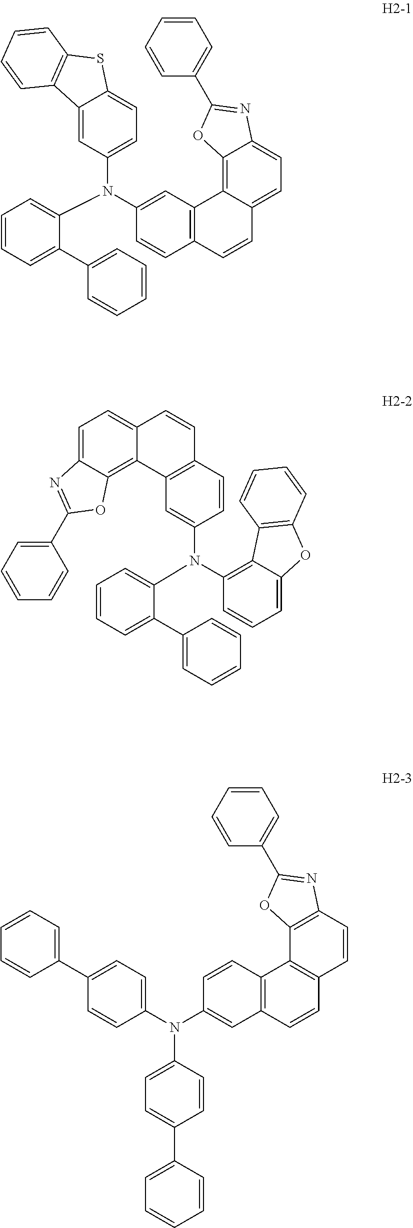 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

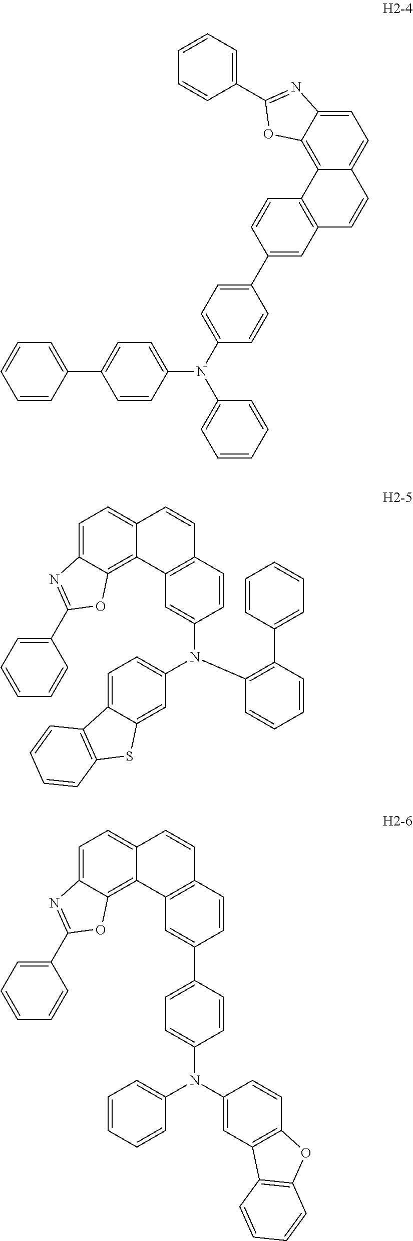 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

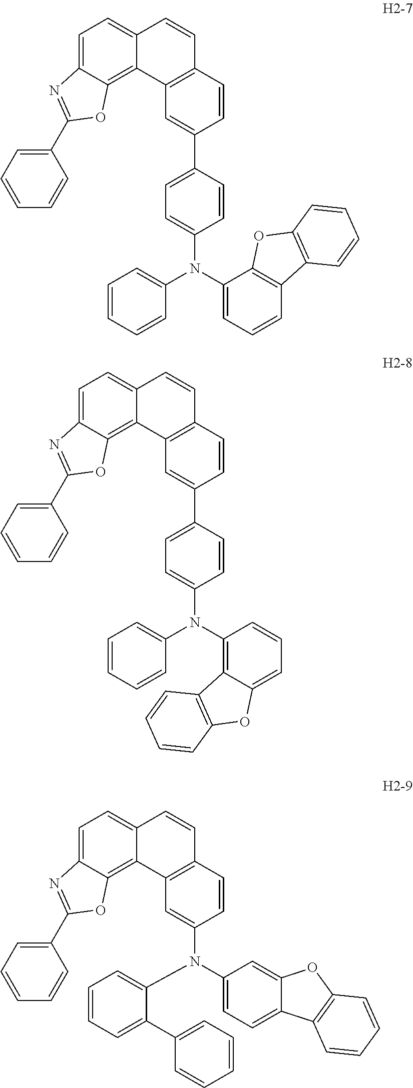 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccc7c(c6)oc6ccccc67)c6ccccc6-c6ccccc6)cc5c4c3o2)cc1]

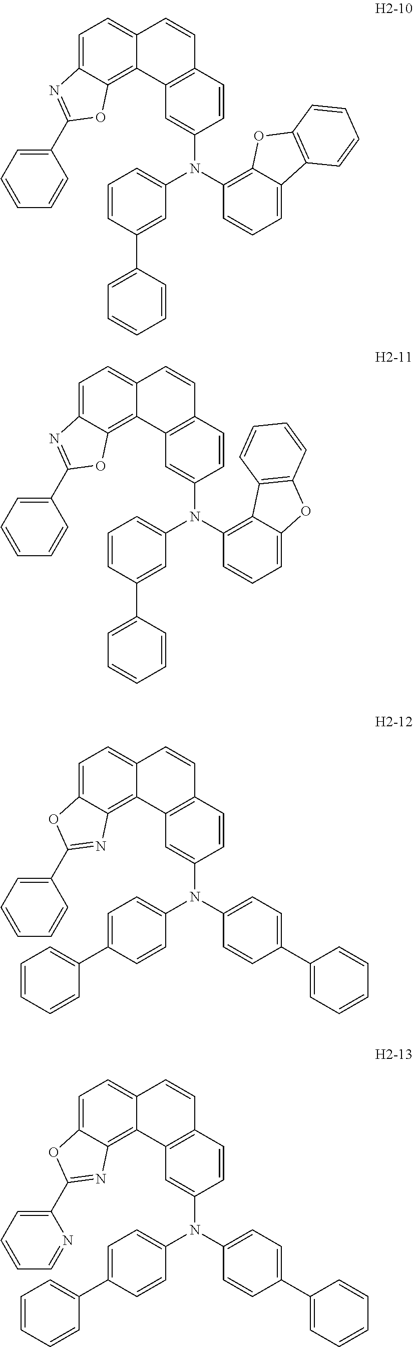

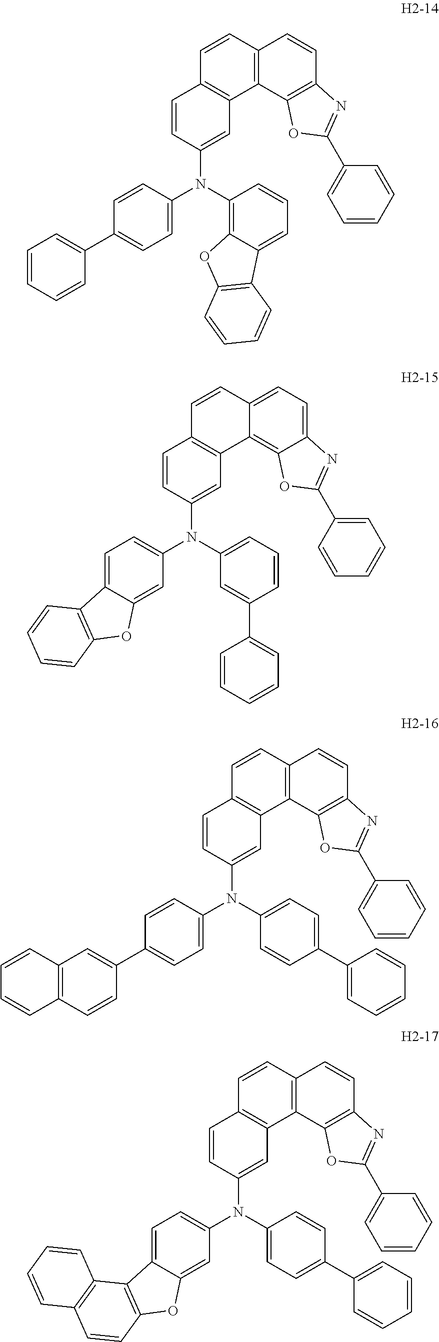

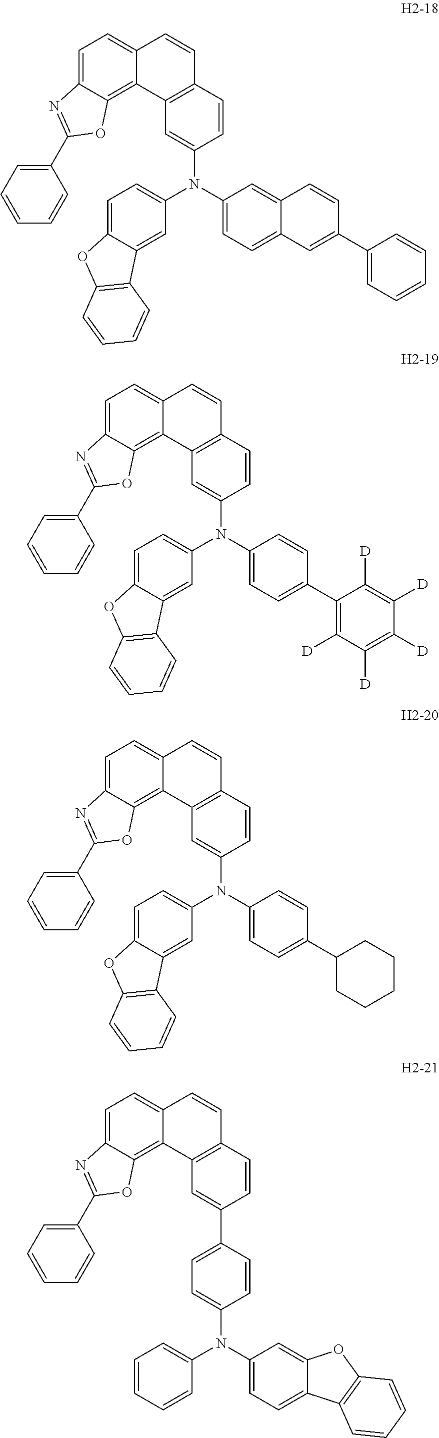

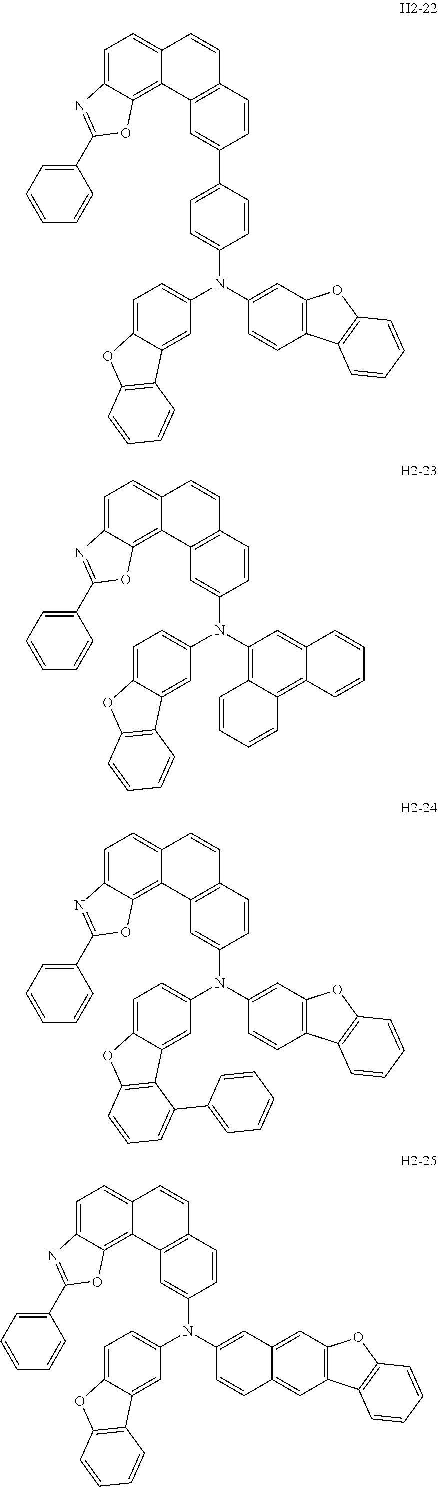

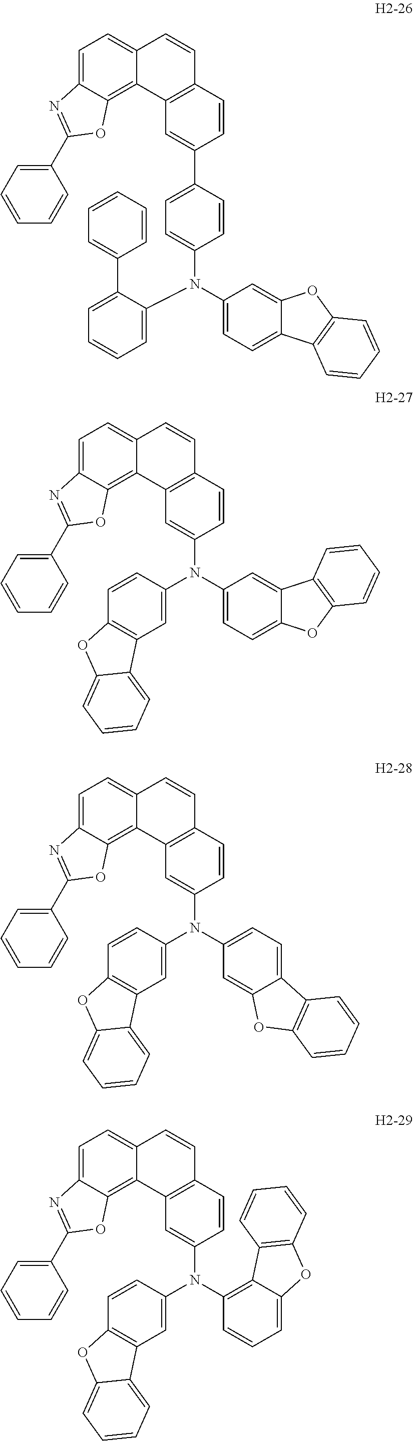

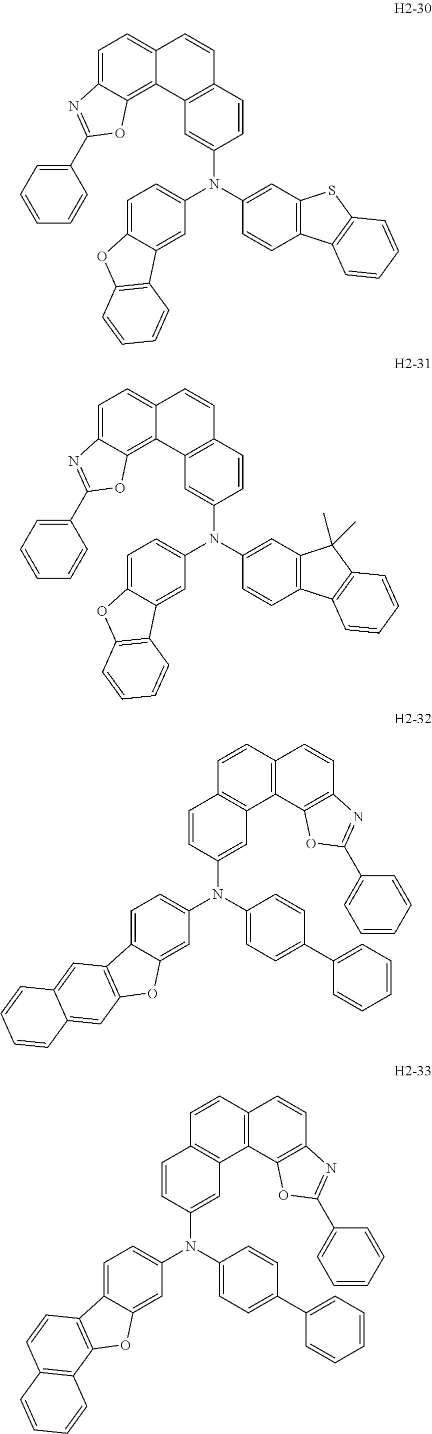

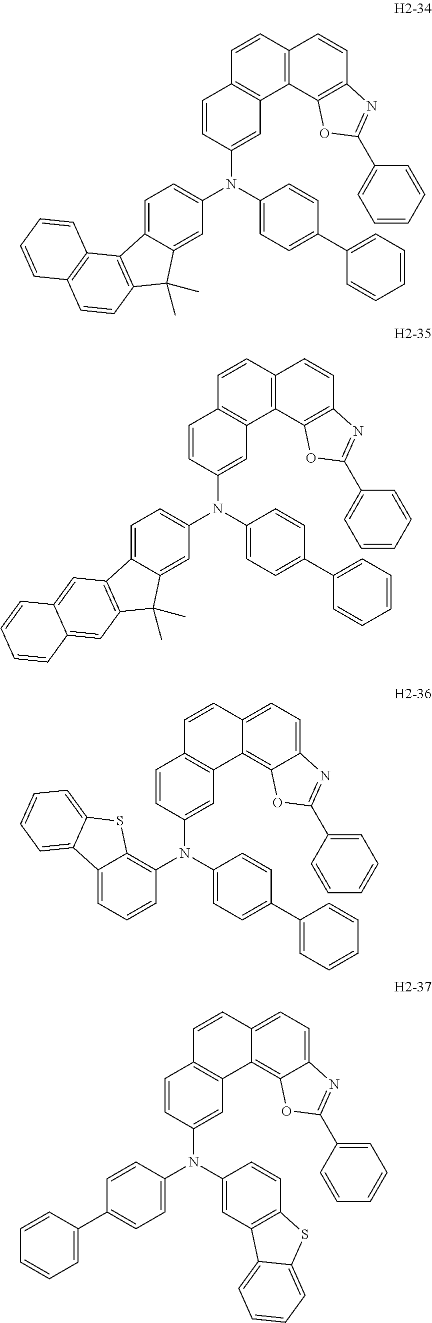 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

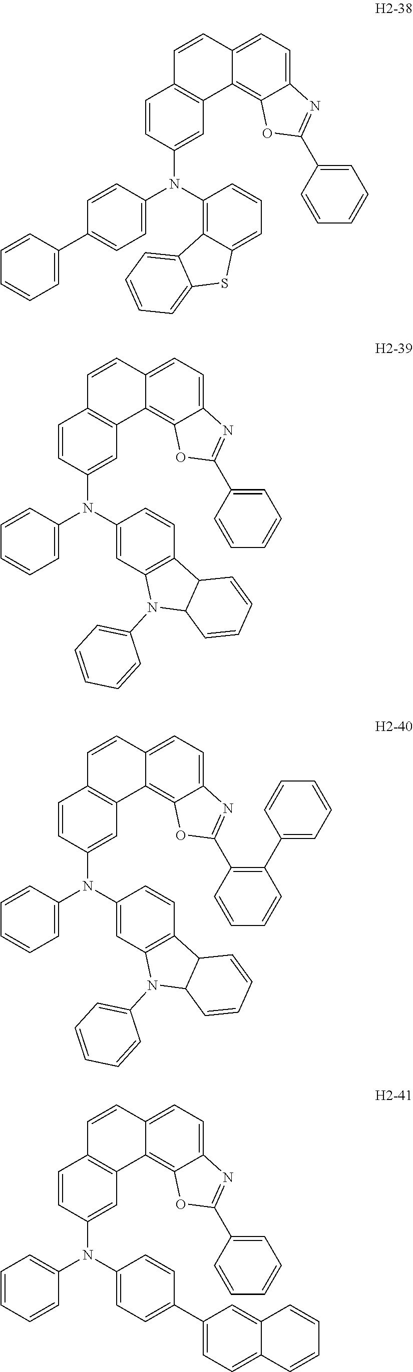

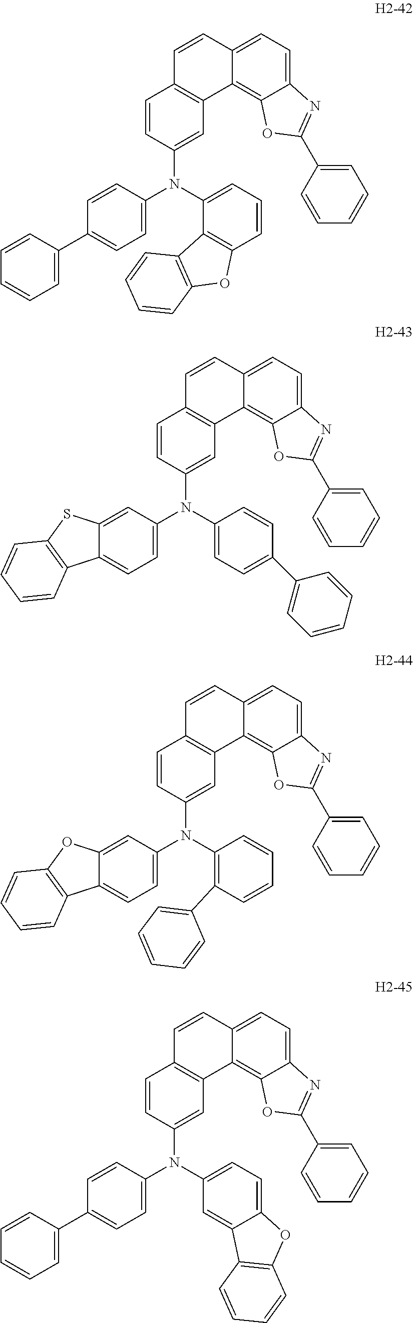 [SMILES: c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1]

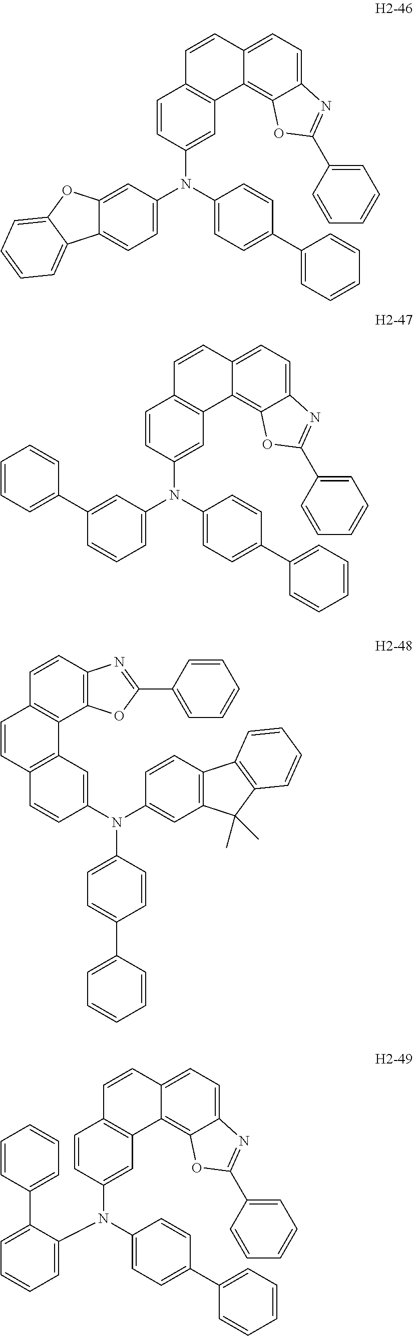 [SMILES: c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1]

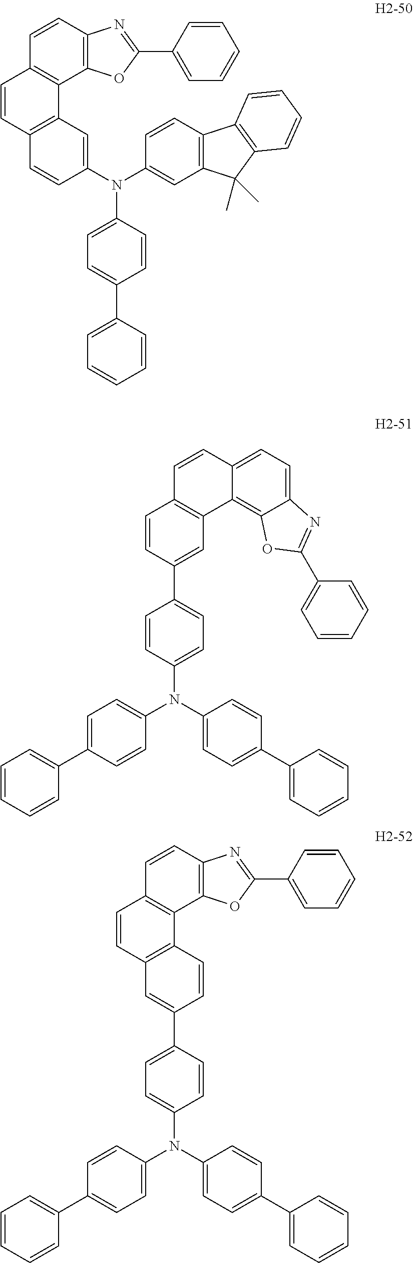

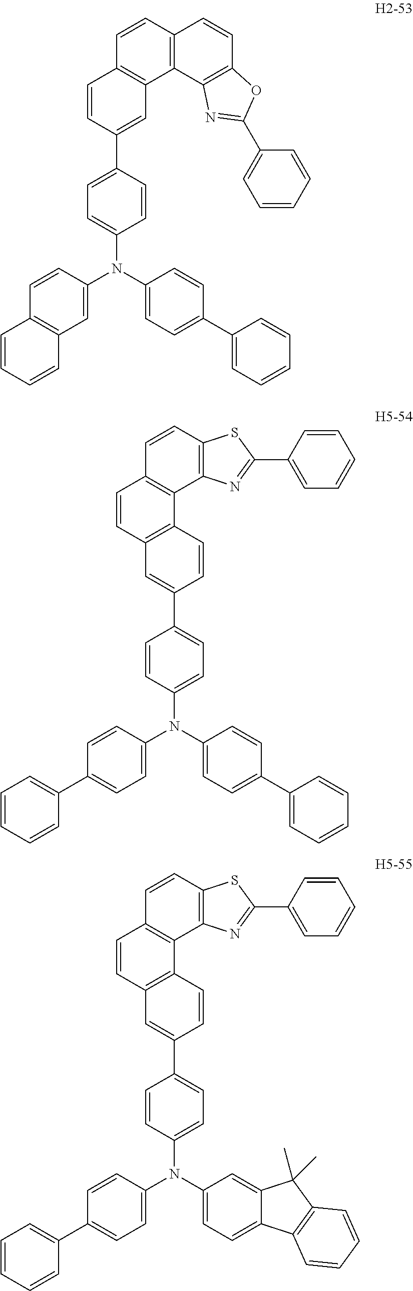

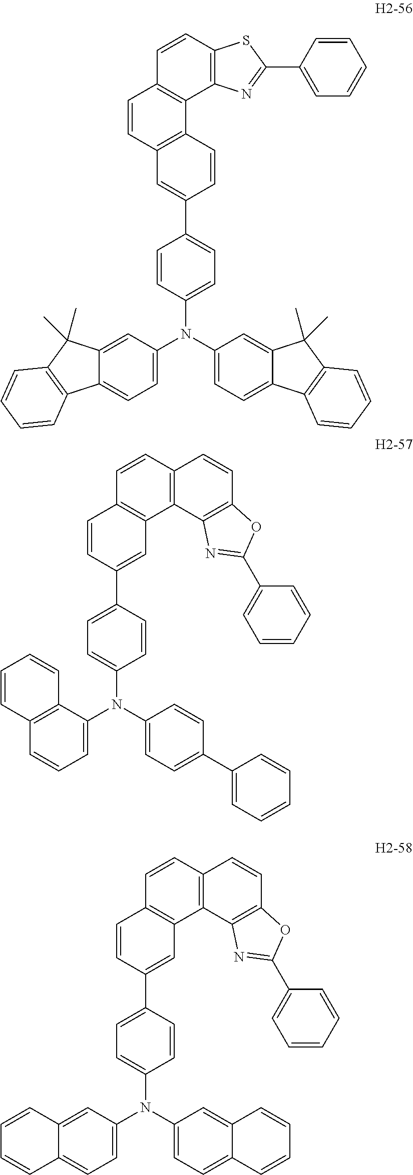 [SMILES: CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1]

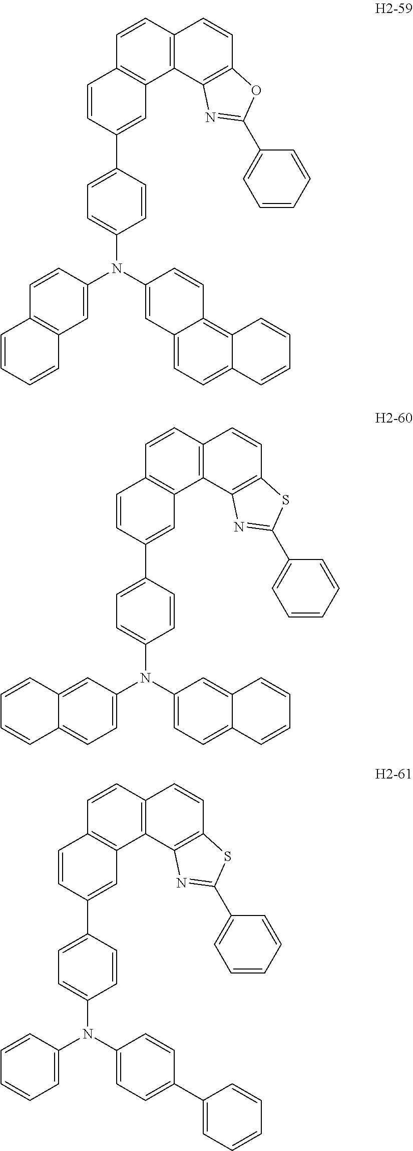 [SMILES: c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1]

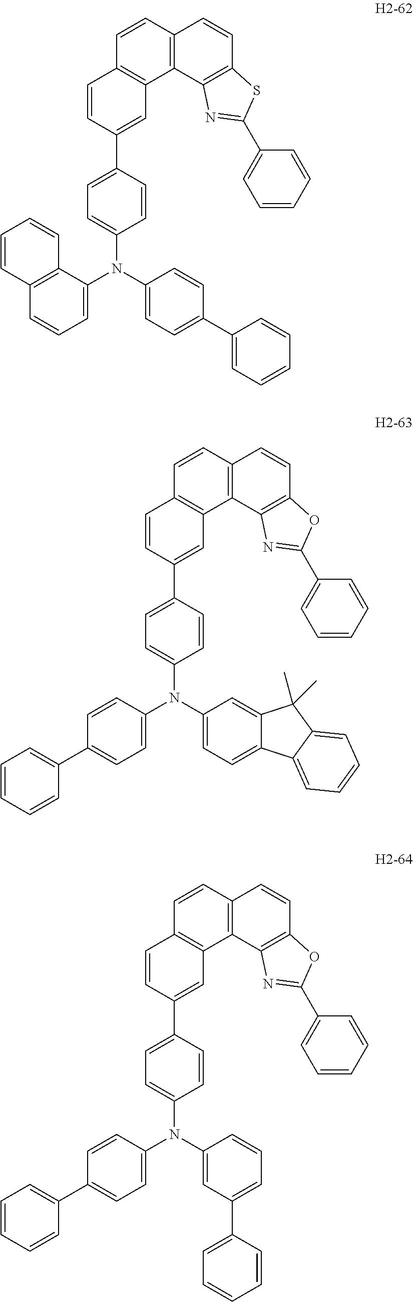 [SMILES: c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1]

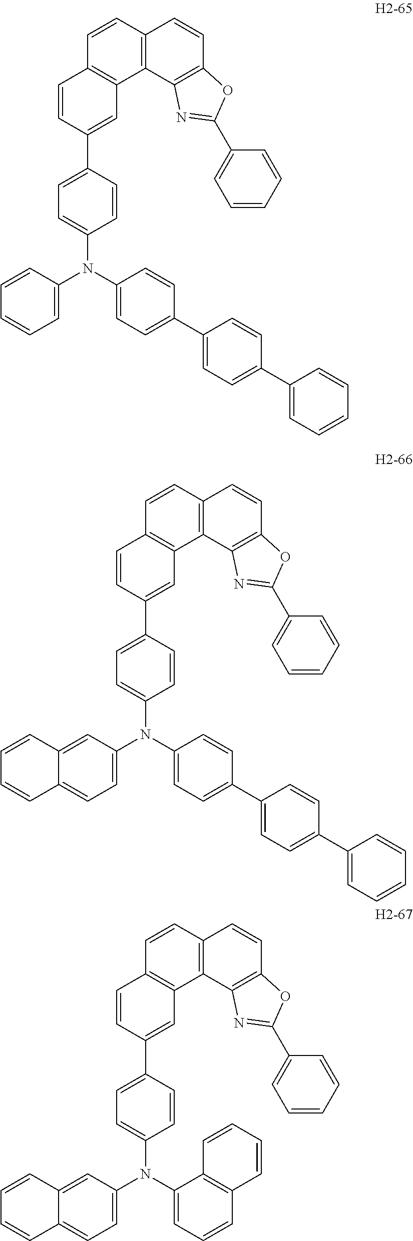 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

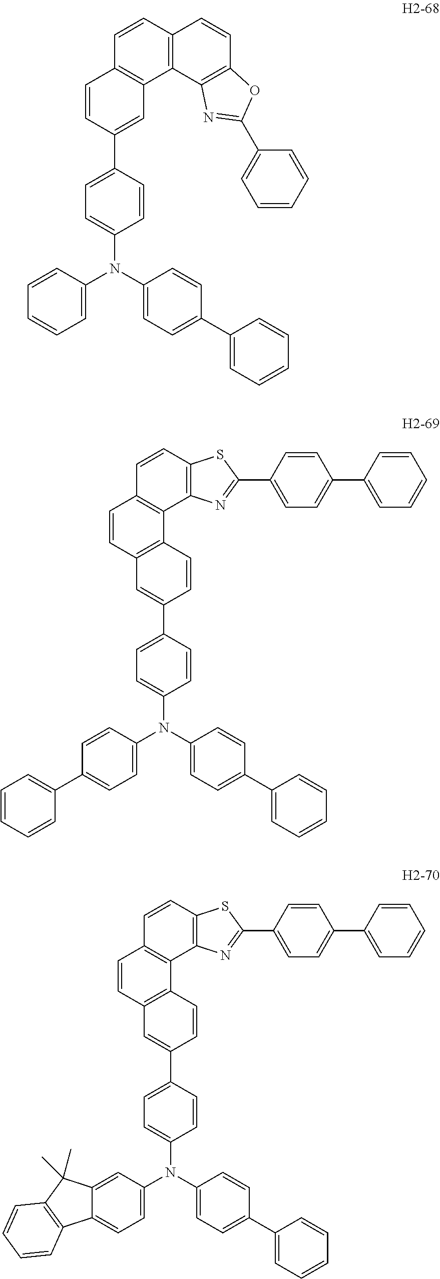 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

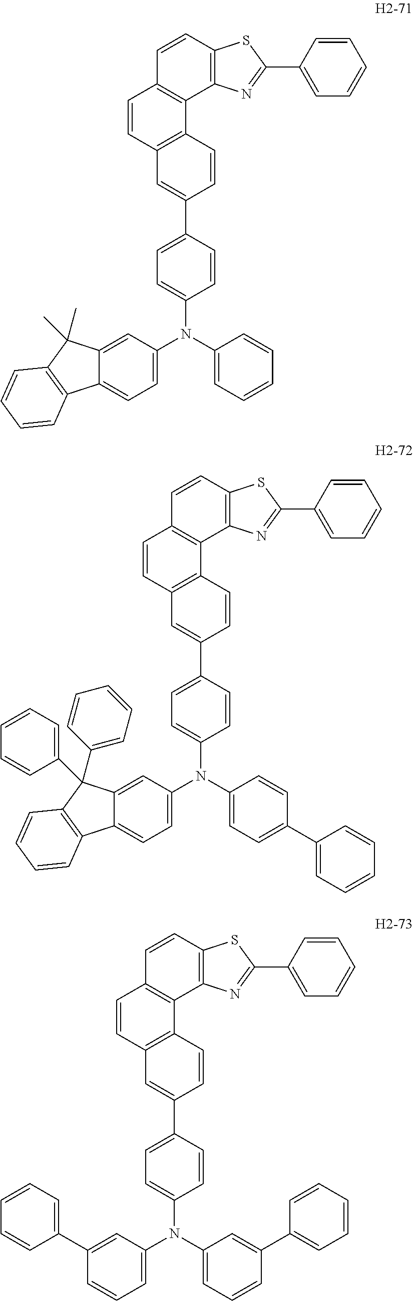

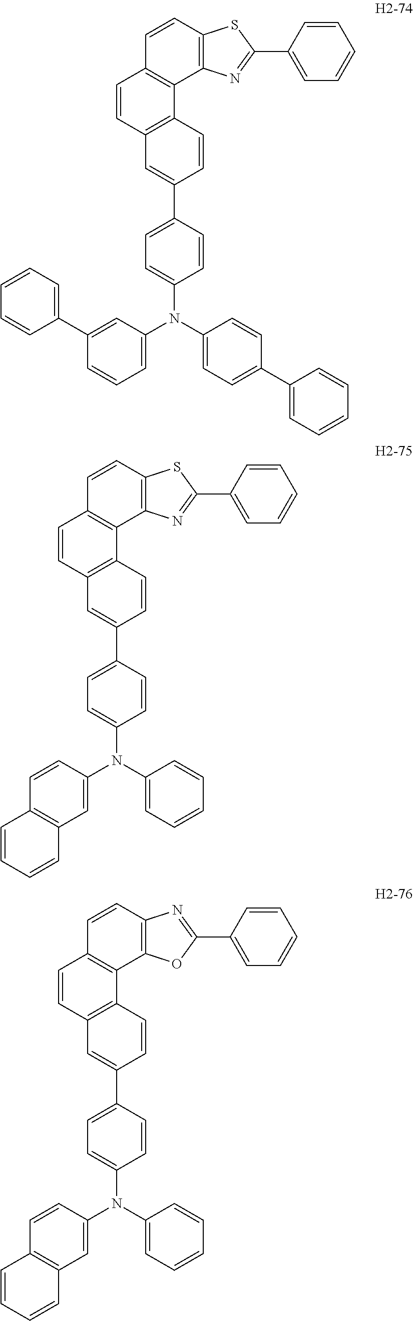 [SMILES: c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1]

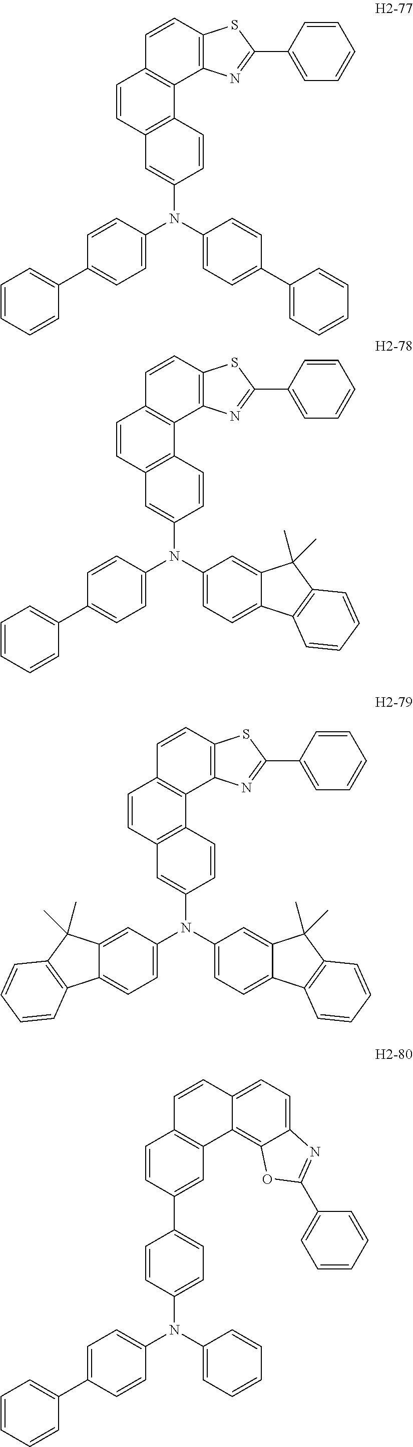

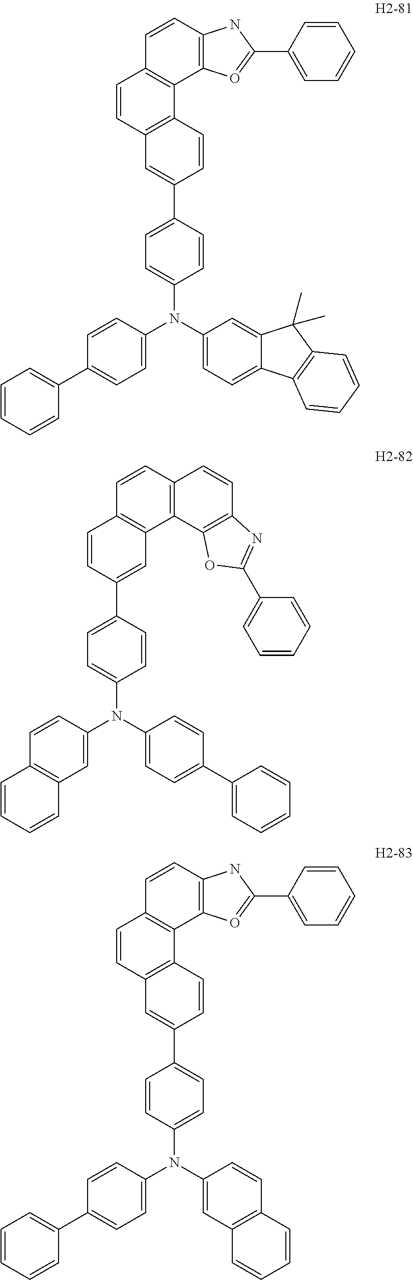

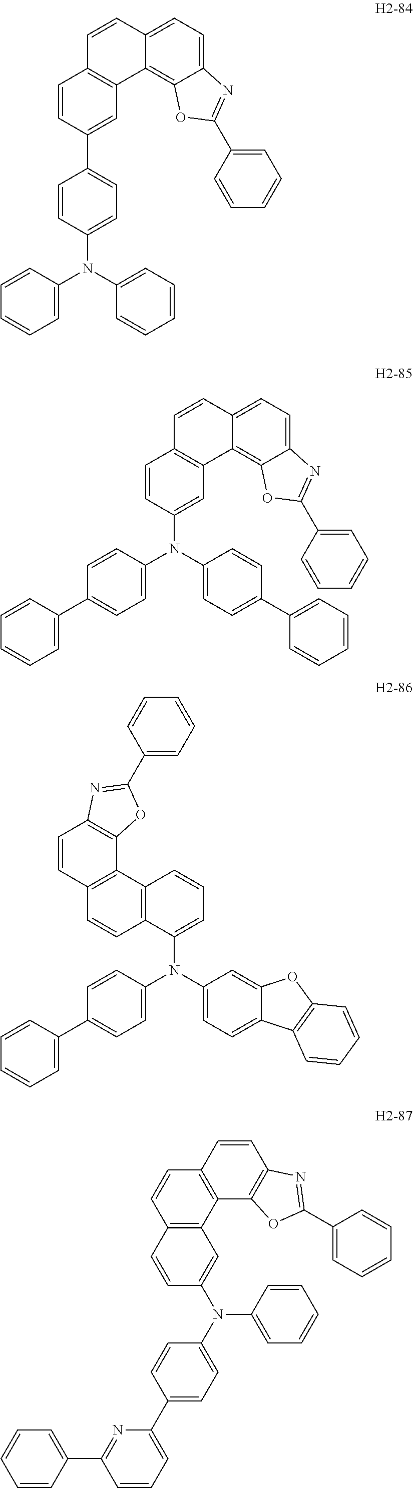 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1]

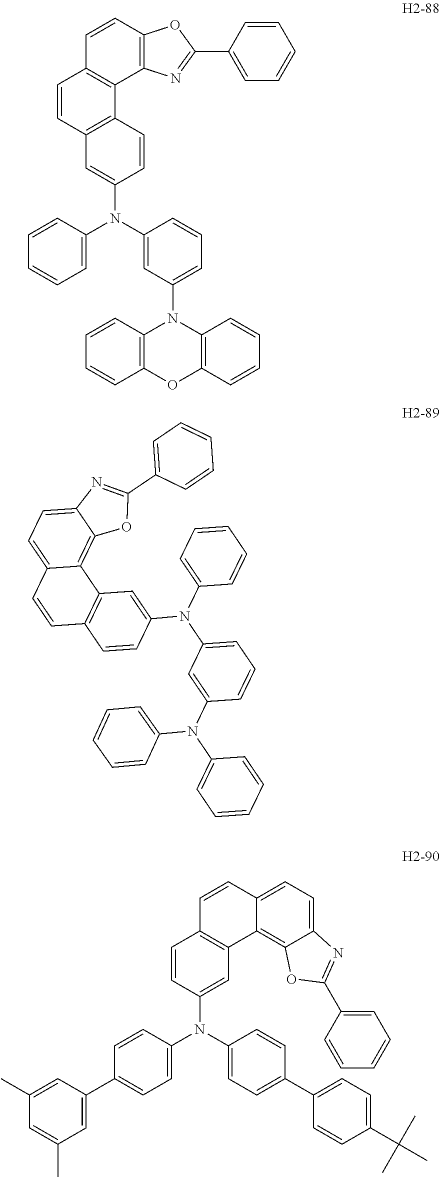

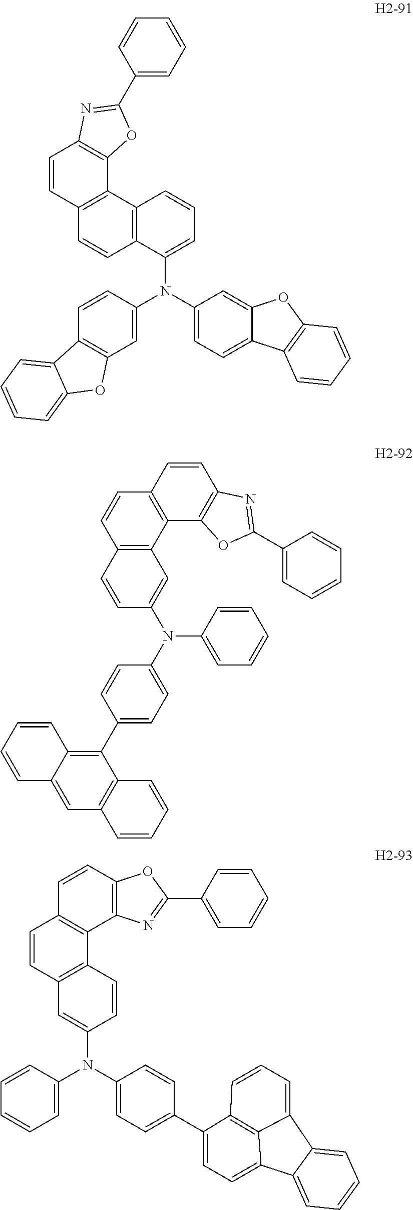

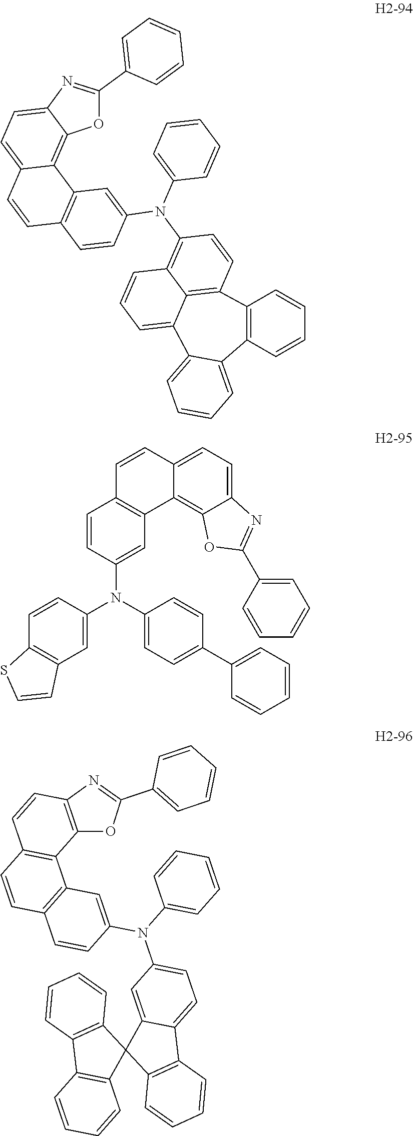

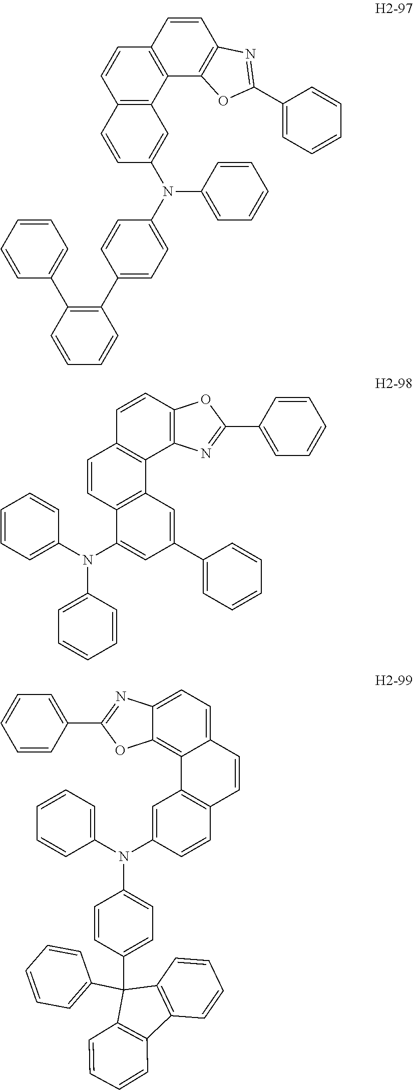 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

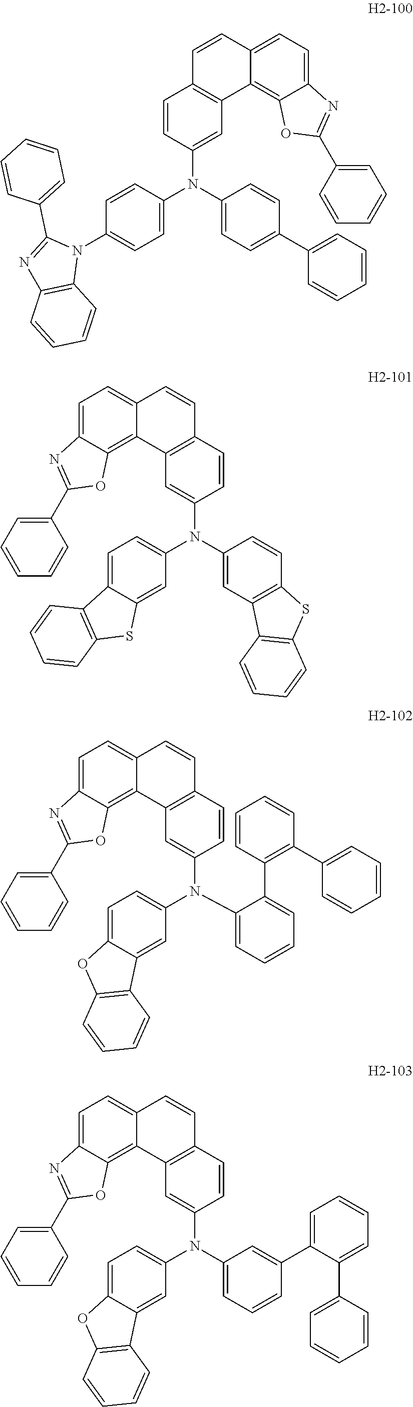

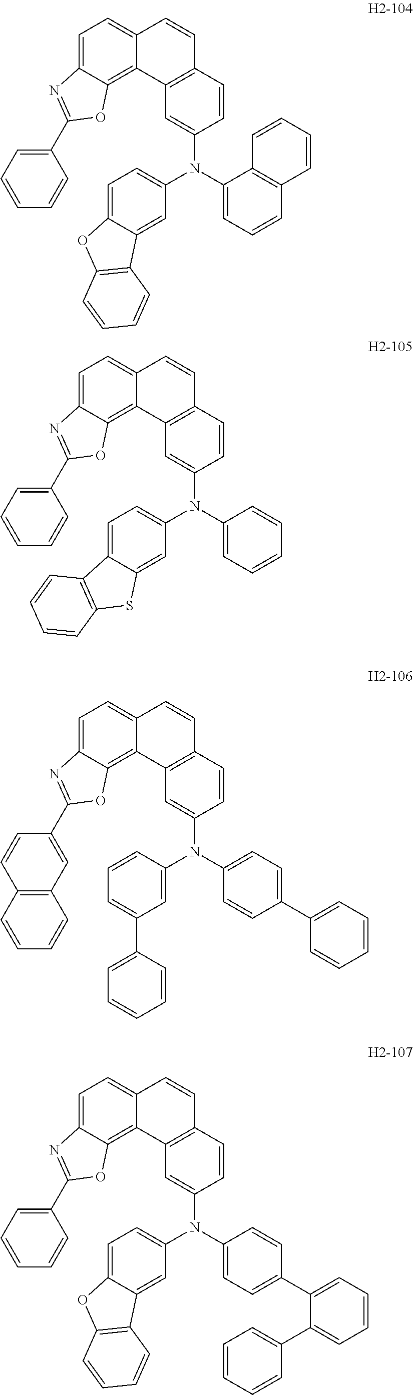

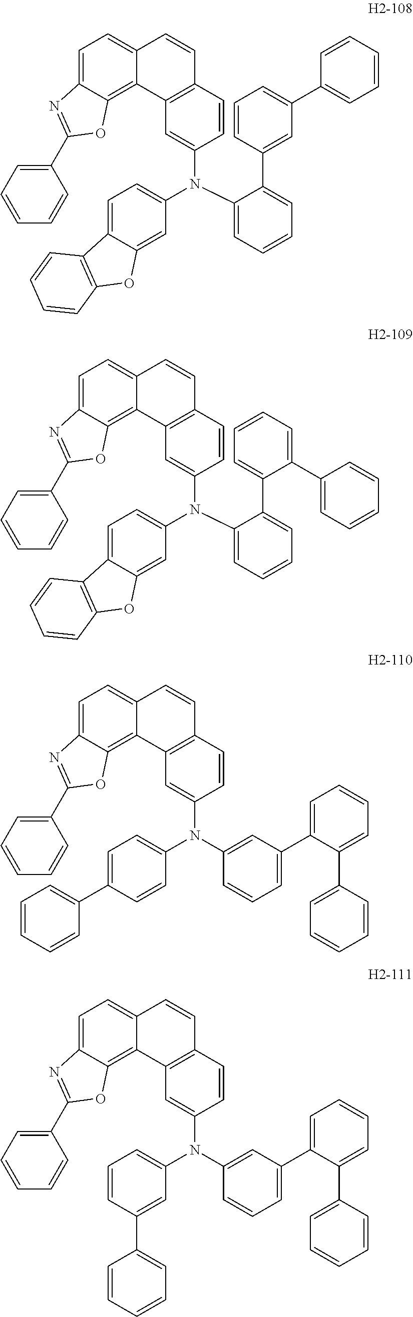 [SMILES: c1ccc(-c2cccc(-c3ccccc3N(c3ccc4c(c3)oc3ccccc34)c3ccc4ccc5ccc6nc(-c7ccccc7)oc6c5c4c3)c2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2ccc(N(c3cccc(-c4ccccc4-c4ccccc4)c3)c3ccc4ccc5ccc6nc(-c7ccccc7)oc6c5c4c3)cc2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

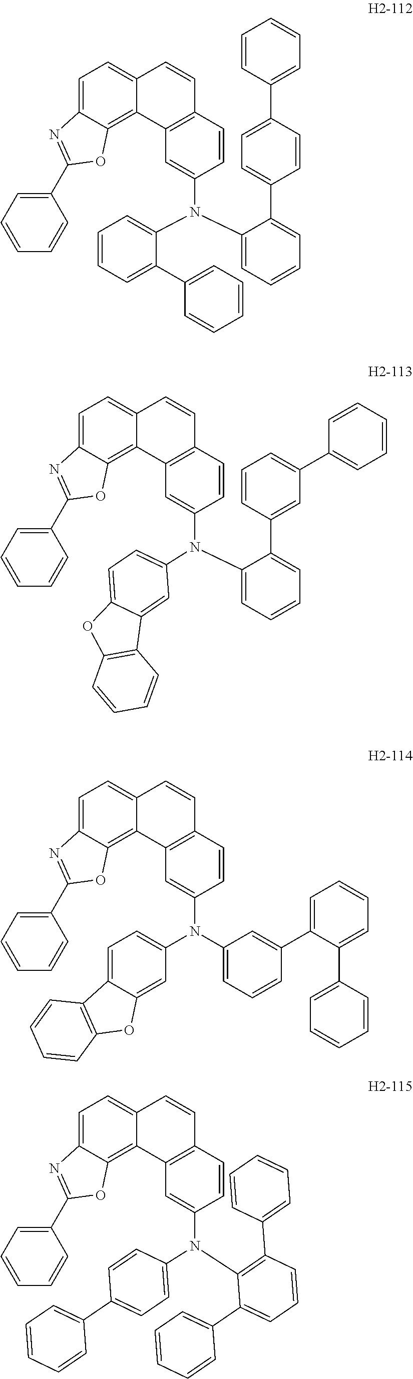

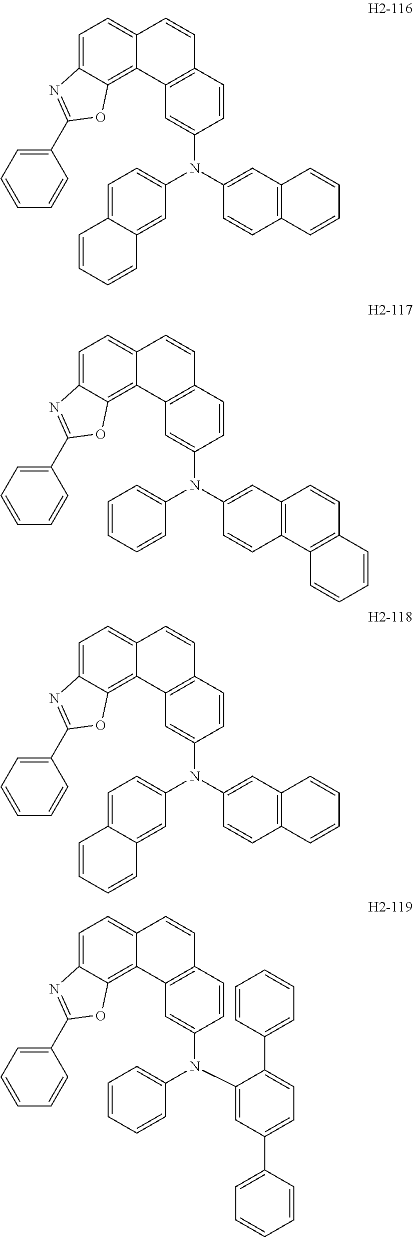

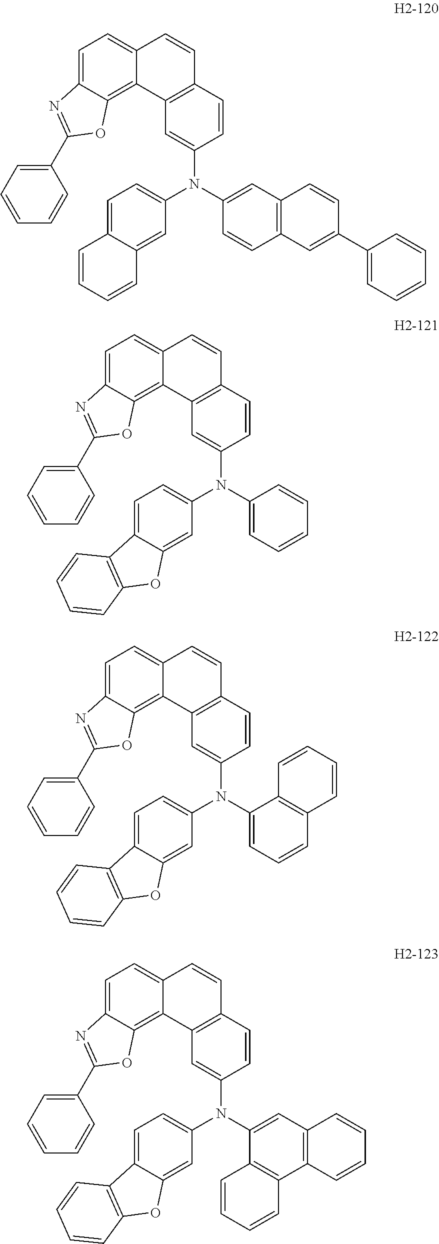

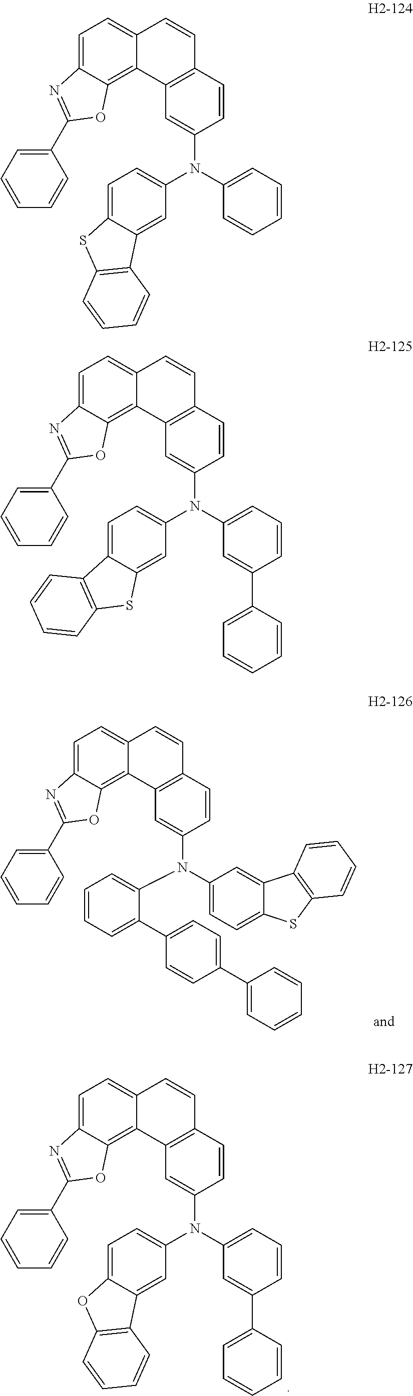

9. The plurality of host materials according to claim 6, wherein the second host material comprises an organic electroluminescent compound represented by the following formula 3: 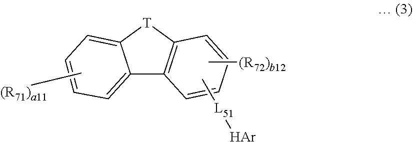
in formula 3,
T represents —O— or —S—:
L₅₁ represents a single bond, a substituted or unsubstituted (C6-C30)arylene, or a substituted or unsubstituted (3- to 30-membered)heteroarylene;
HAr represents a substituted or unsubstituted (3- to 30-membered)heteroaryl containing a nitrogen(s);
R₇₁ and R₇₂, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di- (C2-C30)alkenylamino, a substituted or unsubstituted mono- or di- (C6-C30)arylamino, a substituted or unsubstituted mono- or di- (3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, or a substituted or unsubstituted (C6-C30)aryl(3- to 30-membered)heteroarylamino; or may be linked to an adjacent substituent(s) to form a ring(s); and
a₁₁ represents an integer of 1 to 4, and b₁₂ represents an integer of 1 to 3, where if a₁₁ and b₁₂ are each an integer of 2 or more, each of R₇₁ and each of R₇₂ may be the same or different from each other.

10. The plurality of host materials according to claim 9, wherein the compound represented by formula 3 is at least one selected from the group consisting of the following compounds:

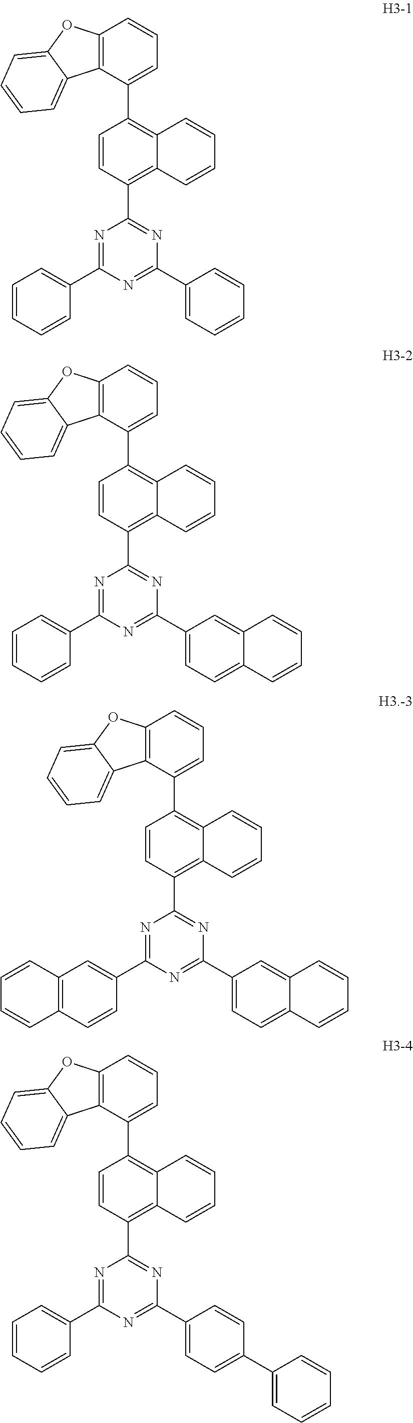

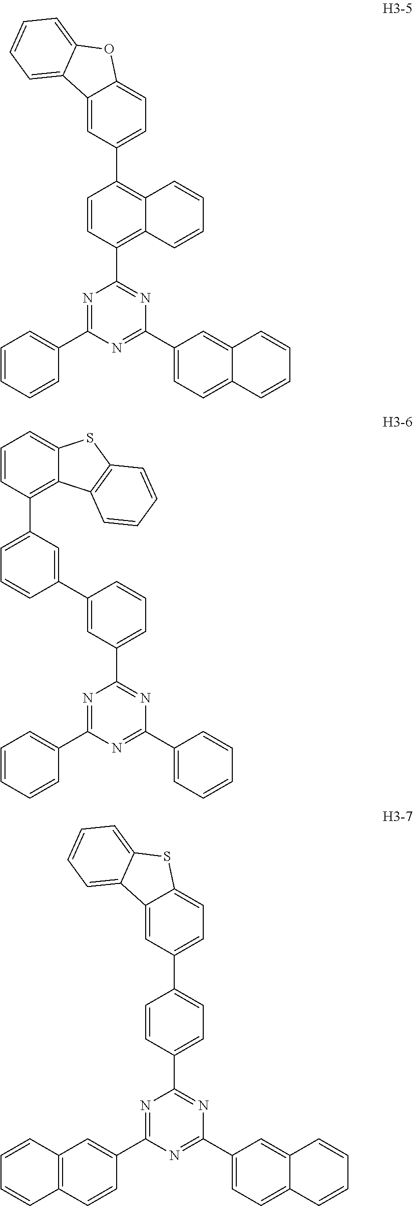

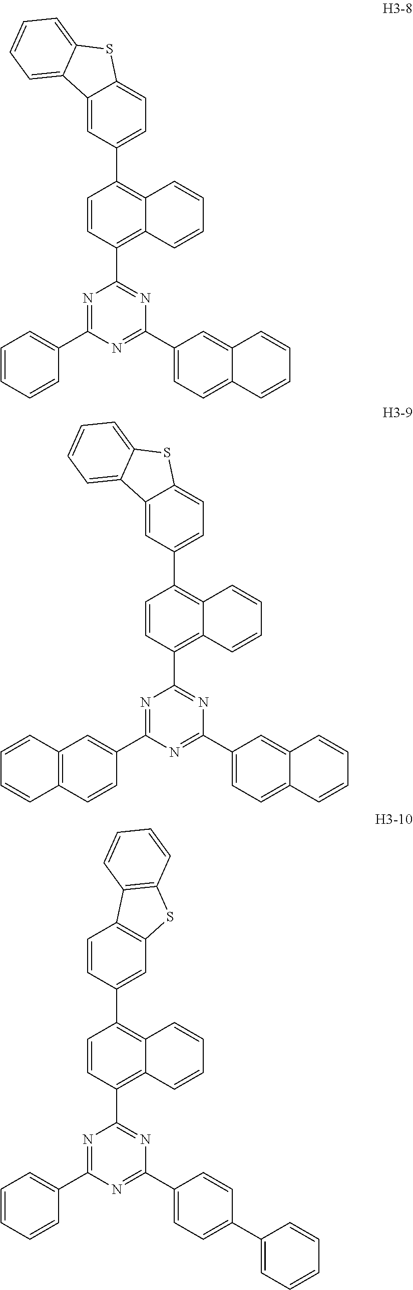

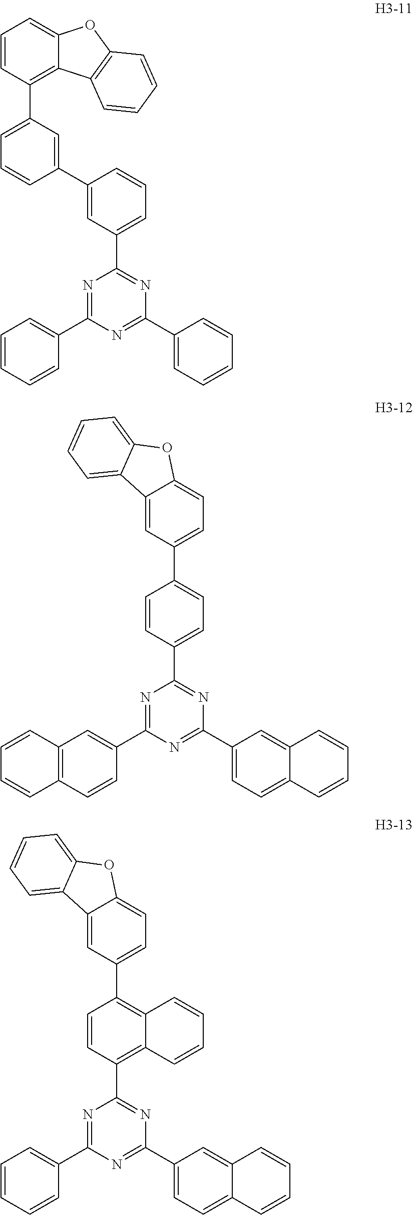

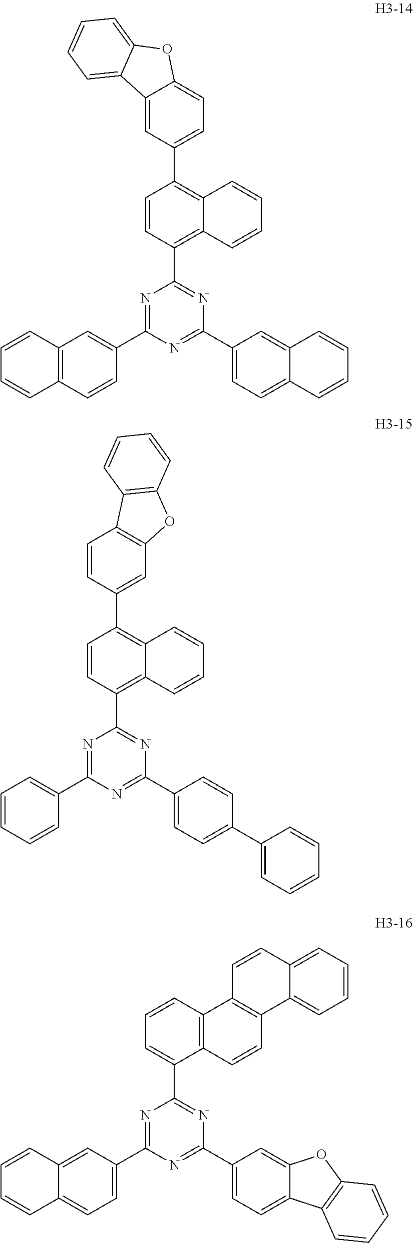 [SMILES: c1ccc2cc(-c3nc(-c4ccc5ccccc5c4)nc(-c4ccc(-c5ccc6oc7ccccc7c6c5)c5ccccc45)n3)ccc2c1 ; c1ccc(-c2ccc(-c3nc(-c4ccccc4)nc(-c4ccc(-c5ccc6c(c5)oc5ccccc56)c5ccccc45)n3)cc2)cc1 ; c1ccc2cc(-c3nc(-c4ccc5c(c4)oc4ccccc45)nc(-c4cccc5c4ccc4c6ccccc6ccc54)n3)ccc2c1]

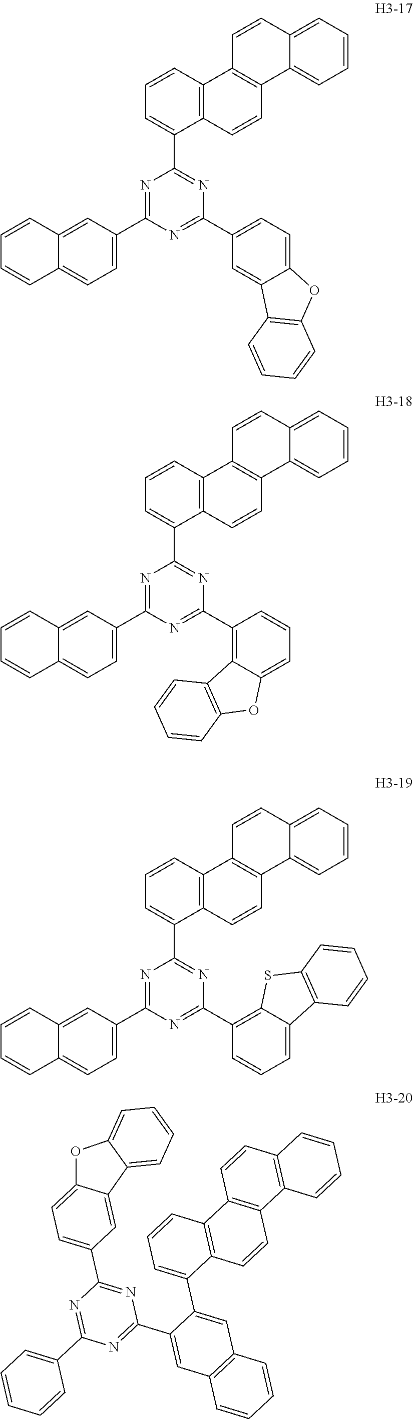

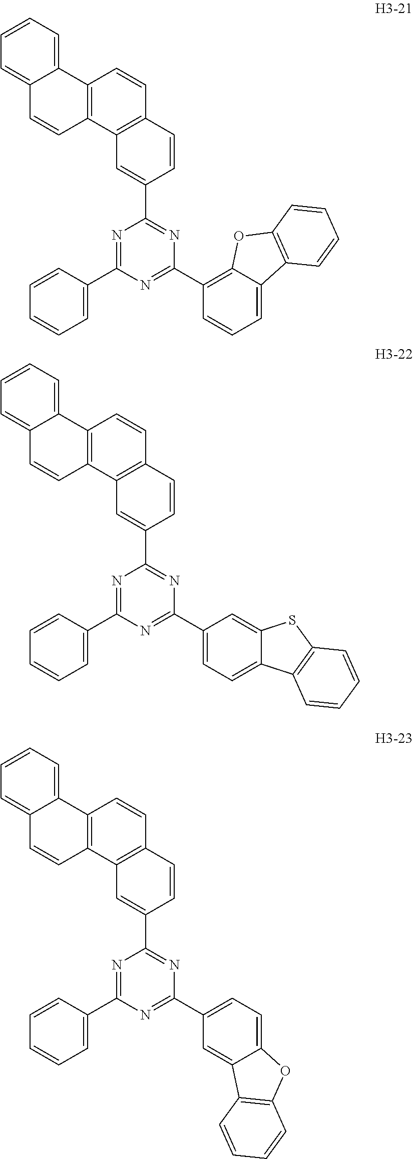

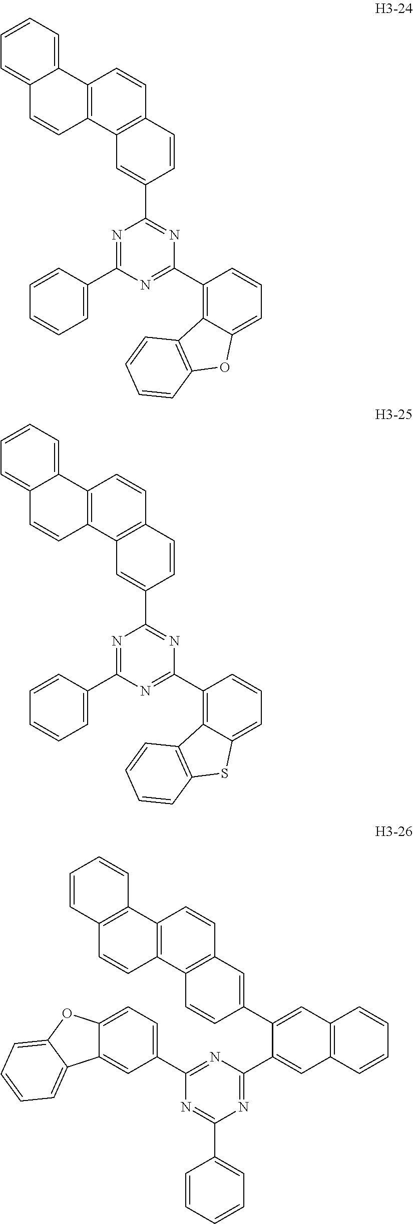 [SMILES: c1ccc(-c2nc(-c3ccc4ccc5c6ccccc6ccc5c4c3)nc(-c3cccc4oc5ccccc5c34)n2)cc1 ; c1ccc(-c2nc(-c3ccc4ccc5c6ccccc6ccc5c4c3)nc(-c3cccc4sc5ccccc5c34)n2)cc1 ; c1ccc(-c2nc(-c3ccc4oc5ccccc5c4c3)nc(-c3cc4ccccc4cc3-c3ccc4c(ccc5c6ccccc6ccc45)c3)n2)cc1]

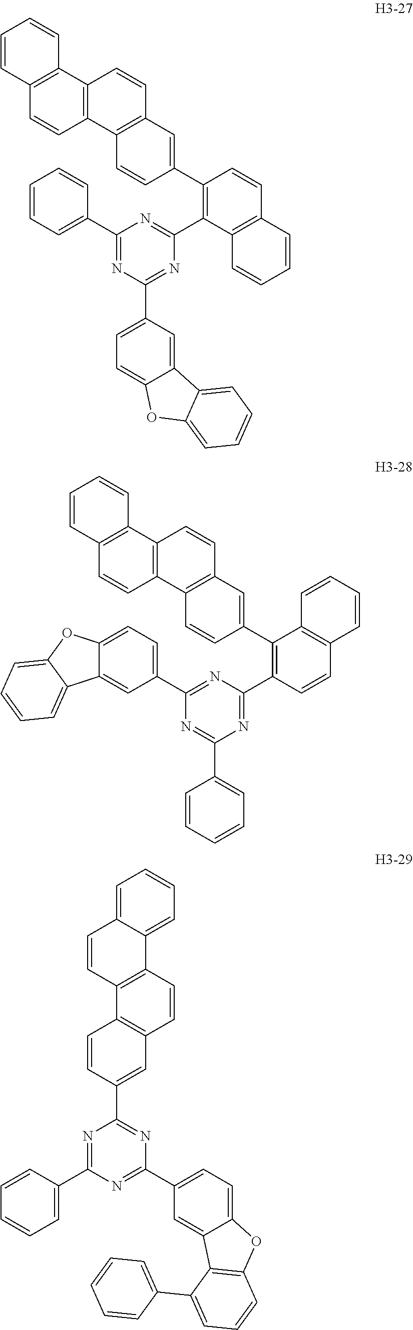 [SMILES: c1ccc(-c2nc(-c3ccc4oc5ccccc5c4c3)nc(-c3c(-c4ccc5c(ccc6c7ccccc7ccc56)c4)ccc4ccccc34)n2)cc1 ; c1ccc(-c2nc(-c3ccc4oc5ccccc5c4c3)nc(-c3ccc4ccccc4c3-c3ccc4c(ccc5c6ccccc6ccc45)c3)n2)cc1 ; c1ccc(-c2nc(-c3ccc4c(ccc5c6ccccc6ccc45)c3)nc(-c3ccc4oc5cccc(-c6ccccc6)c5c4c3)n2)cc1]

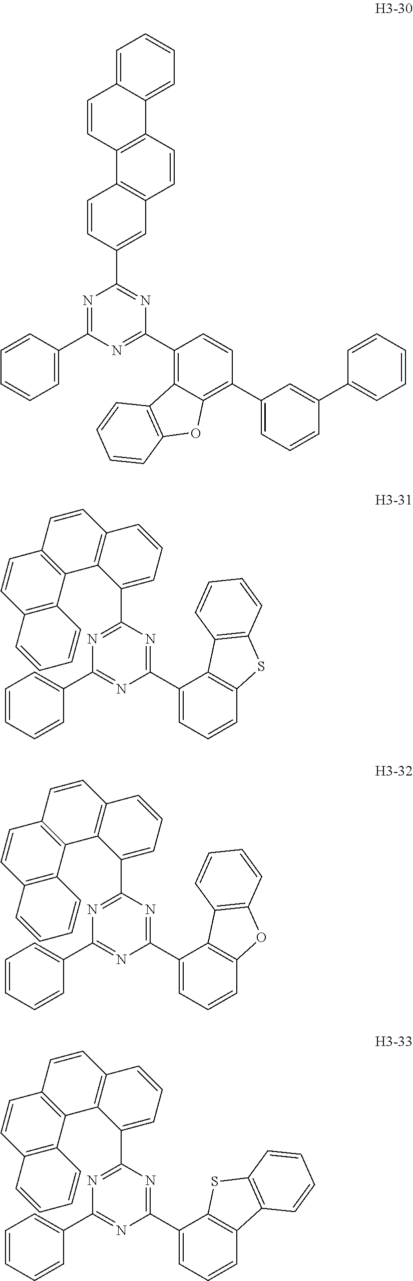 [SMILES: c1ccc(-c2cccc(-c3ccc(-c4nc(-c5ccccc5)nc(-c5ccc6c(ccc7c8ccccc8ccc67)c5)n4)c4c3oc3ccccc34)c2)cc1 ; c1ccc(-c2nc(-c3cccc4sc5ccccc5c34)nc(-c3cccc4ccc5ccc6ccccc6c5c34)n2)cc1 ; c1ccc(-c2nc(-c3cccc4oc5ccccc5c34)nc(-c3cccc4ccc5ccc6ccccc6c5c34)n2)cc1 ; c1ccc(-c2nc(-c3cccc4c3sc3ccccc34)nc(-c3cccc4ccc5ccc6ccccc6c5c34)n2)cc1]

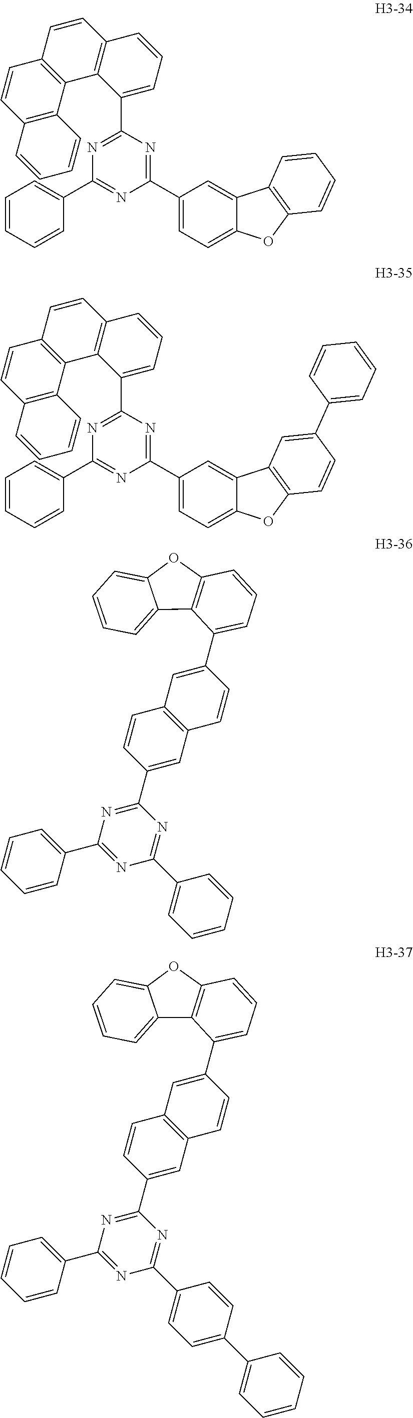

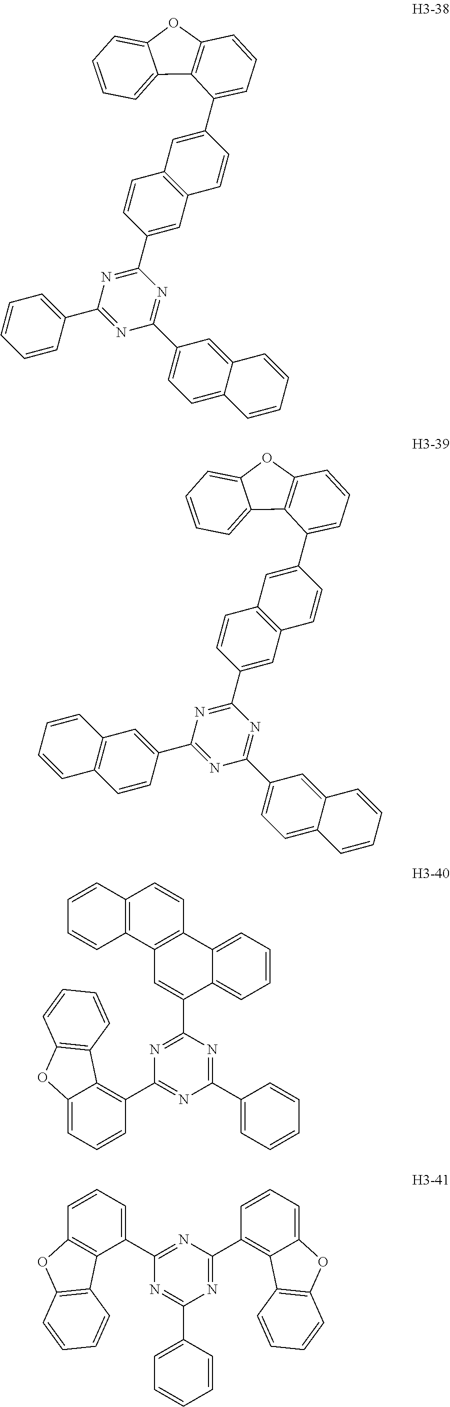

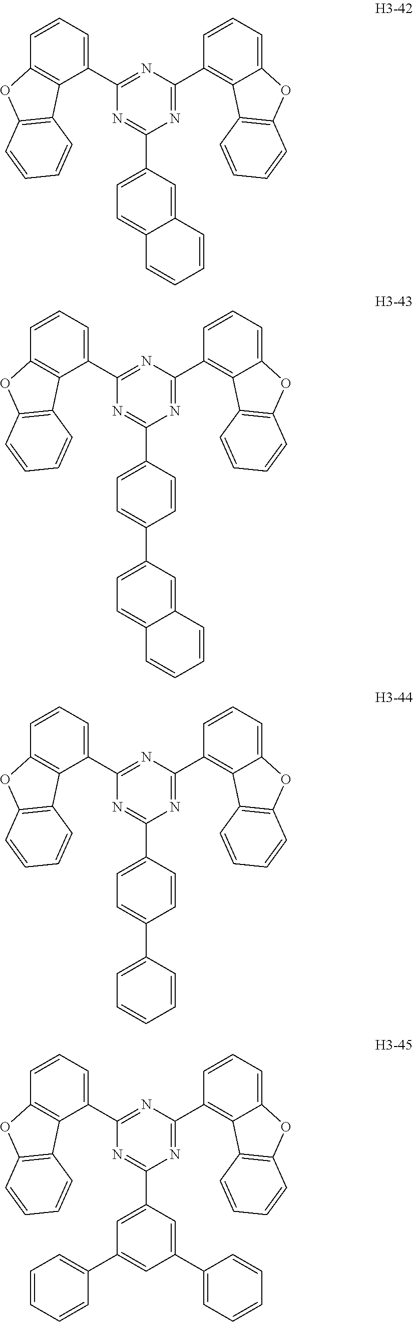 [SMILES: c1ccc2cc(-c3nc(-c4cccc5oc6ccccc6c45)nc(-c4cccc5oc6ccccc6c45)n3)ccc2c1 ; c1ccc2cc(-c3ccc(-c4nc(-c5cccc6oc7ccccc7c56)nc(-c5cccc6oc7ccccc7c56)n4)cc3)ccc2c1 ; c1ccc(-c2ccc(-c3nc(-c4cccc5oc6ccccc6c45)nc(-c4cccc5oc6ccccc6c45)n3)cc2)cc1 ; c1ccc(-c2cc(-c3ccccc3)cc(-c3nc(-c4cccc5oc6ccccc6c45)nc(-c4cccc5oc6ccccc6c45)n3)c2)cc1]

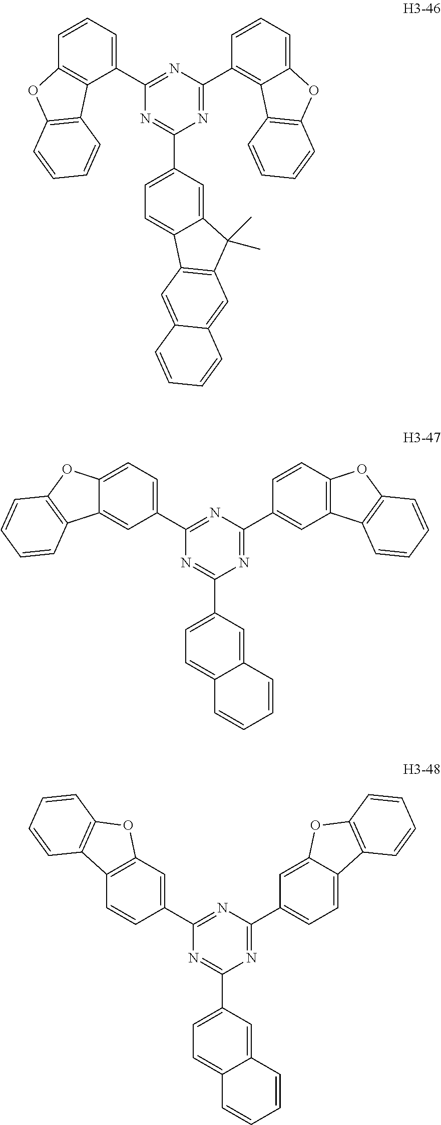 [SMILES: CC1(C)c2cc(-c3nc(-c4cccc5oc6ccccc6c45)nc(-c4cccc5oc6ccccc6c45)n3)ccc2-c2cc3ccccc3cc21 ; c1ccc2cc(-c3nc(-c4ccc5oc6ccccc6c5c4)nc(-c4ccc5oc6ccccc6c5c4)n3)ccc2c1 ; c1ccc2cc(-c3nc(-c4ccc5c(c4)oc4ccccc45)nc(-c4ccc5c(c4)oc4ccccc45)n3)ccc2c1]

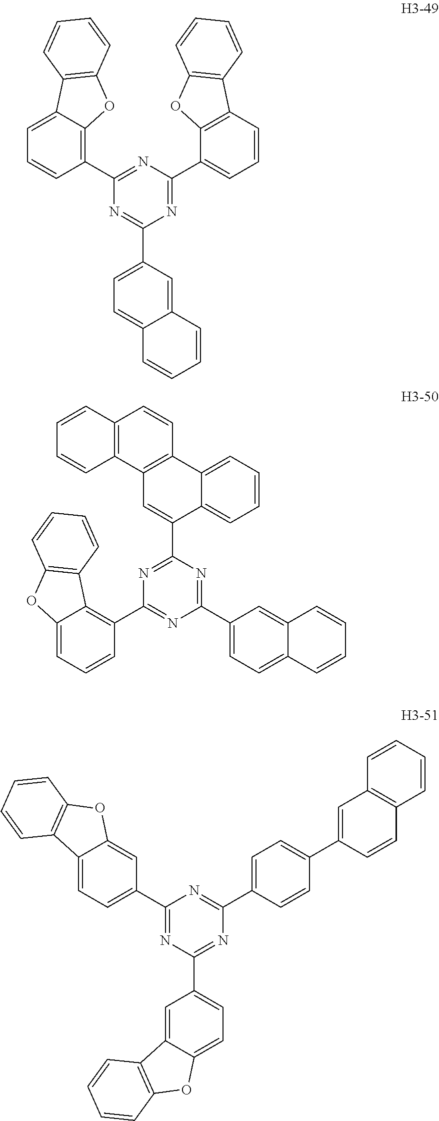

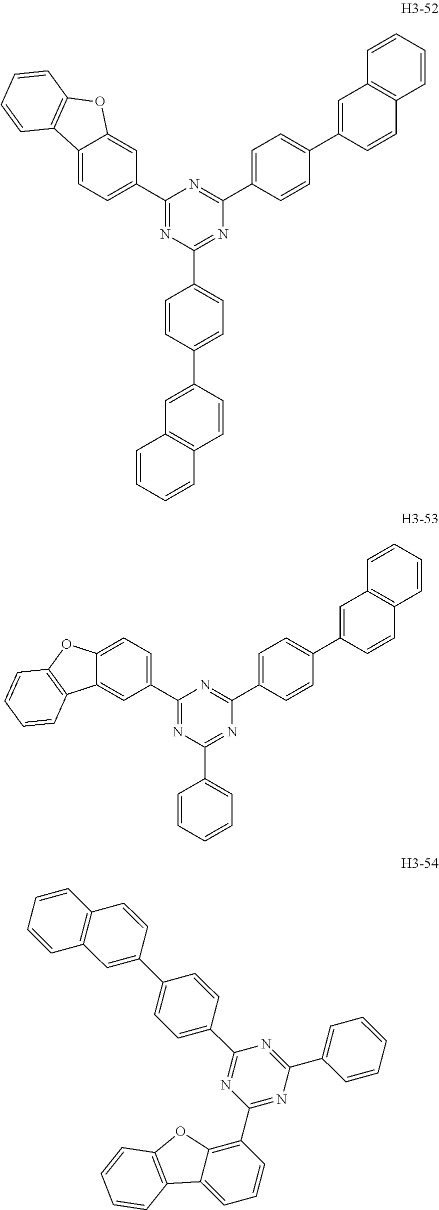 [SMILES: c1ccc2cc(-c3ccc(-c4nc(-c5ccc(-c6ccc7ccccc7c6)cc5)nc(-c5ccc6c(c5)oc5ccccc56)n4)cc3)ccc2c1 ; c1ccc(-c2nc(-c3ccc(-c4ccc5ccccc5c4)cc3)nc(-c3ccc4oc5ccccc5c4c3)n2)cc1 ; c1ccc(-c2nc(-c3ccc(-c4ccc5ccccc5c4)cc3)nc(-c3cccc4c3oc3ccccc34)n2)cc1]

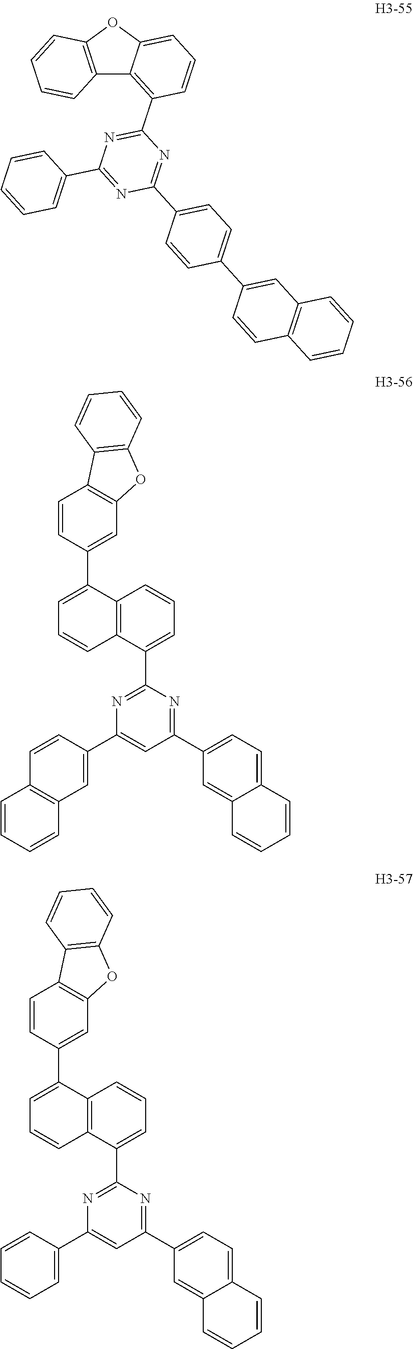 [SMILES: c1ccc(-c2nc(-c3ccc(-c4ccc5ccccc5c4)cc3)nc(-c3cccc4oc5ccccc5c34)n2)cc1 ; c1ccc2cc(-c3cc(-c4ccc5ccccc5c4)nc(-c4cccc5c(-c6ccc7c(c6)oc6ccccc67)cccc45)n3)ccc2c1 ; c1ccc(-c2cc(-c3ccc4ccccc4c3)nc(-c3cccc4c(-c5ccc6c(c5)oc5ccccc56)cccc34)n2)cc1]

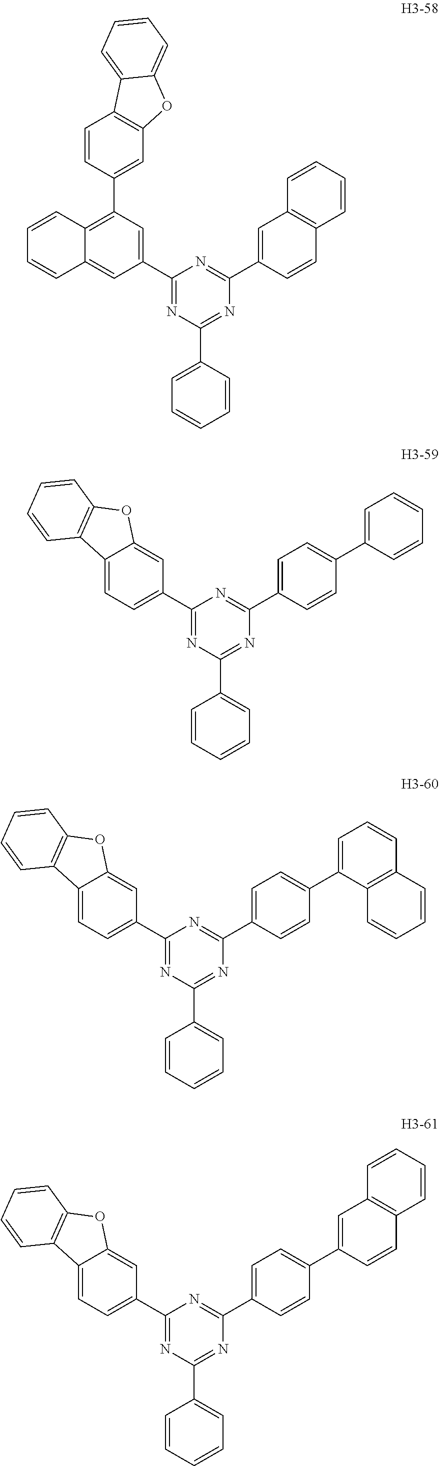 [SMILES: c1ccc(-c2nc(-c3ccc4ccccc4c3)nc(-c3cc(-c4ccc5c(c4)oc4ccccc45)c4ccccc4c3)n2)cc1 ; c1ccc(-c2ccc(-c3nc(-c4ccccc4)nc(-c4ccc5c(c4)oc4ccccc45)n3)cc2)cc1 ; c1ccc(-c2nc(-c3ccc(-c4cccc5ccccc45)cc3)nc(-c3ccc4c(c3)oc3ccccc34)n2)cc1 ; c1ccc(-c2nc(-c3ccc(-c4ccc5ccccc5c4)cc3)nc(-c3ccc4c(c3)oc3ccccc34)n2)cc1]

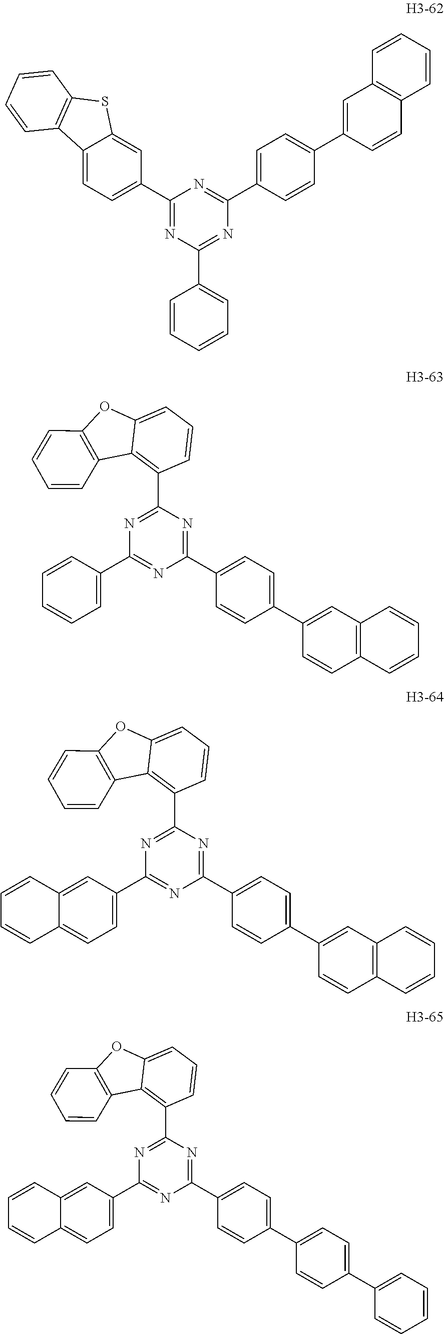 [SMILES: c1ccc(-c2nc(-c3ccc(-c4ccc5ccccc5c4)cc3)nc(-c3ccc4c(c3)sc3ccccc34)n2)cc1 ; c1ccc(-c2nc(-c3ccc(-c4ccc5ccccc5c4)cc3)nc(-c3cccc4oc5ccccc5c34)n2)cc1 ; c1ccc2cc(-c3ccc(-c4nc(-c5ccc6ccccc6c5)nc(-c5cccc6oc7ccccc7c56)n4)cc3)ccc2c1 ; c1ccc(-c2ccc(-c3ccc(-c4nc(-c5ccc6ccccc6c5)nc(-c5cccc6oc7ccccc7c56)n4)cc3)cc2)cc1]

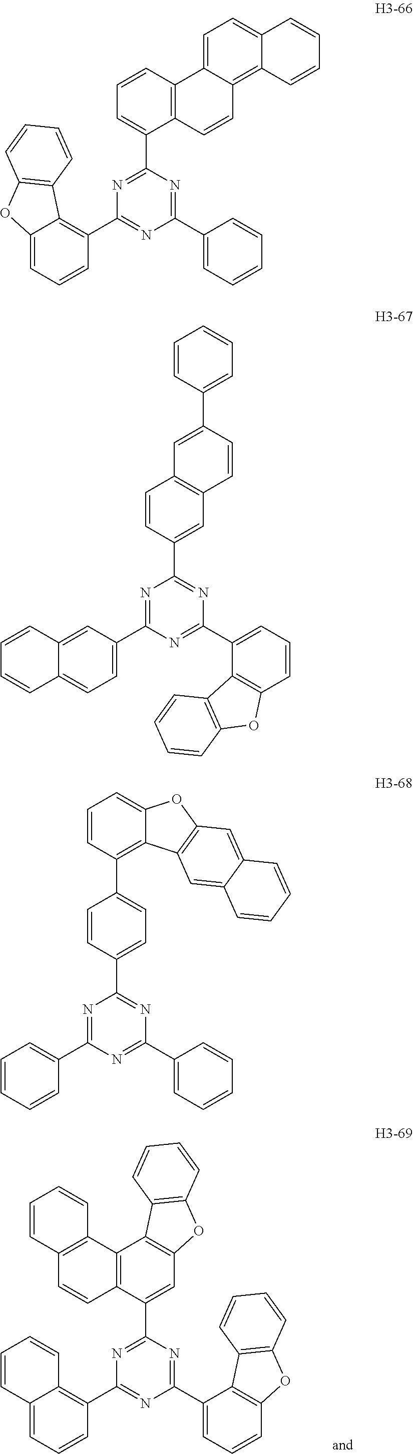

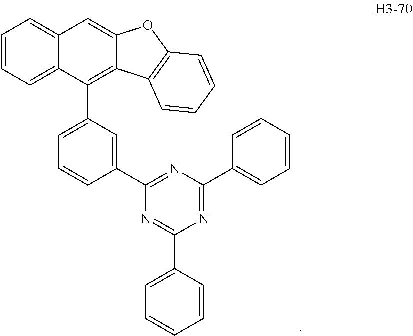 [SMILES: c1ccc(-c2nc(-c3ccccc3)nc(-c3cccc(-c4c5ccccc5cc5oc6ccccc6c45)c3)n2)cc1]

11. An organic electroluminescent device comprising an anode, a cathode, and at least one light-emitting layer between the anode and the cathode, wherein at least one of the light-emitting layers comprises the plurality of host materials according to claim 6.
